(19) United States
(12) Patent Application Publication
(10) Pub. No.: US 2023/0265449 A1
(43) Pub. Date: Aug. 24, 2023

(54) CANNABIS WITH ALTERED CANNABINOID CONTENT

(71) Applicant: BETTERSEEDS LTD, Givat Chen (IL)

(72) Inventors: Tal SHERMAN, Rehovot (IL); Ido MARGALIT, Gan Yavne (IL); Shira COREM, Sdei Hemed (IL)

(73) Assignee: BETTERSEEDS LTD, Givat Chen (IL)

(21) Appl. No.: 18/005,453

(22) PCT Filed: Jul. 11, 2021
(86) PCT No.: PCT/IL2021/050847

§ 371 Date: Jan. 13, 2023

Related U.S. Application Data
(60) Provisional application No. 62/705,719, filed on Jul. 13, 2020.

(51) Int. Cl. C12N 15/82; A61K 31/00; C12N 9/02; C12N 15/11; C12N 9/22

(52) U.S. Cl. CPC C12N 15/8243; A61K 31/658; C12N 9/0004; C12Y 121/03007; C12Y 121/03008; C12N 15/11; C12N 9/22; C12N 15/8213; C12N 15/111; C12N 2310/20; C12N 2800/80

(57) ABSTRACT

Provided is a Cannabis plant exhibiting altered tetrahydrocannabinolic acid (THCA) and/or cannabidiolic acid (CBDA) content. The plant includes a modified genomic locus involved in tetrahydrocannabinolic acid synthase (THCAS) and/or cannabidiolic acid synthase (CBDAS) gene expression, the genomic locus includes at least one targeted nucleotide modification within a regulatory region modulating the expression of at least one allele of the THCAS and/or CBDAS genes. Further provided are methods for producing the aforementioned Cannabis plant.

### BACKGROUND OF THE INVENTION

### Field of the Invention

The present invention generally relates to Cannabis plants with altered expression of cannabinoids and/or altered expression of cannabinoid synthesizing enzymes. More specifically, the present disclosure relates to methods for controlling genes associated with cannabinoids synthesis in Cannabis plants.

### Background Art

Cannabis has been cultivated throughout human history as a source of fiber, oil, food, and for its medicinal properties. Selective breeding has produced Cannabis plants for specific uses, including high-potency marijuana strains and hemp cultivars for fiber and seed production. The molecular biology underlying cannabinoid biosynthesis and other traits of interest is largely unexplored.

There is therefore an urgent need to develop innovative approaches to accelerate Cannabis improvement and make its outcomes more predictable. Significant obstacles in plant breeding include limited sources of genetic variation underlying quantitative traits and the time-consuming and labor-intensive phenotypic and molecular evaluation of breeding germplasm required to select plants with desirable properties.

There are over 113 known cannabinoids, but the two most abundant natural derivatives are tetrahydrocannabinol (THC) and cannabidiol (CBD). THC is responsible for the well-known psychoactive effects of Cannabis consumption, but CBD, while nonpsycoative, also has therapeutic properties, for example it is investigated as a treatment for both schizophrenia and Alzheimer's disease. Cannabis has traditionally been classified as having a drug (“marijuana”) or hemp chemotype based on the relative proportion of THC to CBD; types grown for recreational use produce relatively large amounts of both. Cannabis containing high levels of CBD is increasingly grown for medical use.

Heat converts the cannabinoid acids (e.g., tetrahydrocannabinolic acid [THCA]) to neutral molecules (e.g., (−)-trans-Δ9-tetrahydrocannabinol [THC]) that bind to endocannabinoid receptors found in the vertebrate nervous system. The cannabinoid acids THCA and CBDA are both synthesized from cannabigerolic acid (CBGA) by the related enzymes THCA synthase (THCAS) and CBDA synthase (CBDAS), respectively. Expression of THCAS and CBDAS appear to be the major factor determining cannabinoid content, but the mechanisms that underlie the expression of these enzymes remain unresolved.

As the cultivation of Cannabis intensifies, breeding and farming techniques fail to provide the level of control of cannabinoid production and yield needed.

Selective breeding has been used to control the genetics of plants and modify the cannabinoid profile. For example, strains that are used as fiber (commonly called hemp) are usually bred such that they are low in psychoactive chemicals like THC. Strains used in medicine are often bred for high CBD content, and strains used for recreational purposes are usually bred for high THC content or for a specific chemical balance.

Enhancing genetic and phenotypic variation in crops has relied on intercrossing with wild relatives to introduce allelic diversity, creating novel alleles by random mutagenesis, and genetic engineering. However, these approaches are inefficient, particularly for providing variants that cause changes in complex traits that are most desired by breeders.

A powerful approach to create novel allelic variation is through genome editing. In plants, this technology has primarily been used to engineer mutations in coding sequences, with the goal of creating null alleles for functional studies.

Compared to mutations in coding sequences that alter protein structure, cis-regulatory variants are frequently less pleiotropic and often cause subtle phenotypic change by modifying the timing, pattern, or level of gene expression. A major explanation for this is the complexity of transcriptional control, which includes redundancy and modular organization of the many cis-regulatory elements (CREs) in promoters and other regulatory regions, the majority of which remain poorly characterized.

In view of the above, there is an unmet and long felt need for an approach that allows efficient and rapid generation of novel alleles in non-GMO Cannabis plants. In particular, there is a need for manipulating Cannabis plants for selectively producing predetermined ratios and/or concentrations of cannabinoids for medical use.

### SUMMARY OF THE INVENTION

It is one object of the present invention to disclose a Cannabis plant exhibiting altered tetrahydrocannabinolic acid (THCA) and/or cannabidiolic acid (CBDA) content, wherein said plant comprises a modified genomic locus involved in tetrahydrocannabinolic acid synthase (THCAS) and/or cannabidiolic acid synthase (CBDAS) gene expression, said genomic locus comprises at least one targeted nucleotide modification within a regulatory region modulating the expression of at least one allele of said THCAS and/or CBDAS genes.

It is a further object of the present invention to disclose a Cannabis plant or a cell thereof comprising a genetically modified genomic locus involved in tetrahydrocannabinolic acid synthase (THCAS) and/or cannabidiolic acid synthase (CBDAS) gene expression, wherein said genomic locus comprises at least one targeted nucleotide modification within a regulatory region modulating the expression of at least one allele of said THCAS and/or CBDAS genes.

It is a further object of the present invention to disclose the Cannabis plant or a cell thereof as defined in any of the above, wherein said targeted nucleotide modification confers altered tetrahydrocannabinolic acid (THCA) and/or cannabidiolic acid (CBDA) content as compared to a comparable control Cannabis plant lacking said at least one targeted nucleotide modification.

It is a further object of the present invention to disclose the Cannabis plant or a cell thereof as defined in any of the above, wherein said plant comprises elevated THCA, CBDA and/or Cannabigerolic acid (CBGA) content relative to a comparable control Cannabis plant.

It is a further object of the present invention to disclose the Cannabis plant or a cell thereof as defined in any of the above, wherein said comparable control Cannabis plant is of a similar genotype and/or chemotype and/or genetic background and is lacking said at least one targeted nucleotide modification.

It is a further object of the present invention to disclose the Cannabis plant or a cell thereof as defined in any of the above, wherein said targeted nucleotide modification is introduced through a genome editing at said regulatory region of said THCAS and/or CBDAS genomic locus.

It is a further object of the present invention to disclose the Cannabis plant or a cell thereof as defined in any of the above, wherein said plant comprises an expression cassette encoding a RNA-guided endonuclease and at least one guide RNA (gRNA) having a sequence that is complementary to a target sequence within said regulatory region of said at least one THCAS and/or CBDAS allele.

It is a further object of the present invention to disclose the Cannabis plant or a cell thereof as defined in any of the above, wherein said regulatory region is a promoter region or terminator region, operably linked to the coding region of the at least one THCAS and/or CBDAS allele.

It is a further object of the present invention to disclose the Cannabis plant or a cell thereof as defined in any of the above, wherein the regulatory region is upstream of the 5′ end of the coding sequence of the THCAS and/or CBDAS allele or is downstream of the 3′ end of the coding sequence of the THCAS and/or CBDAS allele.

It is a further object of the present invention to disclose the Cannabis plant or a cell thereof as defined in any of the above, wherein the regulatory region comprises a transcription factor binding site, an RNA polymerase binding site, a TATA box, or a combination of structural variations thereof.

It is a further object of the present invention to disclose the Cannabis plant or a cell thereof as defined in any of the above, wherein said targeted nucleotide modification interrupts or interferes or down regulate or silence transcription and/or translation of the Cannabis allele sequence encoding THCAS and/or CBDAS enzyme.

It is a further object of the present invention to disclose the Cannabis plant or a cell thereof as defined in any of the above, wherein said targeted nucleotide modification enhance or induce or increase transcription and/or translation of the Cannabis allele sequence encoding THCAS and/or CBDAS enzyme.

It is a further object of the present invention to disclose the Cannabis plant or a cell thereof as defined in any of the above, wherein the THCAS allele is selected from a THCAS variant or homologue of ‘Finola’ (FN) Cannabis strain (FNTHCAS), or a THCAS variant or homologue of Purple Kush (PK) Cannabis strain (PKTHCAS).

It is a further object of the present invention to disclose the Cannabis plant or a cell thereof as defined in any of the above, wherein the CBDAS allele is selected from a cannabidiolic acid synthase-like 1 (CBDAS2), CBDAS variant or homologue of ‘Finola’ (FN) Cannabis strain (FNCBDAS), or a CBDAS variant or homologue of Purple Kush (PK) Cannabis strain (PKCBDAS).

It is a further object of the present invention to disclose the Cannabis plant or a cell thereof as defined in any of the above, wherein the FNTHCAS is selected from FNTHCAS-1 and FNTHCAS-2 alleles, and the PKTHCAS is selected from PKTHCAS-1, PKTHCAS-2, PKTHCAS-3 and PKTHCAS-4 alleles.

It is a further object of the present invention to disclose the Cannabis plant or a cell thereof as defined in any of the above, wherein the PKCBDAS is selected from PKCBDAS and PKCBDAS1 alleles.

It is a further object of the present invention to disclose the Cannabis plant or a cell thereof as defined in any of the above, wherein the regulatory region comprises a sequence of the promoter region of a THCAS allele selected from: promoter of FNTHCAS-1 (pFNTHCAS-1) having at least 70% sequence identity or similarity to a nucleic acid sequence as set forth in SEQ ID NO:1 or a functional variant thereof, promoter of FNTHCAS-2 (pFNTHCAS-2) having at least 70% sequence identity or similarity to a nucleic acid sequence as set forth in SEQ ID NO:2 or a functional variant thereof, promoter of PKTHCAS-1 (pPKTHCAS-1) having at least 70% sequence identity or similarity to a nucleic acid sequence as set forth in SEQ ID NO:3 or a functional variant thereof, promoter of PKTHCAS-2 (pPKTHCAS-2) having at least 70% sequence identity or similarity to a nucleic acid sequence as set forth in SEQ ID NO:4 or a functional variant thereof, promoter of PKTHCAS-3 (pPKTHCAS-3) having at least 70% sequence identity or similarity to a nucleic acid sequence as set forth in SEQ ID NO:5 or a functional variant thereof, promoter of PKTHCAS-4 (pPKTHCAS-4) having at least 70% sequence identity or similarity to a nucleic acid sequence as set forth in SEQ ID NO:6 or a functional variant thereof, a promoter sequence of any other THCAS allele, and any combination thereof.

It is a further object of the present invention to disclose the Cannabis plant or a cell thereof as defined in any of the above, wherein the regulatory region comprises a sequence of the promoter region of a CBDAS allele selected from: promoter of CBDAS2 (pCBDAS2) having at least 70% sequence identity or similarity to a nucleic acid sequence as set forth in SEQ ID NO:1150 or a functional variant thereof, promoter of FNCBDAS (pFNCBDAS) having at least 70% sequence identity or similarity to a nucleic acid sequence as set forth in SEQ ID NO:1151 or a functional variant thereof, promoter of PKCBDAS (pPKCBDAS) having at least 70% sequence identity or similarity to a nucleic acid sequence as set forth in SEQ ID NO:1152 or a functional variant thereof, promoter of PKCBDAS1 (pPKCBDAS1) having at least 70% sequence identity or similarity to a nucleic acid sequence as set forth in SEQ ID NO:1153 or a functional variant thereof, a promoter sequence of any other CBDAS allele, and any combination thereof.

It is a further object of the present invention to disclose the Cannabis plant or a cell thereof as defined in any of the above, wherein the targeted nucleotide modification is induced by a gRNA sequence comprising at least 70% sequence identity or similarity to a target sequence within said regulatory region sequence or a codon degenerate nucleotide sequence thereof.

It is a further object of the present invention to disclose the Cannabis plant or a cell thereof as defined in any of the above, wherein the guide RNA (gRNA) nucleotide sequence is selected from the group consisting of: (a) a gRNA targeting pFNTHCAS-1 and comprising a nucleotide sequence that is at least 80% identical to a nucleotide sequence selected from the group consisting of SEQ ID NO: 7-91 and any combination thereof; (b) a gRNA targeting pFNTHCAS-2 and comprising a nucleotide sequence that is at least 80% identical to the nucleotide sequence selected from the group consisting of SEQ ID NO.: 92-176 and any combination thereof; (c) a gRNA targeting pPKTHCAS-1 and comprising a nucleotide sequence that is at least 80% identical to the nucleotide sequence selected from the group consisting of SEQ ID NO.: 177-278 and any combination thereof; (d) a gRNA targeting pPKTHCAS-2 and comprising a nucleotide sequence that is at least 80% identical to the nucleotide sequence selected from the group consisting of SEQ ID NO.: 279-419 and any combination thereof; (e) a gRNA targeting pPKTHCAS-3 and comprising a nucleotide sequence that is at least 80% identical to the nucleotide sequence selected from the group consisting of SEQ ID NO.: 420-560 and any combination thereof; (f) a gRNA targeting pPKTHCAS-4 and comprising a nucleotide sequence that is at least 80% identical to the nucleotide sequence selected from the group consisting of SEQ ID NO.: 561-681 and any combination thereof; (g) a gRNA targeting pCBDAS2 and comprising a nucleotide sequence that is at least 80% identical to the nucleotide sequence selected from the group consisting of SEQ ID NO.: 682-794 and any combination thereof; (h) a gRNA targeting pFNCBDAS and comprising a nucleotide sequence that is at least 80% identical to the nucleotide sequence selected from the group consisting of SEQ ID NO.: 795-895 and any combination thereof; (i) a gRNA targeting pPKCBDAS and comprising a nucleotide sequence that is at least 80% identical to the nucleotide sequence selected from the group consisting of SEQ ID NO.: 896-1016 and any combination thereof; and (j) a gRNA targeting pPKCBDAS1 and comprising a nucleotide sequence that is at least 80% identical to the nucleotide sequence selected from the group consisting of SEQ ID NO.: 1017-1149 and any combination thereof.

It is a further object of the present invention to disclose the Cannabis plant or a cell thereof as defined in any of the above, wherein the targeted nucleotide modification is selected from the group consisting of insertion, deletion, single nucleotide polymorphism (SNP), and a polynucleotide modification, such that the expression of the THCAS and/or CBDAS polynucleotide is reduced or affected.

It is a further object of the present invention to disclose the Cannabis plant or a cell thereof as defined in any of the above, wherein said nucleotide modification is a missense mutation, nonsense mutation, insertion, deletion, indel, substitution or duplication.

It is a further object of the present invention to disclose the Cannabis plant or a cell thereof as defined in any of the above, wherein the insertion or the deletion produces a gene comprising a frameshift.

It is a further object of the present invention to disclose the Cannabis plant or a cell thereof as defined in any of the above, wherein said nucleotide modification is a silencing mutation, a knockdown mutation, a knockout mutation, a loss of function mutation, or a mutation causing enhanced allele expression.

It is a further object of the present invention to disclose the Cannabis plant or a cell thereof as defined in any of the above, wherein said plant is heterozygous or homozygous for the at least one nucleotide modification.

It is a further object of the present invention to disclose the Cannabis plant or a cell thereof as defined in any of the above, wherein the targeted nucleotide modification targets the genomic locus of at least one THCAS and/or CBDAS polynucleotide allele such that the one or more nucleotide modifications are present within (a) the coding region; (b) non-coding region; (c) regulatory sequence; or (d) untranslated region, of an endogenous polynucleotide encoding THCAS and/or CBDAS enzyme.

It is a further object of the present invention to disclose the Cannabis plant or a cell thereof as defined in any of the above, wherein the at least one targeted nucleotide modification results in one or more of the following: (a) reduced or elevated expression of the THCAS and/or CBDAS polynucleotide; (b) reduced or elevated enzymatic activity of the protein encoded by the THCAS and/or CBDAS polynucleotide; (c) generation of one or more non-functional alternative spliced transcripts of the THCAS and/or CBDAS polynucleotide; (d) deletion of a substantial portion or of the full-length open reading frame of the THCAS and/or CBDAS polynucleotide; (e) repression or enhancement of an enhancer motif present within the regulatory region controlling the expression of said THCAS and/or CBDAS polynucleotide; (f) repression or enhancement of a repressor motif present within a regulatory region controlling the expression of the THCAS and/or CBDAS polynucleotide; (g) modification of one or more nucleotides or deletion of a regulatory element operably linked to the expression of the THCAS and/or CBDAS allele polynucleotide, wherein the regulatory element is present within a promoter, intron, 3′UTR, terminator or a combination thereof.

It is a further object of the present invention to disclose the Cannabis plant or a cell thereof as defined in any of the above, wherein the targeted DNA modification is introduced through a genome modification technique selected from the group consisting polynucleotide-guided endonuclease, CRISPR (Clustered Regularly Interspaced Short Palindromic Repeats) and CRISPR-associated (Cas) (CRISPR-Cas) endonucleases, base editing deaminases, zinc finger nuclease (ZFN), a transcription activator-like effector nuclease (TALEN), engineered site-specific meganucleases, Argonaute or any combination thereof.

It is a further object of the present invention to disclose the Cannabis plant or a cell thereof as defined in any of the above, wherein said Cas genes or proteins are selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, Cu1966, bacteriophages Cas such as CasΦ (to (Cas-phi), and any combination thereof.

It is a further object of the present invention to disclose the Cannabis plant or a cell thereof as defined in any of the above, wherein said plant has THCA (and/or THC) and/or CBDA (and/or CBD) content of up to 30% by weight, particularly between about 0% to about 30% by weight, more particularly between about 0.3% to about 30%, even more particularly between about 0.1% to about 10% by weight.

It is a further object of the present invention to disclose the Cannabis plant or a cell thereof as defined in any of the above, wherein said plant has a THCA (and/or THC) and/or CBDA (and/or CBD) content of not more than about 0.3% by weight.

It is a further object of the present invention to disclose the Cannabis plant or a cell thereof as defined in any of the above, wherein said plant has a THCA (and/or THC) and/or CBDA (and/or CBD) content of at least 20% by weight.

It is a further object of the present invention to disclose the Cannabis plant or a cell thereof as defined in any of the above, wherein said plant is THCA (or THC) and/or CBDA (or CBD) free.

It is a further object of the present invention to disclose the Cannabis plant or a cell thereof as defined in any of the above, wherein the plant is a hybrid or an inbred line.

It is a further object of the present invention to disclose the Cannabis plant or a cell thereof as defined in any of the above, wherein said nucleotide modification is generated in planta via introduction of a construct comprising (a) Cas DNA and gRNA sequence having at least 80% sequence similarity to a nucleotide sequence selected from the group consisting of SEQ ID NO:7-SEQ ID NO:1149 and any combination thereof, or (b) a ribonucleoprotein (RNP) complex comprising Cas protein and gRNA sequence having at least 80% sequence similarity to a nucleotide sequence selected from the group consisting of SEQ ID NO:7-1149 and any combination thereof.

It is a further object of the present invention to disclose the Cannabis plant or a cell thereof as defined in any of the above, wherein said plant comprises at least one targeted genome modification in a polynucleotide selected from the group consisting of pFNTHCAS-1 having at least 70% sequence similarity to the nucleotide sequence as set forth in SEQ ID NO:1, pFNTHCAS-2 having at least 70% sequence similarity to the nucleotide sequence as set forth in SEQ ID NO:2, pPKTHCAS-1 having at least 70% sequence similarity to the nucleotide sequence as set forth in SEQ ID NO:3, pPKTHCAS-2 comprising a nucleic acid sequence having at least 70% sequence similarity to the nucleotide sequence as set forth in SEQ ID NO:4, pPKTHCAS-3 having at least 70% sequence similarity to the nucleotide sequence as set forth in SEQ ID NO:5, pPKTHCAS-4 having at least 70% sequence similarity to the nucleotide sequence as set forth in SEQ ID NO:6, pCBDAS2 having at least 70% sequence similarity to the nucleotide sequence as set forth in SEQ ID NO:1150, pFNCBDAS having at least 70% sequence similarity to the nucleotide sequence as set forth in SEQ ID NO:1151, pPKCBDAS having at least 70% sequence similarity to the nucleotide sequence as set forth in SEQ ID NO:1152, pPKCBDAS1 having at least 70% sequence similarity to the nucleotide sequence as set forth in SEQ ID NO:1153 and any combination thereof, and said plant exhibits reduced expression of THCA, THC, CBDA and/or CBD relative to a Cannabis plant of a similar genotype or genetic background lacking said targeted genome modification.

It is a further object of the present invention to disclose a recombinant DNA construct comprising a polynucleotide sequence encoding a RNA-guided endonuclease and at least one guide RNA (gRNA) having a sequence that is complementary to a target sequence within a regulatory region operably linked to the expression of at least one tetrahydrocannabinolic acid synthase (THCAS) and/or cannabidiolic acid synthase (CBDAS) allele.

It is a further object of the present invention to disclose the recombinant DNA construct as defined in any of the above, wherein the gRNA sequence comprising a polynucleotide sequence complementary to at least one of pFNTHCAS-1 having at least 70% sequence similarity to the nucleotide sequence as set forth in SEQ ID NO:1, pFNTHCAS-2 having at least 70% sequence similarity to the nucleotide sequence as set forth in SEQ ID NO:2, pPKTHCAS-1 having at least 70% sequence similarity to the nucleotide sequence as set forth in SEQ ID NO:3, pPKTHCAS-2 comprising a nucleic acid sequence having at least 70% sequence similarity to the nucleotide sequence as set forth in SEQ ID NO:4, pPKTHCAS-3 having at least 70% sequence similarity to the nucleotide sequence as set forth in SEQ ID NO:5, pPKTHCAS-4 having at least 70% sequence similarity to the nucleotide sequence as set forth in SEQ ID NO:6, pCBDAS2 having at least 70% sequence similarity to the nucleotide sequence as set forth in SEQ ID NO:1150, pFNCBDAS having at least 70% sequence similarity to the nucleotide sequence as set forth in SEQ ID NO:1151, pPKCBDAS having at least 70% sequence similarity to the nucleotide sequence as set forth in SEQ ID NO:1152, pPKCBDAS1 having at least 70% sequence similarity to the nucleotide sequence as set forth in SEQ ID NO:1153.

It is a further object of the present invention to disclose the recombinant DNA construct as defined in any of the above, wherein the gRNA sequence comprising a polynucleotide sequence having at least 80% sequence similarity to a nucleotide sequence selected from SEQ ID NO:7-SEQ ID NO:1149 and any combination thereof.

It is a further object of the present invention to disclose the recombinant DNA construct as defined in any of the above, wherein said gRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9) and TBN (Cas-phi).

It is a further object of the present invention to disclose a plant cell comprising the recombinant construct as defined in any of the above.

It is a further object of the present invention to disclose a guide RNA (gRNA) sequence that targets a tetrahydrocannabinolic acid synthase (THCAS) and/or cannabidiolic acid synthase (CBDAS) genomic locus of a plant cell, wherein the gRNA is complementary to at least one target sequence of a regulatory region within said genomic locus, said regulatory region operably linked to the expression of at least one THCAS and/or CBDAS allele.

It is a further object of the present invention to disclose the gRNA sequence as defined in any of the above, wherein said gRNA comprises a polynucleotide sequence selected from the group consisting of pFNTHCAS-1 having at least 70% sequence similarity to the nucleotide sequence as set forth in SEQ ID NO:1, pFNTHCAS-2 having at least 70% sequence similarity to the nucleotide sequence as set forth in SEQ ID NO:2, pPKTHCAS-1 having at least 70% sequence similarity to the nucleotide sequence as set forth in SEQ ID NO:3, pPKTHCAS-2 comprising a nucleic acid sequence having at least 70% sequence similarity to the nucleotide sequence as set forth in SEQ ID NO:4, pPKTHCAS-3 having at least 75% sequence similarity to the nucleotide sequence as set forth in SEQ ID NO:5, pPKTHCAS-4 having at least 70% sequence similarity to the nucleotide sequence as set forth in SEQ ID NO:6, pCBDAS2 having at least 70% sequence similarity to the nucleotide sequence as set forth in SEQ ID NO:1150, pFNCBDAS having at least 70% sequence similarity to the nucleotide sequence as set forth in SEQ ID NO:1151, pPKCBDAS having at least 70% sequence similarity to the nucleotide sequence as set forth in SEQ ID NO:1152, and pPKCBDAS1 having at least 70% sequence similarity to the nucleotide sequence as set forth in SEQ ID NO:1153.

It is a further object of the present invention to disclose the gRNA sequence as defined in any of the above, wherein the nucleotide sequence of said gRNA is selected from the group consisting of a nucleotide sequence that is at least 80% identical to the nucleotide sequence as set forth in SEQ ID NO.: 7-1149 and any combination thereof.

It is a further object of the present invention to disclose a recombinant DNA construct that expresses the guide RNA as defined in any of the above.

It is a further object of the present invention to disclose a plant cell or host cell comprising the guide RNA as defined in any of the above.

It is a further object of the present invention to disclose a plant cell or a host cell comprising the recombinant DNA construct as defined in any of the above.

It is a further object of the present invention to disclose a plant having stably incorporated into its genome the recombinant DNA construct as defined in any of the above.

It is a further object of the present invention to disclose a transgenic Cannabis plant comprising an endonuclease-mediated stably inherited genomic modification of regulatory region of a tetrahydrocannabinol acid synthase (THCAS) and/or cannabidiolic acid synthase (CBDAS) gene allele, said modification resulting in altered tetrahydrocannabinolic acid (THCA) and/or cannabidiolic acid (CBDA) content as compared to a comparable control Cannabis plant lacking said at least one targeted nucleotide modification.

It is a further object of the present invention to disclose a seed produced by the plant as defined in any of the above.

It is a further object of the present invention to disclose the Cannabis plant as defined in any of the above, further comprising a heterologous nucleic acid sequence selected from the group consisting of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance, a gene involved in salt resistance in plants and any combination thereof.

It is a further object of the present invention to disclose the Cannabis plant as defined in any of the above, wherein said plant does not comprise within its genome exogenous genetic material and said plant is a non-naturally occurring Cannabis plant or cell thereof.

It is a further object of the present invention to disclose a plant part, plant cell or plant seed of a plant as defined in any of the above.

It is a further object of the present invention to disclose a tissue culture of regenerable cells, protoplasts or callus obtained from the Cannabis plant or a cell thereof as defined in any of the above.

It is a further object of the present invention to disclose a non-living product or medical composition derived from the Cannabis plant as defined in any of the above.

It is a further object of the present invention to disclose the non-living product or medical composition as defined in any of the above, comprising a combined delta-9-tetrahydrocannabinol and tetrahydrocannabinolic acid concentration of between about 0% to about 30% by weight and/or cannabidiol and cannabidiolic acid concentration of between about 0% to about 30% by weight.

It is a further object of the present invention to disclose the non-living product or medical composition as defined in any of the above, comprising a THCA (or THC) and/or CBDA (or CBD) content of not more than about 0.3% by weight.

It is a further object of the present invention to disclose the non-living product or medical composition as defined in any of the above, comprising a THCA (and/or THC) and/or CBDA (or CBD) content of at least 20% by weight.

It is a further object of the present invention to disclose the non-living product or medical composition as defined in any of the above, comprising Cannabis oil, Cannabis tincture, dried Cannabis flowers, and/or dried Cannabis leaves for medical use.

It is a further object of the present invention to disclose the non-living product or medical composition as defined in any of the above, formulated for inhalation, oral consumption, sublingual consumption, or topical consumption.

It is a further object of the present invention to disclose a method for producing a Cannabis plant or a cell thereof as defined in any of the above, comprising steps of introducing one or more nucleotide modifications through a targeted genome modification at a regulatory region modulating the expression of at least one THCAS and/or CBDAS allele.

It is a further object of the present invention to disclose the method as defined above, wherein the regulatory region comprises a nucleotide sequence selected from the group consisting of pFNTHCAS-1 having a nucleic acid sequence as set forth in SEQ ID NO:1 or a functional variant thereof, pFNTHCAS-2 having a nucleic acid sequence as set forth in SEQ ID NO:2 or a functional variant thereof, pPKTHCAS-1 having a nucleic acid sequence as set forth in SEQ ID NO:3 or a functional variant thereof, pPKTHCAS-2 having a nucleic acid sequence as set forth in SEQ ID NO:4 or a functional variant thereof, pPKTHCAS-3 having a nucleic acid sequence as set forth in SEQ ID NO:5 or a functional variant thereof, pPKTHCAS-4 having a nucleic acid sequence as set forth in SEQ ID NO:6 or a functional variant thereof, pCBDAS2 having a nucleic acid sequence as set forth in SEQ ID NO:1150 or a functional variant thereof, pFNCBDAS having a nucleic acid sequence as set forth in SEQ ID NO:1151 or a functional variant thereof, pPKCBDAS having a nucleic acid sequence as set forth in SEQ ID NO:1152 or a functional variant thereof, pPKCBDAS1 having a nucleic acid sequence as set forth in SEQ ID NO:1153 or a functional variant thereof, and any combination thereof.

It is a further object of the present invention to disclose the method as defined in any of the above, wherein said functional variant has at least 70% sequence identity or similarity to the nucleic acid sequence of said regulatory region sequence or a codon degenerate nucleotide sequence thereof.

It is a further object of the present invention to disclose the method as defined in any of the above, wherein the targeted nucleotide modification is induced by a guide RNA that corresponds to a target sequence comprising a polynucleotide selected from the group consisting of pFNTHCAS-1 having at least 70% sequence similarity to the nucleotide sequence as set forth in SEQ ID NO:1, pFNTHCAS-2 having at least 70% sequence similarity to the nucleotide sequence as set forth in SEQ ID NO:2, pPKTHCAS-1 having at least 70% sequence similarity to the nucleotide sequence as set forth in SEQ ID NO:3, pPKTHCAS-2 comprising a nucleic acid sequence having at least 70% sequence similarity to the nucleotide sequence as set forth in SEQ ID NO:4, pPKTHCAS-3 having at least 70% sequence similarity to the nucleotide sequence as set forth in SEQ ID NO:5, pPKTHCAS-4 having at least 70% sequence similarity to the nucleotide sequence as set forth in SEQ ID NO:6, pCBDAS2 having at least 70% sequence similarity to the nucleotide sequence as set forth in SEQ ID NO:1150, pFNCBDAS having at least 70% sequence similarity to the nucleotide sequence as set forth in SEQ ID NO:1151, pPKCBDAS having at least 70% sequence similarity to the nucleotide sequence as set forth in SEQ ID NO:1152, pPKCBDAS1 having at least 70% sequence similarity to the nucleotide sequence as set forth in SEQ ID NO:1153, and any combination thereof.

It is a further object of the present invention to disclose the method as defined in any of the above, wherein the guide RNA (gRNA) nucleotide sequence is selected from the group consisting of: (a) a gRNA targeting pFNTHCAS-1 and comprising a nucleotide sequence that is at least 80% identical to a nucleotide sequence selected from the group consisting of SEQ ID NO: 7-91 and any combination thereof; (b) a gRNA targeting pFNTHCAS-2 and comprising a nucleotide sequence that is at least 80% identical to the nucleotide sequence selected from the group consisting of SEQ ID NO.: 92-176 and any combination thereof; (c) a gRNA targeting pPKTHCAS-1 and comprising a nucleotide sequence that is at least 80% identical to the nucleotide sequence selected from the group consisting of SEQ ID NO.: 177-278 and any combination thereof; (d) a gRNA targeting pPKTHCAS-2 and comprising a nucleotide sequence that is at least 80% identical to the nucleotide sequence selected from the group consisting of SEQ ID NO.: 279-419 and any combination thereof; (e) a gRNA targeting pPKTHCAS-3 and comprising a nucleotide sequence that is at least 80% identical to the nucleotide sequence selected from the group consisting of SEQ ID NO.: 420-560 and any combination thereof; (f) a gRNA targeting pPKTHCAS-4 and comprising a nucleotide sequence that is at least 80% identical to the nucleotide sequence selected from the group consisting of SEQ ID NO.: 561-681 and any combination thereof; (g) a gRNA targeting pCBDAS2 and comprising a nucleotide sequence that is at least 80% identical to the nucleotide sequence selected from the group consisting of SEQ ID NO.: 682-794 and any combination thereof; (h) a gRNA targeting pFNCBDAS and comprising a nucleotide sequence that is at least 80% identical to the nucleotide sequence selected from the group consisting of SEQ ID NO.: 795-895 and any combination thereof; (i) a gRNA targeting pPKCBDAS and comprising a nucleotide sequence that is at least 80% identical to the nucleotide sequence selected from the group consisting of SEQ ID NO.: 896-1016 and any combination thereof; and (j) a gRNA targeting pPKCBDAS1 and comprising a nucleotide sequence that is at least 80% identical to the nucleotide sequence selected from the group consisting of SEQ ID NO.: 1017-1149 and any combination thereof.

It is a further object of the present invention to disclose the method as defined in any of the above, wherein the targeted nucleotide modification is selected from the group consisting of insertion, deletion, single nucleotide polymorphism (SNP), a missense mutation, nonsense mutation, indel, substitution or duplication and a polynucleotide modification, such that the expression of the THCAS and/or CDBAS polynucleotide is reduced or affected.

It is a further object of the present invention to disclose the method as defined in any of the above, wherein the Cannabis plant exhibits reduced THCA and/or CDBA content when the targeted DNA modification within the regulatory region results in reduced expression or activity of protein encoded by the at least one THCAS and/or CDBAS allele polynucleotide.

It is a further object of the present invention to disclose the method as defined in any of the above, wherein the Cannabis plant exhibits elevated THCA and/or CDBA content when the targeted DNA modification within the regulatory region results in increased expression or activity of protein encoded by the at least one THCAS and/or CDBA allele polynucleotide, respectively.

It is a further object of the present invention to disclose the method as defined in any of the above, wherein the targeted DNA modification is through a genome modification technique selected from the group consisting of polynucleotide-guided endonuclease, CRISPR (Clustered Regularly Interspaced Short Palindromic Repeats) and CRISPR-associated (Cas) (CRISPR-Cas) endonucleases, base editing deaminases, zinc finger nuclease (ZFN), a transcription activator-like effector nuclease (TALEN), engineered site-specific meganucleases, Argonaute or any combination thereof.

It is a further object of the present invention to disclose the method as defined in any of the above, wherein said plant has THCA (or THC) and/or CBDA (or CBD) content of up to 30% by weight, particularly between about 0% to about 30% by weight, more particularly between about 0.3% to about 30%, even more particularly between about 0.1% to about 10% by weight.

It is a further object of the present invention to disclose the method as defined in any of the above, wherein said plant has a THCA (or THC) and/or CBDA (or CBD) content of not more than about 0.3% by weight.

It is a further object of the present invention to disclose the method as defined in any of the above, wherein said plant has a THCA (or THC) and/or CBDA (or CBD) content of at least 20% by weight.

It is a further object of the present invention to disclose the method as defined in any of the above, wherein said plant is THCA or THC and/or CBDA or CBD free.

It is a further object of the present invention to disclose a method for altering tetrahydrocannabinolic acid (THCA) and/or cannabidiolic acid (CBDA) content in a Cannabis plant as defined in any of the above, by modifying a genomic locus involved in tetrahydrocannabinolic acid synthase (THCAS) and/or cannabidiolic acid synthase (CBDAS) gene expression relative to a comparable control Cannabis plant, wherein said method comprises introducing one or more nucleotide modifications through a targeted DNA modification at a regulatory region modulating expression of at least one THCAS and/or CBDAS allele.

It is a further object of the present invention to disclose a plant part, plant cell or plant seed produced by the method as defined in any of the above.

It is a further object of the present invention to disclose a method for producing a non-living product or medical Cannabis composition, the method comprising: (a) obtaining the Cannabis plant as defined in any of the above; and (b) formulating a non-ling product or medical Cannabis composition from said plant.

It is a further object of the present invention to disclose an isolated nucleotide sequence having at least 70% sequence identity to a nucleotide sequence selected from the group consisting of SEQ ID NO:1 to SEQ ID NO:6 and SEQ ID NO:1150 to SEQ ID NO:1153.

It is a further object of the present invention to disclose an isolated nucleotide sequence having at least 70% sequence identity to a nucleotide sequence selected from the group consisting of SEQ ID NO:7 to SEQ ID NO:1149.

It is a further object of the present invention to disclose a vector, construct or expression system or cassette comprising a nucleic acid sequence having at least 70% sequence identity to a nucleotide sequence selected from the group consisting of SEQ ID NO:1 to SEQ ID NO:1153.

It is a further object of the present invention to disclose use of a nucleotide sequence having at least 70% sequence identity to a nucleotide sequence as set forth in at least one of SEQ ID NO: 7-91 and any combination thereof for targeted genome modification of pFNTHCAS-1 gene.

It is a further object of the present invention to disclose use of a nucleotide sequence having at least 70% sequence identity to a nucleotide sequence as set forth in at least one of SEQ ID NO: 92-176 and any combination thereof for targeted genome modification of pFNTHCAS-2 gene.

It is a further object of the present invention to disclose use of a nucleotide sequence having at least 70% sequence identity to a nucleotide sequence as set forth in at least one of SEQ ID NO: 177-278 and any combination thereof for targeted genome modification of pPKTHCAS-1 gene.

It is a further object of the present invention to disclose use of a nucleotide sequence having at least 70% sequence identity to a nucleotide sequence as set forth in at least one of SEQ ID NO: 279-419 and any combination thereof for targeted genome modification of pPKTHCAS-2 gene.

It is a further object of the present invention to disclose use of a nucleotide sequence having at least 70% sequence identity to a nucleotide sequence as set forth in at least one of SEQ ID NO: 420-560 and any combination thereof for targeted genome modification of pPKTHCAS-3 gene.

It is a further object of the present invention to disclose use of a nucleotide sequence having at least 70% sequence identity to a nucleotide sequence as set forth in at least one of SEQ ID NO: 561-681 and any combination thereof for targeted genome modification of pPKTHCAS-4 gene.

It is a further object of the present invention to disclose use of a nucleotide sequence having at least 70% sequence identity to a nucleotide sequence as set forth in at least one of SEQ ID NO: 682-794 and any combination thereof for targeted genome modification of pCBDAS2 gene.

It is a further object of the present invention to disclose use of a nucleotide sequence having at least 70% sequence identity to a nucleotide sequence as set forth in at least one of SEQ ID NO: 795-895 and any combination thereof for targeted genome modification of pFNCBDAS gene.

It is a further object of the present invention to disclose use of a nucleotide sequence having at least 70% sequence identity to a nucleotide sequence as set forth in at least one of SEQ ID NO: 896-1016 and any combination thereof for targeted genome modification of pPKCBDAS gene.

It is a further object of the present invention to disclose use of a nucleotide sequence having at least 70% sequence identity to a nucleotide sequence as set forth in at least one of SEQ ID NO: 1017-1149 and any combination thereof for targeted genome modification of pPKCBDAS1 gene.

It is a further object of the present invention to disclose use of a nucleotide sequence having at least 70% sequence identity to a nucleotide sequence as set forth in at least one of SEQ ID NO: 7-681 and any combination thereof for reducing THCA content in a Cannabis plant.

It is a further object of the present invention to disclose use of a nucleotide sequence having at least 70% sequence identity to a nucleotide sequence as set forth in at least one of SEQ ID NO: 682-1149 and any combination thereof for reducing CBDA content in a Cannabis plant.

It is a further object of the present invention to disclose the Cannabis plant as defined in any of the above wherein said plant genotype is obtainable by deposit under accession number with NCIMB Aberdeen AB21 9YA, Scotland, UK.

It is a further object of the present invention to disclose a method for producing a Cannabis plant exhibiting altered tetrahydrocannabinolic acid (THCA) and/or cannabidiolic acid (CBDA) content and comprising a modified genomic locus involved in tetrahydrocannabinolic acid synthase (THCAS) and/or cannabidiolic acid synthase (CBDAS) gene expression as compared to a control Cannabis plant, said method comprises steps of introducing one or more nucleotide modifications through a targeted genome modification at a regulatory region modulating at least one THCAS and/or CBDAS allele expression.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings; wherein:

FIG. 1 is schematically presenting CRISPR/Cas9 mode of action as depicted by Xie, Kabin, and Yinong Yang. “RNA-guided genome editing in plants using a CRISPR-Cas system.” Molecular plant 6.6 (2013): 1975-1983;

FIG. 2 is schematically illustrating the cannabinoid biosynthesis pathway as depicted by the C. sativa (Cannabis) Genome Browser internet site;

FIG. 3 is presenting regenerated transformed Cannabis tissue;

FIG. 4 is photographically presenting PCR detection of Cas9 DNA in shoots of Cannabis plants transformed using biolistics; and

FIG. 5 is illustrating in vitro cleavage activity of CRISPR/Cas9; FIG. 5A is a scheme of genomic area targeted for editing; and FIG. 5B is a gel showing digestion of PCR amplicon containing RNP complex of Cas9 and gene specific gRNA.

It will be appreciated that, for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention.

Some features or elements described with respect to one embodiment may be combined with features or elements described with respect to other embodiments. For the sake of clarity, discussion of same or similar features or elements may not be repeated.

Plant breeding is currently limited by improvements in quantitative traits that often rely on laborious selection of rare naturally occurring mutations in gene-regulatory regions.

By the current invention, CRISPR/Cas9 genome editing of promoters of genes encoding cannabinoid synthesis proteins (e.g. THCAS and/or CBDAS) generate diverse cis-regulatory alleles that provide beneficial quantitative variation for breeding. This approach allows immediate selection and fixation of novel alleles in transgene-free plants and manipulation of cannabinoid (e.g. THCA, CBDA and CBGA) content in the Cannabis plant.

The present invention thus provides a platform to enhance variation and control of THC, CBD and/or other cannabinoids expression in the Cannabis plant.

The present invention discloses manipulation of the biosynthesis pathways of a Cannabis plant of genus Cannabis. Accordingly, Cannabis plants of the present invention having a modified therapeutic component(s) profile may be useful in the production of medical Cannabis and/or may also be useful in the production of specific components or therapeutic formulations derived therefrom.

According to one embodiment, a Cannabis plant exhibiting altered tetrahydrocannabinolic acid (THCA) and/or cannabidiolic acid (CBDA) content is provided. The aforementioned plant comprises a modified genomic locus involved in tetrahydrocannabinolic acid synthase (THCAS) and/or cannabidiolic acid synthase (CBDAS) gene expression, said genomic locus comprises at least one targeted nucleotide modification within a regulatory region modulating the expression of at least one allele of said THCAS and/or CBDAS genes.

According to further embodiment, the present invention provides a recombinant DNA construct comprising a polynucleotide sequence encoding a RNA-guided endonuclease and at least one guide RNA (gRNA) having a sequence that is complementary to a target sequence within a regulatory region operably linked to the expression of at least one tetrahydrocannabinolic acid synthase (THCAS) and/or cannabidiolic acid synthase (CBDAS) allele.

According to further embodiment, a guide RNA (gRNA) sequence that targets a tetrahydrocannabinolic acid synthase (THCAS) and/or cannabidiolic acid synthase (CBDAS) genomic locus of a plant cell is herein disclosed. The gRNA is complementary to at least one target sequence of a regulatory region within said genomic locus, said regulatory region operably linked to the expression of at least one THCAS and/or CBDAS allele.

It is further within the scope of the current invention to provide a recombinant DNA construct that expresses the guide RNA as defined in any of the above.

It is further within the scope to provide a plant cell comprising the guide RNA as defined in any of the above.

According to further embodiments, a plant cell comprising the recombinant DNA construct as defined above is provided.

According to other embodiments, a plant having stably incorporated into its genome the recombinant DNA construct as defined in any of the above is disclosed.

It is further within the scope to provide a seed produced by the plant of the present invention.

It is further within the scope to provide a plant part, plant cell or plant seed of a plant as defined above. The plant part may include a tissue culture of regenerable cells, protoplasts or callus obtained from the Cannabis plant provided by the present invention.

According to an embodiment of the present invention, the genotype of the Cannabis plant comprising at least one targeted nucleotide modification within a regulatory region modulating the expression of at least one allele of said THCAS and/or CBDAS genes is obtainable by seed deposit under accession number with NCIMB Aberdeen AB21 9YA, Scotland, UK.

According to some further embodiments of the present invention, a product such as a medical composition, derived from the Cannabis plant as defined in any of the above is provided.

The present invention further provides a method for producing a Cannabis plant exhibiting altered tetrahydrocannabinolic acid (THCA) and/or cannabidiolic acid (CBDA) content and comprising a modified genomic locus involved in tetrahydrocannabinolic acid synthase (THCAS) and/or cannabidiolic acid synthase (CBDAS) gene expression as compared to a control Cannabis plant, said method comprises steps of introducing one or more nucleotide modifications through a targeted genome modification at a regulatory region modulating at least one THCAS and/or CBDAS allele expression.

According to a further aspect of the present invention, a method for altering tetrahydrocannabinolic acid (THCA) and/or cannabidiolic acid (CBDA) content in a Cannabis plant by modifying a genomic locus involved in tetrahydrocannabinolic acid synthase (THCAS) and/or cannabidiolic acid synthase (CBDAS) gene expression as compared to a control Cannabis plant is provided. The method comprises introducing one or more nucleotide modifications through a targeted DNA modification at a regulatory region modulating expression of at least one THCAS and/or CBDAS allele.

The present invention further provides a plant part, plant cell or plant seed produced by the method as defined in any of the above.

According to some other embodiments, the present invention provides a method for producing a medical Cannabis composition. The method comprises (a) obtaining the Cannabis plant of the present invention comprising at least one targeted nucleotide modification within a regulatory region modulating the expression of at least one allele of said THCAS and/or CBDAS genes; and (b) formulating a medical Cannabis composition from said plant.

According to further embodiments, the present invention provides an isolated nucleotide sequence having at least 70% sequence identity to a nucleotide sequence selected from the group consisting of SEQ ID NO:1 to SEQ ID NO:1153.

According to further embodiments of the present invention, a vector, construct or expression system or cassette comprising a nucleic acid sequence having at least 70% sequence identity to a nucleotide sequence selected from the group consisting of SEQ ID NO:1 to SEQ ID NO:1153 is herein provided.

Other embodiments of the present invention include the use of a nucleotide sequence having at least 70% sequence identity to a nucleotide sequence as set forth in at least one of SEQ ID NO: 7-681 and any combination thereof and/or at least one of SEQ ID NO: 682-1149 and any combination thereof, for reducing THCA content and/or CBDA content, respectively, in a Cannabis plant.

According to further main aspects of the present invention, THC free Cannabis plants for seeds, fiber and/or medical use are provided. This may be achieved by using genome editing techniques to target regulatory regions controlling expression of THCAS alleles, and selecting for mutations significantly reducing THCAS content within the plant. In this way, any high level THC variety (e.g. Purple Kush) can be converted into a low level THC (below 0.3% THC by weight) or even THC free Cannabis variety.

According to other main aspects of the invention, Cannabis plants with reduced CBDA or CBD content are produced by genome modification targeted to regulatory regions, i.e. promoter regions of genes or alleles encoding CBDAS enzyme.

According to other main aspects of the invention, Cannabis plants comprising silencing mutation conferring significantly reduced CBDA (or CBD) and THCA (or THC) content are produced by genome modification targeted to regulatory regions, i.e. promoter regions of genes or alleles encoding CBDAS and THCAS enzymes. Such plants may be essentially absent of both enzymes and may have elevated or enhanced content of CBGA, which is a substrate for CBDAS and THCAS enzymes and a precursor for CBDA and THCA cannabinoids.

It is acknowledged that regulatory regions of genes may have relatively less conserved among different alleles or homologues or varieties of the same gene. Thus specific sequences should be designed for targeting regulatory regions of different alleles of a gene of interest, i.e. THCAS and CBDAS gene alleles of different Cannabis varieties.

It is herein acknowledged that though widely favored in plant and animal evolution and domestication, cis-regulatory variants are far from being saturated and thus represent an unexplored resource for expanding allelic diversity and for controlling and/or altering gene expression.

The present invention uses genome editing techniques at target sites located outside the coding region, i.e. cis-regulatory elements, of Cannabis tetrahydrocannabinolic acid synthase (CsTHCAS) and/or Cannabis cannabidiolic acid synthase (CsCBDAS) gene variants, to delete the full-length gene and/or silence its expression, resulting in significantly reduced THC and/or CBD content in the modified plant.

It is emphasized that alterations in gene-regulatory elements may result in linear or non-linear effect between transcriptional and phenotypic change, and it is unexpected how such responses vary for different genes. Thus, by exploring targeted cis-regulatory variation, new opportunities for altering gene expression to obtain desirable phenotypes and traits could be achieved by the current invention.

It is also disclosed that up until now, in plants, and especially in Cannabis plants, genome editing technology was mainly used to engineer mutations in coding sequences, with the goal of creating null alleles for functional studies.

The current invention hypothesized that elements of CRISPR/Cas9 technology could be integrated to engineer cis-regulatory mutations targeted to modify, and more specifically silence THCAS and/or CBDAS expression. In this way Cannabis plants or strains with reduced content, or even substantially free of, THCA and/or THC and/or CBDA and/or CBD are generated, useful for medical purposes.

According to main embodiments, the present invention provides a Cannabis plant exhibiting reduced tetrahydrocannabinolic acid (THCA) and/or cannabidiolic acid (CBDA) content, wherein said plant comprises a modified genomic locus involved in tetrahydrocannabinolic acid synthase (THCAS) and/or cannabidiolic acid synthase (CBDAS) gene expression, said genomic locus comprises at least one targeted nucleotide modification as compared to a control Cannabis plant, within a regulatory region controlling said THCAS and/or CBDAS expression. The present invention further provides methods for production the aforementioned Cannabis plant and polynucleotide sequences for generating the targeted mutations.

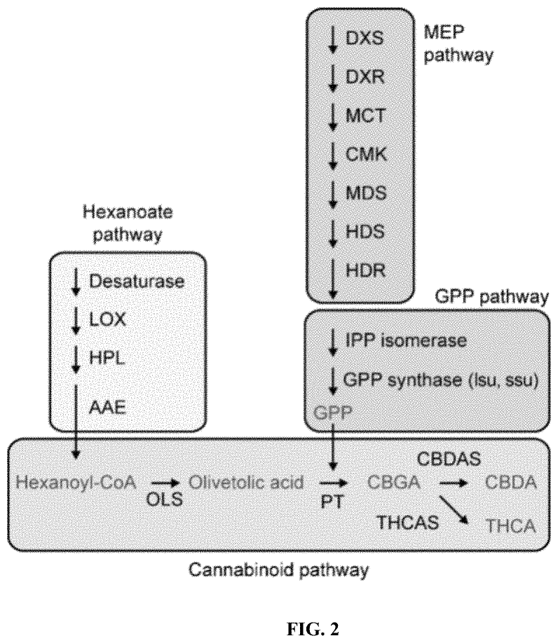

Reference is now made to FIG. 2 schematically illustrating the proposed pathway leading to the major cannabinoids A 9-tetrahydrocannabinolic acid (THCA) and cannabidiolic acid (CBDA), which decarboxylate to yield A 9-tetrahydrocannabinol (THC) and cannabidiol (CBD), respectively. The biosynthesis of THC and CBD in Cannabis follows a similar pathway.

As described in FIG. 2, cannabinoid biosynthesis genes are generally unlinked. The gene encoding aromatic prenyltransferase (AP), produces the substrate (Cannabigerolic acid, CBGA) for both THCA and CBDA synthases (THCAS and CBDAS, respectively).

Cannabigerolic acid (CBGA), the precursor to all natural cannabinoids, is cyclized into tetrahydrocannabinolic acid (THCA) and cannabidiolic acid (CBDA) by THCA and CBDA synthase (THCAS and CBDAS in FIG. 2), respectively. The final products of THC and CBD are formed via decarboxylation of these acidic forms. Structurally, there is an important difference between these major cannabinoids. Where THC contains a cyclic ring, CBD contains a hydroxyl group. This seemingly small difference in molecular structure may give the two compounds their different pharmacological properties.

In FIG. 2, AAE, refers to acyl-activating enzyme; CBD: cannabidiol; CYP76F39, α/β-santalene monooxygenase; GPP synthase small subunit; OLS, olivetol synthase; P450: haemoprotein cytochrome P450; PT, prenyltransferase; STS, santalene synthase; TS, gamma-terpinene synthase; and TXS, taxadiene synthase.

Here, we describe regulating THCAS and/or CBDAS cannabinoid biosynthesis protein expression by targeted genome editing at the promoter regions of THCAS and/or CBDAS gene alleles, for example, of the drug-type strain Purple Kush′ and the hemp variety “Finola”

It is within the scope that a system that exploits heritability of CRISPR/Cas9 transgenes carrying gRNAs in F1 populations, to rapidly and efficiently generate novel cis-regulatory alleles for THCAS and/or CBDAS gene variants in Cannabis is provided.

In this way, stabilized promoter alleles that provides a reduced THCAS and/or CBDAS expression is generated. It should be noted that transcriptional change may be a poor predictor of phenotypic effect, showing the complexity in how regulatory variation impacts quantitative traits.

By targeting gene promoter regions with guide RNAs (gRNAs) corresponding to the regulatory regions of various THCAS homologue sequences from the ‘drug-type’ strain Purple Kush (PKTHCAS-1, PKTHCAS-2, PKTHCAS-3 and PKTHCAS-4) and the hemp variety “Finola” (FNTHCAS-1 and FNTHCAS-2), a range of lower THCA-content phenotypic changes could be obtained in Cannabis.

In addition, by targeting gene promoter regions with guide RNAs (gRNAs) corresponding to the regulatory regions of various CBDAS homologue sequences such as of cannabidiolic acid synthase-like 1 (CBDAS2) homologue, CBDAS variant or homologue of ‘Finola’ (FN) Cannabis strain (FNCBDAS) or a CBDAS variant or homologue of Purple Kush (PK) Cannabis strain (PKCBDAS) or PKCBDAS1, a range of lower CBDA-content phenotypic changes could be obtained in Cannabis.

Through Cas9-gRNA directed cleavage and imprecise repair at each target site, an array of mutation types could be induced in the promoter sequences of THCAS gene variants (pFNTHCAS-1, pFNTHCAS-2, pPKTHCAS-1, pPKTHCAS-2, pPKTHCAS-3 and pPKTHCAS-4) and/or CBDAS gene variant (pCBDAS2, pFNCBDAS, pPKCBDAS and pPKCBDAS1), including deletions of various sizes and small indels at target sites.

The resulting alleles, having mutations that might impact cis-regulatory elements (CREs) or cis-regulatory modules, could then be evaluated for phenotypic changes by generating stable homozygous mutants in subsequent generations.

According to one embodiment, a CRISPR/Cas9 construct with gRNA designed to target the promoter region upstream of at least one of FNTHCAS-1, FNTHCAS-2, PKTHCAS-1, PKTHCAS-2, PKTHCAS-3, PKTHCAS-4, CBDAS2, FNCBDAS, PKCBDAS and PKCBDAS1 coding sequence was generated, without considering any predicted cis-regulatory element or module in the promoter sequence. First-generation transgenic plants (TO) were generated and PCR genotyping was performed to reveal mutations/deletions of various sizes in the target region, causing reduced or knockdown of THCAS and/or CBDAS expression and/or function.

It is herein acknowledged that as the pharma industry is interested in extracting the cannabinoids from the Cannabis plant, individual Cannabis plants or strains or varieties containing modulated levels of such cannabinoids can be developed, tailored to the specific needs of the pharma industry thereby increasing the cost effectiveness and attractiveness of this crop.

The solution proposed by the current invention is using genome editing such as the CRISPR/Cas system in order to create cultivated Cannabis plants with modulated levels or ratios of cannabinoids. More specifically alternation of specific cannabinoids, i.e. THCA or THC, CBDA or CBD is achieved by using genome editing targeted to the regulatory regions of these genes to reduce their expression at the transcription (RNA) and/or translation (protein) levels of the cannabinoid biosynthesis pathway.

Breeding using genome editing allows a precise and significantly shorter breeding process in order to achieve these goals with a much higher success rate. Thus genome editing, has the potential to generate improved varieties faster and at a lower cost.

The current invention discloses the generation of non GMO Cannabis plants with manipulated and controlled cannabinoid content, using the genome editing technology, e.g., the CRISPR/Cas9 highly precise tool targeted to regulatory regions of genes of interest. The generated mutations can be introduced into elite or locally adapted Cannabis lines rapidly, with relatively minimal effort and investment.

Genome editing is an efficient and useful tool for increasing crop productivity traits, and there is particular interest in advancing manipulation of genes controlling cannabinoids biosynthesis in Cannabis species, to produce strains which are adapted to specific therapeutic or regulatory needs.

A major obstacle for CRISPR-Cas9 plant genome editing is lack of efficient tissue culture and transformation methodologies. The present invention achieves these aims and surprisingly provides transformed and regenerated Cannabis plants with modified desirable cannabinoids content.

To that end, guide RNAs (gRNAs) were designed for each of the target gene promoters herein identified in Cannabis to induce mutations in at least one Cannabis tetrahydrocannabinolic acid synthase (CsTHCAS)) and/or cannabidiolic acid synthase (CBDAS) gene variant or homologue.

The present invention shows that Cannabis plants which contain genome editing events with at least one promoter of THCAS allele, express not more than 0.5% THC (or THCA) by weight. In specific embodiments, such plants express less than 0.5%, preferably less than 0.4%, less than 0.3%, less than 0.2%, less than 0.1% or 0% THC and/or THCAS by weight (e.g. by dry weight).

In further embodiments, the present invention shows that Cannabis plants which contain genome editing events with at least one promoter of CBDAS allele, express not more than 0.5% CBD (or CBDA) by weight. In specific embodiments, such plants express less than 0.5%, preferably less than 0.4%, less than 0.3%, less than 0.2%, less than 0.1% or 0% CBD and/or CBDAS by weight (e.g. by dry weight).

The present invention further shows that Cannabis plants containing genome editing events within the THCAS genomic locus express higher levels of CBD (or CBDA) compared to non-edited plants. In a further embodiment, the CsTHCAS edited plants contain very low levels of THCA (or THC), preferably not more than 0.5% by weight.

The present invention further shows that Cannabis plants containing genome editing events within the CBDAS genomic locus express higher levels of THC (or THCA) compared to non-edited plants. In a further embodiment, the CsCBDAS edited plants contain very low levels of CBDA (or CBD), preferably not more than 0.5% by weight.

The present invention further shows that Cannabis plants containing knockdown genome editing events within THCAS and CBDAS genomic locus express higher levels of CBG (or CBGA) compared to non-edited plants. In a further embodiment, the edited plants contain very low levels of CBDA (or CBD) and THCA (or THC), preferably not more than 0.5% by weight.

It is a further aspect of the present invention to provide the Cannabis plant as defined in any of the above, wherein said plant has a THC and/or CBD content of up to 30% by weight, particularly between about 0% to about 30% by weight, more particularly between about 0.3% to about 30%, even more particularly between about 0.3% to about 10% by weight.

According to a further embodiment, the Cannabis plant of the present invention has a THCA (and/or THC) and/or CBDA (and/or CBD) content of not more than about 0.3% by weight.

According to a further embodiment, the Cannabis plant of the present invention has a THCA (and/or THC) and/or CBDA (and/or CBD) content of at least 20% by weight.

According to a further embodiment, the Cannabis plant of the present invention is THCA (or THC) and/or CBDA (or CBD) free.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same Cannabis species or strain or variety or to sibling plant, or one or more individuals having one or both parents in common.

As used herein, the phrase “consisting essentially of” or “essentially” generally refers to a composition, method that includes materials, steps, features, components, or elements, in addition to those literally disclosed, provided that these additional materials, steps, features, components, or elements do not materially affect the basic and novel characteristic(s) of the claimed subject matter.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds.

The term “Cannabis” refers hereinafter to a genus of flowering plants in the family Cannabaceae. Cannabis is an annual, dioecious, flowering herb that includes, but is not limited to three different species, Cannabis sativa, Cannabis indica and Cannabis ruderalis. The term also refers to hemp. Cannabis plants produce a group of chemicals called cannabinoids. Cannabinoids, terpenoids, and other compounds are secreted by glandular trichomes that occur most abundantly on the floral calyxes and bracts of female Cannabis plants.

It is herein acknowledge that Cannabis has traditionally been classified as having a drug-type (“marijuana”) or hemp-type chemotype based on the relative proportion of THC to CBD.

The term “chemotype” also sometime used as “chemovar” refers herein to a chemically distinct entity in a plant, with differences in the composition of the secondary metabolites. It is herein acknowledged that minor genetic and epigenetic changes with little or no effect on morphology or anatomy may produce large changes in the chemical phenotype. Chemotypes are often defined by the most abundant chemical produced by that individual.

According to further aspects, a chemotype describes the subspecies of a plant that have the same morphological characteristics (relating to form and structure) but produce different quantities of chemical components in their essential oils.

In certain embodiments of the invention, chemical phenotypes (chemotypes) can be useful to classify C. sativa as drug- or fiber-type varieties. It is suggested that drug-type or chemotype I C. sativa cultivars contain high levels of 49-THC, while CBD-rich cultivars containing low levels of THC are regarded as fiber-type or chemotype III cultivars. Hemp- and drug-type cultivars are members of both C. sativa sativa and C. sativa indica subspecies. Chemotaxonomic analysis is useful to differentiate hemp from drug-type C. sativa based on acceptable levels of 49-THC established by regulating bodies.

The term “Finola” or “FN” as used herein refers to a hemp-type Cannabis Sativa L. strain or cultivar. It is known for its high CBD content and low THC levels (e.g. low THC<0.2%).

The term “Purple Kush” or “PK” herein refers to a marijuana or drug or potent-type C. sativa strain. It is known for its relatively high THC content.

It is within the scope of the present invention that THCAS and CBDAS (which determine the drug vs hemp chemotype) are highly non-homologous between drug- and hemp-type alleles. The current invention offers for the first time THCAS and/or CBDAS alleles derived from FN and PK strains that are modified at their promoter region by targeted genome modification to down regulate or silence their expression. The THCAS genes modified at their promoter-site include FNTHCAS-1, FNTHCAS-2, PKTHCAS-1, PKTHCAS-2, PKTHCAS-3 and PKTHCAS-4. The sequences of the THCAS promoter-sites targeted by genome modification include sequence corresponding to pFNTHCAS-1 having a polynucleotide sequence as set forth in SEQ ID NO: 1, pFNTHCAS-2 having a polynucleotide sequence as set forth in SEQ ID NO: 2, pPKTHCAS-1 having a polynucleotide sequence as set forth in SEQ ID NO: 3, pPKTHCAS-2 having a polynucleotide sequence as set forth in SEQ ID NO: 4, pPKTHCAS-3 having a polynucleotide sequence as set forth in SEQ ID NO: 5 and pPKTHCAS-4 having a polynucleotide sequence as set forth in SEQ ID NO: 6.

The CBDAS genes modified at their promoter-site include cannabidiolic acid synthase-like 1 (CBDAS2), FNCBDAS, PKCBDAS and PKCBDAS1. The sequences of the CBDAS promoter-sites targeted by genome modification include sequence corresponding to pCBDAS2 having a polynucleotide sequence as set forth in SEQ ID NO: 1150, pFNCBDAS having a polynucleotide sequence as set forth in SEQ ID NO:

1151, pPKCBDAS having a polynucleotide sequence as set forth in SEQ ID NO: 1152 and pPKCBDAS1 having a polynucleotide sequence as set forth in SEQ ID NO: 1153.

The term “nonpsychoactive” refers hereinafter to products or compositions or elements or components of Cannabis not significantly affecting the mind or mental processes.

The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids.

The main cannabinoids are concentrated in a viscous resin produced in structures known as glandular trichomes. Up until now, at least 113 different cannabinoids have been isolated from the Cannabis plant. The main classes of cannabinoids from Cannabis are THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran) and any combination thereof.

The best studied cannabinoids include tetrahydrocannabinol (THC), cannabidiol (CBD) and cannabinol (CBN).

Reference is now made to Tetrahydrocannabinol (THC), the primary psychoactive component of the Cannabis plant. Delta-9-tetrahydrocannabinol (Δ9-THC, THC) and delta-8-tetrahydrocannabinol (Δ8-THC), through intracellular CB1 activation, induce anandamide and 2-arachidonoylglycerol synthesis produced naturally in the body and brain. These cannabinoids produce the effects associated with Cannabis by binding to the CB1 cannabinoid receptors in the brain. Tetrahydrocannabinolic acid (THCA, 2-COOH-THC; conjugate base tetrahydrocannabinolate) is a precursor of tetrahydrocannabinol (THC), the active component of cannabis. THCA is found in variable quantities in fresh, undried cannabis, but is progressively decarboxylated to THC with drying, and especially under intense heating such as when cannabis is smoked or cooked into cannabis edibles. In the context of the present invention, the term THC also refers to THCA and vice versa.

Reference is now made to Cannabidiol (CBD) which is considered as non-psychotropic. Cannabidiol has little affinity for CB1 and CB2 receptors but acts as an indirect antagonist of cannabinoid agonists. It is further acknowledged herein that it is an antagonist at the putative cannabinoid receptor, GPR55, a GPCR expressed in the caudate nucleus and putamen. Cannabidiol has also been shown to act as a 5-HT1A receptor agonist. Cannabis produces CBD-carboxylic acid through the same metabolic pathway as THC, until the next to last step, where CBDA synthase performs catalysis instead of THCA synthase. CBDA is converted into CBD by decarboxylation. In the context of the present invention, the term CBD also refers to CBDA and vice versa.

CBD shares a precursor with THC and is the main cannabinoid in CBD-dominant Cannabis strains.

In the context of the current invention, enzymes within the biosynthetic pathway of THC and/or CBD, especially THCAS and/or CBDAS (depicted in FIG. 2), is down regulated to control and the content of THCA (and/or THC) and/or CBDA (or CBD) in the Cannabis plant.

Provided herein are methods for modifying the content of THC and/or THCA and/or CBDA and/or CBD compound of a Cannabis plant, comprising, consisting essentially of, or consisting of introducing one or more nucleotide modifications, through targeted DNA modification at a genomic locus of the plant, preferably at the regulatory region operably linked to at least one THCAS and/or CBDAS gene variant coding region.

As used herein, the term “modifying” or “modulation,” or the like, refers to any detectable change in the genotype and/or phenotype of a plant, as compared to a control plant (e.g., a wild-type plant that does not comprise the DNA modification).

The term “altered” as used herein generally means to become different, changed or modified in some particular or trait, or in other words, to cause the characteristics of something to change. In the context of the present invention it means to reduce (decrease) or increase (elevate) THC or THCA and/or CBD or CBDA content in a Cannabis plant by targeted genome modification, as compared to a control Cannabis plant lacking the genomic modification and having a similar genotype or genetic background or chemotype.

As used herein the term “genetic modification” or “genome modification” or “genomic modification” or “nucleotide modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Cannabis plants with altered cannabinoid content traits are generated using genome editing mechanism. This technique enables to achieve in planta modification of specific genes that control the biosynthesis of main cannabinoids, namely, THC and/or CBD in the Cannabis plant.

The term “genome editing”, or “genome/genetic modification” or “genome engineering” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:

### Genome Editing Glossary:

| | |
|---|---|
| Cas = CRISPR-associated genes | Indel = insertion and/or deletion |
| Cas9, Csn1 = a CRISPR-associated protein | NHEJ = Non-Homologous End Joining |
| containing two nuclease domains, that is | PAM = Protospacer-Adjacent Motif |
| programmed by small RNAs to cleave DNA | RuvC = an endonuclease domain named for |
| crRNA = CRISPR RNA | an E. coli protein involved in DNA repair |
| dCAS9 = nuclease-deficient Cas9 | sgRNA = single guide RNA |
| DSB = Double-Stranded Break | tracrRNA, trRNA = trans-activating crRNA |
| gRNA = guide RNA | TALEN = Transcription-Activator Like |
| HDR = Homology-Directed Repair | Effector Nuclease |
| HNH = an endonuclease domain named | ZFN = Zinc-Finger Nuclease |
| for characteristic histidine and asparagine | |
| residues | |

According to aspects of the present invention, the CRISPR (Clustered Regularly Interspaced Short Palindromic Repeats) and CRISPR-associated (Cas) genes are used for the first time for generating genome modification targeted at regulatory sites, e.g. promoter, non-coding sites of genes in the Cannabis plant.

According to further aspects of the invention, Cas protein, such as Cas9 (also known as Csn1) is required for gene silencing. Cas9 participates in the processing of CRISPR RNA (crRNAs), and is responsible for the destruction of the target DNA. To achieve site-specific DNA recognition and cleavage, Cas9 is complexed with both a crRNA and a separate trans-activating crRNA (tracrRNA or trRNA), that is partially complementary to the crRNA.

Without wishing to be bound by theory, it is herein acknowledged that during the destruction of target DNA, nuclease domains cut both DNA strands, generating double-stranded breaks (DSBs) at sites defined by a 20-nucleotide target sequence within an associated crRNA transcript. It is further noted that the double-stranded endonuclease activity of Cas9 also requires that a short conserved sequence, (2-5 nts) known as protospacer-associated motif (PAM), follows immediately 3′-of the crRNA complementary sequence.

20 According to further aspects of the invention, a two-component system may be used by the current invention, combining trRNA and crRNA into a single synthetic single guide RNA (sgRNA) for guiding targeted gene or genomic locus alterations.

It is further within the scope that Cas9 nuclease variants include wild-type Cas9, Cas9D10A and nuclease-deficient Cas9 (dCas9).

According to further aspects of the present invention, non-limiting examples of Cas genes or proteins are selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, Cu1966, bacteriophages Cas such as CasΦ (to (Cas-phi), and any combination thereof.

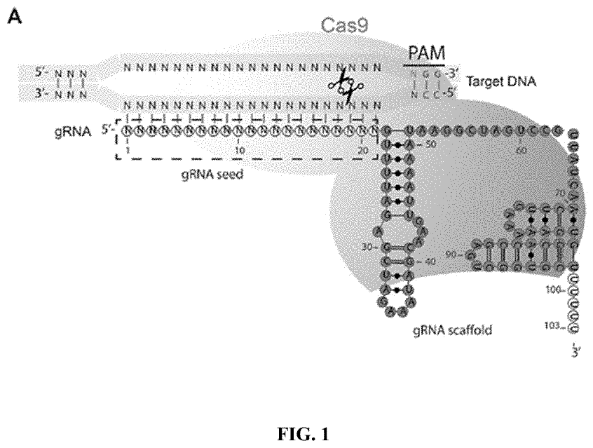

Reference is now made to FIG. 1 schematically presenting an example of CRISPR/Cas9 mechanism of action as depicted by Xie, Kabin, and Yinong Yang. “RNA-guided genome editing in plants using a CRISPR-Cas system.” Molecular plant 6.6 (2013): 1975-1983. As shown in this figure, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

TAL effector nucleases (TALEN) are a class of sequence-specific nucleases that can be used to make double-strand breaks at specific target sequences in the genome of a plant or other organism.

Endonucleases are enzymes that cleave the phosphodiester bond within a polynucleotide chain. Endonucleases include restriction endonucleases, which cleave DNA at specific sites without damaging the bases, and meganucleases, also known as homing endonucleases, which like restriction endonucleases, bind and cut at a specific recognition site, however the recognition sites for meganucleases are typically longer, about 18 bp or more.

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes. The naming convention for meganuclease is similar to the convention for other restriction endonuclease.

One step in the recombination process involves polynucleotide cleavage at or near the recognition site. The cleaving activity can be used to produce a double-strand break.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. ZFNs include an engineered DNA-binding zinc finger domain linked to a non-specific endonuclease domain. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. Examples of a Cas endonuclease of the disclosure includes a Cas9 protein, a Cpf1 protein, a C2c1 protein, a C2c2 protein, a C2c3 protein, Cas3, Cas 5, Cas7, Cas8, Casio, or complexes of these.

The commonly-used Cas9 from Streptococcus pyogenes (SpCas9) recognizes the PAM sequence 5′-NGG-3′ (where “N” can be any nucleotide base).

Other Cas variants and their PAM sequences (5′ to 3′) within the scope of the current invention include NmeCas9 (isolated from Neisseria meningitides) recognizing NNNNGATT, StCas9 (isolated from Streptococcus thermophiles) recognizing NNAGAAW, TdCas9 (isolated from Treponema denticola) recognizing NAAAAC and SaCas9 (isolated from Staphylococcus aureus) recognizing NNGRRT or NGRRT or NGRRN.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any CRISPR system such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component.

A guide polynucleotide/Cas endonuclease complex can cleave one or both strands of a DNA target sequence.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

The guide polynucleotide can also be a single molecule (also referred to as single guide polynucleotide) comprising a crNucleotide sequence linked to a tracrNucleotide sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides).

The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site.

The terms “single guide RNA” or “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA” or “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease” are used interchangeably herein and refer to at least one RNA component preferably with at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the known CRISPR systems such as a type I, II, or III CRISPR system. A guide RNA/Cas endonuclease complex can comprise a Type II Cas9 endonuclease and at least one RNA component (e.g., a crRNA and tracrRNA, or a gRNA).

The guide polynucleotide of the methods and compositions described herein may be any polynucleotide sequence that targets the genomic loci of a plant cell comprising a polynucleotide having a nucleic acid sequence that is at least 75% (e.g., 80%, 85% 90%, 91%, 92%, 93%, 94%, 95%, 96%, 97%, 98%, 99%, or 100%) similar or identical to a sequence selected from the group consisting of SEQ ID NOs: 1-6 and SEQ ID NOs: 1150-1153.

In certain embodiments, the guide polynucleotide is a guide RNA (gRNA). The gRNA that targets SEQ ID NO: 1 comprises at least 90% similarity to a nucleotide sequence selected from the group consisting of SEQ ID NO:7-91.

According to further embodiments, the gRNA that targets SEQ ID NO: 2 comprises at least 90% similarity to a nucleotide sequence selected from the group consisting of SEQ ID NO:92-176.

According to further embodiments, the gRNA that targets SEQ ID NO: 3 comprises at least 90% similarity to a nucleotide sequence selected from the group consisting of SEQ ID NO:177-278.

According to further embodiments, the gRNA that targets SEQ ID NO: 4 comprises at least 90% similarity to a nucleotide sequence selected from the group consisting of SEQ ID NO:279-419.

According to further embodiments, the gRNA that targets SEQ ID NO: 5 comprises at least 90% similarity to a nucleotide sequence selected from the group consisting of SEQ ID NO:420-560.

According to further embodiments, the gRNA that targets SEQ ID NO: 6 comprises at least 90% similarity to a nucleotide sequence selected from the group consisting of SEQ ID NO:561-681.

According to further embodiments, the gRNA that targets SEQ ID NO: 1150 comprises at least 90% similarity to a nucleotide sequence selected from the group consisting of SEQ ID NO:682-794.

According to further embodiments, the gRNA that targets SEQ ID NO: 1151 comprises at least 90% similarity to a nucleotide sequence selected from the group consisting of SEQ ID NO:795-895.

According to further embodiments, the gRNA that targets SEQ ID NO: 1152 comprises at least 90% similarity to a nucleotide sequence selected from the group consisting of SEQ ID NO:896-1016.

According to further embodiments, the gRNA that targets SEQ ID NO: 1153 comprises at least 90% similarity to a nucleotide sequence selected from the group consisting of SEQ ID NO:1017-1149.

The guide polynucleotide can be introduced into a cell transiently, as single stranded polynucleotide or a double stranded polynucleotide, using any method known in the art such as, but not limited to, particle bombardment or Agrobacterium transformation. The guide polynucleotide can also be introduced indirectly into a cell by introducing a recombinant DNA molecule (via methods such as, but not limited to, particle bombardment or Agro bacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter.

The terms “target site”, “target sequence”, “target site sequence”, target DNA″, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length.

Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an endonuclease (e.g. Cas). Assays to measure the single or double-strand break of a target site by an endonuclease are known in the art and generally measure the overall activity and specificity of the agent on DNA substrates containing recognition sites.

The term “protospacer adjacent motif” or “PAM” as used herein refers hereinafter to a short (e.g. 2-6 base pair) adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

It is within the scope of the present invention that gRNA sequences complementary or corresponding to the target sequence to be modified comprises a 3′ Protospacer Adjacent Motif (PAM) selected from the group consisting of NGG (SpCas), NNNNGATT (NmeCas9), NNAGAAW (StCas9), NAAAAC (TdCas9) and NNGRRT (SaCas9).

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

A “genomic locus” of a plant as used herein, generally refers to the location on a chromosome of the plant where a gene, such as a polynucleotide involved in THCA synthesis (THCAS) and/or CBDA synthesis (CBDAS), is found. It is within the scope of the current invention that the genetic locus includes the coding sequence of a gene and the regulatory regions (non-coding sequences) located upstream (e.g. promoter sequences or elements) and/or downstream (e.g. terminator sequences or elements) to the coding region and controlling its expression, namely transcription and/or translation. As used herein, “gene” includes a nucleic acid fragment that expresses or encodes a functional molecule such as, but not limited to, a specific protein coding sequence, and regulatory elements, regions or sequences, such as those preceding (5′ non-coding sequences) and following (3′ non-coding sequences) the coding sequence.

In general, a locus is the specific physical location of a gene or other DNA sequence on a chromosome. The plural of locus is “loci”.

According to some aspects of the invention, a functional locus, as used herein means the whole set of genomic regions that are alternatively used to carry out the same function. Regulatory regions may be also an example of different DNA regions belonging to the same functional locus. Different elements such as promoters and enhancers regulate gene expression, and a given gene may be under the control of multiple of such regulatory elements.

According to further aspects of the present invention, the promoter sequence includes the region upstream to 5′UTR, preceding the first exon of the open reading frame (ORF) or CDS.

A “regulatory region” or “regulatory element” or “regulatory sequence” generally refers to a transcriptional regulatory element involved in regulating the transcription of a nucleic acid molecule such as a gene or a target gene. The regulatory region or element comprises nucleic acids and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located. According to further aspects, a regulatory region is operably linked to the coding region of a gene and controlling its expression (transcription and/or translation).

A regulatory sequence is a segment of a nucleic acid molecule which is capable of increasing or decreasing the expression of specific genes within an organism.

An “enhancer” element is any nucleic acid molecule that increases transcription of a nucleic acid molecule when functionally linked to a promoter regardless of its relative position.

It is noted that the sequence of a regulatory region or element may be less conserved among different variants or alleles of the same gene.

A “repressor” (also herein referred to as silencer) is defined as any nucleic acid molecule which inhibits the transcription when functionally linked to a promoter regardless of relative position.

A “promoter” generally refers to a nucleic acid fragment capable of controlling transcription of another nucleic acid fragment. A promoter generally includes a core promoter (also known as minimal promoter) sequence that includes a minimal regulatory region to initiate transcription that is a transcription start site. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs (e.g. 5′ untranslated region, also known as a leader sequence or leader RNA, directly upstream from the initiation codon.). According to some aspects of the invention, a regulatory region comprises a transcription factor binding site, an RNA polymerase binding site, a TATA box, or a combination of structural variations thereof.

The term “cis-element” or “cis-regulatory region” or “cis-regulatory element (CRE)” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription. Cis-regulatory elements (CREs) are regions of non-coding DNA which regulate the transcription of neighboring genes.

An “intron” is an intervening sequence in a gene that is transcribed into RNA but is then excised in the process of generating the mature mRNA. The term is also used for the excised RNA sequences.

An “exon” is a portion of the sequence of a gene that is transcribed and is found in the mature messenger RNA derived from the gene but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” generally refers to a product resulting from RNA polymerase-catalyzed transcription of a DNA sequence. When an RNA transcript is a perfect complimentary copy of a DNA sequence, it is referred to as a primary transcript or it may be a RNA sequence derived from posttranscriptional processing of a primary transcript and is referred to as a mature RNA. “Messenger RNA” (“mRNA”) generally refers to RNA that is without introns and that can be translated into protein by the cell. “cDNA” generally refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA generally refers to RNA transcript that includes mRNA and so can be translated into protein within a cell or in vitro. “Antisense RNA” generally refers to a RNA transcript that is complementary to all or part of a target primary transcript or mRNA and that blocks expression or transcripts accumulation of a target gene. The complementarity of an antisense RNA may be with any part of the specific gene transcript, i.e. at the 5′ non-coding sequence, 3′ non-coding sequence, introns, or the coding sequence. “Functional RNA” generally refers to antisense RNA, ribozyme RNA, or other RNA that may not be translated but yet has an effect on cellular processes.

“Targeted DNA modification” can be used synonymously with targeted DNA mutation or targeted nucleotide modification and refers to the introduction of a site specific modification that alters or changes the nucleotide sequence at a specific genomic locus of the plant (e.g., Cannabis).

In certain embodiments, the targeted DNA modification occurs at a genomic locus that comprises a regulatory region (e.g. promoter region) involved in controlling expression of THCAS and/or CBDAS polynucleotide variant or homologue and thus affecting THCA and/or CBDA content or concentration in the Cannabis plant.

According to main embodiments, the THCAS polynucleotide variant or homologue comprises “Finola” (FN) variety FNTHCAS-1 and FNTHCAS-2 genes, and Purple Kush (PK) strain PKTHCAS-1, PKTHCAS-2, PKTHCAS-3 and PKTHCAS-4 genes.

In certain embodiments, the polynucleotide sequence of the regulatory regions operably linked to the THCAS genes FNTHCAS-1, FNTHCAS-2, PKTHCAS-1, PKTHCAS-2, PKTHCAS-3 and PKTHCAS-4 comprising a nucleic acid sequence that is at least 75% (e.g., 80%, 85%, 90%, 91%, 92%, 93%, 94%, 95%, 96%, 97%, 98%, 99%, or 100%) similar or identical to a nucleic acid sequence selected from the group consisting of SEQ ID NOs: 1-6, respectively.

According to other main embodiments, the CBDAS polynucleotide variant or homologue comprises Cannabidiolic acid synthase-like 1 (CBDAS2), “Finola” (FN) variety FNCBDAS gene, and Purple Kush (PK) strain PKCBDAS and PKCBDAS1 genes.

In certain embodiments, the polynucleotide sequence of the regulatory regions operably linked to the CBDAS genes selected from Cannabidiolic acid synthase-like 1 (CBDAS2), “Finola” (FN) variety FNCBDAS gene, and Purple Kush (PK) strain PKCBDAS and PKCBDAS1, comprising a nucleic acid sequence that is at least 75% (e.g., 80%, 85%, 90%, 91%, 92%, 93%, 94%, 95%, 96%, 97%, 98%, 99%, or 100%) similar or identical to a nucleic acid sequence selected from the group consisting of SEQ ID NOs: 1150-1153, respectively.

Sequence alignments and percent identity or similarity calculations may be determined using a variety of comparison methods and bioinformatics computing methods designed to detect similar or identical sequences, known to the skilled person in the relevant field.

In one embodiment the sequence identity or similarity percentage (%) is determined over the entire length of the molecule (nucleotide or amino acid).

The targeted DNA or genome modification described herein may be any modification known in the art such as, for example, insertion, deletion or indel, single nucleotide polymorphism (SNP), and or a polynucleotide modification. Additionally, the targeted DNA modification in the genomic locus may be located anywhere in the genomic locus, such as, for example, a coding region of the encoded polypeptide (e.g., exon), a non-coding region (e.g., intron), a regulatory element, or untranslated region. In preferred embodiments, the modification is in a regulatory element or region controlling a gene targeted for down regulation, knockdown or silencing, knockout mutation, a loss of function mutation or any combination thereof.

The specific location of the targeted DNA modification within the regulatory region polynucleotide of the THCAS and/or CBDAS gene is not particularly limited, as long as the targeted DNA modification results in reduced expression or activity of the protein encoded by the THCAS and/or CBDAS gene. In certain embodiments the targeted DNA modification is a deletion of one or more nucleotides, preferably contiguous, of the genomic locus.

As used herein “reduced”, “reduction”, “decrease” or the like refers to any detectable decrease in an experimental group (e.g., Cannabis plant with a targeted DNA modification described herein) as compared to a control group (e.g., wild-type Cannabis plant, preferably of similar genetic background, that does not comprise the targeted DNA modification).

Accordingly, reduced expression of a gene or protein comprises any detectable decrease in the total level of the RNA or protein in a sample and can be determined using routine methods in the art such as, for example, PCR, Northern blot, Western blotting, ELISA as well as methods described in Rodriguez-Leal, Daniel, et al. “Engineering quantitative trait variation for crop improvement by genome editing.” Cell 171.2 (2017): 470-480, which is incorporated herein by reference.

In certain embodiments, a reduction in the expression or activity of the THCAS and/or CBDAS polynucleotide is due to a targeted DNA modification at regulatory region operably linked to the THCAS and/or CBDAS coding region that results in one or more of the following:
a) reduced expression of the THCAS and/or CBDAS polynucleotide;
b) generation of one or more alternatively spliced transcripts of the THCAS and/or CBDAS polynucleotide;
c) frameshift mutation in one or more exons of the THCAS and/or CBDAS polynucleotide;
d) deletion of a substantial portion of the THCAS and/or CBDAS polynucleotide or deletion of the full open reading frame of the THCAS and/or CBDAS polynucleotide;
e) repression of an enhancer motif present within the regulatory region encoding the THCAS and/or CBDAS polynucleotide; and/or
f) modification of one or more nucleotides or deletion of a regulatory element operably linked to the expression of the THCAS and/or CBDAS polynucleotide wherein the regulatory element is present within a promoter, intron, 3′UTR, terminator or a combination thereof.

In certain embodiments, the targeted DNA modification at a genomic locus involved in THCAS and/or CBDAS expression results in Cannabis plants that exhibit reduced THCA or THC and/or CBDA or CBD content, or free of THCA and/or THC and/or CBDA and/or CBD compared to or relative to comparable control plants lacking the targeted DNA modification. Such comparable control plants may be of a similar genotype or chemotype or genetic background, but lacking the at least one targeted nucleotide modification.

For example, in certain embodiments, the modified Cannabis plant has THCA and/or THC and/or CBDA and/or CBD content of up to 30% by weight, particularly between about 0% to about 30% by weight, more particularly between about 0.3% to about 30%, even more particularly between about 0.3% to about 10% by weight.

In other embodiments, the targeted DNA modification results in Cannabis plants that exhibit a THCA and/or THC and/or CBDA and/or CBD content of not more than about 0.3% by weight.

In yet other embodiments, the targeted DNA modification results in THCAS and/or CBDAS promoter region results in THCA and/or CBDA free Cannabis plant.

In certain embodiments, the regulatory region within THCAS and/or CBDAS genomic locus has more than one (e.g., 2, 3, 4, 5, 6, 7, 8, 9, or 10) targeted DNA modification.

In certain embodiments, the plant may have targeted DNA modifications at more than one (e.g., 2, 3, 4, 5, 6, 7, 8, 9, or 10) genomic loci that are involved in regulation of THCAS and/or CBDAS expression in the plant (e.g., Cannabis).

The targeted DNA modification of the genomic locus may be done using any genome modification technique known in the art. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, bioloistics and transformation by Agrobacterium and viral based vectors.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished.

The polynucleotide modification template may be present transiently in the cell or it can be introduced via a viral replicon.

A “modified nucleotide” or “edited nucleotide” or “genome modification” refers to a nucleotide sequence of interest that comprises at least one alteration when compared to its non-modified nucleotide sequence form, for example in a control Cannabis plant which does not comprise the alternation. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP). Transformation can be performed using a DNA plasmid such as a plant codon optimized Streptococcus pyogenes Cas9 (pcoSpCas9) plasmid. The plasmid contains the plant codon optimized SpCas9 and the above mentioned at least one sgRNA. DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein-RNP's) in order to bypass the need for in vivo transcription and translation of the Cas9+gRNA plasmid in planta to achieve gene editing. Insertion of the aforementioned plasmid DNA can be done, but is not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

The terms “targeting”, “gene targeting” and “DNA targeting” are used interchangeably herein. DNA targeting herein may be the specific introduction of a knock out, knock down, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method in which multiple different RNA components are provided, each designed to guide a guide polynucleotide/Cas endonuclease complex to a unique DNA target site.

The terms “knock-out”, “gene knock-out” and “genetic knock-out” are used interchangeably herein. A knock-out represents a DNA sequence of a cell that has been rendered partially or completely inoperative by targeting with a guide polynucleotide/endonuclease complex such as Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence e.g. through NHEJ), or by specific removal of sequence that reduces or completely destroys the function of sequence at or near the targeting site.

The guide polynucleotide/Cas endonuclease system can be used in combination with a co-delivered polynucleotide modification template to allow for editing (modification) of a genomic nucleotide sequence of interest.

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA or at the level of protein. It is within the scope of the present invention that the term gene knockdown also refers to a loss of function mutation and/or gene knockout mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonyms of the term included within the scope of the present invention is null mutation.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term “genotype” or “genetic background” refers hereinafter to the genetic constitution of a cell or organism. An individual's genotype includes the specific alleles, for one or more genetic marker loci, present in the individual's haplotype. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest. Thus, in some embodiments a genotype comprises a summary of one or more alleles present within an individual at one or more genetic loci. In some embodiments, a genotype is expressed in terms of a haplotype. It further refers to any inbreeding group, including taxonomic subgroups such as subspecies, taxonomically subordinate to species and superordinate to a race or subrace and marked by a pre-determined profile of latent factors of hereditary traits.

The term “orthologue” as used herein refers hereinafter to one of two or more homologous gene sequences found in different species.

The term “functional variant” or “functional variant of a nucleic acid or amino acid sequence” as used herein, for example with reference to SEQ ID NOs: 1-6 and 1150-1153 refers to a variant of a sequence or part of a sequence which retains the biological function of the full non-variant allele and hence has the activity of the expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

The term “homology” is meant DNA sequences that are similar. For example, a “region of homology to a genomic region” is a region of DNA that has a similar sequence to a given “genomic region” in the cell or organism genome. A region of homology can be of any length that is sufficient to promote homologous recombination at the cleaved target site, such that the region of homology has sufficient homology to undergo homologous recombination with the corresponding genomic region. “Sufficient homology” indicates that two polynucleotide sequences have sufficient structural similarity to act as substrates for a homologous recombination reaction or o have the same function. The structural similarity includes overall length of each polynucleotide fragment, as well as the sequence similarity of the polynucleotides.

“Sequence similarity” can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range. The amount of homology can also be described by percent sequence identity over the full aligned length of the two polynucleotides which includes percent sequence identity (or similarity) of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology-Hybridization with Nucleic Acid Probes, (Elsevier, New York).

The structural similarity between a given genomic region and the corresponding region of homology (e.g. found on the donor DNA) can be any degree of sequence identity that allows for homologous recombination to occur. For example, the amount of homology or sequence identity shared by the “region of homology” a corresponding DNA (e.g. of the donor DNA) and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

As used herein, “homologous recombination” includes the exchange of DNA fragments between two DNA molecules at the sites of homology.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) plant. Specifically, the endogenous nucleic acid sequences of the promoter or regulatory regions of one or more of Cannabis tetrahydrocannabinolic acid synthase (CsTHCAS) homologues or variants, particularly pFNTHCAS-1 having a nucleic acid sequence as set forth in SEQ ID NO:1 or a functional variant thereof, pFNTHCAS-2 having a nucleic acid sequence as set forth in SEQ ID NO:2 or a functional variant thereof, pPKTHCAS-1 having a nucleic acid sequence as set forth in SEQ ID NO:3 or a functional variant thereof, pPKTHCAS-2 having a nucleic acid sequence as set forth in SEQ ID NO:4 or a functional variant thereof, pPKTHCAS-3 having a nucleic acid sequence as set forth in SEQ ID NO:5 or a functional variant thereof, pPKTHCAS-4 having a nucleic acid sequence as set forth in SEQ ID NO:6 or a functional variant thereof and any combination thereof, have been altered compared to wild type sequences using mutagenesis and/or genome editing methods as described herein. This causes inactivation of at least one of these endogenous genes and thus downregulate their function in production of THCA and/or THC.

In other embodiments of the present invention, the endogenous nucleic acid sequences of the promoter or regulatory regions of one or more of Cannabis cannabidiolic acid synthase (CsCBDAS) homologues or variants, particularly pCBDAS2 having a nucleic acid sequence as set forth in SEQ ID NO:1150 or a functional variant thereof, pFNCBDAS having a nucleic acid sequence as set forth in SEQ ID NO:1151 or a functional variant thereof, pPKCBDAS having a nucleic acid sequence as set forth in SEQ ID NO:1152 or a functional variant thereof and pPKCBDAS1 having a nucleic acid sequence as set forth in SEQ ID NO:1153 or a functional variant thereof, have been altered compared to wild type sequences using mutagenesis and/or genome editing methods as described herein. This causes inactivation of at least one of these endogenous genes and thus downregulate their function in production of CBDA and/or CBD.

Such plants have an altered cannabinoid profile which may be suitable for treatment of different medical conditions or diseases. Therefore, the cannabinoid profile is affected by the presence of at least one mutated endogenous cannabinoid biosynthesis enzyme gene (e.g. THCAS and/or CBDAS) in the Cannabis plant genome which has been specifically targeted using genome editing techniques.

As used herein, the term “cannabinoid biosynthesis enzyme” refers to a protein acting as a catalyst for producing one or more cannabinoids in a plant of genus Cannabis.

Examples of cannabinoid biosynthesis enzymes within the context of this disclosure include, but are not limited to: tetrahydrocannabinolic acid synthase (THCAS), cannabidiolic acid synthase (CBDAS), aromatic prenyltransferase (PT), olivetol synthase (OLS), acyl-activating enzyme 1 (AAE1), polyketide synthase (PKS), olivetolic acid cyclase (OAC), tetraketide synthase (TKS), type III PKS, chalcone synthase (CHS), prenyltransferase, CBCA synthase, GPP synthase, FPP synthase, Limonene synthase, aromatic prenyltransferase, and geranylphosphate: olivetolate geranyltrasferase.

In certain embodiments of the present invention, the promoter region controlling expression of THCAS variants of “Finola” cultivar, i.e. FNTHCAS-1 and FNTHCAS-2, as well as of “Purple Kush” strain, i.e. PKTHCAS-1, PKTHCAS-2, PKTHCAS-3 and PKTHCAS-4 have been modified by targeted genome modification to downregulate their expression.

In certain other embodiments of the present invention, the promoter region controlling expression of CBDAS variants of cannabidiolic acid synthase-like 1 (CBDAS2), “Finola” cultivar, i.e. FNCBDAS, as well as of “Purple Kush” strain, i.e. PKCBDAS and PKCBDAS1 have been modified by targeted genome modification to downregulate their expression.

Disclosed herein, is a method of controlling THCA and/or CBDA synthesis in a plant of genus Cannabis. In some embodiments, the method comprising: Manipulating expression of a gene coding for the cannabinoid biosynthesis enzyme THCAS and/or CBDAS by targeting its promoter region controlling its expression using CRISPR/cas system with suitable corresponding gRNA sequences.

As used herein, the term “controlling” refers to directing, governing, steering, and/or manipulating, specifically reducing, decreasing or down regulating or silencing the amount of a cannabinoid or cannabinoids produced in a plant of genus Cannabis. In one embodiment, controlling means affecting the expression of a coding region of a gene using cis and/or trans genomic elements, particularly, causing loss of function or down regulation of the gene's function. In other embodiments, controlling means inducing, increasing, enhancing or elevating the amount or expression of genes encoding a cannabinoid or cannabinoids produced in a plant of genus Cannabis.

According to further aspects, controlling comprises modifying a plant of genus Cannabis to produce an unnaturally occurring concentration of a first cannabinoid, e.g. THCA and/or CBDA. In one embodiment, controlling comprises modifying a plant of genus Cannabis to produce an unnaturally occurring ratio of a first cannabinoid, e.g. THCA and/or CBDA to the other cannabinoids.

As used herein, the term “expression of a gene” or “gene expression” refers hereinafter to a plant's ability to utilize information from genetic material for producing functional gene products. Within the context of this disclosure, expression is meant to encompass the plant's ability to produce proteins, such as enzymes, and various other molecules from the plant's genetic material. In one embodiment, the plant expresses mutated or modified cannabinoid biosynthesis enzymes for cannabinoid biosynthesis. It is further within the scope that it refers to transcription (RNA) or translation (protein) levels of gene expression.

In the context of the present invention, a regulatory sequence (e.g. promoter region), targeted by genome modification, is capable of increasing or decreasing the expression of specific genes, e.g. THCAS and/or CBDAS gene expression.

As used herein, the term “manipulating expression of a gene” refers in the context of the present invention, to intentionally changing the genome of a plant of genus Cannabis, within regulatory regions of certain genes, to control the expression of certain features or characteristics of the plant.

In one embodiment, the plant's genome is manipulated to express less THCA synthase and/or CBDA synthase by mutating the promoter region operably linked to the gene using genome modification.

As used herein, the term “coding” refers to storing genetic information and accessing the genetic information for producing functional gene products.

According to further aspects of the present invention, the altered THC and/or CBD content trait is not conferred by the presence of transgenes expressed in Cannabis.

Cannabis plants of the invention are modified plants, compared to wild type plants, which comprise and express at least one mutant Cannabis tetrahydrocannabinolic acid synthase (CsTHCAS) and/or cannabidiolic acid synthase (CBDAS) allele.

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art.

Recombinant DNA constructs comprising one or more of the polynucleotide sequences set forth in SEQ ID NOs: 1-6, SEQ ID NOs: 1150-1153 and SEQ ID NOs: 7-1149 are provided herein.

Also provided are plants, plant cells, and/or seeds introduced with a polynucleotide described herein. In certain embodiments the plant, plant cell, or seed comprises a recombinant DNA construct comprising one or more of the polynucleotide sequences set forth in SEQ ID NOs: 1-1153.

In certain embodiments, the plant, plant cell, or seed comprises a recombinant DNA construct comprising one or more guide polynucleotides comprising a nucleic acid sequence selected from the group consisting of SEQ ID NO: 7-1149, that target the genomic loci of a plant cell comprising a polynucleotide sequence that is at least 75% (e.g., 80%, 85%, 90%, 91%, 92%, 93%, 94%, 95%, 96%, 97%, 98%, 99%, or 100%) identical to a sequence selected from the group consisting of SEQ ID NOs: 1-6 and SEQ ID NOs: 1150-1153.

The polynucleotide of the plant, plant cell, or seed can be stably introduced or can be transiently expressed by the plant, plant cell, or seed. In certain embodiments, the polynucleotide is stably introduced into the plant, plant cell, or seed.

The terms “polynucleotide”, “polynucleotide sequence”, “nucleic acid sequence”, “nucleotide sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof.

The term “recombinant DNA construct” or “recombinant expression construct” is used interchangeably and generally refers to a discrete polynucleotide into which a nucleic acid sequence or fragment can be moved. Preferably, it is a plasmid vector or a fragment thereof comprising the promoters of the present disclosure. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or viral nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The promoters for use in the vector may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Core promoters are often modified to produce artificial, chimeric, or hybrid promoters, and can further be used in combination with other regulatory elements, such as cis-elements, 5′UTRs, enhancers, or introns, that are either heterologous to an active core promoter or combined with its own partial or complete regulatory elements. In certain embodiments the promoter of the recombinant DNA construct may be a tissue-specific promoter, developmental regulated promoter, or a constitutive promoter.

“Tissue-specific promoter” and “tissue-preferred promoter” are used interchangeably to refer to a promoter that is expressed predominantly but not necessarily exclusively in one tissue or organ, but that may also be expressed in one specific cell. “Developmentally regulated promoter” generally refers to a promoter whose activity is determined by developmental events. “Constitutive promoter” generally refers to promoters active in all or most tissues or cell types of a plant at all or most developing stages. As with other promoters classified as “constitutive”, some variation in absolute levels of expression can exist among different tissues or stages. The term “constitutive promoter” or “tissue-independent” are used interchangeably herein.

In certain embodiments the promoter of the recombinant DNA construct is heterologous to the expressed nucleotide sequence. A “heterologous nucleotide sequence” generally refers to a sequence that is not naturally occurring with the sequence of the disclosure. While this nucleotide sequence is heterologous to the sequence, it may be homologous, or native, or heterologous, or foreign, to the plant host. However, it is recognized that the instant sequences may be used with their native coding sequences to increase or decrease expression resulting in a change in phenotype in the transformed seed.

The terms “heterologous nucleotide sequence”, “heterologous sequence”, “heterologous nucleic acid fragment”, and “heterologous nucleic acid sequence” are used interchangeably herein.

The term “operably linked” or “functionally linked” generally refers to the association of nucleic acid sequences on a single nucleic acid fragment so that the function of one is affected by the other. In other aspects, the ability of a regulatory nucleic acid sequence to drive the expression of a coding sequence. For example, a promoter is operably linked with a coding sequence when it is capable of affecting the expression of that coding sequence (i.e. that the coding sequence is under the transcriptional control of the promoter). Coding sequences can be operably linked to regulatory sequences in sense or antisense orientation.

The terms “initiate transcription”, “initiate expression”, “control expression”, “drive transcription”, and “drive expression” are used interchangeably herein and all refer to the primary function of a promoter. As detailed in this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide. Thus the term “expression”, as used herein, generally refers to the production of a functional end-product e.g., an mRNA or a protein (precursor or mature).

The term “expression cassette” as used herein, generally refers to a discrete nucleic acid fragment into which a nucleic acid sequence or fragment can be cloned or synthesized through molecular biology techniques.

The expression cassette (e.g., CRISPR/RNA-guided endonuclease expression cassette such as a CRISPR/Cas9 expression cassette or a CRISPR/Cpf 1 expression cassette) may be introduced into a plant using any method known in the art or described herein, e.g., by such as Agrobacterium-mediated recombination, viral-vector mediated recombination, microinjection, gene gun bombardment/biolistic particle delivery, or electroporation of plant protoplasts. The expression cassette (e.g., CRISPR/RNA-guided endonuclease expression cassette such as a CRISPR/Cas9 expression cassette or a CRISPR/Cpf1 expression cassette) may be integrated onto the same chromosome or a different chromosome than the gene of interest. In some embodiments, integration of the expression cassette (CRISPR/RNA-guided endonuclease expression cassette such as a CRISPR/Cas9 expression cassette or a CRISPR/Cpf1 expression cassette) onto a different chromosome than the gene of interest is preferable so that the expression cassette can later be removed through a self-cross or a cross with another plant without having to undergo homologous recombination to separate the expression cassette from the gene of interest.

In some embodiments, the allele of the gene of interest contains the target region against which the gRNAs (e.g., sgRNAs) are designed such that mutations can be introduced into the target region of the allele using the RNA-guided endonuclease (e.g., Cas9, Cpf1, or Csm1 endonuclease). In some embodiments, the target region or a portion thereof, is part of a regulatory, non-coding region of the allele.

In some embodiments, the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene or allele of interest.

In some embodiments, the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene or allele of interest.

In some embodiments, the target region comprises a regulatory region of the gene or allele of interest, i.e. THCAS and/or CBDAS. As used herein, a “regulatory region” of a gene of interest contains one or more nucleotide sequences that, alone or in combination, are capable of modulating expression of the gene of interest. Regulatory regions include, for example, promoters, enhancers, terminators and introns. In some embodiments, the regulatory region comprises a transcription factor binding site, an RNA polymerase binding site, a TATA box, or a combination thereof. In some embodiments, the regulatory region is within a certain distance of the gene of interest, e.g., 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest, or 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest.

In some embodiments, a regulatory region may be identified using databases or other information available in the art.

In some embodiments, a regulatory region can be identified, e.g., by analyzing the sequences within a certain distance of the gene of interest (e.g., within 2-5 kilobases) for one or more of transcription factor binding sites, RNA polymerase binding sites, TATA boxes, reduced SNP density or conserved non-coding sequences.

In some embodiments, the target region may be larger, e.g., 0 to 100 kilobases (e.g., 0 to 100, 0 to 90, 0 to 80, 0 to 70, 0 to 60, 0 to 50, 0 to 40, 0 to 30, 0 to 20 or 0 to 10 kilobases) upstream of the 5′ end of the coding sequence of the gene of interest, or 0 to 60 kilobases (e.g., 0 to 60, 0 to 50, 0 to 40, 0 to 30, 0 to 20 or 0 to 10 kilobases) base pairs downstream of the 3′ end of the coding sequence of the gene of interest. Such larger regions may include both proximal promoter regions (e.g., within 1 to 3 Kb of the 5′ end of the coding sequence) and distal enhancer regions.

“Transformation” as used herein generally refers to both stable transformation and transient transformation. “Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms.

“Transient transformation” generally refers to the introduction of a nucleic acid fragment into the nucleus, or DNA-containing organelle, of a host organism resulting in gene expression without genetically stable inheritance.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The heterologous polynucleotide can be stably integrated within the genome such that the polynucleotide is passed on to successive generations. The heterologous polynucleotide may be integrated into the genome alone or as part of a recombinant DNA construct.

“Transient expression” generally refers to the temporary expression of often reporter genes such as b-glucuronidase (GUS), fluorescent protein genes ZS-GREEN1, ZS-YELLOW1 N1, AM-CYAN 1, DS-RED in selected certain cell types of the host organism in which the transgenic gene is introduced temporally by a transformation method. The transformed materials of the host organism are subsequently discarded after the transient gene expression assay.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

The plant, plant cell, and seed, of the compositions and methods described herein may further comprise a heterologous nucleic acid sequence that confers advantageous properties, such as improved agronomics, to the plant, plant cell, and/or seed. The heterologous nucleic acid sequences are known to those of ordinary skill in the art, and can be routinely incorporated in the plant, plant cell, and/or seeds described herein using routine methods in the art, such as those described herein.

In certain embodiments the heterologous nucleic acid sequence is selected from the group consisting of a reporter gene, a selection marker, a disease resistance gene, a herbicide resistance gene, an insect resistance gene, a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

In certain embodiments, the present disclosure contemplates the transformation of a recipient cell with more than one advantageous gene. Two or more genes can be supplied in a single transformation event using either distinct gene-encoding vectors, or a single vector incorporating two or more gene coding sequences. Any two or more genes of any description, such as those conferring herbicide, insect, disease (viral, bacterial, fungal, and nematode), or drought resistance, oil quantity and quality, or those increasing yield or nutritional quality may be employed as desired.

Main aspects of the invention involve targeted mutagenesis methods, specifically genome editing, and exclude embodiments that are solely based on generating plants by traditional breeding methods.

Described are polynucleotides as well as methods for modifying metabolite biosynthesis pathways in Cannabis plants and/or Cannabis plant cells, Cannabis plants and/or plant cells exhibiting modified metabolite biosynthesis pathways. In particular, described are methods for modifying production of THC and/or THCA, CBD and/or CBDA in Cannabis plants by modulating the expression and/or activity of at least one Cannabis tetrahydrocannabinolic acid synthase (CsTHCAS) and/or cannabidiolic acid synthase (CsCBDAS) gene and Cannabis plants having modified expression and/or activity of at least one of these genes/proteins.

Accordingly, in certain embodiments, the present invention provides methods of downregulating production of THC and/or THCA, CBD and/or CBDA. In particular embodiments, there is provided methods of downregulating expression and/or activity of Cannabis THCA synthase and/or CBDA synthase by CRISPR/Cas9 assisted targeted genome editing within the regulatory region controlling the expression of the gene. This strategy enables to knock-down expression of the target genes and to generate knockout allele variations directly in the elite target germplasm with minimal genetic drag associated with conventional breeding material.

Down regulation of key steps in metabolic pathway re-directs intermediates and energy to alternative metabolic pathways and results in increased production and accumulation or reduced production and elimination of other end products. THC, CBD and other Cannabis metabolites share a biosynthetic pathway; that cannabigerolic acid (CBGA) is a precursor of THC, CBD and Cannabichromene. In particular, THCA synthase catalyzes the production of delta-9-tetrahydrocannabinolic acid from cannabigerolic acid; delta-9-tetrahydrocannabinolic undergoes thermal conversion to form THC. CRISPR/Cas9 genome editing has been widely adopted in plants as a tool for understanding fundamental biological processes. With this disclosure it is demonstrated that genome editing is useful for agricultural applications. It is herein shown that by targeting the promoters of genes involved in cannabinoid biosynthesis, e.g. THCA synthase and/or CBDA synthase, Cannabis plants with altered (e.g. reduced) THCA and/or CBDA content could be achieved.

The approach of the current invention enable production by CRISPR/Cas9 gene editing regulatory mutations associated with phenotypic variation. Such streamlined trait improvement is expected for the gene targeted. The phenotypic variation achieved by engineering regulatory alleles for a single gene (i.e. THCAS and/or CBDAS) previously required multiple natural and induced mutations within the gene or several genes.

The effects may be dominant or semi dominant or co-dominant. There is also potential for engineering, not only loss of function mutations, but also gain-of-function alleles.

It is further acknowledged that breeders expend great time and effort to adapt beneficial allelic variants to diverse breeding germplasm. By the current invention this constraint is bypassed by directly generating and selecting for the most desirable regulatory variant in the context of modified THCA and/or CBDA content in the Cannabis plant.

As the medical Cannabis pharmaceutical industry is focusing on developing new cannabinoid based drugs, and these are mostly extracted from the Cannabis plant, there is a growing need for Cannabis plants bred for producing high levels of specific cannabinoids. In addition, there is a need for advanced breeding programs for food and fiber (Hemp) as well.

The present invention is aimed at enhancing cannabinoid breeding capabilities by using advanced molecular genome editing technologies in order to maximize the plants' phyto-chemical molecules production potential.

According to a further aspect of the present invention, a method or a tool is provided that enables the regulation in planta or the production of specific cannabinoid molecules.

It is further within the scope of the present invention to provide means and methods for in planta modification of specific genes' regulatory regions that relate to and/or control the cannabinoid biosynthesis pathways (as indicated in FIG. 2). More specifically, but not limited to, the present invention achieves the use of the CRISPR/Cas technology (see FIG. 1), such as, but not limited to Cas9 or Cpf1, in order to generate knockout alleles of the genes depicted in FIG. 2, rendering the enzymes inactive thereby controlling in planta the production of the resulting cannabinoid products depicted in FIG. 2.

According to a main embodiment, the present invention provides a Cannabis plant exhibiting reduced tetrahydrocannabinolic acid (THCA) content, wherein said plant comprises a modified genomic locus involved in tetrahydrocannabinolic acid synthase (THCAS) gene expression, said genomic locus comprises at least one targeted nucleotide modification as compared to a control Cannabis plant, within the regulatory region controlling said THCAS expression.

According to a further main embodiment, the present invention provides a Cannabis plant exhibiting reduced cannabidiolic acid (CBDA) content, wherein said plant comprises a modified genomic locus involved in cannabidiolic acid synthase (CBDAS) gene expression, said genomic locus comprises at least one targeted nucleotide modification as compared to a control Cannabis plant, within the regulatory region controlling said CBDAS expression.

According to a further embodiment of the present invention, cannabinoid biosynthesis enzyme Δ9-tetrahydrocannabinolic acid (THCA) synthase (THCAS) is down regulated using targeted genome modification in a regulatory region operably linked to the coding region of the gene (e.g. gene editing techniques as inter alia presented). As a result, the production of the major cannabinoid THCA (converted into THC by decarboxylation) is significantly reduced and/or totally abolished.

According to a further embodiment of the present invention, cannabinoid biosynthesis enzyme cannabidiolic acid (CBDA) synthase (CBDAS) is down regulated using targeted genome modification in a regulatory region operably linked to the coding region of the gene (e.g. gene editing techniques as inter alia presented). As a result, the production of the major cannabinoid CBDA (converted into CBD by decarboxylation) is significantly reduced and/or totally abolished.

According to a further embodiment of the present invention, targeted genome modification of the herein identified Cannabis gene encoding cannabinoid synthesis enzyme CsTHCAS results in reduced or no production of THCA and thus Cannabis plants with reduced content, or free of, THCA and/or THC are provided by the present invention.

According to a further embodiment of the present invention, the THCAS gene is a THCAS variant or homologue of ‘Finola’ (FN) Cannabis strain (FNTHCAS) or a THCAS variant or homologue of Purple Kush (PK) Cannabis strain (PKTHCAS).

According to a further embodiment of the present invention, the FNTHCAS homologue is selected from the group consisting of FNTHCAS-1 and FNTHCAS-2 genes and the PKTHCAS homologue is selected from the group consisting of PKTHCAS-1, PKTHCAS-2, PKTHCAS-3 and PKTHCAS-4 genes.

According to a further embodiment of the present invention, the regulatory region comprises a nucleotide sequence selected from the group consisting of pFNTHCAS-1 having a nucleic acid sequence as set forth in SEQ ID NO:1 or a functional variant thereof, pFNTHCAS-2 having a nucleic acid sequence as set forth in SEQ ID NO:2 or a functional variant thereof, pPKTHCAS-1 having a nucleic acid sequence as set forth in SEQ ID NO:3 or a functional variant thereof, pPKTHCAS-2 having a nucleic acid sequence as set forth in SEQ ID NO:4 or a functional variant thereof, pPKTHCAS-3 having a nucleic acid sequence as set forth in SEQ ID NO:5 or a functional variant thereof, pPKTHCAS-4 having a nucleic acid sequence as set forth in SEQ ID NO:6 or a functional variant thereof and any combination thereof.

According to a further embodiment of the present invention, the functional variant has at least 75% sequence identity or similarity to the nucleic acid sequence of said regulatory region sequence or a codon degenerate nucleotide sequence thereof.

According to a further embodiment of the present invention, targeted genome modification of the herein identified Cannabis gene encoding cannabinoid synthesis enzyme CsCBDAS results in reduced or no production of CBDA and thus Cannabis plants with reduced content, or free of, CBDA and/or CBD are provided by the present invention.

According to a further embodiment of the present invention, the CBDAS gene is a cannabidiolic acid synthase-like 1 (CBDAS2) variant or homologue, CBDAS variant or homologue of ‘Finola’ (FN) Cannabis strain (FNCBDAS) or a CBDAS variant or homologue of Purple Kush (PK) Cannabis strain (PKCBDAS or PKCBDAS1).

According to a further embodiment of the present invention, the regulatory region comprises a nucleotide sequence selected from the group consisting of pCBDAS2 having a nucleic acid sequence as set forth in SEQ ID NO:1150 or a functional variant thereof, pFNCBDAS having a nucleic acid sequence as set forth in SEQ ID NO:1151 or a functional variant thereof, pPKCBDAS having a nucleic acid sequence as set forth in SEQ ID NO:1152 or a functional variant thereof and pPKCBDAS1 having a nucleic acid sequence as set forth in SEQ ID NO:1153 or a functional variant thereof and any combination thereof.

According to a further embodiment of the present invention, the functional variant has at least 75% sequence identity or similarity to the nucleic acid sequence of said regulatory region sequence or a codon degenerate nucleotide sequence thereof.

According to a further embodiment of the present invention, the targeted nucleotide modification is selected from the group consisting of insertion, deletion, single nucleotide polymorphism (SNP), and a polynucleotide modification, such that the expression of the THCAS and/or CBDAS polynucleotide is reduced or affected.

According to a further embodiment of the present invention, the nucleotide modification is a missense mutation, nonsense mutation, insertion, deletion, indel, substitution or duplication.

According to a further embodiment of the present invention, the insertion or the deletion produces a gene comprising a frameshift.

According to a further embodiment of the present invention, the nucleotide modification is a silencing mutation, a knockdown mutation, a knockout mutation, a loss of function mutation or any combination thereof.

It is further within the scope of the current invention to provide a method for producing a Cannabis plant exhibiting reduced tetrahydrocannabinolic acid (THCA) content and comprising a modified genomic locus involved in tetrahydrocannabinolic acid synthase (THCAS) gene expression as compared to a control Cannabis plant, said method comprises steps of introducing one or more nucleotide modifications through a targeted DNA modification at a regulatory region controlling THCAS expression.

According to a further aspect, the present invention provides a method for reducing tetrahydrocannabinolic acid (THCA) content in a Cannabis plant by modifying genomic locus involved in tetrahydrocannabinolic acid synthase (THCAS) gene expression as compared to a control Cannabis plant, wherein the method comprises introducing one or more nucleotide modifications through a targeted DNA modification at a regulatory region controlling THCAS expression.

It is further within the scope of the current invention to provide a method for producing a Cannabis plant exhibiting reduced cannabidiolic acid (CBDA) content and comprising a modified genomic locus involved in cannabidiolic acid synthase (CBDAS) gene expression as compared to a control Cannabis plant, said method comprises steps of introducing one or more nucleotide modifications through a targeted DNA modification at a regulatory region controlling CBDAS expression.

According to a further aspect, the present invention provides a method for reducing cannabidiolic acid (CBDA) content in a Cannabis plant by modifying genomic locus involved in cannabidiolic acid synthase (CBDAS) gene expression as compared to a control Cannabis plant, wherein the method comprises introducing one or more nucleotide modifications through a targeted DNA modification at a regulatory region controlling CBDAS expression.

According to a further aspect, the present invention provides a method for producing a medical Cannabis composition, the method comprising: (a) obtaining the Cannabis plant as defined in any of the above; and (b) formulating a medical Cannabis composition from said plant.

The present invention further provides an isolated nucleotide sequence having at least 75% sequence identity to a nucleotide sequence selected from the group consisting of SEQ ID NO:1 to SEQ ID NO:6 and SEQ ID NO:1150 to SEQ ID NO:1153.

According to further aspects, the present invention provides an isolated nucleotide sequence having at least 75% sequence identity to a nucleotide sequence selected from the group consisting of SEQ ID NO:7 to SEQ ID NO:1149.

According to further aspects, the present invention provides a vector, construct or expression system or cassette comprising a nucleic acid sequence having at least 75% sequence identity to a nucleotide sequence selected from the group consisting of SEQ ID NO:1 to SEQ ID NO:1153.

Other embodiments of the present invention include the use of a nucleotide sequence having at least 75% sequence identity to a nucleotide sequence as set forth in at least one of SEQ ID NO: 7-91, 92-176, 177-278, 279-419, 420-560 and 561-681 for targeted genome modification of pFNTHCAS-1, pFNTHCAS-2, pPKTHCAS-1, pPKTHCAS-2, pPKTHCAS-3 and pPKTHCAS-4 gene, respectively.

Yet other embodiments of the present invention include the use of a nucleotide sequence having at least 75% sequence identity to a nucleotide sequence as set forth in at least one of SEQ ID NO: 682-794, 795-895, 896-1016 and 1017-1149 for targeted genome modification of pCBDAS2, pFNCBDAS, pPKCBDAS and pPKCBDAS 1 gene, respectively.

It is also within the scope of the present invention to provide the use of a nucleotide sequence having at least 75% sequence identity to a nucleotide sequence as set forth in at least one of SEQ ID NO: 7-681 and any combination thereof for reducing THCA content in a Cannabis plant.

It is also within the scope of the present invention to provide the use of a nucleotide sequence having at least 75% sequence identity to a nucleotide sequence as set forth in at least one of SEQ ID NO: 682-1149 and any combination thereof for reducing CBDA content in a Cannabis plant.

According to some embodiments of the present invention, the above in planta modification can be based on alternative gene silencing technologies such as Zinc Finger Nucleases (ZFN's), Transcription activator-like effector nucleases (TALEN's), RNA silencing, amiRNA or any other gene silencing technique known in the art.

According to some other embodiments of the present invention, DNA introduction into the plant cells can be done by Agrobacterium infiltration, viral based plasmids for virus induced gene silencing (VIGS) and by mechanical insertion of DNA (PEG mediated DNA transformation, biolistics, etc.).

In addition, it is within the scope of the present invention that the Cas9 protein is directly inserted together with a gRNA (ribonucleoprotein-RNP's) in order to bypass the need for in vivo transcription and translation of the Cas9+gRNA plasmid in planta to achieve gene editing.

It is a core aspect of the present invention that the above CRISPR/Cas system allows the modification of specific DNA sequences. This is achieved by combining the Cas nuclease (Cas9, Cpf1 or the like) with a guide RNA molecule (gRNA). The gRNA is designed such that it should be complementary to a specific DNA sequence targeted for editing in the plant genome and which guides the Cas nuclease to a specific nucleotide sequence (see FIG. 1). Gene specific gRNA's are cloned into the same plasmid as the Cas gene and this plasmid is inserted into plant cells. Insertion of this plasmid DNA can be done, but not limited to, by different delivery systems biological and or mechanical.

Without wishing to be bound by theory, according to further specific aspects of the present invention, upon reaching the specific DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually creates a mutation around the cleavage site. The deletion form of the mutation consists of at least 1 base pair deletion. As a result of this base pair deletion the gene coding sequence is disrupted and the translation of the encoded protein is compromised either by a premature stop codon or disruption of a functional or structural property of the protein.

It is further within the scope that by introducing a gRNA with homology to a specific site of a gene described in FIG. 2, and sub cloning this gRNA into a plasmid containing the Cas9 gene, and upon insertion of the described plasmid into the plant cells, site specific mutations are generated in the regulatory region (promoter) of genes herein described. Thus effectively creating non-active proteins in the cannabinoid biosynthesis pathway, results in inactivation of their enzymatic activity. As a result, the present disclosure enables altering cannabinoid content in the genome edited plant. This alteration of cannabinoid content can result in a plant with significantly reduced synthesis of the molecules depicted in FIG. 2 and/or of one or more cannabinoids produced by these enzymes.

A reduction in the production of THC, CBD, or Cannabichromene will enhance production of the remaining metabolites in this shared pathway. For example, production of CBD and/or Cannabichromene is enhanced by inhibiting production of THC. THC production may be inhibited by inhibiting expression and/or activity of tetrahydrocannabinolic acid (THCA) synthase enzyme.

Described are certain embodiments of enhancing production of one or more secondary metabolites by downregulation of the production of one or more metabolites having a shared biosynthetic pathway.

The plant of the invention includes plants wherein the plant is heterozygous for the each of the mutations. In other embodiment however, the plant is homozygous for the mutations. Progeny that is also homozygous can be generated from these plants according to methods known in the art.

It is further within the scope that variants of a particular nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant nucleotide sequence of the Cannabis tetrahydrocannabinolic acid synthase (CsTHCAS) allele as shown in any one of SEQ ID NO 1-6. Sequence alignment programs to determine sequence identity are well known in the art.

It is further within the scope that variants of a particular nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant nucleotide sequence of the Cannabis cannabidiolic acid synthase (CsCBDAS) allele as shown in any one of SEQ ID NO 1150-1153. Sequence alignment programs to determine sequence identity are well known in the art.

Also, the various aspects of the invention encompass not only a Cannabis tetrahydrocannabinolic acid synthase (CsTHCAS) and/or cannabidiolic acid synthase (CsCBDAS) promoter sequence, but also fragments thereof. By “fragment” is intended a portion of the nucleotide sequence or a portion of the amino acid sequence and hence of the protein encoded thereby. Fragments of a nucleotide sequence may encode protein fragments that retain the biological activity of the native protein, in this case cannabinoid biosynthesis enzymes.

It is further within the scope that manipulation of cannabinoid biosynthesis enzymes in Cannabis plants is herein achieved by generating gRNA with homology to a specific site of predetermined gene promoters in the Cannabis genome i.e. Cannabis tetrahydrocannabinolic acid synthase (CsTHCAS) and/or cannabidiolic acid synthase (CsCBDAS) homologue or variant, sub cloning this gRNA into a plasmid containing the Cas9 gene, and insertion of the plasmid into the Cannabis plant cells. In this way site specific mutations in the promoter regions controlling the aforementioned genes are generated thus effectively creating non-active molecules, resulting in loss of function of at least one of the THCAS and/or CBDAS enzymes and reduced content of THCA (and/or THC) and/or CBDA (and/or CBD) in the genome edited plant.

In order to understand the invention and to see how it may be implemented in practice, a plurality of preferred embodiments will now be described, by way of non-limiting example only, with reference to the following examples.

### Example 1

Identifying Cannabis THCA Synthase (THCAS) and CBDA Synthase (CBDAS) Alleles for Targeted Genome Editing

This example demonstrates the identification of Cannabis genomic loci, particularly, promoter regions, targets for modulating THCA and/or CBDA content in the plant.

In order to downregulate the production of THCA and/or CBDA in the plant, Cannabis THCA synthesis (THCAS) and CBDA synthase (CBDAS) gene alleles or homologues/variants were identified. Particularly, the promoter regions of Cannabis THCA synthesis (THCAS) and CBDA synthase (CBDAS) gene alleles or homologues/variants were identified. The used strategy was to modify the expression of the identified genes by introducing targeted mutations at the promoter regions of THCAS and/or CBDAS variants by CRISPR/Cas technology. In this way, knock-down or loss-of-function mutations in regulatory sequences or elements necessary for the expression of one or more of the identified THCAS and/or CBDAS genes or alleles could be achieved, to substantially reduce the cannabinoid biosynthesis enzymes production in the plant, specifically in high THC or low THC Cannabis varieties.

The promoter regions (about 2 Kb upstream to the first ATG within the gene or allele of interest) of various THCAS and/or CBDAS gene variants have been identified in different Cannabis sativa (C. sativa) strains or varieties. The promoter regions were cloned and sequenced.

The following promoter sequences are herein exemplified:

pFNTHCAS-1: The promoter region of THCAS-1 variant of the hemp-type “Finola” cultivar was mapped to Chromosome 6 CM011610.1:22241180-22244169 and has a nucleic acid sequence as set forth in SEQ ID NO:1.

pFNTHCAS-2: The promoter region of THCAS-2 variant of “Finola” cultivar was mapped to Chromosome 3 CM011607.1:10147821-10150821 and has a nucleic acid sequence as set forth in SEQ ID NO:2.

pPKTHCAS-1: The promoter region of THCAS-1 variant of “Purple Kush” cultivar was mapped to Chromosome 8 CM010797.2:28650050-28653053 and has a nucleic acid sequence as set forth in SEQ ID NO:3.

pPKTHCAS-2: The promoter region of THCAS-2 variant of “Purple Kush” cultivar was mapped to Chromosome 7 CM010796.2:62086454-62089454 and has a nucleic acid sequence as set forth in SEQ ID NO:4.

pPKTHCAS-3: The promoter region of THCAS-3 variant of “Purple Kush” cultivar was mapped to Chromosome 4 CM010793.2:49188167-49191167 and has a nucleic acid sequence as set forth in SEQ ID NO:5.

pPKTHCAS-4: The promoter region of THCAS-4 variant of “Purple Kush” cultivar was mapped to Chromosome 3 CM010792.2:58197740-58200740 and has a nucleic acid sequence as set forth in SEQ ID NO:6.

pCBDAS2: The promoter region of Cannabidiolic acid synthase-like 1 (CBDAS2) variant was mapped to CM010797.2_52807720-52810660 and has a nucleic acid sequence as set forth in SEQ ID NO:1150.

pFNCBDAS: The promoter region of CBDAS variant of “Finola” cultivar was mapped to CM011610.1_21834038-21837038 and has a nucleic acid sequence as set forth in SEQ ID NO:1151.

pPKCBDAS: The promoter region of CBDAS variant of “Purple Kush” cultivar was mapped to CM010792.2_58197740-58200740 and has a nucleic acid sequence as set forth in SEQ ID NO:1152.

pPKCBDAS1: The promoter region of CBDAS1 variant of “Purple Kush” cultivar was mapped to CM010796.2_62086535-62089454 and has a nucleic acid sequence as set forth in SEQ ID NO:1153.

### Example 2

Targeted DNA Modification of Genomic Loci Involved in THCA and/or CBDA Synthesis

Targeted DNA modification of promoter regions encoding identified THCAS and/or CBDAS alleles was performed to affect cannabinoid profile and specifically THCA and/or CBDA content in Cannabis.

Toward this end, CRISPR/Cas9 assisted targeted genome editing in gene regulatory regions was used to alter the expression, or more specifically, knock-out any gene sequence of interest and generate knock-outs through small deletions, internal small fragment deletions within the target genetic locus, or full-length gene deletion.

The Cannabis plants of the present invention comprise CRISPR/Cas9 expression cassette that encodes a Cas9 endonuclease and at least one guide RNA (gRNA), each gRNA containing a sequence that is complementary to a target sequence within a regulatory region of an allele of a gene of interest, wherein the target region is 0 to 2000 base pairs upstream of the 5′ end of the coding sequence of the gene of interest or wherein the target region is 0 to 2000 base pairs downstream of the 3′ end of the coding sequence of the gene of interest. Through CRISPR-Cas genome editing targeted to the regulatory sequences of a gene of interest, new variations of the identified alleles are introduced directly in the elite target germplasm with minimal genetic drag associated with conventional breeding material. Examples of target THCAS gene alleles within the scope of the current invention include the “Finola” THCAS-1 and THCAS-2 variants (FNTHCAS-1 and FNTHCAS-2, respectively), and the Purple Kush THCAS-1, THCAS-2, THCAS-3 and THCAS-4 variants (PKTHCAS-1, PKTHCAS-2, PKTHCAS-3 and PKTHCAS-4, respectively).

Examples of target CBDAS gene alleles within the scope of the current invention include Cannabidiolic acid synthase-like 1 (CBDAS2) variant, the “Finola” CBDAS variant (FNCBDAS), and the Purple Kush CBDAS and CBDAS1 variants (PKCBDAS, and PKCBDAS1, respectively). Tables 1-10 below provide the guide RNA (gRNA) polynucleotide sequences and targeting strategies to knock-down expression of the target genes, and more specifically the promoter regions of the genes.

Production of Cannabis lines with mutated Cannabis tetrahydrocannabinolic acid synthase (CsTHCAS) and/or mutated Cannabidiolic acid synthase (CsCBDAS) may be achieved by at least one of the following breeding/cultivation schemes:

Scheme 1:
line stabilization by self pollination
Generation of F6 parental lines
Genome editing of parental lines
Crossing edited parental lines to generate an F1 hybrid plant

Scheme 2:
Identifying genes/alleles of interest
Designing gRNA targeted to regulatory (e.g. promoter) regions of the identified genes
Transformation of plants with Cas9+gRNA constructs
Screening and identifying editing events
Genome editing of parental lines

It is noted that line stabilization may be performed by the following:
Induction of male flowering on female (XX) plants
Self pollination

According to certain embodiments of the present invention, line stabilization requires about 6 self-crossing (6 generations) and done through a single seed descent (SSD) approach.

F1 hybrid seed production: Novel hybrids are produced by crosses between different Cannabis strains.

According to further aspects of the current invention, shortening line stabilization period is performed by Doubled Haploids (DH). More specifically, the CRISPR-Cas9 system is transformed into microspores to achieve DH homozygous parental lines. It is noted that a doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for Cannabis are developed and provided by the current invention:
Sex markers—molecular markers are used for identification and selection of female vs male plants in the herein disclosed breeding program.
Genotyping markers—germplasm used in the current invention is genotyped using molecular markers, in order to allow a more efficient breeding process and identification of the Cannabis tetrahydrocannabinolic acid synthase (CsTHCAS) and/or Cannabidiolic acid synthase (CsCBDAS) editing event.

It is further within the scope of the current invention that allele and genetic variation is analyzed for the Cannabis strains or cultivars used.

Reference is now made to optional stages that have been used for the production of Cannabis plants with mutated tetrahydrocannabinolic acid synthase (CsTHCAS) and/or mutated Cannabidiolic acid synthase (CsCBDAS) by genome editing targeted to the promoter region of the gene:

Stage 1: Identifying Cannabis sativa (C. sativa) tetrahydrocannabinolic acid synthase (THCAS) and Cannabidiolic acid synthase (CBDAS) orthologues/homologs or alleles from various cultivars/strains characterized by different THCA and or CBDA content or profile, for example “Finola” (FN) hemp-type cultivar with relatively low THCA level and “Purple Kush” (PK) potent-type strain with relatively high THCA level.

The following homologs have herein been identified in Cannabis sativa (C. sativa). These genes have been sequenced and mapped.

pFNTHCAS-1: The promoter region of THCAS-1 variant or allele of the hemp-type “Finola” cultivar was mapped to Chromosome 6 CM011610.1:22241180-22244169 and has a nucleic acid sequence as set forth in SEQ ID NO:1.

pFNTHCAS-2: The promoter region of THCAS-2 variant of “Finola” cultivar was mapped to Chromosome 3 CM011607.1:10147821-10150821 and has a nucleic acid sequence as set forth in SEQ ID NO:2.

pPKTHCAS-1: The promoter region of THCAS-1 variant of “Purple Kush” cultivar was mapped to Chromosome 8 CM010797.2:28650050-28653053 and has a nucleic acid sequence as set forth in SEQ ID NO:3.

pPKTHCAS-2: The promoter region of THCAS-2 variant of “Purple Kush” cultivar was mapped to Chromosome 7 CM010796.2:62086454-62089454 and has a nucleic acid sequence as set forth in SEQ ID NO:4.

pPKTHCAS-3: The promoter region of THCAS-3 variant of “Purple Kush” cultivar was mapped to Chromosome 4 CM010793.2:49188167-49191167 and has a nucleic acid sequence as set forth in SEQ ID NO:5.

pPKTHCAS-4: The promoter region of THCAS-4 variant of “Purple Kush” cultivar was mapped to Chromosome 3 CM010792.2:58197740-58200740 and has a nucleic acid sequence as set forth in SEQ ID NO:6.

pCBDAS2: The promoter region of Cannabidiolic acid synthase-like 1 (CBDAS2) variant was mapped to CM010797.2_52807720-52810660 and has a nucleic acid sequence as set forth in SEQ ID NO:1150.

pFNCBDAS: The promoter region of CBDAS variant of “Finola” cultivar was mapped to CM011610.1_21834038-21837038 and has a nucleic acid sequence as set forth in SEQ ID NO:1151.

pPKCBDAS: The promoter region of CBDAS variant of “Purple Kush” cultivar was mapped to CM010792.2_58197740-58200740 and has a nucleic acid sequence as set forth in SEQ ID NO:1152.

pPKCBDAS1: The promoter region of CBDAS1 variant of “Purple Kush” cultivar was mapped to CM010796.2_62086535-62089454 and has a nucleic acid sequence as set forth in SEQ ID NO:1153.

Stage 2: Designing and synthesizing gRNA molecules corresponding to the sequences targeted for editing, i.e. sequences of the promoter region of each of the Cannabis tetrahydrocannabinolic acid synthase (CsTHCAS) and/or Cannabidiolic acid synthase (CsCBDAS) gene variants or homologues. It is noted that the editing event is preferably targeted to a unique restriction site sequence to allow easier screening for plants carrying an editing event within their genome.

According to some aspects of the invention, the nucleotide sequence of the gRNAs should be compatible with the genomic sequence of the target genomic loci sequence. Therefore, for example, suitable gRNA molecules should be constructed for different Cannabis tetrahydrocannabinolic acid synthase (CsTHCAS) and/or Cannabidiolic acid synthase (CsCBDAS) homologues of different Cannabis strains.

Reference is now made to Tables 1-10 presenting gRNA nucleic acid sequences targeted to the promoter region of various herein identified Cannabis tetrahydrocannabinolic acid synthase (CsTHCAS) and/or Cannabidiolic acid synthase (CsCBDAS) homologues. In the aforementioned tables, the term ‘PAM’ refers to ‘protospacer adjacent motif’, which is a 2-6 base pair DNA sequence immediately following the DNA sequence targeted by the Cas9 nuclease in the CRISPR bacterial adaptive immune system. The CsTHCAS and/or CsCBDAS genomic DNA sense strand is marked as “1”, and the antisense strand is marked as “−1”.

TABLE 1
gRNA sequences targeted for the promoter region of FNTHCAS-1
(pFNTHCAS-1)

| Position | | | | | | |
|---|---|---|---|---|---|---|
| on SEQ | | | | Specificity | Efficiency | SEQ |
| ID NO 1 | Strand | Sequence | PAM | Score | Score | ID NO |
| 57 | 1 | AATTAATAAAATATACATAA | AGG | 37.30045 | 44.44996 | 7 |
| 73 | −1 | ATGCCACTTAGACTCTTCTT | AGG | 83.8106 | 35.69232 | 8 |
| 81 | 1 | AAGCCTAAGAAGAGTCTAAG | TGG | 88.99535 | 55.97398 | 9 |
| 96 | −1 | AATAGGAAAGAAAAAAAAAT | AGG | 47.57658 | 40.80125 | 10 |
| 113 | −1 | TGTTAGAATAAAGGAAAAAT | AGG | 44.90739 | 36.03156 | 11 |
| 122 | -1 | AAATAGATGTGTTAGAATAA | AGG | 62.49713 | 37.42861 | 12 |
| 179 | 1 | ACAAACTAAAAGTCCCACAT | TGG | 62.48411 | 63.86964 | 13 |
| 181 | −1 | TAAAAGTTTTTAACCAATGT | GGG | 70.88673 | 41.59538 | 14 |
| 182 | −1 | TTAAAAGTTTTTAACCAATG | TGG | 69.82372 | 56.08351 | 15 |
| 211 | −1 | ACTATTATTTATAACTTACA | TGG | 59.0868 | 43.27014 | 16 |
| 245 | −1 | AATATTTCTTAGACAAATGT | AGG | 55.19085 | 49.9328 | 17 |
| 267 | 1 | CTAAGAAATATTATTTTTTA | TGG | 31.03341 | 15.74631 | 18 |
| 268 | 1 | TAAGAAATATTATTTTTTAT | GGG | 24.99497 | 20.05015 | 19 |
| 271 | 1 | GAAATATTATTTTTTATGGG | TGG | 56.92163 | 46.98163 | 20 |
| 298 | −1 | TTTATGTATAACAATTTATT | GGG | 41.20847 | 23.13908 | 21 |
| 299 | −1 | CTTTATGTATAACAATTTAT | TGG | 49.88155 | 23.79478 | 22 |
| 311 | 1 | CAATAAATTGTTATACATAA | AGG | 49.20722 | 45.13024 | 23 |
| 327 | 1 | ATAAAGGAAAGCCTAAGAAG | AGG | 69.34745 | 61.94475 | 24 |
| 327 | −1 | TGCCACTTAGACCTCTTCTT | AGG | 89.47488 | 43.8227 | 25 |
| 336 | 1 | AGCCTAAGAAGAGGTCTAAG | TGG | 78.44404 | 57.51686 | 26 |
| 351 | −1 | AAATAGGAAGAAAAAAAAAT | AGG | 53.38316 | 40.43731 | 27 |
| 367 | −1 | TGTTAGAATAAAGGAAAAAT | AGG | 44.90739 | 36.03156 | 28 |
| 376 | −1 | ATAAGAGTGTGTTAGAATAA | AGG | 69.57764 | 38.7884 | 29 |
| 431 | 1 | ATACAACAAAAGTCCCACAT | TGG | 26.40678 | 67.80764 | 30 |
| 433 | −1 | AAAAAGTTTTTAACCAATGT | GGG | 66.88815 | 46.26687 | 31 |
| 434 | −1 | TAAAAAGTTTTTAACCAATG | TGG | 69.44719 | 53.53075 | 32 |
| 465 | −1 | ACTATTATTTATAACTTACA | TGG | 59.0868 | 46.60631 | 33 |
| 499 | −1 | TAATATTTCTTAGACAATGT | AGG | 59.98419 | 56.04282 | 34 |
| 520 | 1 | CTAAGAAATATTATTTTTTA | TGG | 31.03341 | 15.74631 | 35 |
| 521 | 1 | TAAGAAATATTATTTTTTAT | GGG | 24.99497 | 20.05015 | 36 |
| 524 | 1 | GAAATATTATTTTTTATGGG | TGG | 56.92163 | 46.98163 | 37 |
| 551 | −1 | ATATATATAACAAATTTATT | GGG | 32.32636 | 26.76095 | 38 |
| 552 | −1 | CATATATATAACAAATTTAT | TGG | 38.71112 | 14.67985 | 39 |
| 779 | 1 | ATTAAAAATTTATCTATAAG | AGG | 48.23413 | 47.97876 | 40 |
| 794 | −1 | CTCAAATAATCTTATAAGTA | TGG | 56.31656 | 37.96536 | 41 |
| 962 | 1 | TTTAGTTTAAATTATTTAGA | TGG | 41.58344 | 41.01041 | 42 |
| 1005 | −1 | AAAAAAACAAACTATATTTT | AGG | 29.4093 | 13.684 | 43 |
| 1166 | 1 | ATTAATATCAATATATATAA | AGG | 26.64701 | 34.37782 | 44 |
| 1185 | 1 | AAGGAAAGTCTAAACAAAAG | TGG | 41.28903 | 58.98994 | 45 |
| 1392 | 1 | GTATTATTTATTTTCTATGT | AGG | 44.74194 | 35.77706 | 46 |
| 1425 | −1 | TTAAAAAAAATCAAGTAATT | GGG | 40.57689 | 26.83912 | 47 |
| 1426 | −1 | CTTAAAAAAAATCAAGTAAT | TGG | 47.58421 | 16.82191 | 48 |
| 1636 | −1 | ATATAATTTAAATTATTITA | TGG | 27.62763 | 13.91978 | 49 |
| 1702 | −1 | TACATAAAAAGATATATTTA | AGG | 38.52423 | 21.41299 | 50 |
| 1759 | 1 | ATTGTGAATGCTAAACTTAT | AGG | 57.19554 | 24.90842 | 51 |
| 1873 | −1 | AAAAAAAAATTACAACTAAT | TGG | 30.93854 | 35.1758 | 52 |
| 2080 | −1 | TTTTGTGATAAATATTAAAA | TGG | 40.56067 | 25.23162 | 53 |
| 2260 | 1 | ATATAAAATATTACAAAAGT | TGG | 46.10563 | 39.5453 | 54 |
| 2278 | −1 | GAACTAAGCCGCGCTTCGCT | CGG | 67.71118 | 52.13932 | 55 |
| 2281 | 1 | GGACAACACCGAGCGAAGCG | CGG | 98.17644 | 66.66762 | 56 |
| 2300 | −1 | TCTACTTTATTATTCAACTA | GGG | 53.65502 | 49.20125 | 57 |
| 2301 | −1 | ATCTACTTTATTATTCAACT | AGG | 47.80163 | 53.90388 | 58 |
| 2320 | 1 | AATAATAAAGTAGATAGTAG | AGG | 47.07501 | 58.36602 | 59 |
| 2449 | −1 | TATATATTTTATTTTTTATT | TGG | 17.81835 | 14.23412 | 60 |
| 2464 | 1 | ATAAAAAATAAAATATATAT | TGG | 21.34829 | 34.7715 | 61 |
| 2487 | 1 | TACTTCATATTTAGTTTTTA | TGG | 45.55735 | 5.867938 | 62 |
| 2488 | 1 | ACTTCATATTTAGTTTTTAT | GGG | 42.54593 | 22.65668 | 63 |
| 2570 | −1 | TCATTAATATATATTTTTTT | GGG | 37.4834 | 10.47335 | 64 |
| 2571 | −1 | TTCATTAATATATATTTTTT | TGG | 15.15828 | 11.6956 | 65 |
| 2590 | 1 | ATATATATTAATGAAAAAAA | AGG | 41.71363 | 41.92236 | 66 |
| 2593 | 1 | TATATTAATGAAAAAAAAGG | TGG | 54.74449 | 66.16164 | 67 |
| 2597 | 1 | TTAATGAAAAAAAAGGTGGA | AGG | 54.67719 | 59.23138 | 68 |
| 2606 | 1 | AAAAAGGTGGAAGGTGCCAT | AGG | 89.63392 | 60.79526 | 69 |
| 2611 | −1 | TTGTGGGATATAGGTGCCTA | TGG | 93.73732 | 55.85182 | 70 |
| 2620 | −1 | ATAGCTAGTTTGTGGGATAT | AGG | 65.03164 | 40.87556 | 71 |
| 2627 | −1 | ATATCTTATAGCTAGTTTGT | GGG | 63.23979 | 38.36823 | 72 |
| 2628 | −1 | AATATCTTATAGCTAGTTTG | TGG | 62.16786 | 40.97152 | 73 |
| 2666 | −1 | GACACGCAAATATTTATCTA | TGG | 81.37183 | 42.70162 | 74 |
| 2688 | −1 | GAAATTAGATATGAAAAGAA | AGG | 36.30093 | 51.11128 | 75 |
| 2735 | 1 | TTTTTTTCAATAGCCAATTT | CGG | 46.286 | 35.67624 | 76 |
| 2737 | −1 | AGAAGTTAAGCTGCCGAAAT | TGG | 97.88277 | 37.04145 | 77 |
| 2762 | −1 | TAGTTTGGCATCATTATATA | TGG | 62.23903 | 30.33342 | 78 |
| 2777 | −1 | AAATTTTACATTGAATAGTT | TGG | 44.92574 | 27.31643 | 79 |
| 2807 | 1 | ATTTAGATTTATTTTCATTA | AGG | 39.41732 | 31.08135 | 80 |
| 2808 | 1 | TTTAGATTTATTTTCATTAA | GGG | 25.82554 | 17.42162 | 81 |
| 2825 | −1 | AAAAATTAGGCACATTTGTT | AGG | 59.50885 | 31.77485 | 82 |
| 2838 | −1 | TAAAAAAATCCACAAAAATT | AGG | 33.34058 | 50.71886 | 83 |
| 2840 | 1 | ACAAATGTGCCTAATTTTTG | TGG | 66.24559 | 31.13315 | 84 |
| 2861 | −1 | AGTCATTAAATAGTGACATA | TGG | 71.74714 | 50.34693 | 85 |
| 2885 | −1 | ACTTAAATATTTCTATAATT | TGG | 42.92034 | 18.9827 | 86 |
| 2920 | −1 | TATAACTTTATATTGGAGCG | GGG | 91.68505 | 59.10928 | 87 |
| 2921 | −1 | CTATAACTTTATATTGGAGC | GGG | 81.97792 | 39.46084 | 88 |
| 2922 | −1 | TCTATAACTTTATATTGGAG | CGG | 59.73169 | 58.95429 | 89 |
| 2927 | −1 | TCCTTTCTATAACTTTATAT | TGG | 56.2663 | 28.95317 | 90 |
| 2937 | 1 | TCCAATATAAAGTTATAGAA | AGG | 52.27911 | 43.64749 | 91 |

TABLE 2
gRNA sequences targeted for the promoter region of FNTHCAS-2
(pFNTHCAS-2)

| Position | | | | | | |
|---|---|---|---|---|---|---|
| on SEQ | | | | Specificity | Efficiency | SEQ |
| ID NO 2 | Strand | Sequence | PAM | Score | Score | ID NO |
| 47 | −1 | GGTGAAATGGGTGTGGCTAG | TGG | 93.06675 | 60.02599 | 92 |
| 54 | −1 | TGAGTGAGGTGAAATGGGTG | TGG | 87.28814 | 57.92724 | 93 |
| 59 | −1 | GAGAGTGAGTGAGGTGAAAT | GGG | 88.7199 | 44.71439 | 94 |
| 60 | −1 | TGAGAGTGAGTGAGGTGAAA | TGG | 72.47844 | 45.94887 | 95 |
| 68 | −1 | TTGATTGGTGAGAGTGAGTG | AGG | 86.4586 | 70.34416 | 96 |
| 83 | −1 | AAAGTGGATGTGCTCTTGAT | TGG | 84.6305 | 42.06345 | 97 |
| 99 | −1 | ACCTATAATTCCTTATAAAG | TGG | 57.72583 | 45.00558 | 98 |
| 100 | 1 | AAGAGCACATCCACTTTATA | AGG | 86.1605 | 31.47348 | 99 |
| 109 | 1 | TCCACTTTATAAGGAATTAT | AGG | 59.74398 | 28.69535 | 100 |
| 117 | 1 | ATAAGGAATTATAGGTCATT | TGG | 61.75744 | 27.78855 | 101 |
| 118 | 1 | TAAGGAATTATAGGTCATTT | GGG | 39.73871 | 32.93543 | 102 |
| 130 | −1 | TCTCTCCAAATTGGGCATGT | AGG | 91.34126 | 58.73687 | 103 |
| 136 | 1 | TTGGGCCTACATGCCCAATT | TGG | 93.59257 | 28.91465 | 104 |
| 138 | −1 | CAACTAATTCTCTCCAAATT | GGG | 45.25987 | 36.61505 | 105 |
| 139 | −1 | ACAACTAATTCTCTCCAAAT | TGG | 67.35778 | 28.64271 | 106 |
| 163 | 1 | AATTAGTTGTCTTAGCCCAA | TGG | 89.28118 | 56.65087 | 107 |
| 164 | 1 | ATTAGTTGTCTTAGCCCAAT | GGG | 83.31915 | 50.07837 | 108 |
| 167 | −1 | ACGTCTTTGGTCAGCCCATT | GGG | 95.81976 | 40.04369 | 109 |
| 168 | −1 | AACGTCTTTGGTCAGCCCAT | TGG | 92.20885 | 46.53134 | 110 |
| 180 | −1 | GACACACTCTAAAACGTCTT | TGG | 92.74599 | 44.98216 | 111 |
| 196 | 1 | GACGTTTTAGAGTGTGTCCA | TGG | 91.64096 | 50.93123 | 112 |
| 197 | 1 | ACGTTTTAGAGTGTGTCCAT | GGG | 84.07056 | 62.31399 | 113 |
| 202 | −1 | ACTAACACGCTCGGGGCCCA | TGG | 99.96175 | 52.7168 | 114 |
| 209 | −1 | ATAATGTACTAACACGCTCG | GGG | 97.9132 | 68.93021 | 115 |
| 210 | −1 | CATAATGTACTAACACGCTC | GGG | 95.93056 | 45.4712 | 116 |
| 211 | −1 | ACATAATGTACTAACACGCT | CGG | 90.5421 | 56.14222 | 117 |
| 234 | −1 | ACCATCGAGCCATAATGGGA | TGG | 94.16587 | 61.94864 | 118 |
| 236 | 1 | ACATTATGTCCATCCCATTA | TGG | 82.60926 | 39.12025 | 119 |
| 238 | −1 | TATTACCATCGAGCCATAAT | GGG | 86.88214 | 30.99927 | 120 |
| 239 | −1 | GTATTACCATCGAGCCATAA | TGG | 90.85214 | 42.92587 | 121 |
| 244 | 1 | TCCATCCCATTATGGCTCGA | TGG | 95.25786 | 58.92677 | 122 |
| 261 | −1 | TATATATAATCGGTAATGCA | TGG | 87.07542 | 62.23998 | 123 |
| 271 | −1 | TAATGATAACTATATATAAT | CGG | 39.86535 | 33.5985 | 124 |
| 338 | −1 | TCTTGTTAGAATATTTTATG | TGG | 50.49141 | 51.20583 | 125 |
| 402 | 1 | AGTACAATAGCAGAAATTCT | TGG | 70.83348 | 45.22282 | 126 |
| 403 | 1 | GTACAATAGCAGAAATTCTT | GGG | 85.9079 | 39.00634 | 127 |
| 589 | 1 | AAAGTGACTTTATTGTGTAG | AGG | 78.56754 | 54.22294 | 128 |
| 619 | −1 | TAACAATGACGTATACAAAG | AGG | 76.01634 | 66.8923 | 129 |
| 646 | 1 | ATTGTTATTATTAAGCTTTC | AGG | 55.7787 | 21.94542 | 130 |
| 705 | −1 | AAAGCAAACAGTTTTATACA | TGG | 56.07626 | 56.68599 | 131 |
| 745 | 1 | TCTTTACTTTTATTCTTTGT | TGG | 47.39883 | 17.03976 | 132 |
| 782 | 1 | TTACACACTACTATACGAAA | AGG | 86.93125 | 50.04312 | 133 |
| 783 | 1 | TACACACTACTATACGAAAA | GGG | 87.00754 | 40.1819 | 134 |
| 784 | 1 | ACACACTACTATACGAAAAG | GGG | 85.43565 | 55.242 | 135 |
| 803 | −1 | ATAAAATGCCCATAAAAATT | GGG | 49.75479 | 36.58467 | 136 |
| 804 | −1 | TATAAAATGCCCATAAAAAT | TGG | 52.22068 | 22.52606 | 137 |
| 805 | 1 | GGCTTTAATCCCAATTTTTA | TGG | 69.43957 | 11.96029 | 138 |
| 806 | 1 | GCTTTAATCCCAATTTTTAT | GGG | 58.45044 | 23.09451 | 139 |
| 842 | 1 | TTTTTGCTAAAATAAAAATT | AGG | 32.92785 | 35.08424 | 140 |
| 855 | 1 | AAAAATTAGGATATATGTCA | TGG | 52.2864 | 59.69357 | 141 |
| 878 | 1 | ATAAAAGCTCATATAAAAAG | TGG | 47.26925 | 50.20043 | 142 |
| 879 | 1 | TAAAAGCTCATATAAAAAGT | GGG | 50.8648 | 55.91161 | 143 |
| 913 | 1 | TTTTTAATTTCGATTATTAT | AGG | 41.75496 | 15.96412 | 144 |
| 920 | 1 | TTTCGATTATTATAGGAAAA | CGG | 54.56637 | 33.26003 | 145 |
| 921 | 1 | TTCGATTATTATAGGAAAAC | GGG | 73.30407 | 31.99965 | 146 |
| 951 | −1 | CTGAGCTTTCAATTAAAACT | GGG | 77.94849 | 42.63435 | 147 |
| 952 | −1 | TCTGAGCTTTCAATTAAAAC | TGG | 82.4031 | 33.75771 | 148 |
| 984 | −1 | ATGGAAAAATTGGAATTAAA | GGG | 45.02752 | 25.2724 | 149 |
| 985 | −1 | TATGGAAAAATTGGAATTAA | AGG | 55.18792 | 19.66529 | 150 |
| 994 | −1 | TCCAGTTTTTATGGAAAAAT | TGG | 53.70117 | 22.02845 | 151 |
| 1003 | −1 | AGTATTAATTCCAGTTTTTA | TGG | 70.45929 | 7.473108 | 152 |
| 1004 | 1 | TCCAATTTTTCCATAAAAAC | TGG | 61.11629 | 27.91589 | 153 |
| 1060 | −1 | TAATGAAGAACATAGTATCT | TGG | 53.95887 | 41.43487 | 154 |
| 1172 | −1 | GATATGATTTCATTAATTAA | TGG | 42.29853 | 21.33147 | 155 |
| 1228 | −1 | TATTGTAATGTTAATATTTA | TGG | 39.32494 | 11.27835 | 156 |
| 1246 | 1 | TATTAACATTACAATATCAA | TGG | 47.35739 | 48.37131 | 157 |
| 1332 | −1 | GCTTCATGGTTCATGGTTCA | TGG | 82.39858 | 38.56957 | 158 |
| 1339 | −1 | TGACATTGCTTCATGGTTCA | TGG | 82.57519 | 36.0907 | 159 |
| 1346 | −1 | TCGCCATTGACATTGCTTCA | TGG | 87.62043 | 42.64503 | 160 |
| 1354 | 1 | GAACCATGAAGCAATGTCAA | TGG | 83.65606 | 55.97267 | 161 |
| 1410 | 1 | AGAGAGAGAGAGAGAGCTTC | AGG | 76.13449 | 38.56159 | 162 |
| 1422 | −1 | GAGAAAACTCTTGCTGAAAA | AGG | 69.56911 | 48.05928 | 163 |
| 1444 | −1 | CTATCTTTACCCATCACTGT | CGG | 89.36193 | 60.0946 | 164 |
| 1445 | 1 | AGAGTTTTCTCCGACAGTGA | TGG | 93.53635 | 57.57352 | 165 |
| 1446 | 1 | GAGTTTTCTCCGACAGTGAT | GGG | 94.87723 | 52.71107 | 166 |
| 1461 | 1 | GTGATGGGTAAAGATAGAAG | TGG | 75.21052 | 65.88804 | 167 |
| 1466 | 1 | GGGTAAAGATAGAAGTGGAG | AGG | 78.2412 | 50.82358 | 168 |
| 1476 | 1 | AGAAGTGGAGAGGAAGAAGA | AGG | 24.95318 | 59.37288 | 169 |
| 1488 | −1 | CGATCTCAGCCACGCACGAA | GGG | 99.11323 | 57.88212 | 170 |
| 1489 | −1 | GCGATCTCAGCCACGCACGA | AGG | 99.27134 | 61.26077 | 171 |
| 1490 | 1 | AGAAGAAGGCCCTTCGTGCG | TGG | 96.01766 | 59.70369 | 172 |
| 1502 | 1 | TTCGTGCGTGGCTGAGATCG | CGG | 99.24529 | 60.96408 | 173 |
| 1514 | 1 | TGAGATCGCGGCCAAGAATC | CGG | 97.1039 | 44.941 | 174 |
| 1514 | −1 | TTTCTGCATTGCCGGATTCT | TGG | 91.62651 | 30.80071 | 175 |
| 1522 | −1 | CCCAATTCTTTCTGCATTGC | CGG | 84.98718 | 20.07995 | 176 |

TABLE 3
gRNA sequences targeted for the promoter region of PKTHCAS-1
(pPKTHCAS-1)

| Position | | | | | | |
|---|---|---|---|---|---|---|
| on SEQ | | | | Specificity | Efficiency | SEQ |
| ID NO 3 | Strand | Sequence | PAM | Score | Score | ID NO |
| 16 | −1 | TTCATCCTCAAATAGACTTA | TGG | 76.22659 | 46.66036 | 177 |
| 22 | 1 | ATTGACCATAAGTCTATTTG | AGG | 79.38498 | 49.08821 | 178 |
| 35 | 1 | CTATTTGAGGATGAATTCTT | TGG | 61.89627 | 29.64954 | 179 |
| 42 | 1 | AGGATGAATTCTTTGGCAAG | AGG | 59.33175 | 55.07154 | 180 |
| 45 | 1 | ATGAATTCTTTGGCAAGAGG | TGG | 85.34579 | 52.65131 | 181 |
| 46 | 1 | TGAATTCTTTGGCAAGAGGT | GGG | 89.22847 | 60.51966 | 182 |
| 64 | 1 | GTGGGAAAATGATAATTTGT | TGG | 60.38704 | 48.17156 | 183 |
| 65 | 1 | TGGGAAAATGATAATTTGTT | GGG | 56.21042 | 34.06247 | 184 |
| 122 | 1 | TITTATAAGTAACACCCCTA | AGG | 87.85281 | 59.6849 | 185 |
| 125 | −1 | CCTCTATACTTCTTCCTTAG | GGG | 68.11223 | 49.01085 | 186 |
| 126 | −1 | GCCTCTATACTTCTTCCTTA | GGG | 84.90014 | 28.37812 | 187 |
| 127 | −1 | GGCCTCTATACTTCTTCCTT | AGG | 79.76857 | 34.89908 | 188 |
| 136 | 1 | CCCCTAAGGAAGAAGTATAG | AGG | 88.67694 | 54.85618 | 189 |
| 148 | −1 | CTATCAATAATATCTATTTC | AGG | 60.93833 | 18.63942 | 190 |
| 168 | 1 | GATATTATTGATAGTGATCA | AGG | 72.41144 | 48.51067 | 191 |
| 169 | 1 | ATATTATTGATAGTGATCAA | GGG | 65.34852 | 61.42779 | 192 |
| 234 | 1 | AATAGAGAACGTAGTTATGA | TGG | 79.94231 | 44.2004 | 193 |
| 238 | 1 | GAGAACGTAGTTATGATGGC | TGG | 94.26923 | 58.42022 | 194 |
| 252 | 1 | GATGGCTGGTATAGAGTATT | AGG | 89.77914 | 24.54742 | 195 |
| 253 | 1 | ATGGCTGGTATAGAGTATTA | GGG | 87.21477 | 40.54756 | 196 |
| 262 | 1 | ATAGAGTATTAGGGTATCCA | TGG | 90.91746 | 55.76289 | 197 |
| 263 | 1 | TAGAGTATTAGGGTATCCAT | GGG | 85.19985 | 56.11212 | 198 |
| 268 | −1 | CACCAAACAATAAATACCCA | TGG | 77.58331 | 64.02404 | 199 |
| 277 | 1 | ATCCATGGGTATTTATTGTT | TGG | 62.78175 | 25.101 | 200 |
| 304 | −1 | GAAAAAGATTCTAATATCTT | GGG | 55.11911 | 36.84466 | 201 |
| 305 | −1 | GGAAAAAGATTCTAATATCT | TGG | 69.54019 | 28.7464 | 202 |
| 326 | −1 | AATCATAGAGGCAACATGTT | TGG | 76.99676 | 37.83753 | 203 |
| 338 | −1 | TTGAATTCTAGAAATCATAG | AGG | 61.07034 | 58.46187 | 204 |
| 355 | 1 | TGATTTCTAGAATTCAAAAT | TGG | 41.32821 | 29.49464 | 205 |
| 370 | 1 | AAAATTGGTCAAATGATGTT | AGG | 49.31514 | 47.60662 | 206 |
| 397 | −1 | AATCTCAATTCCTTCAAACA | TGG | 64.7464 | 51.91395 | 207 |
| 398 | 1 | GATGCAAACGCCATGTTTGA | AGG | 90.97367 | 43.95033 | 208 |
| 436 | −1 | CTTAACATTACCATCTTCAG | AGG | 82.71192 | 60.09172 | 209 |
| 437 | 1 | TTTTTATTTGCCTCTGAAGA | TGG | 79.31428 | 46.1021 | 210 |
| 501 | −1 | ATTTATAAATATCATGTAAG | AGG | 51.97239 | 58.5279 | 211 |
| 599 | 1 | TTAGAACTAGAGTATAAAAT | TGG | 52.99189 | 32.62103 | 212 |
| 614 | 1 | AAAATTGGTCTATGTAATTT | AGG | 53.89258 | 19.29819 | 213 |
| 615 | 1 | AAATTGGTCTATGTAATTTA | GGG | 44.52603 | 16.16035 | 214 |
| 642 | −1 | TATCAAAACATTTAAACAAC | AGG | 52.28777 | 42.71263 | 215 |
| 710 | −1 | ATTCAAAAGCGTTAAATATT | TGG | 56.29078 | 17.65532 | 216 |
| 735 | −1 | ATATAGATAGATATTGATGA | AGG | 52.92217 | 55.76927 | 217 |
| 764 | 1 | TATATCTATATATAATATAA | AGG | 18.68702 | 35.86662 | 218 |
| 785 | 1 | GGAAAGCACAAAATGAGTTT | AGG | 74.41382 | 41.90897 | 219 |
| 788 | 1 | AAGCACAAAATGAGTTTAGG | TGG | 64.50592 | 60.17806 | 220 |
| 890 | −1 | ACATCATTTATAAACAATGT | GGG | 49.08517 | 62.37136 | 221 |
| 891 | −1 | TACATCATTTATAAACAATG | TGG | 58.08491 | 60.91614 | 222 |
| 904 | 1 | ACATTGTTTATAAATGATGT | AGG | 53.79121 | 57.46885 | 223 |
| 919 | 1 | GATGTAGGCATCATCCATGT | AGG | 83.3533 | 63.49715 | 224 |
| 922 | −1 | AATAATATTTATAACCTACA | TGG | 62.40285 | 54.73867 | 225 |
| 968 | −1 | CCCATGAACAAAAATACTTT | TGG | 71.03634 | 30.11921 | 226 |
| 978 | 1 | TCCAAAAGTATTTTTGTTCA | TGG | 69.38426 | 35.79391 | 227 |
| 979 | 1 | CCAAAAGTATTTTTGTTCAT | GGG | 67.87166 | 35.88578 | 228 |
| 982 | 1 | AAAGTATTTTTGTTCATGGG | TGG | 64.23178 | 59.7122 | 229 |
| 1009 | −1 | TAAAATAATAAAAACTTATA | GGG | 39.04604 | 32.49632 | 230 |
| 1010 | −1 | ATAAAATAATAAAAACTTAT | AGG | 39.69306 | 26.07249 | 231 |
| 1223 | −1 | TTTAGTITGAATATTGTTTT | GGG | 41.6746 | 21.94432 | 232 |
| 1224 | −1 | ATTTAGTTTGAATATTGTTT | TGG | 29.19977 | 21.75731 | 233 |
| 1519 | −1 | ATATTAATTTTTGGACAAAT | CGG | 33.43457 | 40.05836 | 234 |
| 1528 | −1 | AGAGAAACAATATTAATTTT | TGG | 34.95541 | 13.63258 | 235 |
| 1548 | 1 | AATATTGTTTCTCTTTATAT | AGG | 44.14179 | 27.62736 | 236 |
| 1549 | 1 | ATATTGTTTCTCTTTATATA | GGG | 35.30454 | 30.45974 | 237 |
| 1550 | 1 | TATTGTTTCTCTTTATATAG | GGG | 47.60729 | 44.86893 | 238 |
| 1572 | −1 | TATATAAAATTAATTGGATA | AGG | 47.43438 | 42.21643 | 239 |
| 1578 | −1 | TATAAATATATAAAATTAAT | TGG | 15.56643 | 21.68107 | 240 |
| 1687 | −1 | TTACAAATATTATAAGTAAA | TGG | 42.9377 | 35.80111 | 241 |
| 1781 | 1 | TTAATTGAGTTTAAAAAATG | TGG | 46.39659 | 50.12076 | 242 |
| 1847 | −1 | ATTTGTATCAATTTTTAAGT | TGG | 50.70586 | 38.4329 | 243 |
| 1979 | −1 | TAACGTTTTTATTGAATGAG | AGG | 63.62482 | 56.15934 | 244 |
| 2170 | −1 | AAATGAATCAAATATTATAA | TGG | 41.31625 | 34.07693 | 245 |
| 2439 | −1 | GAATAGATCCGCGCTTCACG | CGG | 86.93957 | 67.14846 | 246 |
| 2442 | 1 | TATTAAAACCGCGTGAAGCG | CGG | 81.07225 | 61.07471 | 247 |
| 2461 | −1 | TCTACTTTATTATTCAACTA | GGG | 53.65502 | 49.20125 | 248 |
| 2462 | −1 | ATCTACTTTATTATTCAACT | AGG | 47.80163 | 53.90388 | 249 |
| 2481 | 1 | AATAATAAAGTAGATAGTAG | AGG | 47.07501 | 58.36602 | 250 |
| 2484 | 1 | AATAAAGTAGATAGTAGAGG | AGG | 74.31028 | 56.87514 | 251 |
| 2610 | −1 | TATCTATTTTACTTTTTATT | TGG | 38.13357 | 17.56456 | 252 |
| 2625 | 1 | ATAAAAAGTAAAATAGATAT | TGG | 36.88532 | 45.24505 | 253 |
| 2648 | 1 | TACTTGATATTCACTCTTTA | TGG | 71.83957 | 19.70709 | 254 |
| 2649 | 1 | ACTTGATATTCACTCTTTAT | GGG | 72.80124 | 25.77884 | 255 |
| 2663 | −1 | ATGACTTTTATAGTTTATTA | TGG | 43.41333 | 25.25828 | 256 |
| 2696 | 1 | TTATGTGTACTTGCTACCAT | AGG | 88.026 | 60.31532 | 257 |
| 2701 | −1 | TTGTGGGATATAGGTGCCTA | TGG | 93.73732 | 50.13745 | 258 |
| 2710 | −1 | GTAGCTAGTTTGTGGGATAT | AGG | 73.48955 | 42.82095 | 259 |
| 2717 | −1 | GGCTATGGTAGCTAGTTTGT | GGG | 87.81635 | 36.55875 | 260 |
| 2718 | −1 | TGGCTATGGTAGCTAGTTTG | TGG | 91.02847 | 48.10622 | 261 |
| 2732 | −1 | AAAAAACAAGAAATTGGCTA | TGG | 59.43617 | 53.40395 | 262 |
| 2738 | −1 | GGAAACAAAAAACAAGAAAT | TGG | 44.17194 | 35.01751 | 263 |
| 2759 | −1 | CATCAATAAAAATTGGATAT | TGG | 61.68844 | 31.90158 | 264 |
| 2766 | −1 | AGTTTGGCATCAATAAAAAT | TGG | 64.41129 | 33.20242 | 265 |
| 2782 | −1 | ACATTGTACATTGAATAGTT | TGG | 49.93549 | 30.58453 | 266 |
| 2812 | 1 | ATGTACATTTATTTTCAATA | AGG | 47.98481 | 35.71245 | 267 |
| 2813 | 1 | TGTACATTTATTTTCAATAA | GGG | 38.90366 | 31.47042 | 268 |
| 2829 | 1 | ATAAGGGCTTCACCTAACAA | AGG | 90.10665 | 63.81456 | 269 |
| 2830 | −1 | TAAAATTAGGCACCTTTGTT | AGG | 73.18171 | 31.62394 | 270 |
| 2843 | −1 | AAAATAAATCAACTAAAATT | AGG | 36.70261 | 49.89944 | 271 |
| 2926 | −1 | AATATTATATATTGGAGTTG | GGG | 62.77123 | 46.22677 | 272 |
| 2927 | −1 | TAATATTATATATTGGAGTT | GGG | 54.53941 | 28.87773 | 273 |
| 2928 | −1 | ATAATATTATATATTGGAGT | TGG | 55.83549 | 31.02418 | 274 |
| 2934 | −1 | CTATTTATAATATTATATAT | TGG | 21.73378 | 27.17729 | 275 |
| 2946 | 1 | CAATATATAATATTATAAAT | AGG | 30.03862 | 28.7367 | 276 |
| 2971 | −1 | TAATGATTTTTTGAATTATT | AGG | 30.86793 | 21.28812 | 277 |
| 2984 | 1 | TAATAATTCAAAAAATCATT | AGG | 37.28455 | 36.07488 | 278 |

TABLE 4
gRNA sequences targeted for the promoter region of PKTHCAS-2
(pPKTHCAS-2)

| Position on | | | | Specificity | Efficiency | SEQ |
|---|---|---|---|---|---|---|
| SEQ ID NO 4 | Strand | Sequence | PAM | Score | Score | ID NO |
| 10 | 1 | AATGTATGTGGTGCTTTGTT | CGG | 66.67228 | 43.66271 | 279 |
| 22 | −1 | TTCGTCCGAGCAAATGTATG | TGG | 94.05462 | 58.15859 | 280 |
| 28 | 1 | AAGCACCACATACATTTGCT | CGG | 89.89544 | 62.26872 | 281 |
| 47 | 1 | TCGGACGAAGTCCTCTTAGC | CGG | 97.59389 | 51.05675 | 282 |
| 47 | −1 | TTGCTCACTGCCCGGCTAAG | AGG | 98.46684 | 48.53528 | 283 |
| 48 | 1 | CGGACGAAGTCCTCTTAGCC | GGG | 98.84971 | 56.72004 | 284 |
| 55 | −1 | TGCGCGGATTGCTCACTGCC | CGG | 91.61699 | 48.48856 | 285 |
| 71 | −1 | CTAAGTGTTGGCGTGCTGCG | CGG | 99.346 | 59.08716 | 286 |
| 83 | −1 | AAGCTGAATTTGCTAAGTGT | TGG | 68.733 | 53.35574 | 287 |
| 121 | −1 | CAAAGTTATAGGGATTTCTT | TGG | 68.68829 | 35.72969 | 288 |
| 131 | −1 | GGCCTGATCCCAAAGTTATA | GGG | 91.91771 | 35.70959 | 289 |
| 132 | −1 | TGGCCTGATCCCAAAGTTAT | AGG | 89.38245 | 30.95953 | 290 |
| 133 | 1 | CAAAGAAATCCCTATAACTT | TGG | 62.41844 | 32.63986 | 291 |
| 134 | 1 | AAAGAAATCCCTATAACTTT | GGG | 61.048 | 36.88213 | 292 |
| 140 | 1 | ATCCCTATAACTTTGGGATC | AGG | 82.46335 | 39.50266 | 293 |
| 152 | −1 | CGAATCTTGCCATATCTCTA | TGG | 94.08479 | 43.38427 | 294 |
| 154 | 1 | GGGATCAGGCCATAGAGATA | TGG | 95.94324 | 56.85926 | 295 |
| 205 | −1 | AAAAGTTTATCACTTTATTT | GGG | 44.67817 | 30.16463 | 296 |
| 206 | −1 | TAAAAGTTTATCACTTTATT | TGG | 49.3526 | 6.844194 | 297 |
| 235 | 1 | TTTTAAACAAAGAAGCAACA | AGG | 45.27843 | 67.83789 | 298 |
| 258 | 1 | CGTACATTGTCGACCTCAGA | AGG | 92.55616 | 52.08572 | 299 |
| 260 | −1 | CCTTTTTCCTTATCCTTCTG | AGG | 78.91542 | 53.70836 | 300 |
| 264 | 1 | TTGTCGACCTCAGAAGGATA | AGG | 95.38754 | 49.66444 | 301 |
| 271 | 1 | CCTCAGAAGGATAAGGAAAA | AGG | 70.13819 | 45.02106 | 302 |
| 285 | −1 | CAGTGACCGTGATAGCGAGC | TGG | 97.6093 | 52.28843 | 303 |
| 290 | 1 | AAGGCTCCAGCTCGCTATCA | CGG | 96.63158 | 43.3947 | 304 |
| 297 | 1 | CAGCTCGCTATCACGGTCAC | TGG | 99.57416 | 51.18988 | 305 |
| 311 | 1 | AGGATGTCCGTACAGGAGCT | TGG | 97.72549 | 54.61793 | 306 |
| 315 | 1 | ACTGGAACCAAGCTCCTGTA | CGG | 94.50349 | 54.2677 | 307 |
| 318 | −1 | GTGTCTGAGGATGTCCGTAC | AGG | 99.44745 | 57.59087 | 308 |
| 331 | −1 | ATCTTTAATTATTGTGTCTG | AGG | 81.0707 | 48.15802 | 309 |
| 380 | −1 | TGTGATCCTCCCGCATTTAT | TGG | 92.45626 | 25.43056 | 310 |
| 381 | 1 | TCATTTTGATCCAATAAATG | CGG | 56.40448 | 55.04425 | 311 |
| 382 | 1 | CATTTTGATCCAATAAATGC | GGG | 73.83298 | 55.63264 | 312 |
| 385 | 1 | TTTGATCCAATAAATGCGGG | AGG | 93.24632 | 60.57337 | 313 |
| 426 | −1 | CATATCACGTTGTGTAAATG | CGG | 79.85051 | 55.96468 | 314 |
| 458 | −1 | CGATCATCCCCTAAAATCAT | GGG | 86.72966 | 51.8821 | 315 |
| 459 | −1 | ACGATCATCCCCTAAAATCA | TGG | 87.357 | 46.86483 | 316 |
| 460 | 1 | TAATCAAATCCCATGATTTT | AGG | 50.37986 | 21.19347 | 317 |
| 461 | 1 | AATCAAATCCCATGATTTTA | GGG | 62.68383 | 22.50742 | 318 |
| 462 | 1 | ATCAAATCCCATGATTTTAG | GGG | 64.186 | 53.63938 | 319 |
| 517 | −1 | ACTATTTATAATCACATAGT | AGG | 64.91395 | 53.22792 | 320 |
| 530 | 1 | TACTATGTGATTATAAATAG | TGG | 58.1575 | 46.87963 | 321 |
| 551 | 1 | GGCAAGTAAGATCAAAAAAG | TGG | 70.88507 | 57.05661 | 322 |
| 574 | 1 | ACGAAAAAAGCATACAAAAA | AGG | 47.92631 | 46.19218 | 323 |
| 654 | −1 | AACACACAGGATTTTTTACG | TGG | 84.81338 | 64.37507 | 324 |
| 667 | −1 | CAATGAAAATATGAACACAC | AGG | 68.12405 | 64.19248 | 325 |
| 719 | 1 | TATGTGAGATTGTCACTGTT | AGG | 88.47807 | 42.77358 | 326 |
| 770 | 1 | TAACACAATCTAATTTATTT | TGG | 44.39155 | 13.9135 | 327 |
| 775 | 1 | CAATCTAATTTATTTTGGAT | TGG | 46.62025 | 40.88386 | 328 |
| 850 | −1 | TGGCGACATAAAACAATATT | GGG | 80.18368 | 27.85863 | 329 |
| 851 | −1 | TTGGCGACATAAAACAATAT | TGG | 72.98906 | 32.4639 | 330 |
| 870 | 1 | TATTTATTTTAATTTGTTGT | TGG | 36.4323 | 38.0834 | 331 |
| 906 | 1 | ACAAAAAAATAACTAACCCA | AGG | 61.20981 | 63.9156 | 332 |
| 907 | 1 | CAAAAAAATAACTAACCCAA | GGG | 58.96803 | 66.25841 | 333 |
| 911 | −1 | TATAATTTTTTTCTTCCCTT | GGG | 55.09525 | 44.82545 | 334 |
| 912 | −1 | ATATAATTTTTTTCTTCCCT | TGG | 61.56082 | 40.67156 | 335 |
| 1013 | 1 | TTTATTGTTGAAAAATTTAT | TGG | 33.01617 | 17.1718 | 336 |
| 1061 | −1 | TAATTATAAGACGTATAACA | TGG | 66.47955 | 57.95244 | 337 |
| 1114 | 1 | ATTCAATTTTAATGAGATCG | AGG | 80.50678 | 59.86873 | 338 |
| 1115 | 1 | TTCAATTTTAATGAGATCGA | GGG | 75.12932 | 58.31985 | 339 |
| 1302 | 1 | TATTTGTTAATATATATTGA | TGG | 40.89142 | 38.76283 | 340 |
| 1340 | −1 | TAAAATTTTAAAGTTATGTG | TGG | 52.40048 | 61.65078 | 341 |
| 1379 | −1 | TTTTTTAAGATTAATTACTA | TGG | 31.82942 | 37.65256 | 342 |
| 1548 | 1 | AAAAGAAATTCAAATTATTA | AGG | 31.02728 | 26.49749 | 343 |
| 1551 | 1 | AGAAATTCAAATTATTAAGG | TGG | 52.5525 | 53.17187 | 344 |
| 1558 | 1 | CAAATTATTAAGGTGGCGTT | TGG | 89.27118 | 32.49361 | 345 |
| 1743 | 1 | TTTAATCAATGTTTTAGATT | AGG | 49.50645 | 34.18499 | 346 |
| 1754 | 1 | TTTTAGATTAGGACCAGACC | CGG | 95.99237 | 63.53117 | 347 |
| 1756 | −1 | AGGGAATTTGGGTCCGGGTC | TGG | 97.27293 | 39.29344 | 348 |
| 1761 | −1 | TAGGGAGGGAATTTGGGTCC | GGG | 91.34337 | 47.67576 | 349 |
| 1762 | −1 | GTAGGGAGGGAATTTGGGTC | CGG | 85.60468 | 42.65646 | 350 |
| 1767 | −1 | GGGCCGTAGGGAGGGAATTT | GGG | 95.06688 | 30.25891 | 351 |
| 1768 | −1 | AGGGCCGTAGGGAGGGAATT | TGG | 97.13429 | 25.17024 | 352 |
| 1775 | 1 | GGACCCAAATTCCCTCCCTA | CGG | 80.05958 | 62.73298 | 353 |
| 1775 | −1 | TAGCGCCAGGGCCGTAGGGA | GGG | 99.07174 | 49.60487 | 354 |
| 1776 | −1 | TTAGCGCCAGGGCCGTAGGG | AGG | 99.1334 | 53.67964 | 355 |
| 1779 | −1 | GGTTTAGCGCCAGGGCCGTA | GGG | 98.90292 | 51.98898 | 356 |
| 1780 | −1 | GGGTTTAGCGCCAGGGCCGT | AGG | 100 | 44.03016 | 357 |
| 1781 | 1 | AAATTCCCTCCCTACGGCCC | TGG | 96.93833 | 49.55893 | 358 |
| 1787 | −1 | AAAAGTCGGGTTTAGCGCCA | GGG | 99.08891 | 57.95828 | 359 |
| 1788 | −1 | CAAAAGTCGGGTTTAGCGCC | AGG | 99.27358 | 41.30613 | 360 |
| 1800 | −1 | TCGGGTTCAAGTCAAAAGTC | GGG | 75.64821 | 53.54228 | 361 |
| 1801 | −1 | TTCGGGTTCAAGTCAAAAGT | CGG | 86.31458 | 50.22631 | 362 |
| 1818 | 1 | TTTAGGTGCAAGTCGGATTC | GGG | 89.02798 | 34.98531 | 363 |
| 1819 | −1 | ATTTAGGTGCAAGTCGGATT | CGG | 93.31064 | 32.88236 | 364 |
| 1825 | −1 | AAAATAATTTAGGTGCAAGT | CGG | 35.09441 | 57.94599 | 365 |
| 1835 | −1 | TTAAGCTTTCAAAATAATTT | AGG | 40.3362 | 29.98844 | 366 |
| 1943 | 1 | AAGATAATTTTACCACTTAC | AGG | 79.14851 | 37.53132 | 367 |
| 1944 | −1 | TAATATAATCATCCTGTAAG | TGG | 74.62414 | 52.61125 | 368 |
| 1978 | 1 | TTGATTGTATTGATTATTAT | AGG | 44.19527 | 16.26497 | 369 |
| 2031 | 1 | TAGCTACAATTATTAATGAG | TGG | 64.838 | 56.04522 | 370 |
| 2058 | 1 | TAAAATTGAAGTGTGTTTTT | TGG | 40.6438 | 14.81896 | 371 |
| 2081 | −1 | ATATTTCAAACTTATAGCTT | AGG | 8.149386 | 38.28176 | 372 |
| 2138 | 1 | ACCAATTAGAAATGGGCACG | TGG | 95.28749 | 62.25444 | 373 |
| 2145 | −1 | AACTTAGACCAATTAGAAAT | GGG | 65.4901 | 41.42269 | 374 |
| 2146 | −1 | TAACTTAGACCAATTAGAAA | TGG | 58.68146 | 32.21124 | 375 |
| 2148 | 1 | ACCACGTGCCCATTTCTAAT | TGG | 92.02936 | 31.88023 | 376 |
| 2207 | −1 | TCCACGTGTCATTTTCTTCT | TGG | 71.79975 | 23.64376 | 377 |
| 2217 | 1 | ACCAAGAAGAAAATGACACG | TGG | 74.47341 | 72.79231 | 378 |
| 2239 | 1 | GATAATGACTTAATATTTAA | TGG | 48.48566 | 22.44283 | 379 |
| 2243 | 1 | ATGACTTAATATTTAATGGT | CGG | 41.26547 | 59.98016 | 380 |
| 2257 | 1 | TAATATTTTGGGAACTCTGT | AGG | 72.14999 | 53.58079 | 381 |
| 2268 | −1 | TAATACCTAAGTAATATTTT | GGG | 48.11281 | 24.67228 | 382 |
| 2269 | 1 | ATAATACCTAAGTAATATTT | TGG | 38.0272 | 13.08769 | 383 |
| 2274 | 1 | GAGTTCCCAAAATATTACTT | AGG | 69.85882 | 47.17027 | 384 |
| 2297 | −1 | AAATACCTACGTTTTATTTT | TGG | 58.56158 | 11.94795 | 385 |
| 2303 | 1 | TAGCGCCAAAAATAAAACGT | AGG | 89.70273 | 70.02414 | 386 |
| 2320 | 1 | CGTAGGTATTTATTTGCAAC | TGG | 85.66234 | 32.92943 | 387 |
| 2375 | −1 | CACAAAACATCTAAAAAAAA | TGG | 60.41049 | 33.67954 | 388 |
| 2387 | 1 | CATTTTTTTTAGATGTTTTG | TGG | 48.53328 | 26.9668 | 389 |
| 2400 | −1 | ACGCCAACTCATATACAAAA | TGG | 61.68887 | 40.39333 | 390 |
| 2408 | 1 | GGACCATTTTGTATATGAGT | TGG | 77.98036 | 60.94691 | 391 |
| 2453 | 1 | TGTTGAATCTCTAGCTCTTT | TGG | 72.86273 | 25.56256 | 392 |
| 2498 | 1 | GCTTTATTGTCTAAATTTCT | TGG | 58.05884 | 20.64172 | 393 |
| 2499 | 1 | CTTTATTGTCTAAATTTCTT | GGG | 43.90099 | 21.40116 | 394 |
| 2500 | 1 | TTTATTGTCTAAATTTCTTG | GGG | 43.25675 | 58.49927 | 395 |
| 2521 | −1 | CTAAAGTAAGCGAGCAACAT | GGG | 77.07228 | 56.12375 | 396 |
| 2522 | 1 | TCTAAAGTAAGCGAGCAACA | TGG | 79.35915 | 74.55214 | 397 |
| 2564 | 1 | GTTTGACAAAACATGCTATT | CGG | 73.08159 | 34.18684 | 398 |
| 2592 | 1 | CAATGAGCTATCCTAGTTCA | AGG | 88.72397 | 42.25734 | 399 |
| 2592 | −1 | CACAGAAATCTCCTTGAACT | AGG | 82.91262 | 56.77193 | 400 |
| 2613 | 1 | GGAGATTTCTGTGCTATTTG | TGG | 82.13188 | 46.71018 | 401 |
| 2686 | −1 | CGCTTGCAAATATTTATCTA | TGG | 83.83601 | 35.67387 | 402 |
| 2730 | 1 | ATAGCAAATTTTTTTTCCAT | AGG | 61.28847 | 41.9361 | 403 |
| 2735 | −1 | TGATTTTTTTAAATTTCCTA | TGG | 51.61273 | 37.98135 | 404 |
| 2804 | −1 | TTATTAAAAATAAATGTACA | TGG | 43.9121 | 64.33675 | 405 |
| 2817 | 1 | ATGTACATTTATTTTTAATA | AGG | 24.89831 | 24.97531 | 406 |
| 2818 | 1 | TGTACATTTATTTTTAATAA | GGG | 34.15151 | 33.69876 | 407 |
| 2834 | 1 | ATAAGGGCTGCACCTAACAA | AGG | 96.81063 | 57.21703 | 408 |
| 2835 | −1 | CAAAATTAGGCACCTTTGTT | AGG | 77.37957 | 31.41724 | 409 |
| 2847 | 1 | CTAACAAAGGTGCCTAATTT | TGG | 83.80673 | 24.89512 | 410 |
| 2848 | −1 | ATTTCTTTTTTACCAAAATT | AGG | 37.31223 | 45.67433 | 411 |
| 2864 | 1 | TTTTGGTAAAAAAGAAATTA | CGG | 30.76292 | 33.2113 | 412 |
| 2929 | −1 | AGCTTTATAGATTGGAGTGG | GGG | 87.14891 | 53.84871 | 413 |
| 2930 | −1 | TAGCTTTATAGATTGGAGTG | GGG | 85.54316 | 59.39764 | 414 |
| 2931 | −1 | ATAGCTTTATAGATTGGAGT | GGG | 78.79847 | 41.54822 | 415 |
| 2932 | 1 | TATAGCTTTATAGATTGGAG | TGG | 77.9249 | 55.63853 | 416 |
| 2937 | −1 | CTATTTATAGCTTTATAGAT | TGG | 65.91175 | 33.39825 | 417 |
| 2949 | 1 | CAATCTATAAAGCTATAAAT | AGG | 65.17611 | 28.20163 | 418 |
| 2969 | −1 | ATGTTGGAAATTACTATGAA | TGG | 60.42925 | 41.042 | 419 |

TABLE 5
gRNA sequences targeted for the promoter region of PKTHCAS-3
(pPKTHCAS-3)

| Position on | | | | Efficiency | |
|---|---|---|---|---|---|
| SEQ ID NO 5 | Strand | Sequence | PAM | Score | SEQ ID NO |
| 27 | 1 | TTACACTAAAGTGACTCTAT | AGG | 51.37357 | 420 |
| 39 | −1 | GTAACGTGGAACGCGGCGTA | TGG | 48.17963 | 421 |
| 46 | 1 | ATCTCTGGTAACGTGGAACG | CGG | 61.66603 | 422 |
| 53 | −1 | TATTTTTATCTCTGGTAACG | TGG | 65.40893 | 423 |
| 61 | −1 | CTTTTTCATATTTTTATCTC | TGG | 28.83085 | 424 |
| 134 | −1 | AGTTGTGATTTATGAATTTA | TGG | 20.17187 | 425 |
| 179 | 1 | AATTTTAGTATAGATAATTA | GGG | 37.39108 | 426 |
| 180 | −1 | TAATTTTAGTATAGATAATT | AGG | 19.83561 | 427 |
| 196 | 1 | TTATCTATACTAAAATTAAG | CGG | 55.9595 | 428 |
| 298 | −1 | ACTTGAAGTCGGAGACAAAT | TGG | 38.30882 | 429 |
| 309 | −1 | ACATTACTCGCACTTGAAGT | CGG | 62.58106 | 430 |
| 334 | 1 | GAGTAATGTAACTCCTGACA | TGG | 70.0695 | 431 |
| 336 | −1 | TAAACCAATACTTCCATGTC | AGG | 48.71834 | 432 |
| 343 | 1 | AACTCCTGACATGGAAGTAT | TGG | 53.35151 | 433 |
| 362 | 1 | TTGGTTTATGTTATCTTAAT | AGG | 15.08297 | 434 |
| 381 | 1 | TAGGTAAAGCTAATGATGTG | AGG | 66.30855 | 435 |
| 403 | −1 | AAAGGCATTATGAGTATGGG | TGG | 52.15349 | 436 |
| 406 | 1 | GATAAAGGCATTATGAGTAT | GGG | 60.53238 | 437 |
| 407 | −1 | TGATAAAGGCATTATGAGTA | TGG | 35.45017 | 438 |
| 421 | −1 | TTGAATTTTTAAGGTGATAA | AGG | 41.46482 | 439 |
| 430 | −1 | GGAAAGTCATTGAATTTTTA | AGG | 16.23795 | 440 |
| 451 | 1 | ACAAGCTAATAATCAAACAT | GGG | 63.7036 | 441 |
| 452 | 1 | CACAAGCTAATAATCAAACA | TGG | 50.58249 | 442 |
| 468 | 1 | TTTGATTATTAGCTTGTGTA | AGG | 53.03735 | 443 |
| 475 | 1 | ATTAGCTTGTGTAAGGAAGT | TGG | 56.0886 | 444 |
| 501 | 1 | TTTGATGAAAATTATATCTA | AGG | 39.71886 | 445 |
| 533 | 1 | AGCTATTTTATTAACCATGT | TGG | 40.1999 | 446 |
| 534 | 1 | GCTATTTTATTAACCATGTT | GGG | 40.69564 | 447 |
| 536 | −1 | AAGGGCCTACAGTCCCAACA | TGG | 63.97764 | 448 |
| 542 | 1 | ATTAACCATGTTGGGACTGT | AGG | 54.20652 | 449 |
| 554 | −1 | CAATAGAAGAAGAAGAAGAA | GGG | 55.20735 | 450 |
| 555 | −1 | GCAATAGAAGAAGAAGAAGA | AGG | 57.8543 | 451 |
| 587 | 1 | GCAAGTCTTTTCTCTAATGC | AGG | 46.59569 | 452 |
| 596 | 1 | TTCTCTAATGCAGGTTCTTT | AGG | 18.90613 | 453 |
| 597 | 1 | TCTCTAATGCAGGTTCTTTA | GGG | 27.8296 | 454 |
| 609 | 1 | GTTCTTTAGGGCCCACTTCT | AGG | 23.19224 | 455 |
| 609 | −1 | TAAAGGGGAGGCCTAGAAGT | GGG | 63.19205 | 456 |
| 610 | 1 | ATAAAGGGGAGGCCTAGAAG | TGG | 48.70311 | 457 |
| 621 | −1 | TATACTAAAAAATAAAGGGG | AGG | 63.25379 | 458 |
| 624 | 1 | CTATATACTAAAAAATAAAG | GGG | 53.81166 | 459 |
| 625 | 1 | ACTATATACTAAAAAATAAA | GGG | 30.22749 | 460 |
| 626 | −1 | TACTATATACTAAAAAATAA | AGG | 31.91763 | 461 |
| 639 | 1 | TTTATTTTTTAGTATATAGT | AGG | 47.7652 | 462 |
| 656 | −1 | ATAAAGCACAAAACTAAATA | TGG | 29.52971 | 463 |
| 705 | −1 | AACAACTTGGCTTAGCATAA | AGG | 41.95634 | 464 |
| 718 | −1 | ACAACATCCTAAAAACAACT | TGG | 59.05996 | 465 |
| 722 | 1 | TGCTAAGCCAAGTTGTTTTT | AGG | 19.91181 | 466 |
| 799 | 1 | GAATATATTGTTTGCATACT | TGG | 38.79224 | 467 |
| 821 | −1 | AGGAAACACAAATAATACGA | TGG | 66.97425 | 468 |
| 841 | −1 | ACGAAATAAAAAATTCAATC | AGG | 49.13621 | 469 |
| 866 | 1 | TTATTTCGTTCAAATGAGTT | TGG | 34.66748 | 470 |
| 867 | 1 | TATTTCGTTCAAATGAGTTT | GGG | 34.21176 | 471 |
| 893 | −1 | TTTATTCGAAAATCATGATT | AGG | 36.25314 | 472 |
| 918 | −1 | AATAAAAACTTTCAAAAATA | AGG | 33.80141 | 473 |
| 974 | 1 | AAAATTATTATGAAATGAAA | AGG | 40.47294 | 474 |
| 993 | 1 | AAGGTAATTATCTTAGAACT | AGG | 59.15509 | 475 |
| 1028 | −1 | TATTCCTAAGCGTGTCATCT | AGG | 49.921 | 476 |
| 1035 | 1 | AAATCCTAGATGACACGCTT | AGG | 45.06465 | 477 |
| 1048 | 1 | CACGCTTAGGAATATAATAT | AGG | 43.35339 | 478 |
| 1089 | −1 | AGAATTAACATAACAAAAGT | TGG | 43.55435 | 479 |
| 1159 | 1 | AGGTTCAATTAGGCTTAAAT | GGG | 26.79549 | 480 |
| 1160 | −1 | GAGGTTCAATTAGGCTTAAA | TGG | 11.9482 | 481 |
| 1169 | −1 | ATGTTAAAAGAGGTTCAATT | AGG | 41.35663 | 482 |
| 1179 | −1 | AGAGAATGGAATGTTAAAAG | AGG | 60.13345 | 483 |
| 1193 | −1 | TATGGCATTATTCAAGAGAA | TGG | 45.92532 | 484 |
| 1211 | −1 | ATCAATCAATAAAAAACGTA | TGG | 52.76058 | 485 |
| 1225 | 1 | TACGTTTTTTATTGATTGAT | TGG | 31.04597 | 486 |
| 1226 | 1 | ACGTTTTTTATTGATTGATT | GGG | 34.68951 | 487 |
| 1231 | 1 | TTTTATTGATTGATTGGGTA | TGG | 41.27641 | 488 |
| 1241 | 1 | TGATTGGGTATGGTTCGCGA | TGG | 44.28314 | 489 |
| 1242 | 1 | GATTGGGTATGGTTCGCGAT | GGG | 46.17496 | 490 |
| 1243 | 1 | ATTGGGTATGGTTCGCGATG | GGG | 56.39744 | 491 |
| 1259 | 1 | GATGGGGTATAATGAAAAGT | TGG | 53.8834 | 492 |
| 1329 | −1 | TTAATTTATTTTTTTTAACA | AGG | 44.14949 | 493 |
| 1428 | 1 | TCTTAGAAATGAAAGCAGTT | TGG | 37.46623 | 494 |
| 1429 | 1 | CTTAGAAATGAAAGCAGTTT | GGG | 24.88759 | 495 |
| 1430 | 1 | TTAGAAATGAAAGCAGTTTG | GGG | 67.75029 | 496 |
| 1441 | 1 | AGCAGTTTGGGGATTGTTAT | TGG | 39.20928 | 497 |
| 1442 | 1 | GCAGTTTGGGGATTGTTATT | GGG | 35.45483 | 498 |
| 1520 | −1 | ACAATAGCTTAGGTATGGGT | AGG | 60.08521 | 499 |
| 1524 | −1 | TGTAACAATAGCTTAGGTAT | GGG | 53.78138 | 500 |
| 1525 | −1 | TTGTAACAATAGCTTAGGTA | TGG | 40.81399 | 501 |
| 1530 | −1 | TAGACTTGTAACAATAGCTT | AGG | 43.2657 | 502 |
| 1583 | 1 | TGAACAAGAGTTGTCTACAT | TGG | 42.41447 | 503 |
| 1593 | 1 | TTGTCTACATTGGTAGAGAA | TGG | 53.92749 | 504 |
| 1635 | 1 | AAATATTAAATTTACTTCTT | TGG | 19.35483 | 505 |
| 1686 | 1 | ATATGAAAAAATAGAAGACT | TGG | 48.77194 | 506 |
| 1698 | −1 | AATAATTTTTACAGCATTTT | TGG | 10.15331 | 507 |
| 1720 | 1 | GTAAAAATTATTGTTTCTAA | AGG | 35.98177 | 508 |
| 1746 | 1 | TCTAAGAATGCACACTTATT | TGG | 16.66544 | 509 |
| 1751 | 1 | GAATGCACACTTATTTGGAG | TGG | 60.73712 | 510 |
| 1822 | 1 | ACTATTTTTGTCTATTCGAT | TGG | 45.93432 | 511 |
| 1841 | 1 | TTGGCAAAAAAGTCGAGCTT | AGG | 49.177 | 512 |
| 1863 | 1 | GTCTTAATTGCGATTTTGAG | AGG | 56.52866 | 513 |
| 1864 | 1 | TCTTAATTGCGATTTTGAGA | GGG | 53.67368 | 514 |
| 1865 | 1 | CTTAATTGCGATTTTGAGAG | GGG | 57.93698 | 515 |
| 1888 | 1 | AGACTTAATTTTTTGACGAC | TGG | 43.39505 | 516 |
| 1894 | 1 | AATTTTTTGACGACTGGAAC | TGG | 36.33687 | 517 |
| 1919 | 1 | AGAATCGTTGAGAAGTGCTT | TGG | 41.45829 | 518 |
| 1930 | 1 | GAAGTGCTTTGGATGAAGAC | TGG | 56.27803 | 519 |
| 1995 | 1 | AAATGTTAATGTCATGTTTA | TGG | 17.79064 | 520 |
| 2158 | −1 | TTCATTAATAGCATGTAAAA | AGG | 37.95665 | 521 |
| 2193 | −1 | TTAGCATATTCAATTCTCGC | AGG | 52.13678 | 522 |
| 2251 | 1 | GTAGAACAAAAGTATCAAAT | CGG | 49.05578 | 523 |
| 2266 | 1 | CAAATCGGTCTATATAATTT | AGG | 29.67435 | 524 |
| 2353 | −1 | TTCAAAACCGTTTAATATTC | GGG | 20.99959 | 525 |
| 2354 | −1 | ATTCAAAACCGTTTAATATT | CGG | 23.00901 | 526 |
| 2357 | 1 | TTCTTAACCCGAATATTAAA | CGG | 25.1721 | 527 |
| 2395 | 1 | TCATTGATTGAATAATAAAG | TGG | 49.52431 | 528 |
| 2405 | 1 | AATAATAAAGTGGATAGTAG | AGG | 48.17978 | 529 |
| 2406 | 1 | ATAATAAAGTGGATAGTAGA | GGG | 54.07497 | 530 |
| 2427 | −1 | AATATATAAATAAAAAATTA | TGG | 29.46324 | 531 |
| 2477 | −1 | CAAATAGCAAAATTAAATGA | AGG | 54.43039 | 532 |
| 2541 | 1 | TACAAAATAAAAAATAAGAT | AGG | 55.17445 | 533 |
| 2548 | 1 | TAAAAAATAAGATAGGATAT | TGG | 48.58712 | 534 |
| 2571 | 1 | TACTTGATAAGTCTTCTTTG | TGG | 47.38768 | 535 |
| 2584 | 1 | TTCTTTGTGGAAACGATAAT | CGG | 36.83527 | 536 |
| 2594 | 1 | AAACGATAATCGGTATTATT | AGG | 27.52107 | 537 |
| 2651 | 1 | AAATATATTAATAAATAAAG | TGG | 52.88204 | 538 |
| 2655 | 1 | ATATTAATAAATAAAGTGGA | AGG | 57.70377 | 539 |
| 2664 | 1 | AATAAAGTGGAAGGTGCCAT | AGG | 60.86993 | 540 |
| 2669 | −1 | TTTGTGGATATAGGTACCTA | TGG | 50.76575 | 541 |
| 2678 | 1 | ATATGCTAGTTTGTGGATAT | AGG | 42.19221 | 542 |
| 2685 | −1 | TCTTTCAATATGCTAGTTTG | TGG | 36.59093 | 543 |
| 2702 | 1 | ACTAGCATATTGAAAGAAAA | TGG | 39.16614 | 544 |
| 2710 | 1 | ATTGAAAGAAAATGGATCCA | TGG | 52.22709 | 545 |
| 2716 | −1 | GACTTGCAAATATTTATCCA | TGG | 53.99659 | 546 |
| 2756 | −1 | ATAATAATAAAAAAAGAAAT | TGG | 41.85341 | 547 |
| 2789 | 1 | TTTTAATAGAATATTTCAAA | AGG | 36.12475 | 548 |
| 2790 | 1 | TTTAATAGAATATTTCAAAA | GGG | 41.68774 | 549 |
| 2815 | 1 | TCTAACATTTATTTTTAATA | AGG | 24.023 | 550 |
| 2832 | 1 | ATAAGGACTGCACCTAACAA | AGG | 57.56215 | 551 |
| 2833 | −1 | AAAAATTAGGCACCTTTGTT | AGG | 32.66743 | 552 |
| 2846 | −1 | AAAAAAAGTTCACAAAAATT | AGG | 49.38359 | 553 |
| 2871 | −1 | ACTCATTAAATAGTCACATG | TGG | 66.07407 | 554 |
| 2927 | −1 | AGCATTATATATTGGAGCGG | GGG | 55.99668 | 555 |
| 2928 | −1 | TAGCATTATATATTGGAGCG | GGG | 55.8564 | 556 |
| 2929 | 1 | ATAGCATTATATATTGGAGC | GGG | 42.32237 | 557 |
| 2930 | 1 | TATAGCATTATATATTGGAG | CGG | 65.27564 | 558 |
| 2935 | −1 | CTATTTATAGCATTATATAT | TGG | 27.35132 | 559 |
| 2984 | 1 | ATAGTAATTCAAAAATCATT | AGG | 42.02543 | 560 |

TABLE 6
gRNA sequences targeted for the promoter region of PKTHCAS-4
(PPKTHCAS-4)

| Position on | | | | Specificity | Efficiency | SEQ |
|---|---|---|---|---|---|---|
| SEQ ID NO 6 | Strand | Sequence | PAM | Score | Score | ID NO |
| 15 | 1 | AAATTTATAGGAAACCCCTA | TGG | 79.05992 | 60.82868 | 561 |
| 27 | −1 | AAAAATTTTAAAAAATTTAT | AGG | 24.88478 | 12.51009 | 562 |
| 153 | 1 | GCTCTAAGTGTTTGTATATT | AGG | 71.02887 | 19.72184 | 563 |
| 213 | −1 | TAAAAATGATACTAAAATAC | TGG | 50.14456 | 45.12403 | 564 |
| 487 | 1 | TACATTTAACTTTTATAATA | TGG | 40.94089 | 13.19144 | 565 |
| 488 | 1 | ACATTTAACTTTTATAATAT | GGG | 39.82131 | 31.88376 | 566 |
| 685 | −1 | AATTACAAAAATGGTCTATT | GGG | 67.45602 | 31.22343 | 567 |
| 686 | 1 | AAATTACAAAAATGGTCTAT | TGG | 69.20416 | 32.73834 | 568 |
| 694 | −1 | CCAAAAAAAAATTACAAAAA | TGG | 40.17525 | 32.6503 | 569 |
| 705 | 1 | CCATTTTTGTAATTTTTTTT | TGG | 55.03665 | 4.39017 | 570 |
| 754 | −1 | TTAGAATAATATTAATACGT | AGG | 60.19719 | 55.44839 | 571 |
| 786 | 1 | AAAATTACTCTAAGTATTTA | AGG | 47.79289 | 10.74525 | 572 |
| 851 | −1 | AGCACATAATTTTTTGTATA | AGG | 56.48597 | 28.75291 | 573 |
| 880 | −1 | TTTATCGAAATTGACTTTAT | CGG | 49.07938 | 20.52118 | 574 |
| 905 | −1 | TTCTCTTAAACTTGGTTGTT | AGG | 71.94596 | 30.47243 | 575 |
| 913 | −1 | ATTTTAGTTTCTCTTAAACT | TGG | 58.60494 | 44.10799 | 576 |
| 946 | −1 | GCCGAAATTTCGGTAGAATT | AGG | 93.91936 | 38.76708 | 577 |
| 956 | 1 | TCCTAATTCTACCGAAATTT | CGG | 81.17653 | 33.0812 | 578 |
| 956 | −1 | CCGTTATGCTGCCGAAATTT | CGG | 96.80906 | 25.73719 | 579 |
| 967 | 1 | CCGAAATTTCGGCAGCATAA | CGG | 97.15419 | 49.31822 | 580 |
| 995 | −1 | ATTAATAGGTTTGAATTTTT | TGG | 32.90635 | 14.06121 | 581 |
| 1009 | −1 | TGTGTTTTGTTGTTATTAAT | AGG | 28.42475 | 19.4856 | 582 |
| 1084 | −1 | ATGTACTGTAGTCGGATGGG | TGG | 97.93272 | 65.48715 | 583 |
| 1087 | −1 | TACATGTACTGTAGTCGGAT | GGG | 96.82472 | 62.50227 | 584 |
| 1088 | −1 | ATACATGTACTGTAGTCGGA | TGG | 95.56897 | 64.70083 | 585 |
| 1092 | −1 | GTGAATACATGTACTGTAGT | CGG | 78.04598 | 58.39075 | 586 |
| 1119 | −1 | ATATTCATCTGTAGTGAAGT | AGG | 85.75811 | 56.2417 | 587 |
| 1181 | −1 | TTGTACTATGTCGGATCGAT | GGG | 97.03269 | 54.25148 | 588 |
| 1182 | −1 | ATTGTACTATGTCGGATCGA | TGG | 97.34184 | 52.78956 | 589 |
| 1190 | −1 | TACAATATATTGTACTATGT | CGG | 64.42547 | 46.16301 | 590 |
| 1216 | −1 | ATATTCATTTGTAGTGAAGT | AGG | 70.68127 | 55.06829 | 591 |
| 1310 | −1 | TTTTGTGTTGATCGGTTTCT | AGG | 87.0286 | 32.77909 | 592 |
| 1318 | −1 | CCAACTTATTTTGTGTTGAT | CGG | 62.34539 | 41.27599 | 593 |
| 1329 | 1 | CCGATCAACACAAAATAAGT | TGG | 70.50758 | 47.47113 | 594 |
| 1343 | −1 | GGTTGTGAGGTCAATTTGCA | AGG | 84.69011 | 57.79606 | 595 |
| 1356 | 1 | TCTTTATGTTGAAGGTTGTG | AGG | 73.63441 | 63.60809 | 596 |
| 1364 | −1 | GTAGTATTTCTTTATGTTGA | AGG | 51.8172 | 34.05116 | 597 |
| 1394 | −1 | AAGTAGCTAAATAAAAAAAT | TGG | 53.29758 | 40.44954 | 598 |
| 1546 | −1 | ATGTGTTTTATTTCTTTAGT | AGG | 56.40591 | 41.80989 | 599 |
| 1676 | −1 | ATTGTGAATGAGAATGAGAT | AGG | 56.94192 | 50.18484 | 600 |
| 1761 | −1 | GTGAATGAGAAATGTAATAT | AGG | 48.21007 | 37.67949 | 601 |
| 1854 | −1 | TGTGTATATCTATTGTGAAT | GGG | 55.62819 | 40.32213 | 602 |
| 1855 | −1 | ATGTGTATATCTATTGTGAA | TGG | 62.59737 | 53.71475 | 603 |
| 1924 | −1 | TTATTTTATAAATTTTTTTA | GGG | 42.0296 | 11.47894 | 604 |
| 1925 | −1 | GTTATTTTATAAATTTTTTT | AGG | 42.51547 | 8.149528 | 605 |
| 1993 | 1 | AATTAGATTTATACCTTAAT | AGG | 56.58292 | 19.71885 | 606 |
| 1995 | −1 | TTGTATCTCAACGCCTATTA | AGG | 90.0411 | 28.28075 | 607 |
| 2026 | −1 | CCTCCGGCCACCGTTTTTAG | TGG | 98.38454 | 39.87017 | 608 |
| 2027 | 1 | AATGTTTTCTCCACTAAAAA | CGG | 65.26185 | 40.5693 | 609 |
| 2030 | 1 | GTTTTCTCCACTAAAAACGG | TGG | 93.09942 | 69.96043 | 610 |
| 2034 | 1 | TCTCCACTAAAAACGGTGGC | CGG | 94.89369 | 50.91334 | 611 |
| 2037 | 1 | CCACTAAAAACGGTGGCCGG | AGG | 99.55525 | 64.66545 | 612 |
| 2042 | −1 | GGTAGTGGTGATTATACCTC | CGG | 93.74824 | 56.29174 | 613 |
| 2057 | −1 | TAAACAAAAAGTAAAGGTAG | TGG | 55.48546 | 59.33194 | 614 |
| 2063 | −1 | TTGGGGTAAACAAAAAGTAA | AGG | 58.11796 | 48.18294 | 615 |
| 2080 | 1 | ACATTTTTCCTCATTTTTTG | GGG | 44.42423 | 47.03086 | 616 |
| 2081 | −1 | TACATTTTTCCTCATTTTTT | GGG | 37.42586 | 10.97553 | 617 |
| 2082 | −1 | TTACATTTTTCCTCATTTTT | TGG | 35.35375 | 20.4246 | 618 |
| 2083 | 1 | TTTGTTTACCCCAAAAAATG | AGG | 36.92673 | 56.26361 | 619 |
| 2103 | 1 | AGGAAAAATGTAATCTTTTC | AGG | 52.84995 | 15.51548 | 620 |
| 2117 | 1 | CTTTTCAGGTATATAGTTTT | AGG | 63.77883 | 14.06283 | 621 |
| 2160 | 1 | GAAATAAACATGAGCTAAAA | TGG | 47.4092 | 24.47995 | 622 |
| 2179 | 1 | ATGGTGAAAAAATAGTGAAA | TGG | 54.77197 | 46.19193 | 623 |
| 2182 | 1 | GTGAAAAAATAGTGAAATGG | AGG | 44.30837 | 65.41596 | 624 |
| 2195 | 1 | GAAATGGAGGTGATTTTTCG | TGG | 81.00618 | 53.92372 | 625 |
| 2198 | 1 | ATGGAGGTGATTTTTCGTGG | TGG | 82.72984 | 52.56797 | 626 |
| 2202 | 1 | AGGTGATTTTTCGTGGTGGT | TGG | 86.11241 | 42.57064 | 627 |
| 2205 | 1 | TGATTTTTCGTGGTGGTTGG | TGG | 75.63488 | 46.27217 | 628 |
| 2210 | 1 | TTTCGTGGTGGTTGGTGGAG | AGG | 80.1843 | 44.50687 | 629 |
| 2211 | 1 | TTCGTGGTGGTTGGTGGAGA | GGG | 78.7611 | 44.13182 | 630 |
| 2216 | 1 | GGTGGTTGGTGGAGAGGGTT | TGG | 86.23921 | 17.86371 | 631 |
| 2217 | 1 | GTGGTTGGTGGAGAGGGTTT | GGG | 66.99371 | 30.55058 | 632 |
| 2218 | 1 | TGGTTGGTGGAGAGGGTTTG | GGG | 83.51557 | 40.53515 | 633 |
| 2227 | 1 | GAGAGGGTTTGGGGTTTCTT | TGG | 69.82314 | 32.5218 | 634 |
| 2232 | 1 | GGTTTGGGGTTTCTTTGGTT | TGG | 72.13771 | 28.46218 | 635 |
| 2233 | 1 | GTTTGGGGTTTCTTTGGTTT | GGG | 66.86859 | 22.12199 | 636 |
| 2234 | 1 | TTTGGGGTTTCTTTGGTTTG | GGG | 58.23159 | 28.12934 | 637 |
| 2235 | 1 | TTGGGGTTTCTTTGGTTTGG | GGG | 57.48842 | 39.0667 | 638 |
| 2242 | 1 | TTCTTTGGTTTGGGGGTTTC | TGG | 83.88754 | 0 | 639 |
| 2243 | 1 | TCTTTGGTTTGGGGGTTTCT | GGG | 80.35777 | 23.9674 | 640 |
| 2247 | 1 | TGGTTTGGGGGTTTCTGGGT | TGG | 83.09585 | 35.24111 | 641 |
| 2251 | 1 | TTGGGGGTTTCTGGGTTGGA | TGG | 88.79911 | 44.85733 | 642 |
| 2261 | 1 | CTGGGTTGGATGGTCGAATG | AGG | 91.90423 | 60.84874 | 643 |
| 2271 | 1 | TGGTCGAATGAGGAAGATGA | AGG | 79.39574 | 57.05756 | 644 |
| 2272 | 1 | GGTCGAATGAGGAAGATGAA | GGG | 69.42839 | 63.03577 | 645 |
| 2276 | 1 | GAATGAGGAAGATGAAGGGC | TGG | 78.66432 | 47.60694 | 646 |
| 2277 | 1 | AATGAGGAAGATGAAGGGCT | GGG | 72.2919 | 56.72909 | 647 |
| 2282 | 1 | GGAAGATGAAGGGCTGGGCT | AGG | 93.44725 | 38.24711 | 648 |
| 2283 | 1 | GAAGATGAAGGGCTGGGCTA | GGG | 92.30786 | 55.02592 | 649 |
| 2299 | 1 | GCTAGGGTTATAAAACCTTT | TGG | 83.70648 | 36.77708 | 650 |
| 2303 | −1 | AGAAGATAACCGACGCCAAA | AGG | 96.06026 | 48.17387 | 651 |
| 2305 | 1 | GTTATAAAACCTTTTGGCGT | CGG | 89.1036 | 50.38023 | 652 |
| 2359 | 1 | TAGTTTTTAATATAATTGTA | AGG | 43.6866 | 38.49408 | 653 |
| 2360 | 1 | AGTTTTTAATATAATTGTAA | GGG | 35.17553 | 44.01737 | 654 |
| 2361 | 1 | GTTTTTAATATAATTGTAAG | GGG | 52.01174 | 53.06262 | 655 |
| 2405 | 1 | ATTTTTTTGTAGATATTTTG | TGG | 40.37926 | 31.16942 | 656 |
| 2418 | −1 | ACACCAACTCATATAACAAA | TGG | 56.53691 | 42.69107 | 657 |
| 2426 | 1 | GGACCATTTGTTATATGAGT | TGG | 83.86505 | 49.29725 | 658 |
| 2485 | 1 | CTCTTTTATTGTGTTGTTTA | AGG | 49.5969 | 8.317329 | 659 |
| 2538 | 1 | CTAAAGTAAGCAAGCAACAT | GGG | 50.21894 | 56.38343 | 660 |
| 2539 | −1 | GCTAAAGTAAGCAAGCAACA | TGG | 64.02802 | 71.00251 | 661 |
| 2609 | 1 | CGATGATCTATCCTAATTCG | AGG | 89.16309 | 52.44567 | 662 |
| 2609 | −1 | TGCAGAAACCTCCTCGAATT | AGG | 75.27613 | 37.9959 | 663 |
| 2612 | 1 | TGATCTATCCTAATTCGAGG | AGG | 84.27752 | 65.69044 | 664 |
| 2706 | −1 | GACTAGCAAATATTTATCTA | TGG | 71.26871 | 42.60356 | 665 |
| 2728 | −1 | GAAGTTGACTATGGAAAGAA | AGG | 68.31233 | 50.43304 | 666 |
| 2737 | −1 | TGCCATATTGAAGTTGACTA | TGG | 80.8359 | 46.74391 | 667 |
| 2746 | 1 | TTCCATAGTCAACTTCAATA | TGG | 80.26648 | 36.21752 | 668 |
| 2773 | −1 | AGTTGAGCATCATTTGTTGA | TGG | 67.66315 | 40.13285 | 669 |
| 2819 | 1 | ATTTATATTTATTTTTAGTA | AGG | 33.72914 | 28.85349 | 670 |
| 2820 | 1 | TTTATATTTATTTTTAGTAA | GGG | 30.35197 | 42.37447 | 671 |
| 2837 | −1 | CAAAATTAGGCATCATTGTT | AGG | 78.91428 | 34.86543 | 672 |
| 2849 | 1 | CTAACAATGATGCCTAATTT | TGG | 71.83239 | 32.31041 | 673 |
| 2850 | −1 | AAAAAAAATTCACCAAAATT | AGG | 40.46491 | 47.7423 | 674 |
| 2875 | −1 | TGATATCATTAAGTCACATG | TGG | 76.59019 | 59.28563 | 675 |
| 2893 | 1 | TGACTTAATGATATCAAATT | AGG | 39.65251 | 38.32681 | 676 |
| 2927 | −1 | AGCTTTTTATAATGGAGCAG | GGG | 87.33466 | 59.01784 | 677 |
| 2928 | −1 | TAGCTTTTTATAATGGAGCA | GGG | 86.07683 | 56.81693 | 678 |
| 2929 | −1 | ATAGCTTTTTATAATGGAGC | AGG | 67.82726 | 40.53187 | 679 |
| 2935 | −1 | CTATTTATAGCTTTTTATAA | TGG | 47.40677 | 25.75732 | 680 |
| 2947 | 1 | CATTATAAAAAGCTATAAAT | AGG | 53.188 | 27.28205 | 681 |

TABLE 7
gRNA sequences targeted for the promoter region of Cannabidiolic
acid synthase-like 1 (pCBDAS2)

| Position on | | | | Specificity | Efficiency | SEQ |
|---|---|---|---|---|---|---|
| SEQ ID NO 1150 | Strand | Sequence | PAM | Score | Score | ID NO |
| 3608 | 1 | TCGTAAAGTTTTTGCCTTTT | TGG | 65.39281 | 5.30152 | 682 |
| 3611 | −1 | TGATATTGTACATACCAAAA | AGG | 60.44733 | 45.02422 | 683 |
| 3643 | 1 | TAATAACTTTATATAAATAT | GGG | 36.56014 | 38.32366 | 684 |
| 3644 | −1 | ATAATAACTTTATATAAATA | TGG | 33.32154 | 25.20198 | 685 |
| 3754 | −1 | TTTAACTAGATCAAATAATT | TGG | 48.05611 | 23.07388 | 686 |
| 3776 | 1 | GATCTAGTTAAATGCTTACT | CGG | 73.66535 | 47.36085 | 687 |
| 3846 | 1 | TGTAATTTGTTTTTTAAAAA | AGG | 34.47486 | 27.52468 | 688 |
| 3914 | 1 | TAATAATATAAGCTTTACGT | AGG | 81.26203 | 60.03723 | 689 |
| 3937 | 1 | CACTTTATTCTTATGTAAAA | AGG | 56.2698 | 13.41993 | 690 |
| 3938 | 1 | ACTTTATTCTTATGTAAAAA | GGG | 51.43859 | 39.42698 | 691 |
| 3952 | −1 | GGCTTTGTCCGCTTCAATTT | TGG | 86.87182 | 23.56961 | 692 |
| 3955 | 1 | AAAGGGTACCAAAATTGAAG | CGG | 60.85406 | 66.61581 | 693 |
| 3973 | −1 | AAAAGTCAATATTTTCTTGT | CGG | 46.67105 | 36.98964 | 694 |
| 4084 | 1 | ATAGTAGTCAAATAAAAATT | TGG | 41.98765 | 44.23371 | 695 |
| 4116 | 1 | TTATTCGTTAAACTCAATTA | TGG | 59.8098 | 35.90179 | 696 |
| 4119 | −1 | TTCGTTAAACTCAATTATGG | TGG | 54.61632 | 50.54886 | 697 |
| 4124 | 1 | TAAACTCAATTATGGTGGAT | TGG | 74.65406 | 41.55014 | 698 |
| 4191 | 1 | TATTATATATTAAAATTAGA | CGG | 32.1905 | 42.92174 | 699 |
| 4251 | 1 | AAATTTGTTAAAAAAATAGC | TGG | 60.27741 | 47.43196 | 700 |
| 4433 | −1 | ATAATAATATATATATATAT | AGG | 27.24259 | 41.2241 | 701 |
| 4503 | 1 | TGTTATAAATACTAGAAATT | TGG | 45.04998 | 29.32708 | 702 |
| 4509 | 1 | AAATACTAGAAATTTGGAAC | TGG | 67.41128 | 32.64608 | 703 |
| 4510 | 1 | AATACTAGAAATTTGGAACT | GGG | 57.48187 | 59.20982 | 704 |
| 4526 | −1 | ATTAAAAAATAATAAAAATA | CGG | 17.0049 | 37.02224 | 705 |
| 4716 | −1 | CCGGGATAACCATTAGGAAT | TGG | 91.45553 | 40.10488 | 706 |
| 4718 | 1 | TTAATTCGTCCAATTCCTAA | TGG | 77.25671 | 41.95217 | 707 |
| 4722 | −1 | ATCAAACCGGGATAACCATT | AGG | 87.76223 | 45.74725 | 708 |
| 4727 | 1 | CCAATTCCTAATGGTTATCC | CGG | 91.69682 | 43.65963 | 709 |
| 4734 | −1 | AAATTAACTTTGATCAAACC | GGG | 41.69826 | 57.50517 | 710 |
| 4735 | 1 | CAAATTAACTTTGATCAAAC | CGG | 69.58298 | 37.76064 | 711 |
| 4805 | 1 | TATTATTCATTTTTAATAGA | AGG | 32.81432 | 41.83116 | 712 |
| 4833 | −1 | TTCAAATAATACAATGTAAG | TGG | 58.95513 | 50.28611 | 713 |
| 4874 | 1 | ATACTATTAAATTAGTTATG | TGG | 45.48745 | 51.42514 | 714 |
| 4896 | −1 | TAAATAAAAATACTGAGTCA | TGG | 69.24537 | 58.05747 | 715 |
| 4951 | 1 | TTTTTAGAATTCTCATAATA | TGG | 50.52634 | 25.57262 | 716 |
| 4990 | 1 | ACTAATGACTCATTGAATCT | AGG | 82.39466 | 45.1962 | 717 |
| 4991 | 1 | CTAATGACTCATTGAATCTA | GGG | 79.71165 | 38.20962 | 718 |
| 5017 | 1 | ATTTTAAAGATAAACAAAGT | AGG | 38.70038 | 52.31175 | 719 |
| 5029 | 1 | TAGAGTTGGGTGCTAGGCGT | GGG | 98.04831 | 53.64932 | 720 |
| 5030 | −1 | ATAGAGTTGGGTGCTAGGCG | TGG | 96.44132 | 51.22564 | 721 |
| 5035 | −1 | CCTCAATAGAGTTGGGTGCT | AGG | 94.53012 | 49.14025 | 722 |
| 5042 | −1 | TTCACGGCCTCAATAGAGTT | GGG | 93.15 | 46.68094 | 723 |
| 5043 | −1 | TTTCACGGCCTCAATAGAGT | TGG | 90.46379 | 49.78122 | 724 |
| 5046 | 1 | CCTAGCACCCAACTCTATTG | AGG | 95.79977 | 53.27657 | 725 |
| 5058 | −1 | ATTTGATTTTTATTTTTTCA | CGG | 32.16797 | 32.8882 | 726 |
| 5167 | 1 | CTTTAAAATATCTTTAATTA | TGG | 40.62727 | 22.20639 | 727 |
| 5280 | 1 | TTAATTCACATAATATATAT | CGG | 30.81355 | 41.09798 | 728 |
| 5305 | 1 | TTCCATGAAAGTACAATCAC | GGG | 87.68631 | 56.64862 | 729 |
| 5306 | 1 | ATTCCATGAAAGTACAATCA | CGG | 70.53498 | 55.70196 | 730 |
| 5314 | 1 | GTCCCGTGATTGTACTTTCA | TGG | 94.73198 | 30.29386 | 731 |
| 5334 | −1 | CTAACGGCTGTCGTACATCA | CGG | 98.9975 | 65.28958 | 732 |
| 5350 | −1 | CAAATAACTCCCTCATCTAA | CGG | 74.89986 | 46.73712 | 733 |
| 5351 | 1 | TGTACGACAGCCGTTAGATG | AGG | 95.38156 | 57.02933 | 734 |
| 5352 | 1 | GTACGACAGCCGTTAGATGA | GGG | 96.48181 | 63.86618 | 735 |
| 5367 | 1 | GATGAGGGAGTTATTTGATC | TGG | 51.57045 | 37.53623 | 736 |
| 5368 | 1 | ATGAGGGAGTTATTTGATCT | GGG | 29.67916 | 46.09647 | 737 |
| 5369 | 1 | TGAGGGAGTTATTTGATCTG | GGG | 46.20116 | 60.55723 | 738 |
| 5370 | 1 | GAGGGAGTTATTTGATCTGG | GGG | 57.9185 | 63.73898 | 739 |
| 5386 | 1 | CTGGGGGCTGAGATTGATCT | AGG | 89.29237 | 38.16727 | 740 |
| 5387 | 1 | TGGGGGCTGAGATTGATCTA | GGG | 92.31454 | 46.43138 | 741 |
| 5388 | 1 | GGGGGCTGAGATTGATCTAG | GGG | 86.12787 | 58.2749 | 742 |
| 5396 | 1 | AGATTGATCTAGGGGTAATT | AGG | 73.47678 | 20.39912 | 743 |
| 5397 | 1 | GATTGATCTAGGGGTAATTA | GGG | 74.48836 | 35.45681 | 744 |
| 5427 | 1 | TCAGGGGTACATTAGTGTCA | GGG | 92.96474 | 62.87707 | 745 |
| 5428 | 1 | CTCAGGGGTACATTAGTGTC | AGG | 97.31543 | 39.87957 | 746 |
| 5443 | −1 | TGGTATTAGATGGGTCTCAG | GGG | 95.18347 | 68.57942 | 747 |
| 5444 | 1 | GTGGTATTAGATGGGTCTCA | GGG | 93.76182 | 54.88957 | 748 |
| 5445 | −1 | CGTGGTATTAGATGGGTCTC | AGG | 92.48038 | 38.72919 | 749 |
| 5452 | −1 | TGTATTCCGTGGTATTAGAT | GGG | 81.9675 | 47.38183 | 750 |
| 5453 | 1 | ATGTATTCCGTGGTATTAGA | TGG | 91.87071 | 40.46008 | 751 |
| 5457 | 1 | CTGAGACCCATCTAATACCA | CGG | 91.40663 | 55.19811 | 752 |
| 5463 | −1 | CTTTCACGGAATGTATTCCG | TGG | 98.95836 | 67.8176 | 753 |
| 5477 | −1 | TTTCCCGTGATGTACTTTCA | CGG | 89.22499 | 36.21454 | 754 |
| 5484 | 1 | CATTCCGTGAAAGTACATCA | CGG | 89.16249 | 67.85904 | 755 |
| 5485 | 1 | ATTCCGTGAAAGTACATCAC | GGG | 87.03673 | 61.11098 | 756 |
| 5509 | −1 | AATAGATATATATATATATT | GGG | 32.28234 | 29.08498 | 757 |
| 5510 | −1 | AAATAGATATATATATATAT | TGG | 34.85681 | 25.30293 | 758 |
| 5632 | −1 | AGTTTTTGAAACTCTTCTAA | TGG | 57.81115 | 39.90145 | 759 |
| 5680 | 1 | AAAGTCAAATATTATAATTT | AGG | 33.62202 | 33.13317 | 760 |
| 5694 | −1 | AATTCTCTTAATAAATTATA | GGG | 40.91586 | 31.37454 | 761 |
| 5695 | −1 | AAATTCTCTTAATAAATTAT | AGG | 45.24293 | 17.50146 | 762 |
| 5763 | 1 | ACTTATTCAATCATTAATAA | AGG | 49.02488 | 33.4404 | 763 |
| 5779 | 1 | ATAAAGGTTAACAATGATCA | TGG | 68.22139 | 44.9581 | 764 |
| 5780 | 1 | TAAAGGTTAACAATGATCAT | GGG | 46.84193 | 47.94026 | 765 |
| 5781 | 1 | AAAGGTTAACAATGATCATG | GGG | 61.7463 | 64.41274 | 766 |
| 5793 | −1 | TGTGCCTAATGTTGTAGTTA | AGG | 62.40157 | 36.71787 | 767 |
| 5800 | 1 | GGGGCCTTAACTACAACATT | AGG | 88.21292 | 48.93839 | 768 |
| 5814 | 1 | AACATTAGGCACATTTTCAA | TGG | 62.9861 | 38.06074 | 769 |
| 5833 | −1 | CCCATAATAAAAGTGGCTTT | TGG | 90.66364 | 34.94275 | 770 |
| 5840 | −1 | TCATATCCCCATAATAAAAG | TGG | 66.3258 | 54.42597 | 771 |
| 5843 | 1 | TCCAAAAGCCACTTTTATTA | TGG | 74.43741 | 22.26555 | 772 |
| 5844 | 1 | CCAAAAGCCACTTTTATTAT | GGG | 79.64392 | 20.2432 | 773 |
| 5845 | 1 | CAAAAGCCACTTTTATTATG | GGG | 72.6251 | 45.26761 | 774 |
| 6078 | 1 | TTTTTTTAGTCTTAATTAAG | TGG | 49.00529 | 46.30457 | 775 |
| 6270 | 1 | TATGTGTATTAAAATTAAAT | AGG | 38.04988 | 26.5559 | 776 |
| 6295 | −1 | GTACCGGGTTTTAAATAATT | TGG | 80.18079 | 18.20234 | 777 |
| 6303 | 1 | GAACCAAATTATTTAAAACC | CGG | 56.66524 | 60.70411 | 778 |
| 6310 | −1 | TAGGAGGGTTAAAGAGTACC | GGG | 93.90428 | 56.09004 | 779 |
| 6311 | −1 | GTAGGAGGGTTAAAGAGTAC | CGG | 86.15869 | 52.3736 | 780 |
| 6325 | −1 | TTTTTCAGTGGGTGGTAGGA | GGG | 88.20339 | 40.82475 | 781 |
| 6326 | −1 | GTTTTTCAGTGGGTGGTAGG | AGG | 93.24801 | 47.6339 | 782 |
| 6329 | −1 | ATAGTTTTTCAGTGGGTGGT | AGG | 85.77888 | 47.98267 | 783 |
| 6333 | −1 | TAATATAGTTTTTCAGTGGG | TGG | 86.4104 | 48.96699 | 784 |
| 6336 | −1 | GTGTAATATAGTTTTTCAGT | GGG | 71.9731 | 57.13588 | 785 |
| 6337 | −1 | AGTGTAATATAGTTTTTCAG | TGG | 70.30391 | 60.5271 | 786 |
| 6370 | −1 | TAATTTGCCTTTTATTCTCA | TGG | 68.35563 | 36.47194 | 787 |
| 6374 | 1 | ACTTTAACCATGAGAATAAA | AGG | 67.30249 | 26.61748 | 788 |
| 6388 | 1 | AATAAAAGGCAAATTAAGAG | TGG | 53.31912 | 51.49194 | 789 |
| 6389 | 1 | ATAAAAGGCAAATTAAGAGT | GGG | 65.30809 | 55.95129 | 790 |
| 6428 | 1 | AAAAAAAAGTGAATTTCAAG | AGG | 26.28604 | 64.09909 | 791 |
| 6495 | −1 | ATCACCAAGATTGAAAATGG | TGG | 68.13091 | 60.52911 | 792 |
| 6498 | −1 | CTTATCACCAAGATTGAAAA | TGG | 72.1994 | 36.57177 | 793 |
| 6502 | 1 | AAGTCCACCATTTTCAATCT | TGG | 59.13025 | 33.30747 | 794 |

TABLE 8
gRNA sequences targeted for the promoter region of FNCBDAS
(pFNCBDAS)

| Position on | | | | Specificity | Efficiency | SEQ |
|---|---|---|---|---|---|---|
| SEQ ID NO 1151 | Strand | Sequence | PAM | Score | Score | ID NO |
| 28 | 1 | CTTATATAGTACCGTTAATT | TGG | 87.08584 | 20.41182 | 795 |
| 28 | −1 | TATAGGTATCGCCAAATTAA | CGG | 78.83655 | 33.0987 | 796 |
| 45 | 1 | TAGTTAGAGACTCGGAGTAT | AGG | 94.17688 | 48.77532 | 797 |
| 53 | −1 | TCAAAGAATAGTTAGAGACT | CGG | 69.30951 | 61.13112 | 798 |
| 116 | 1 | TGATATAAATGATACATTAA | TGG | 48.65095 | 21.14936 | 799 |
| 171 | 1 | ATTACACATATTTAGAATGA | AGG | 57.47496 | 47.17923 | 800 |
| 252 | 1 | GAAAATTTTATTTGCATCCC | AGG | 78.33627 | 45.91196 | 801 |
| 258 | −1 | AATATGAGGGTGTACTTCCT | GGG | 91.52919 | 46.25534 | 802 |
| 259 | −1 | TAATATGAGGGTGTACTTCC | TGG | 90.65025 | 35.22345 | 803 |
| 271 | −1 | ATTTTTTTTTTTTAATATGA | GGG | 37.77447 | 36.76134 | 804 |
| 272 | 1 | TATTTTTTTTTTTTAATATG | AGG | 37.51222 | 50.45735 | 805 |
| 346 | 1 | ATTTTTTTTAAATATTATTT | TGG | 16.26903 | 12.53282 | 806 |
| 347 | 1 | TTTTTTTTAAATATTATTTT | GGG | 20.13834 | 12.3568 | 807 |
| 416 | 1 | ATAAAATTTCACATGAATTT | TGG | 40.19066 | 21.11207 | 808 |
| 511 | 1 | ATAATTTATTTTTATTTGAT | AGG | 30.84674 | 34.67275 | 809 |
| 524 | 1 | ATTTGATAGGTATATTTTTT | AGG | 44.20707 | 14.5258 | 810 |
| 613 | −1 | TAAAATAATTTGATGAAAAT | AGG | 36.08971 | 26.2173 | 811 |
| 747 | −1 | CAAGAAACATCCAACTCATA | TGG | 70.9779 | 41.32498 | 812 |
| 748 | 1 | ACGAAGATAGCCATATGAGT | TGG | 90.52873 | 54.2775 | 813 |
| 774 | −1 | AATATAACTTACGCGTCATA | AGG | 89.84602 | 43.41139 | 814 |
| 898 | −1 | AGGTTTTGAGATAAGTATGA | AGG | 64.26412 | 42.76187 | 815 |
| 918 | −1 | AACGTGAAACTGAATAAATT | AGG | 45.72429 | 41.01979 | 816 |
| 949 | 1 | ATAAATCAATTACAATTGAA | AGG | 42.22023 | 41.67737 | 817 |
| 1039 | −1 | AAAGAATAAAAGAACATAAA | AGG | 29.53055 | 31.4109 | 818 |
| 1316 | −1 | TTAATACAAGTAAAAAATAA | AGG | 35.52477 | 38.4953 | 819 |
| 1341 | 1 | TTGTATTAATTTTCAAGATA | CGG | 49.95775 | 49.55918 | 820 |
| 1360 | 1 | ACGGTTGTATATATTATTTA | AGG | 53.27125 | 21.43581 | 821 |
| 1408 | 1 | AACTTTTTTTTTTAAATTTA | CGG | 26.18551 | 3.475133 | 822 |
| 1413 | 1 | TTTTTTTTAAATTTACGGTT | TGG | 64.31414 | 30.24835 | 823 |
| 1414 | 1 | TTTTTTTAAATTTACGGTTT | GGG | 62.59624 | 26.50112 | 824 |
| 1426 | 1 | TACGGTTTGGGTTTCTAAAG | TGG | 84.59146 | 37.30848 | 825 |
| 1448 | 1 | GTTGCAGCGCTAGTTGCAAT | AGG | 93.90842 | 50.57628 | 826 |
| 1449 | 1 | TTGCAGCGCTAGTTGCAATA | GGG | 95.54869 | 45.42281 | 827 |
| 1450 | 1 | TGCAGCGCTAGTTGCAATAG | GGG | 95.24333 | 53.85393 | 828 |
| 1480 | 1 | ACGATTTTTTGTTGCAATTT | AGG | 50.89591 | 22.7977 | 829 |
| 1531 | −1 | TTTTTTTTTTTGCATTTTTA | CGG | 40.607 | 13.20345 | 830 |
| 1596 | −1 | ATATAATAAATAAATAATAG | GGG | 28.96024 | 55.7901 | 831 |
| 1597 | −1 | TATATAATAAATAAATAATA | GGG | 20.66199 | 23.86282 | 832 |
| 1598 | −1 | ATATATAATAAATAAATAAT | AGG | 19.61297 | 17.7088 | 833 |
| 1685 | 1 | CATATTTCTAGAGACTTTGT | TGG | 61.68745 | 34.75339 | 834 |
| 1709 | 1 | GATTGACTTTGTGTCATATA | TGG | 73.26395 | 37.03831 | 835 |
| 1712 | 1 | TGACTTTGTGTCATATATGG | TGG | 77.15193 | 59.23541 | 836 |
| 1731 | 1 | GTGGCAAATATGTACCTTGA | TGG | 29.71746 | 52.90308 | 837 |
| 1734 | −1 | AATCAACCCTAGCTCCATCA | AGG | 89.23227 | 63.37798 | 838 |
| 1738 | 1 | ATATGTACCTTGATGGAGCT | AGG | 80.48791 | 49.63265 | 839 |
| 1739 | 1 | TATGTACCTTGATGGAGCTA | GGG | 89.28452 | 54.26049 | 840 |
| 1754 | 1 | AGCTAGGGTTGATTATGCTA | TGG | 88.11542 | 60.47483 | 841 |
| 1758 | 1 | AGGGTTGATTATGCTATGGT | TGG | 64.87314 | 55.80674 | 842 |
| 1762 | 1 | TTGATTATGCTATGGTTGGT | CGG | 63.38052 | 57.86866 | 843 |
| 1765 | 1 | ATTATGCTATGGTTGGTCGG | TGG | 95.70099 | 64.0234 | 844 |
| 1784 | 1 | GTGGCTAGTTGTACCATGTT | TGG | 88.10271 | 44.60268 | 845 |
| 1786 | −1 | AAAAAAATACCCACCAAACA | TGG | 55.37097 | 54.8231 | 846 |
| 1787 | 1 | GCTAGTTGTACCATGTTTGG | TGG | 78.23537 | 54.11298 | 847 |
| 1788 | 1 | CTAGTTGTACCATGTTTGGT | GGG | 47.82538 | 55.65218 | 848 |
| 1895 | −1 | AATTGTACAAAAAACTAACA | AGG | 58.33307 | 63.51655 | 849 |
| 1933 | 1 | TATAGTTTTGATGCCTTTTT | AGG | 64.563 | 9.48421 | 850 |
| 1934 | 1 | ATAGTTTTGATGCCTTTTTA | GGG | 60.54761 | 26.17371 | 851 |
| 1935 | −1 | ATGGTACAAATGCCCTAAAA | AGG | 80.64249 | 29.63382 | 852 |
| 1954 | −1 | TAAGAGCAAGCCATAAAATA | TGG | 72.71978 | 35.22553 | 853 |
| 1955 | 1 | GGCATTTGTACCATATTTTA | TGG | 64.0604 | 12.56948 | 854 |
| 1987 | 1 | AATTGTTTAGCGTAAATTTG | AGG | 65.13244 | 45.66684 | 855 |
| 1992 | 1 | TTTAGCGTAAATTTGAGGTA | TGG | 70.96348 | 44.18963 | 856 |
| 2004 | −1 | AAAACCCTTCTCTTAATCAT | TGG | 71.42414 | 47.35616 | 857 |
| 2010 | 1 | TATGGCCAATGATTAAGAGA | AGG | 87.94942 | 54.52333 | 858 |
| 2011 | 1 | ATGGCCAATGATTAAGAGAA | GGG | 81.88333 | 54.92321 | 859 |
| 2030 | 1 | AGGGTTTTGTTTTGTAGTCT | TGG | 65.6361 | 33.35379 | 860 |
| 2031 | 1 | GGGTTTTGTTTTGTAGTCTT | GGG | 56.51178 | 39.40289 | 861 |
| 2132 | −1 | ACAATTTAATAAAAAAAAAT | AGG | 45.30986 | 40.83127 | 862 |
| 2194 | 1 | TTTGTAAGTAATTTTTATTT | AGG | 37.24822 | 20.66302 | 863 |
| 2229 | 1 | GCTATTTTTTTTTTTTTTGT | AGG | 53.54846 | 31.17083 | 864 |
| 2233 | 1 | TTTTTTTTTTTTTTGTAGGT | CGG | 48.64907 | 35.49889 | 865 |
| 2244 | 1 | TTTGTAGGTCGGTTTTGTTA | AGG | 72.21784 | 35.77156 | 866 |
| 2245 | 1 | TTGTAGGTCGGTTTTGTTAA | GGG | 83.38201 | 32.18371 | 867 |
| 2361 | 1 | TAGTTTTTAATATAATTGTT | AGG | 39.84944 | 32.39923 | 868 |
| 2362 | 1 | AGTTTTTAATATAATTGTTA | GGG | 37.20696 | 33.4409 | 869 |
| 2363 | 1 | GTTTTTAATATAATTGTTAG | GGG | 53.61389 | 54.79293 | 870 |
| 2407 | 1 | ATTTTTTTGTAGATATTTTG | TGG | 40.37926 | 31.16942 | 871 |
| 2420 | −1 | ACACCAACTCATATAACAAA | TGG | 56.53691 | 42.69107 | 872 |
| 2428 | 1 | GGACCATTTGTTATATGAGT | TGG | 83.86505 | 49.29725 | 873 |
| 2519 | 1 | TTTATTTTCTAAATTTTTAG | TGG | 40.13616 | 39.90673 | 874 |
| 2540 | −1 | CTAAAGTAAGCAAGCAACAT | GGG | 50.21894 | 56.38343 | 875 |
| 2541 | −1 | GCTAAAGTAAGCAAGCAACA | TGG | 64.02802 | 71.00251 | 876 |
| 2582 | 1 | AGTTTGACAAAGCATGCTAT | TGG | 79.83251 | 47.56961 | 877 |
| 2610 | 1 | CGATGATCTATCCTAGTTCG | AGG | 91.72015 | 47.00061 | 878 |
| 2610 | −1 | TGCAGAAACTTCCTCGAACT | AGG | 80.13912 | 61.25399 | 879 |
| 2631 | 1 | GGAAGTTTCTGCAATATTTG | TGG | 39.73765 | 46.79753 | 880 |
| 2726 | −1 | GAAGTTGACTATGGAAAGAA | AGG | 68.31233 | 50.43304 | 881 |
| 2735 | −1 | TGCCATATTGAAGTTGACTA | TGG | 80.8359 | 46.74391 | 882 |
| 2744 | 1 | TTCCATAGTCAACTTCAATA | TGG | 80.26648 | 36.21752 | 883 |
| 2771 | −1 | AGTTGAGCATCATTTGTTGA | TGG | 67.66315 | 40.13285 | 884 |
| 2817 | 1 | ATTTATATTTATTTTTAATA | AGG | 23.11656 | 17.71186 | 885 |
| 2818 | 1 | TTTATATTTATTTTTAATAA | GGG | 19.60135 | 27.46306 | 886 |
| 2835 | −1 | TTAGGCATCATTGTATTGTT | AGG | 79.95832 | 35.76057 | 887 |
| 2853 | −1 | AAAAAAAATTCACAAAAATT | AGG | 34.9378 | 47.23013 | 888 |
| 2877 | −1 | TTTGATATCATTAAGTCATG | TGG | 71.70666 | 57.24842 | 889 |
| 2927 | −1 | AGCTTTATATATTGGAGCAG | GGG | 83.50935 | 54.68597 | 890 |
| 2928 | −1 | TAGCTTTATATATTGGAGCA | GGG | 81.38255 | 56.13836 | 891 |
| 2929 | −1 | ATAGCTTTATATATTGGAGC | AGG | 72.50846 | 39.33975 | 892 |
| 2935 | −1 | CTATTTATAGCTTTATATAT | TGG | 43.77789 | 24.05235 | 893 |
| 2947 | 1 | CAATATATAAAGCTATAAAT | AGG | 55.6662 | 27.93541 | 894 |
| 2971 | −1 | TAATGAATTTTGAATTACTA | TGG | 49.62968 | 34.40115 | 895 |

TABLE 9
gRNA sequences targeted for the promoter region of PKCBDAS
(pPKCBDAS)

| Position on | | | | Specificity | Efficiency | SEQ |
|---|---|---|---|---|---|---|
| SEQ ID NO 1152 | Strand | Sequence | PAM | Score | Score | ID NO |
| 15 | −1 | AAATTTATAGGAAACCCCTA | TGG | 79.05992 | 60.82868 | 896 |
| 27 | −1 | AAAAATTTTAAAAAATTTAT | AGG | 24.88478 | 12.51009 | 897 |
| 153 | 1 | GCTCTAAGTGTTTGTATATT | AGG | 71.02887 | 19.72184 | 898 |
| 213 | −1 | TAAAAATGATACTAAAATAC | TGG | 50.14456 | 45.12403 | 899 |
| 487 | 1 | TACATTTAACTTTTATAATA | TGG | 40.94089 | 13.19144 | 900 |
| 488 | 1 | ACATTTAACTTTTATAATAT | GGG | 39.82131 | 31.88376 | 901 |
| 685 | −1 | AATTACAAAAATGGTCTATT | GGG | 67.45602 | 31.22343 | 902 |
| 686 | −1 | AAATTACAAAAATGGTCTAT | TGG | 69.20416 | 32.73834 | 903 |
| 694 | −1 | CCAAAAAAAAATTACAAAAA | TGG | 40.17525 | 32.6503 | 904 |
| 705 | 1 | CCATTTTTGTAATTTTTTTT | TGG | 55.03665 | 4.39017 | 905 |
| 754 | −1 | TTAGAATAATATTAATACGT | AGG | 60.19719 | 55.44839 | 906 |
| 786 | 1 | AAAATTACTCTAAGTATTTA | AGG | 47.79289 | 10.74525 | 907 |
| 851 | 1 | AGCACATAATTTTTTGTATA | AGG | 56.48597 | 28.75291 | 908 |
| 880 | 1 | TTTATCGAAATTGACTTTAT | CGG | 49.07938 | 20.52118 | 909 |
| 905 | 1 | TTCTCTTAAACTTGGTTGTT | AGG | 71.94596 | 30.47243 | 910 |
| 913 | 1 | ATTTTAGTTTCTCTTAAACT | TGG | 58.60494 | 44.10799 | 911 |
| 946 | −1 | GCCGAAATTTCGGTAGAATT | AGG | 93.91936 | 38.76708 | 912 |
| 956 | 1 | TCCTAATTCTACCGAAATTT | CGG | 81.17653 | 33.0812 | 913 |
| 956 | −1 | CCGTTATGCTGCCGAAATTT | CGG | 96.80906 | 25.73719 | 914 |
| 967 | 1 | CCGAAATTTCGGCAGCATAA | CGG | 97.15419 | 49.31822 | 915 |
| 995 | −1 | ATTAATAGGTTTGAATTTTT | TGG | 32.90635 | 14.06121 | 916 |
| 1009 | 1 | TGTGTTTTGTTGTTATTAAT | AGG | 28.42475 | 19.4856 | 917 |
| 1084 | −1 | ATGTACTGTAGTCGGATGGG | TGG | 97.93272 | 65.48715 | 918 |
| 1087 | 1 | TACATGTACTGTAGTCGGAT | GGG | 96.82472 | 62.50227 | 919 |
| 1088 | −1 | ATACATGTACTGTAGTCGGA | TGG | 95.56897 | 64.70083 | 920 |
| 1092 | −1 | GTGAATACATGTACTGTAGT | CGG | 78.04598 | 58.39075 | 921 |
| 1119 | −1 | ATATTCATCTGTAGTGAAGT | AGG | 85.75811 | 56.2417 | 922 |
| 1181 | −1 | TTGTACTATGTCGGATCGAT | GGG | 97.03269 | 54.25148 | 923 |
| 1182 | −1 | ATTGTACTATGTCGGATCGA | TGG | 97.34184 | 52.78956 | 924 |
| 1190 | −1 | TACAATATATTGTACTATGT | CGG | 64.42547 | 46.16301 | 925 |
| 1216 | 1 | ATATTCATTTGTAGTGAAGT | AGG | 70.68127 | 55.06829 | 926 |
| 1310 | −1 | TTTTGTGTTGATCGGTTTCT | AGG | 87.0286 | 32.77909 | 927 |
| 1318 | −1 | CCAACTTATTTTGTGTTGAT | CGG | 62.34539 | 41.27599 | 928 |
| 1329 | 1 | CCGATCAACACAAAATAAGT | TGG | 70.50758 | 47.47113 | 929 |
| 1343 | −1 | GGTTGTGAGGTCAATTTGCA | AGG | 84.69011 | 57.79606 | 930 |
| 1356 | 1 | TCTTTATGTTGAAGGTTGTG | AGG | 73.63441 | 63.60809 | 931 |
| 1364 | 1 | GTAGTATTTCTTTATGTTGA | AGG | 51.8172 | 34.05116 | 932 |
| 1394 | 1 | AAGTAGCTAAATAAAAAAAT | TGG | 53.29758 | 40.44954 | 933 |
| 1546 | −1 | ATGTGTTTTATTTCTTTAGT | AGG | 56.40591 | 41.80989 | 934 |
| 1676 | −1 | ATTGTGAATGAGAATGAGAT | AGG | 56.94192 | 50.18484 | 935 |
| 1761 | −1 | GTGAATGAGAAATGTAATAT | AGG | 48.21007 | 37.67949 | 936 |
| 1854 | −1 | TGTGTATATCTATTGTGAAT | GGG | 55.62819 | 40.32213 | 937 |
| 1855 | 1 | ATGTGTATATCTATTGTGAA | TGG | 62.59737 | 53.71475 | 938 |
| 1924 | 1 | TTATTTTATAAATTTTTTTA | GGG | 42.0296 | 11.47894 | 939 |
| 1925 | −1 | GTTATTTTATAAATTTTTTT | AGG | 42.51547 | 8.149528 | 940 |
| 1993 | 1 | AATTAGATTTATACCTTAAT | AGG | 56.58292 | 19.71885 | 941 |
| 1995 | −1 | TTGTATCTCAACGCCTATTA | AGG | 90.0411 | 28.28075 | 942 |
| 2026 | −1 | CCTCCGGCCACCGTTTTTAG | TGG | 98.38454 | 39.87017 | 943 |
| 2027 | 1 | AATGTTTTCTCCACTAAAAA | CGG | 65.26185 | 40.5693 | 944 |
| 2030 | 1 | GTTTTCTCCACTAAAAACGG | TGG | 93.09942 | 69.96043 | 945 |
| 2034 | 1 | TCTCCACTAAAAACGGTGGC | CGG | 94.89369 | 50.91334 | 946 |
| 2037 | 1 | CCACTAAAAACGGTGGCCGG | AGG | 99.55525 | 64.66545 | 947 |
| 2042 | 1 | GGTAGTGGTGATTATACCTC | CGG | 93.74824 | 56.29174 | 948 |
| 2057 | −1 | TAAACAAAAAGTAAAGGTAG | TGG | 55.48546 | 59.33194 | 949 |
| 2063 | −1 | TTGGGGTAAACAAAAAGTAA | AGG | 58.11796 | 48.18294 | 950 |
| 2080 | −1 | ACATTTTTCCTCATTTTTTG | GGG | 44.42423 | 47.03086 | 951 |
| 2081 | −1 | TACATTTTTCCTCATTTTTT | GGG | 37.42586 | 10.97553 | 952 |
| 2082 | 1 | TTACATTTTTCCTCATTTTT | TGG | 35.35375 | 20.4246 | 953 |
| 2083 | 1 | TTTGTTTACCCCAAAAAATG | AGG | 36.92673 | 56.26361 | 954 |
| 2103 | 1 | AGGAAAAATGTAATCTTTTC | AGG | 52.84995 | 15.51548 | 955 |
| 2117 | 1 | CTTTTCAGGTATATAGTTTT | AGG | 63.77883 | 14.06283 | 956 |
| 2160 | 1 | GAAATAAACATGAGCTAAAA | TGG | 47.4092 | 24.47995 | 957 |
| 2179 | 1 | ATGGTGAAAAAATAGTGAAA | TGG | 54.77197 | 46.19193 | 958 |
| 2182 | 1 | GTGAAAAAATAGTGAAATGG | AGG | 44.30837 | 65.41596 | 959 |
| 2195 | 1 | GAAATGGAGGTGATTTTTCG | TGG | 81.00618 | 53.92372 | 960 |
| 2198 | −1 | ATGGAGGTGATTTTTCGTGG | TGG | 82.72984 | 52.56797 | 961 |
| 2202 | 1 | AGGTGATTTTTCGTGGTGGT | TGG | 86.11241 | 42.57064 | 962 |
| 2205 | 1 | TGATTTTTCGTGGTGGTTGG | TGG | 75.63488 | 46.27217 | 963 |
| 2210 | 1 | TTTCGTGGTGGTTGGTGGAG | AGG | 80.1843 | 44.50687 | 964 |
| 2211 | 1 | TTCGTGGTGGTTGGTGGAGA | GGG | 78.7611 | 44.13182 | 965 |
| 2216 | 1 | GGTGGTTGGTGGAGAGGGTT | TGG | 86.23921 | 17.86371 | 966 |
| 2217 | 1 | GTGGTTGGTGGAGAGGGTTT | GGG | 66.99371 | 30.55058 | 967 |
| 2218 | 1 | TGGTTGGTGGAGAGGGTTTG | GGG | 83.51557 | 40.53515 | 968 |
| 2227 | 1 | GAGAGGGTTTGGGGTTTCTT | TGG | 69.82314 | 32.5218 | 969 |
| 2232 | 1 | GGTTTGGGGTTTCTTTGGTT | TGG | 72.13771 | 28.46218 | 970 |
| 2233 | 1 | GTTTGGGGTTTCTTTGGTTT | GGG | 66.86859 | 22.12199 | 971 |
| 2234 | 1 | TTTGGGGTTTCTTTGGTTTG | GGG | 58.23159 | 28.12934 | 972 |
| 2235 | 1 | TTGGGGTTTCTTTGGTTTGG | GGG | 57.48842 | 39.0667 | 973 |
| 2242 | 1 | TTCTTTGGTTTGGGGGTTTC | TGG | 83.88754 | 0 | 974 |
| 2243 | 1 | TCTTTGGTTTGGGGGTTTCT | GGG | 80.35777 | 23.9674 | 975 |
| 2247 | 1 | TGGTTTGGGGGTTTCTGGGT | TGG | 83.09585 | 35.24111 | 976 |
| 2251 | 1 | TTGGGGGTTTCTGGGTTGGA | TGG | 88.79911 | 44.85733 | 977 |
| 2261 | 1 | CTGGGTTGGATGGTCGAATG | AGG | 91.90423 | 60.84874 | 978 |
| 2271 | 1 | TGGTCGAATGAGGAAGATGA | AGG | 79.39574 | 57.05756 | 979 |
| 2272 | 1 | GGTCGAATGAGGAAGATGAA | GGG | 69.42839 | 63.03577 | 980 |
| 2276 | 1 | GAATGAGGAAGATGAAGGGC | TGG | 78.66432 | 47.60694 | 981 |
| 2277 | 1 | AATGAGGAAGATGAAGGGCT | GGG | 72.2919 | 56.72909 | 982 |
| 2282 | 1 | GGAAGATGAAGGGCTGGGCT | AGG | 93.44725 | 38.24711 | 983 |
| 2283 | 1 | GAAGATGAAGGGCTGGGCTA | GGG | 92.30786 | 55.02592 | 984 |
| 2299 | 1 | GCTAGGGTTATAAAACCTTT | TGG | 83.70648 | 36.77708 | 985 |
| 2303 | −1 | AGAAGATAACCGACGCCAAA | AGG | 96.06026 | 48.17387 | 986 |
| 2305 | 1 | GTTATAAAACCTTTTGGCGT | CGG | 89.1036 | 50.38023 | 987 |
| 2359 | 1 | TAGTTTTTAATATAATTGTA | AGG | 43.6866 | 38.49408 | 988 |
| 2360 | 1 | AGTTTTTAATATAATTGTAA | GGG | 35.17553 | 44.01737 | 989 |
| 2361 | 1 | GTTTTTAATATAATTGTAAG | GGG | 52.01174 | 53.06262 | 990 |
| 2405 | 1 | ATTTTTTTGTAGATATTTTG | TGG | 40.37926 | 31.16942 | 991 |
| 2418 | −1 | ACACCAACTCATATAACAAA | TGG | 56.53691 | 42.69107 | 992 |
| 2426 | 1 | GGACCATTTGTTATATGAGT | TGG | 83.86505 | 49.29725 | 993 |
| 2485 | 1 | CTCTTTTATTGTGTTGTTTA | AGG | 49.5969 | 8.317329 | 994 |
| 2538 | 1 | CTAAAGTAAGCAAGCAACAT | GGG | 50.21894 | 56.38343 | 995 |
| 2539 | −1 | GCTAAAGTAAGCAAGCAACA | TGG | 64.02802 | 71.00251 | 996 |
| 2609 | 1 | CGATGATCTATCCTAATTCG | AGG | 89.16309 | 52.44567 | 997 |
| 2609 | 1 | TGCAGAAACCTCCTCGAATT | AGG | 75.27613 | 37.9959 | 998 |
| 2612 | 1 | TGATCTATCCTAATTCGAGG | AGG | 84.27752 | 65.69044 | 999 |
| 2706 | −1 | GACTAGCAAATATTTATCTA | TGG | 71.26871 | 42.60356 | 1000 |
| 2728 | −1 | GAAGTTGACTATGGAAAGAA | AGG | 68.31233 | 50.43304 | 1001 |
| 2737 | −1 | TGCCATATTGAAGTTGACTA | TGG | 80.8359 | 46.74391 | 1002 |
| 2746 | 1 | TTCCATAGTCAACTTCAATA | TGG | 80.26648 | 36.21752 | 1003 |
| 2773 | −1 | AGTTGAGCATCATTTGTTGA | TGG | 67.66315 | 40.13285 | 1004 |
| 2819 | 1 | ATTTATATTTATTTTTAGTA | AGG | 33.72914 | 28.85349 | 1005 |
| 2820 | 1 | TTTATATTTATTTTTAGTAA | GGG | 30.35197 | 42.37447 | 1006 |
| 2837 | −1 | CAAAATTAGGCATCATTGTT | AGG | 78.91428 | 34.86543 | 1007 |
| 2849 | 1 | CTAACAATGATGCCTAATTT | TGG | 71.83239 | 32.31041 | 1008 |
| 2850 | −1 | AAAAAAAATTCACCAAAATT | AGG | 40.46491 | 47.7423 | 1009 |
| 2875 | 1 | TGATATCATTAAGTCACATG | TGG | 76.59019 | 59.28563 | 1010 |
| 2893 | 1 | TGACTTAATGATATCAAATT | AGG | 39.65251 | 38.32681 | 1011 |
| 2927 | −1 | AGCTTTTTATAATGGAGCAG | GGG | 87.33466 | 59.01784 | 1012 |
| 2928 | 1 | TAGCTTTTTATAATGGAGCA | GGG | 86.07683 | 56.81693 | 1013 |
| 2929 | −1 | ATAGCTTTTTATAATGGAGC | AGG | 67.82726 | 40.53187 | 1014 |
| 2935 | −1 | CTATTTATAGCTTTTTATAA | TGG | 47.40677 | 25.75732 | 1015 |
| 2947 | 1 | CATTATAAAAAGCTATAAAT | AGG | 53.188 | 27.28205 | 1016 |

TABLE 10
gRNA sequences targeted for the promoter region of PKCBDAS1
(pPKCBDAS1)

| Position on | | | | Efficiency | |
|---|---|---|---|---|---|
| SEQ ID NO 1153 | Strand | Sequence | PAM | Score | SEQ ID NO |
| 40 | −1 | CAAAGTTATAGGGATTTCTT | TGG | 35.72969 | 1017 |
| 50 | −1 | GGCCTGATCCCAAAGTTATA | GGG | 35.70959 | 1018 |
| 51 | −1 | TGGCCTGATCCCAAAGTTAT | AGG | 30.95953 | 1019 |
| 52 | 1 | CAAAGAAATCCCTATAACTT | TGG | 32.63986 | 1020 |
| 53 | 1 | AAAGAAATCCCTATAACTTT | GGG | 36.88213 | 1021 |
| 59 | 1 | ATCCCTATAACTTTGGGATC | AGG | 39.50266 | 1022 |
| 71 | −1 | CGAATCTTGCCATATCTCTA | TGG | 43.38427 | 1023 |
| 73 | 1 | GGGATCAGGCCATAGAGATA | TGG | 56.85926 | 1024 |
| 124 | −1 | AAAAGTTTATCACTTTATTT | GGG | 30.16463 | 1025 |
| 125 | −1 | TAAAAGTTTATCACTTTATT | TGG | 6.844194 | 1026 |
| 154 | 1 | TTTTAAACAAAGAAGCAACA | AGG | 67.83789 | 1027 |
| 177 | 1 | CGTACATTGTCGACCTCAGA | AGG | 52.08572 | 1028 |
| 179 | −1 | CCTTTTTCCTTATCCTTCTG | AGG | 53.70836 | 1029 |
| 183 | 1 | TTGTCGACCTCAGAAGGATA | AGG | 49.66444 | 1030 |
| 190 | 1 | CCTCAGAAGGATAAGGAAAA | AGG | 45.02106 | 1031 |
| 204 | −1 | CAGTGACCGTGATAGCGAGC | TGG | 52.28843 | 1032 |
| 209 | 1 | AAGGCTCCAGCTCGCTATCA | CGG | 43.3947 | 1033 |
| 216 | 1 | CAGCTCGCTATCACGGTCAC | TGG | 51.18988 | 1034 |
| 230 | −1 | AGGATGTCCGTACAGGAGCT | TGG | 54.61793 | 1035 |
| 234 | 1 | ACTGGAACCAAGCTCCTGTA | CGG | 54.2677 | 1036 |
| 237 | −1 | GTGTCTGAGGATGTCCGTAC | AGG | 57.59087 | 1037 |
| 250 | −1 | ATCTTTAATTATTGTGTCTG | AGG | 48.15802 | 1038 |
| 299 | −1 | TGTGATCCTCCCGCATTTAT | TGG | 25.43056 | 1039 |
| 300 | 1 | TCATTTTGATCCAATAAATG | CGG | 55.04425 | 1040 |
| 301 | 1 | CATTTTGATCCAATAAATGC | GGG | 55.63264 | 1041 |
| 304 | 1 | TTTGATCCAATAAATGCGGG | AGG | 60.57337 | 1042 |
| 345 | −1 | CATATCACGTTGTGTAAATG | CGG | 55.96468 | 1043 |
| 377 | −1 | CGATCATCCCCTAAAATCAT | GGG | 51.8821 | 1044 |
| 378 | −1 | ACGATCATCCCCTAAAATCA | TGG | 46.86483 | 1045 |
| 379 | 1 | TAATCAAATCCCATGATTTT | AGG | 21.19347 | 1046 |
| 380 | 1 | AATCAAATCCCATGATTTTA | GGG | 22.50742 | 1047 |
| 381 | 1 | ATCAAATCCCATGATTTTAG | GGG | 53.63938 | 1048 |
| 436 | −1 | ACTATTTATAATCACATAGT | AGG | 53.22792 | 1049 |
| 449 | 1 | TACTATGTGATTATAAATAG | TGG | 46.87963 | 1050 |
| 470 | 1 | GGCAAGTAAGATCAAAAAAG | TGG | 57.05661 | 1051 |
| 493 | 1 | ACGAAAAAAGCATACAAAAA | AGG | 46.19218 | 1052 |
| 573 | −1 | AACACACAGGATTTTTTACG | TGG | 64.37507 | 1053 |
| 586 | −1 | CAATGAAAATATGAACACAC | AGG | 64.19248 | 1054 |
| 638 | 1 | TATGTGAGATTGTCACTGTT | AGG | 42.77358 | 1055 |
| 689 | 1 | TAACACAATCTAATTTATTT | TGG | 13.9135 | 1056 |
| 694 | 1 | CAATCTAATTTATTTTGGAT | TGG | 40.88386 | 1057 |
| 769 | −1 | TGGCGACATAAAACAATATT | GGG | 27.85863 | 1058 |
| 770 | −1 | TTGGCGACATAAAACAATAT | TGG | 32.4639 | 1059 |
| 789 | −1 | TATTTATTTTAATTTGTTGT | TGG | 38.0834 | 1060 |
| 825 | 1 | ACAAAAAAATAACTAACCCA | AGG | 63.9156 | 1061 |
| 826 | 1 | CAAAAAAATAACTAACCCAA | GGG | 66.25841 | 1062 |
| 830 | −1 | TATAATTTTTTTCTTCCCTT | GGG | 44.82545 | 1063 |
| 831 | −1 | ATATAATTTTTTTCTTCCCT | TGG | 40.67156 | 1064 |
| 932 | −1 | TTTATTGTTGAAAAATTTAT | TGG | 17.1718 | 1065 |
| 980 | −1 | TAATTATAAGACGTATAACA | TGG | 57.95244 | 1066 |
| 1033 | 1 | ATTCAATTTTAATGAGATCG | AGG | 59.86873 | 1067 |
| 1034 | 1 | TTCAATTTTAATGAGATCGA | GGG | 58.31985 | 1068 |
| 1221 | −1 | TATTTGTTAATATATATTGA | TGG | 38.76283 | 1069 |
| 1259 | −1 | TAAAATTTTAAAGTTATGTG | TGG | 61.65078 | 1070 |
| 1298 | −1 | TTTTTTAAGATTAATTACTA | TGG | 37.65256 | 1071 |
| 1467 | 1 | AAAAGAAATTCAAATTATTA | AGG | 26.49749 | 1072 |
| 1470 | 1 | AGAAATTCAAATTATTAAGG | TGG | 53.17187 | 1073 |
| 1477 | 1 | CAAATTATTAAGGTGGCGTT | TGG | 32.49361 | 1074 |
| 1662 | 1 | TTTAATCAATGTTTTAGATT | AGG | 34.18499 | 1075 |
| 1673 | 1 | TTTTAGATTAGGACCAGACC | CGG | 63.53117 | 1076 |
| 1675 | −1 | AGGGAATTTGGGTCCGGGTC | TGG | 39.29344 | 1077 |
| 1680 | −1 | TAGGGAGGGAATTTGGGTCC | GGG | 47.67576 | 1078 |
| 1681 | −1 | GTAGGGAGGGAATTTGGGTC | CGG | 42.65646 | 1079 |
| 1686 | −1 | GGGCCGTAGGGAGGGAATTT | GGG | 30.25891 | 1080 |
| 1687 | −1 | AGGGCCGTAGGGAGGGAATT | TGG | 25.17024 | 1081 |
| 1694 | 1 | GGACCCAAATTCCCTCCCTA | CGG | 62.73298 | 1082 |
| 1694 | −1 | TAGCGCCAGGGCCGTAGGGA | GGG | 49.60487 | 1083 |
| 1695 | −1 | TTAGCGCCAGGGCCGTAGGG | AGG | 53.67964 | 1084 |
| 1698 | −1 | GGTTTAGCGCCAGGGCCGTA | GGG | 51.98898 | 1085 |
| 1699 | −1 | GGGTTTAGCGCCAGGGCCGT | AGG | 44.03016 | 1086 |
| 1700 | 1 | AAATTCCCTCCCTACGGCCC | TGG | 49.55893 | 1087 |
| 1706 | −1 | AAAAGTCGGGTTTAGCGCCA | GGG | 57.95828 | 1088 |
| 1707 | −1 | CAAAAGTCGGGTTTAGCGCC | AGG | 41.30613 | 1089 |
| 1719 | 1 | TCGGGTTCAAGTCAAAAGTC | GGG | 53.54228 | 1090 |
| 1720 | −1 | TTCGGGTTCAAGTCAAAAGT | CGG | 50.22631 | 1091 |
| 1737 | −1 | TTTAGGTGCAAGTCGGATTC | GGG | 34.98531 | 1092 |
| 1738 | 1 | ATTTAGGTGCAAGTCGGATT | CGG | 32.88236 | 1093 |
| 1744 | −1 | AAAATAATTTAGGTGCAAGT | CGG | 57.94599 | 1094 |
| 1754 | −1 | TTAAGCTTTCAAAATAATTT | AGG | 29.98844 | 1095 |
| 1862 | 1 | AAGATAATTTTACCACTTAC | AGG | 37.53132 | 1096 |
| 1863 | −1 | TAATATAATCATCCTGTAAG | TGG | 52.61125 | 1097 |
| 1897 | 1 | TTGATTGTATTGATTATTAT | AGG | 16.26497 | 1098 |
| 1950 | 1 | TAGCTACAATTATTAATGAG | TGG | 56.04522 | 1099 |
| 1977 | 1 | TAAAATTGAAGTGTGTTTTT | TGG | 14.81896 | 1100 |
| 2000 | −1 | ATATTTCAAACTTATAGCTT | AGG | 38.28176 | 1101 |
| 2057 | −1 | ACCAATTAGAAATGGGCACG | TGG | 62.25444 | 1102 |
| 2064 | −1 | AACTTAGACCAATTAGAAAT | GGG | 41.42269 | 1103 |
| 2065 | −1 | TAACTTAGACCAATTAGAAA | TGG | 32.21124 | 1104 |
| 2067 | 1 | ACCACGTGCCCATTTCTAAT | TGG | 31.88023 | 1105 |
| 2126 | −1 | TCCACGTGTCATTTTCTTCT | TGG | 23.64376 | 1106 |
| 2136 | 1 | ACCAAGAAGAAAATGACACG | TGG | 72.79231 | 1107 |
| 2158 | 1 | GATAATGACTTAATATTTAA | TGG | 22.44283 | 1108 |
| 2162 | 1 | ATGACTTAATATTTAATGGT | CGG | 59.98016 | 1109 |
| 2176 | −1 | TAATATTTTGGGAACTCTGT | AGG | 53.58079 | 1110 |
| 2187 | −1 | TAATACCTAAGTAATATTTT | GGG | 24.67228 | 1111 |
| 2188 | −1 | ATAATACCTAAGTAATATTT | TGG | 13.08769 | 1112 |
| 2193 | 1 | GAGTTCCCAAAATATTACTT | AGG | 47.17027 | 1113 |
| 2216 | −1 | AAATACCTACGTTTTATTTT | TGG | 11.94795 | 1114 |
| 2222 | 1 | TAGCGCCAAAAATAAAACGT | AGG | 70.02414 | 1115 |
| 2239 | 1 | CGTAGGTATTTATTTGCAAC | TGG | 32.92943 | 1116 |
| 2294 | −1 | CACAAAACATCTAAAAAAAA | TGG | 33.67954 | 1117 |
| 2306 | 1 | CATTTTTTTTAGATGTTTTG | TGG | 26.9668 | 1118 |
| 2319 | −1 | ACGCCAACTCATATACAAAA | TGG | 40.39333 | 1119 |
| 2327 | 1 | GGACCATTTTGTATATGAGT | TGG | 60.94691 | 1120 |
| 2372 | 1 | TGTTGAATCTCTAGCTCTTT | TGG | 25.56256 | 1121 |
| 2417 | 1 | GCTTTATTGTCTAAATTTCT | TGG | 20.64172 | 1122 |
| 2418 | 1 | CTTTATTGTCTAAATTTCTT | GGG | 21.40116 | 1123 |
| 2419 | 1 | TTTATTGTCTAAATTTCTTG | GGG | 58.49927 | 1124 |
| 2440 | −1 | CTAAAGTAAGCGAGCAACAT | GGG | 56.12375 | 1125 |
| 2441 | −1 | TCTAAAGTAAGCGAGCAACA | TGG | 74.55214 | 1126 |
| 2483 | 1 | GTTTGACAAAACATGCTATT | CGG | 34.18684 | 1127 |
| 2511 | 1 | CAATGAGCTATCCTAGTTCA | AGG | 42.25734 | 1128 |
| 2511 | −1 | CACAGAAATCTCCTTGAACT | AGG | 56.77193 | 1129 |
| 2532 | 1 | GGAGATTTCTGTGCTATTTG | TGG | 46.71018 | 1130 |
| 2605 | −1 | CGCTTGCAAATATTTATCTA | TGG | 35.67387 | 1131 |
| 2649 | 1 | ATAGCAAATTTTTTTTCCAT | AGG | 41.9361 | 1132 |
| 2654 | 1 | TGATTTTTTTAAATTTCCTA | TGG | 37.98135 | 1133 |
| 2723 | 1 | TTATTAAAAATAAATGTACA | TGG | 64.33675 | 1134 |
| 2736 | 1 | ATGTACATTTATTTTTAATA | AGG | 24.97531 | 1135 |
| 2737 | 1 | TGTACATTTATTTTTAATAA | GGG | 33.69876 | 1136 |
| 2753 | 1 | ATAAGGGCTGCACCTAACAA | AGG | 57.21703 | 1137 |
| 2754 | −1 | CAAAATTAGGCACCTTTGTT | AGG | 31.41724 | 1138 |
| 2766 | 1 | CTAACAAAGGTGCCTAATTT | TGG | 24.89512 | 1139 |
| 2767 | −1 | ATTTCTTTTTTACCAAAATT | AGG | 45.67433 | 1140 |
| 2783 | 1 | TTTTGGTAAAAAAGAAATTA | CGG | 33.2113 | 1141 |
| 2848 | −1 | AGCTTTATAGATTGGAGTGG | GGG | 53.84871 | 1142 |
| 2849 | −1 | TAGCTTTATAGATTGGAGTG | GGG | 59.39764 | 1143 |
| 2850 | −1 | ATAGCTTTATAGATTGGAGT | GGG | 41.54822 | 1144 |
| 2851 | −1 | TATAGCTTTATAGATTGGAG | TGG | 55.63853 | 1145 |
| 2856 | −1 | CTATTTATAGCTTTATAGAT | TGG | 33.39825 | 1146 |
| 2868 | 1 | CAATCTATAAAGCTATAAAT | AGG | 28.20163 | 1147 |
| 2888 | −1 | ATGTTGGAAATTACTATGAA | TGG | 41.042 | 1148 |
| 2904 | −1 | TTTTTCTTTAGTCGTAATGT | TGG | 47.87998 | 1149 |

Reference is made to Table 11 presenting a summary of the sequences within the scope of the current invention.

TABLE 11
Summary of sequences within the scope of the present invention

| | Targeted promoter | |
|---|---|---|
| Sequence name | sequence | gRNA sequences |
| pFNTHCAS-1 | SEQ ID NO: 1 | SEQ ID NO: 7- |
| | | SEQ ID NO: 91 |
| | | (Table 1) |
| pFNTHCAS-2 | SEQ ID NO: 2 | SEQ ID NO: 92- |
| | | SEQ ID NO: 176 |
| | | (Table 2) |
| pPKTHCAS-1 | SEQ ID NO: 3 | SEQ ID NO: 177- |
| | | SEQ ID NO: 278 |
| | | (Table 3) |
| pPKTHCAS-2 | SEQ ID NO: 4 | SEQ ID NO: 279- |
| | | SEQ ID NO: 419 |
| | | (Table 4) |
| pPKTHCAS-3 | SEQ ID NO: 5 | SEQ ID NO: 420- |
| | | SEQ ID NO: 560 |
| | | (Table 5) |
| pPKTHCAS-4 | SEQ ID NO: 6 | SEQ ID NO: 561- |
| | | SEQ ID NO: 681 |
| | | (Table 6) |
| pCBDAS2 | SEQ ID NO: 1150 | SEQ ID NO: 682- |
| | | SEQ ID NO: 794 |
| | | (Table 7) |
| pFNCBDAS | SEQ ID NO: 1151 | SEQ ID NO: 795- |
| | | SEQ ID NO: 895 |
| | | (Table 8) |
| pPKCBDAS | SEQ ID NO: 1152 | SEQ ID NO: 896- |
| | | SEQ ID NO: 1016 |
| | | (Table 9) |
| pPKCBDAS1 | SEQ ID NO: 1153 | SEQ ID NO: 1017- |
| | | SEQ ID NO: 1149 |
| | | (Table 10) |

The above gRNA polynucleotides have been cloned into suitable vectors and their sequence has been verified. In addition, different Cas9 versions have been analyzed for optimal compatibility between the Cas9 protein activity and the gRNA polynucleotide in the Cannabis plant.

The efficiency of the designed gRNA polynucleotides may be validated by transiently transforming Cannabis tissue culture. A plasmid carrying a predetermined gRNA sequence together with the Cas9 gene has been transformed into Cannabis cells/protoplasts. The protoplast cells have been grown for a short period of time and then were analyzed for existence of genome editing events. The positive constructs were subjected to the herein established stable transformation protocol into Cannabis plant tissue for producing genome edited Cannabis plants within the promoter region of tetrahydrocannabinolic acid synthase (CsTHCAS) and/or Cannabidiolic acid synthase (CsCBDAS) genes.

Stage 3: Transforming Cannabis plants using Agrobacterium or biolistics (gene gun) methods. For Agrobacterium and bioloistics, a DNA plasmid carrying (Cas9+gene specific gRNA) can be used. A vector containing a selection marker, Cas9 gene and relevant gene/promoter specific gRNA's is constructed. For biolistics, Ribonucleoprotein (RNP) complexes carrying (Cas9 protein+gene/promoter specific gRNA) are used. RNP complexes are created by mixing the Cas9 protein with relevant gene specific gRNA's.

According to some embodiments of the present invention, transformation of various Cannabis tissues was performed using particle bombardment of:
DNA vectors
Ribonucleoprotein complex (RNP's)

According to further embodiments of the present invention, transformation of various Cannabis tissues was performed using Agrobacterium (Agrobacterium tumefaciens) by:
Regeneration-based transformation
Floral-dip transformation
Seedling transformation

Transformation efficiency by A. tumefaciens has been compared to the bombardment (biolistics system) method by transient GUS transformation experiment. After transformation, GUS staining of the transformants has been performed.

According to further embodiments of the present invention, additional transformation tools were used in Cannabis, including, but not limited to:
Protoplast PEG transformation
Extend RNP use
Directed editing screening using fluorescent tags
Electroporation

Stage 4: Regeneration in tissue-culture. When transforming DNA constructs into the plant, antibiotics is used for selection of positive transformed plants. An improved regeneration protocol was herein established for the Cannabis plant.

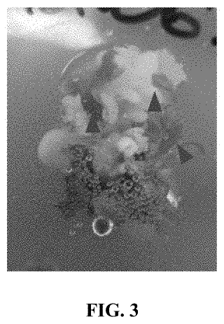

Reference is now made to FIG. 3 presenting regeneration of Cannabis tissue. In this figure, arrows indicate new meristem emergence.

Stage 5: Selection of positive transformants. Once regenerated plants appear in tissue culture, DNA is extracted from leaf sample of the transformed plant and PCR is performed using primers flanking the edited region. PCR products are then digested with enzymes recognizing the restriction site near the original gRNA sequence. If editing event occurred, the restriction site will be disrupted and the PCR product will not be cleaved. No editing event will result in a cleaved PCR product.

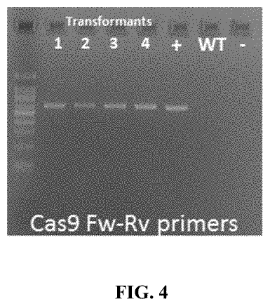

Reference is now made to FIG. 4 showing PCR detection of Cas9 DNA in shoots of transformed Cannabis plants. DNA extracted from shoots of plants transformed with Cas9 using biolistics. This figure shows that three weeks post transformation, Cas9 DNA was detected in shoots of transformed plants.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

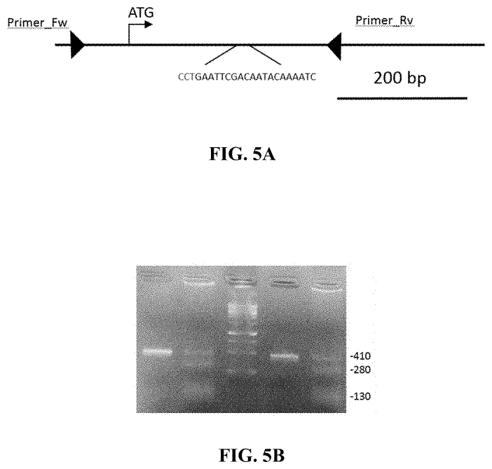

Reference is now made to FIG. 5 presenting results of in vitro analysis of CRISPR/Cas9 cleavage activity. FIG. 5A schematically shows the genomic area targeted for editing (PAM is marked in red) and amplified by the reverse and forward designed primers FIG. 5B photographically presents a gel showing successful digestion of the resulted PCR amplicon containing the gene specific gRNA sequence, by RNP complex containing Cas9. The analysis included the following steps:
1) Amplicon was isolated from two exemplified Cannabis strains by primers flanking the sequence of the gene of interest targeted by the predesigned sgRNA.
2) RNP complex was incubated with the isolated amplicon.
3) The reaction mix was then loaded on agarose gel to evaluate Cas9 cleavage activity at the target site.

Stage 6: Selection of transformed Cannabis plants presenting reduced expression of at least one of Cannabis tetrahydrocannabinolic acid synthase (CsTHCAS) and/or Cannabidiolic acid synthase (CsCBDAS) homologue or variant as described above. It is within the scope that different gRNA promoters were tested in order to maximize editing efficiency.

The present invention offers targeted genome editing within promoter regions of genes or alleles encoding THCAS and/or CBDAS cannabinoid synthesis enzyme that can be used to modulate the cannabinoid profile, particularly, THCA and/or CBDA content in the Cannabis plant. Upon modifying the promoter region of THCAS and/or CBDAS genes by the method of the present invention, a Cannabis plant with desirable phenotype of altered, and more specifically reduced THCA (or THC) and/or CBDA (or CBD) levels, with no known pleotropic effects as compared to a wild-type control Cannabis plant, can be achieved. For example, downregulation of THCAS expression by a knock down mutation in its promoter region, reduce THCA level and may also enhance the level of CBDA synthesis. Downregulation of CBDAS expression by a knock down mutation in its promoter region, reduce CBDA level and may also enhance the level of THCA synthesis. Downregulation of both THCAS and CBDAS expression by a knock down mutation at their promoter region, reduce both THCA and CBDA levels and may also enhance the level of their substrate CBGA in the plant. In this way, the concentration level of predetermined cannabinoids such as THCA, CBDA and/or CBGA in a Cannabis variety can be adjusted (reduced or elevated) as desired. For example, any Cannabis variety or cultivar with a high THCA or THC level can be converted into a low level THCA plant variety.

The guide RNA/Cas9 endonuclease system is used to target and induce a double strand break at a Cas9 endonuclease target site located within the regulatory regions operably linked to the herein identified genes encoding THCA and/or CBDA synthase homologues or variants or alleles. Cannabis cultivars or varieties comprising targeted mutations within the promoter region of THCAS and/or CBDAS gene alleles were selected and evaluated for their phenotypic effect on cannabinoid expression profiled in the plant.

Various embodiments have been presented. Each of these embodiments may of course include features from other embodiments presented, and embodiments not specifically described may include various features described herein.

1.-90. (canceled)

91. A method for altering tetrahydrocannabinolic acid synthase (THCAS) or cannabidiolic acid synthase (CBDAS) gene expression in a Cannabis plant or a cell thereof, the method comprising steps of introducing one or more nucleotide modifications through targeted genome modification at a regulatory region modulating the expression of said THCAS or CBDAS gene.

92. The method according to claim 91, wherein said targeted genome modification confers altered tetrahydrocannabinolic acid (THCA) or cannabidiolic acid (CBDA) content as compared to a comparable control Cannabis plant or a cell thereof lacking said at least one targeted nucleotide modification.

93. The method according to claim 91, wherein said plant or a cell thereof comprises one of the following:
a. reduced THCA or CBDA and/or Cannabigerolic acid (CBGA) content relative to a comparable control Cannabis plant or a cell thereof lacking said at least one targeted nucleotide modification; or
b. elevated THCA or CBDA and/or Cannabigerolic acid (CBGA) content relative to a comparable control Cannabis plant or a cell thereof lacking said at least one targeted nucleotide modification.

94. The method according to claim 92, wherein said comparable control Cannabis plant or a cell thereof is of a similar genotype and/or chemotype and/or genetic background and is lacking said at least one targeted nucleotide modification.

95. The method according to claim 91, wherein at least one of the following holds true:
a. said targeted genome modification is introduced through a genome editing at said regulatory region of said THCAS or CBDAS genomic locus;
b. said method comprises introducing an expression cassette encoding a RNA-guided endonuclease and at least one guide RNA (gRNA) having a sequence that is complementary to a target sequence within said regulatory region of said THCAS or CBDAS gene; and
c. the targeted DNA modification is through a genome modification technique selected from the group consisting of polynucleotide-guided endonuclease, CRISPR (Clustered Regularly Interspaced Short Palindromic Repeats) and CRISPR-associated (Cas) (CRISPR-Cas) endonucleases, base editing deaminases, zinc finger nuclease (ZFN), a transcription activator-like effector nuclease (TALEN), engineered site-specific meganucleases, Argonaute or any combination thereof.

96. The method according to claim 91, wherein said regulatory region is at least one of the following:
a. a promoter region or terminator region, operably linked to the coding region of the THCAS or CBDAS gene;
b. upstream of the 5′ end of the coding sequence of a THCAS or CBDAS gene allele or is downstream of the 3′ end of the coding sequence of a THCAS or CBDAS gene allele; and
c. comprises a transcription factor binding site, an RNA polymerase binding site, a TATA box, or a combination of structural variations thereof.

97. The method according to claim 91, wherein said targeted nucleotide modification is one of the following:
a. interrupts or interferes or down regulate or silence transcription and/or translation of a Cannabis allele sequence encoding THCAS or CBDAS enzyme; or
b. enhance or induce or increase transcription and/or translation of a Cannabis allele sequence encoding THCAS or CBDAS enzyme.

98. The method according to claim 91, wherein the targeted nucleotide modification is induced by a guide RNA that comprises a sequence that corresponds to a target sequence at a polynucleotide selected from the group consisting of pFNTHCAS-1 having at least 70% sequence similarity to the nucleotide sequence as set forth in SEQ ID NO:1, pFNTHCAS-2 having at least 70% sequence similarity to the nucleotide sequence as set forth in SEQ ID NO:2, pPKTHCAS-1 having at least 70% sequence similarity to the nucleotide sequence as set forth in SEQ ID NO:3, pPKTHCAS-2 comprising a nucleic acid sequence having at least 70% sequence similarity to the nucleotide sequence as set forth in SEQ ID NO:4, pPKTHCAS-3 having at least 70% sequence similarity to the nucleotide sequence as set forth in SEQ ID NO:5, pPKTHCAS-4 having at least 70% sequence similarity to the nucleotide sequence as set forth in SEQ ID NO:6, pCBDAS2 having at least 70% sequence similarity to the nucleotide sequence as set forth in SEQ ID NO:1150, pFNCBDAS having at least 70% sequence similarity to the nucleotide sequence as set forth in SEQ ID NO:1151, pPKCBDAS having at least 70% sequence similarity to the nucleotide sequence as set forth in SEQ ID NO:1152, pPKCBDAS1 having at least 70% sequence similarity to the nucleotide sequence as set forth in SEQ ID NO: 1153, and any combination thereof.

99. The method according to claim 98, wherein the guide RNA (gRNA) nucleotide sequence is selected from the group consisting of:
a. a gRNA targeting pFNTHCAS-1 and comprising a nucleotide sequence that is at least 80% identical to a nucleotide sequence selected from the group consisting of SEQ ID NO: 7-91 and any combination thereof;
b. a gRNA targeting pFNTHCAS-2 and comprising a nucleotide sequence that is at least 80% identical to the nucleotide sequence selected from the group consisting of SEQ ID NO.: 92-176 and any combination thereof;
c. a gRNA targeting pPKTHCAS-1 and comprising a nucleotide sequence that is at least 80% identical to the nucleotide sequence selected from the group consisting of SEQ ID NO.: 177-278 and any combination thereof;
d. a gRNA targeting pPKTHCAS-2 and comprising a nucleotide sequence that is at least 80% identical to the nucleotide sequence selected from the group consisting of SEQ ID NO.: 279-419 and any combination thereof;
e. a gRNA targeting pPKTHCAS-3 and comprising a nucleotide sequence that is at least 80% identical to the nucleotide sequence selected from the group consisting of SEQ ID NO.: 420-560 and any combination thereof;
f. a gRNA targeting pPKTHCAS-4 and comprising a nucleotide sequence that is at least 80% identical to the nucleotide sequence selected from the group consisting of SEQ ID NO.: 561-681 and any combination thereof;
g. a gRNA targeting pCBDAS2 and comprising a nucleotide sequence that is at least 80% identical to the nucleotide sequence selected from the group consisting of SEQ ID NO.: 682-794 and any combination thereof;
h. a gRNA targeting pFNCBDAS and comprising a nucleotide sequence that is at least 80% identical to the nucleotide sequence selected from the group consisting of SEQ ID NO.: 795-895 and any combination thereof;
i. a gRNA targeting pPKCBDAS and comprising a nucleotide sequence that is at least 80% identical to the nucleotide sequence selected from the group consisting of SEQ ID NO.: 896-1016 and any combination thereof; and
j. a gRNA targeting pPKCBDAS1 and comprising a nucleotide sequence that is at least 80% identical to the nucleotide sequence selected from the group consisting of SEQ ID NO.: 1017-1149 and any combination thereof.

100. The method according to claim 91, wherein the targeted nucleotide modification is selected from the group consisting of insertion, deletion, single nucleotide polymorphism (SNP), a missense mutation, nonsense mutation, indel, substitution or duplication and a polynucleotide modification, such that the expression of the THCAS or CDBAS polynucleotide is reduced or affected.

101. The method according to claim 91, wherein the Cannabis plant or a cell thereof exhibits one of the following:
a. reduced THCA or CDBA content when the targeted DNA modification within the regulatory region results in reduced expression or activity of protein encoded by the THCAS or CDBAS gene allele polynucleotide, respectively; or
b. elevated THCA or CDBA content when the targeted DNA modification within the regulatory region results in increased expression or activity of protein encoded by the THCAS or CDBA gene allele polynucleotide, respectively.

102. The method according to claim 91, wherein said plant or a cell thereof has THCA (or THC) and/or CBDA (or CBD) content of at least one of the following:
a. up to 30% by weight, particularly between about 0% to about 30% by weight, more particularly between about 0.3% to about 30%, even more particularly between about 0.1% to about 10% by weight;
b. not more than about 0.3% by weight; and
c. at least 20% by weight.

103. The method according to claim 91, wherein said plant or a cell thereof is THCA or THC or CBDA or CBD free.

104. A cannabis plant, plant cell or plant seed produced by the method according to claim 91.

105. A guide RNA (gRNA) sequence that targets a tetrahydrocannabinolic acid synthase (THCAS) or cannabidiolic acid synthase (CBDAS) genomic locus of a plant cell, wherein the gRNA is complementary to at least one target sequence of a regulatory region within said genomic locus, said regulatory region operably linked to the expression of a THCAS or CBDAS gene allele.

106. The gRNA sequence according to claim 105, wherein at least one of the following holds true:
a. said gRNA comprises a polynucleotide sequence selected from the group consisting of pFNTHCAS-1 having at least 70% sequence similarity to the nucleotide sequence as set forth in SEQ ID NO:1, pFNTHCAS-2 having at least 70% sequence similarity to the nucleotide sequence as set forth in SEQ ID NO:2, pPKTHCAS-1 having at least 70% sequence similarity to the nucleotide sequence as set forth in SEQ ID NO:3, pPKTHCAS-2 comprising a nucleic acid sequence having at least 70% sequence similarity to the nucleotide sequence as set forth in SEQ ID NO:4, pPKTHCAS-3 having at least 75% sequence similarity to the nucleotide sequence as set forth in SEQ ID NO:5, pPKTHCAS-4 having at least 70% sequence similarity to the nucleotide sequence as set forth in SEQ ID NO:6, pCBDAS2 having at least 70% sequence similarity to the nucleotide sequence as set forth in SEQ ID NO:1150, pFNCBDAS having at least 70% sequence similarity to the nucleotide sequence as set forth in SEQ ID NO:1151, pPKCBDAS having at least 70% sequence similarity to the nucleotide sequence as set forth in SEQ ID NO:1152, and pPKCBDAS1 having at least 70% sequence similarity to the nucleotide sequence as set forth in SEQ ID NO:1153; and
b. the nucleotide sequence of said gRNA is selected from the group consisting of a nucleotide sequence that is at least 80% identical to the nucleotide sequence as set forth in SEQ ID NO.: 7-1149 and any combination thereof.

107. A plant cell or host cell comprising the guide RNA of claim 105.

108. A plant part, plant cell, tissue culture of regenerable cells, protoplasts, callus or plant seed of a plant according to claim 104.

109. A non-living product or medical composition derived from the Cannabis plant or a cell thereof of claim 104.

110. Use of a nucleotide sequence having at least 70% sequence identity to a nucleotide sequence as set forth in at least one of SEQ ID NO: 7-91, SEQ ID NO: 92-176, SEQ ID NO: 177-278, SEQ ID NO: 279-419, SEQ ID NO: 420-560, SEQ ID NO: 561-681, SEQ ID NO: 682-794, SEQ ID NO: 795-895, SEQ ID NO: 896-1016, SEQ ID NO: 1017-1149, for targeted genome modification of pFNTHCAS-1, pFNTHCAS-2, pPKTHCAS-1, pPKTHCAS-2, pPKTHCAS-3, pPKTHCAS-4, pCBDAS2, pFNCBDAS, pPKCBDAS and pPKCBDAS1 gene, respectively; and/or
use of a nucleotide sequence having at least 70% sequence identity to a nucleotide sequence as set forth in at least one of SEQ ID NO: 7-681 and any combination thereof for reducing THCA content in a Cannabis plant or a cell thereof; and/or
use of a nucleotide sequence having at least 70% sequence identity to a nucleotide sequence as set forth in at least one of SEQ ID NO: 682-1149 and any combination thereof for reducing CBDA content in a Cannabis plant or a cell thereof.


SEQUENCE LISTING (1153 sequences)

SEQ ID NO: 1 - DNA, 2989 bp, Cannabis sativa
tttatctattttttctcatttttaattaataattaataaaattaataaaatatacataaaggaaagcctaagaagagtctaagtggcatcctatttttttttctttcctatttttcctttattctaacacatctattttctttcaaaaatacactacatatacaaactaaaagtcccacattggttaaaaacttttaatgctatccatgtaagttataaataatagttttattatttacctacatttgtctaagaaatattattttttatgggtggttgattaatatcttacccaataaattgttatacataaaggaaagcctaagaagaggtctaagtggcatcctatttttttttcttcctatttttcctttattctaacacactcttattttctttcaaaaatactacatatacaacaaaagtcccacattggttaaaaactttttaagtgctatccatgtaagttataaataatagtttattattttacctacattgtctaagaaatattattttttatgggtggttgattaatatcttacccaataaatttgttatatatatgtataagtagtcatttattttctaataaaataattaactttgtaataattatttttattatacatatatttatgaaattaattttagtttacgaattttattttactattctagatttttatattgttatttgctcattaaaaatatttttttacaactaaaacattttctcttaaacttttattttttcaaaaattaaaaatttatctataagaggtgaccatacttataagattatttgagaattcttgattatatatatattttttaattttttgttactttttttattatttaaattttagaatatagaaaatatctattctacaagttgtttttttttatagtatagcatttttcgtattatattttttaagtttagtttaaattatttagatggttaaaattagagataaaaattgatgttcttacctaaaatatagtttgtttttttgtagttttacattaaaaattaaaaataaaatatgaaatttaaaatgatttatgtagcattacatgaattttagttgacattctttgttttatctatttttctcatttttaattaataaaataaaataattaatatcaatatatataaaggaaagtctaaacaaaagtggtgacttgctatgtattttttattgtagtgtatcttttttcttattttttattttttatacttttagtaacacgagagttatttcgaaaaagtctcacatagtttaaaaactattgtgagtaatatacttctagattatatacatcaatatttttttttttttattttggtctgtgacaaagttagtattatttattttctatgtaggtaattaataatgttaatcttacccaattacttgattttttttaagtgaatgtaaagtaaaattttatatttttcaatctttatcaaattttaaaataattataaaaacatctttaattatttttcatatatcatatttttcaatctttatttttcatataaagtaaatttttttcaatttttctaattataaaaaaaatattattgtcattttatatttaaaatgattatatccataaaataatttaaattatattcatttattaatatatgatattttattttagaataataagactccttaaatatatctttttatgtatatacttttcgtgacatgacactattgtgaatgctaaacttataggttatatatttcactattgtttgttctaaaattagtattatttattaataatgtgattgatttatttatttacaatttgagtaattgataaatttaattttctccaattagttgtaatttttttttaaaaaaattaatgcaacatatattttcgcatttttcaatctttgtcaaatattaattaaaaaaaattcttaaagtttcttaataaaaagaaaaatatattttaattttaaattattaatttttgtagaacatatacaatattattatgtataaaaatacatgtactttttttttgttaattttaccattttaatatttatcacaaaatcactacttttatatattttaaaaatagcaaattatgtttctgatttcaatactattactttatttataataacacacatcaaaatgttatattttaaattatatcactcagtataagtacaatatatatatatatatatatatatatataaaatattacaaaagttggacaacaccgagcgaagcgcggcttagttccctagttgaataataaagtagatagtagaggagattagaactcataatttttcatttatatatttgaatttagtttactgttctttattattcttcatttaattttgctatatgttattttacttttcaaaaattctaattttgattaccaaataaaaaataaaatatatattggtacttcatatttagtttttatgggaatgataatgaatgatattgtaataaactataaaagtaattttgtatacttgcatagttcttaatttttacccaaaaaaatatatattaatgaaaaaaaaggtggaaggtgccataggcacctatatcccacaaactagctataagatattgaaagacaaatagatccatagataaatatttgcgtgtcctttcttttcatatctaatttcttgtttttttatttttttttcaatagccaatttcggcagcttaacttctttccatatataatgatgccaaactattcaatgtaaaatttagatttattttcattaagggcttcacctaacaaatgtgcctaatttttgtggatttttttaccatatgtcactatttaatgactaccaaattatagaaatatttaagtcaatttctcagtccccgctccaatataaagttatagaaaggacaattcttaattcatagtaattcaaaaatcattaagactaaaaaaa

SEQ ID NO: 2 - DNA, 3000 bp, Cannabis sativa
tgcaagggattgatgaacactactcttacattctctcaatccactagccacacccatttcacctcactcactctcaccaatcaagagcacatccactttataaggaattataggtcatttgggcctacatgcccaatttggagagaattagttgtcttagcccaatgggctgaccaaagacgttttagagtgtgtccatgggccccgagcgtgttagtacattatgtccatcccattatggctcgatggtaataccatgcattaccgattatatatagttatcattatatgctaatacgacactgagtgagcaacacttaattatgtcagtccacataaaatattctaacaagaaaagttacatcttttactcacaattttgtaagtacaatagcagaaattcttgggttgaatgaagatttttttttcaagaaatttttttgtgagttgttgtcttatttagattgtgtatgatttgtctaaaatgcatatatttattataaatacacgttttagtagctacgtttagtatatcttttatctctttaaatttaattcttttttgtgagaaaaagtgactttattgtgtagaggagagtgagttcgactctacctctttgtatacgtcattgttattattaagctttcaggagaaagttcttacatttttacaatctatctaatgaatcttactgataccatgtataaaactgtttgcttttaattttctttacttttattctttgttggtttctattttgcagttacacactactatacgaaaaggggctttaatcccaatttttatgggcattttataacgatttttgctaaaataaaaattaggatatatgtcatggataaaagctcatataaaaagtgggataaaaaagtatttttaatttcgattattataggaaaacgggataaaagattttttttatcccagttttaattgaaagctcagatcaaaagttccctttaattccaatttttccataaaaactggaattaatactatatatagcaattcatcattcttcaaagatttagccaagatactatgttcttcattacagaaatttatctatagtttcgtcagataaataaaaacatgttcatagaaaagattaaacatgagatcagatctcaaattacaatatcaccattaattaatgaaatcatatcaaagtctcaaaaataacacaaaatagagacaaaccataaatattaacattacaatatcaatggaagaaaaaaagttaaagtttattacagtatcagcgcgttatatttttcattctcttacttaaaagcttcttcaaccatgaaccatgaaccatgaagcaatgtcaatggcgacgaagatgtaagagagagagagagagagagagagagagagagagagcttcaggcctttttcagcaagagttttctccgacagtgatgggtaaagatagaagtggagaggaagaagaaggcccttcgtgcgtggctgagatcgcggccaagaatccggcaatgcagaaagaattgggagaaacaaagagagagggcgacaaaagagagagaacttggatgagagagataaccctaaacctaaaagtatttataaaaggactagggaaacccttttatcacggaaagggttcccttttacctcgtttggtttcagcaaaatattaaagagtgaaaacttaattttttcgtgaccagccaaacaaggcccttgctccaaagtgtagtagaagacccattaggagtgtgtttggctagtcaataaaaaaataatctttaacctttttcttagtttaatattttcctcttgatagtgttaaaccaaacatggcccttgtatcttatacaaaagtggggtaaaaatttccagcttactttttatctcactttttatttttctaagcataaacaatgagataaaaggttccatttcttgtagtgacaaaatcgtgttctatagttacaaattacctatttttgtattgactttattgtacactgtttaattaatgattaattaaataataaatttataattataaaaaatggaatttcattggtgatctcatggtgtttaggggaggaatgaggatggagtgtgagaaaagtaataattcaaaggtaggaaaattaaaataagttggctataatagaagataattttgatggagagagttgagatctacaatttgagactaactttgatggacaccctaaaaaaatgtaaaaaatctttgtaaatgtatatgtttttttttttatagtaaaataggttgtaattatttaaaatatttttcaaaattttgaaatttttttataaattatcaactacaacatttatgattataaatttttgaatttttaatattttatatgatattttaaataactacaacgtatactgtataaaaaaaattaaacaatttttttatgccaaaataataaaaagtgcatcaatacacatttttctgaggtggacactaaaagtattgtatgataacaaaatttggtttcaatagaataagagagtccatgagatatttatattgagaccctcatcgacctcctccatcaatgatattcactctcttttatccccaggccgagatcccctagtcttactttgggattttcatgaggtcaattttcatttttaaaaaaaaaaaaaatcacccaaagatattcataataattctaaaatcaaacaagtacataaaattaattaacaatagtcaatataatatgtaaaattttatttagaagataacattggcatcatgactattatttaattatacatgaaaaactataatattaaaattagtcaaagtaacaagattattataatgaagtgtgaaatcaatgaaatatataggttccttcctcacccattcatttaaaataatttagttttgtttacctttcccacaaataatgctatatataagaaaaaggtctaaactcatgttaattgcaagaaaa

SEQ ID NO: 3 - DNA, 3003 bp, Cannabis sativa
attaattgaccataagtctatttgaggatgaattctttggcaagaggtgggaaaatgataatttgttgggtttgaagatgattgcaatttacattattcattgtttttataagtaacacccctaaggaagaagtatagaggcctgaaatagatattattgatagtgatcaagggcagattgctcatgcgatcaagatgaagagtaagtgtgagagtaatagagaacgtagttatgatggctggtatagagtattagggtatccatgggtatttattgtttggtgtgatgaatgttgtcccaagatattagaatctttttccaaacatgttgcctctatgatttctagaattcaaaattggtcaaatgatgttaggcatgagatgcaaacgccatgtttgaaggaattgagattgaagattttttatttgcctctgaagatggtaatgttaagtttttttttttattttgtgttatgaatgtgattaacaatgctcctcttacatgatatttataaatttgaatcaaaacataagaagtgagattttaatatattaacaaagtttttgacatagattttaatttagaactagagtataaaattggtctatgtaatttagggttgagttcattgcttcctgttgtttaaatgttttgatagagatattgagaatttacaatagcttgagttgtatatttcttaatccaaatatttaacgcttttgaatatccttcatcaatatctatctatatctatatataatataaaggaaagcacaaaatgagtttaggtggcatcttagattttttatactatttttaaaatttgtagtttttttctattttgtttagaaaacaacaacaaaaatacttgtaactaaatgtcccacattgtttataaatgatgtaggcatcatccatgtaggttataaatattattctttttaagtcatattaattcatccaaaagtatttttgttcatgggtggttgattaatttcttaccctataagtttttattattttatttttaatgttttatagtttttatgtttcatctaatatacatttcattatttatatttttaaaagtaattatttaataaatatttgttttataatttttttatatttgatgtaatatataataataataatatatatttattataagttaattttttttagattttatatataattctatatttttattagcccaaaacaatattcaaactaaattattttaaatgaaactcaacattattttttttctcaatatataaaaaaaatataaataaaatgagatgtcattttatgaatattttttattatttatctattaatattttatctattttataatatgtcttctaagtaatatatattattttgtaagagtctcacattatttacaaactatgtgagtattatacttattaactacaagtaaacacaatatatattatatttcattaaaaaaatacaacaagtaaaatatttcttcattcacaccgatttgtccaaaaattaatattgtttctctttatataggggtaattgaataccttatccaattaattttatatatttatatataaatatatatatattctattgatcaactttatataaatatctaaaaattaatattttttttctacagatttgattatttttgtccatttacttataatatttgtaatttttttatttcataattactttaaatatacattttaattagtttatgtatatatacatcttaattgagtttaaaaaatgtggcactagaaaagagaagagacaaaaatgttcatcaattaaaataataatattagtccaacttaaaaattgatacaaataattaatattataaataataatagtaataaaaaagataacaaatcatgattcattaatttaatgaattgttgcaagaaaaaattaagaaataatattttaaaatatctacctctcattcaataaaaacgttaaaaatttacaatgttaaaattattagtacttaaattaataattttatataaataaaaacaaaactataaaaatatttaatttaaatatatatgaacaaaaaattaagttttgctataaatgatatgaaatgaaaaataacaatttttttaattatagaaaaatttataccattataatatttgattcatttaaaaaaaattatatttttaatagcttttaattattataaataactttattctaaatgtgtttttattttattgtattatgattaaaatttgtgctcatcacaaataaaagaaattttacataaatatattatataattttatattattcataaaaaatataaaatagtattagactgaattgagatttgattcgtaactaaaaattaatttctttaatgtaaaattttaaaaacaaattattaaaaccgcgtgaagcgcggatctattccctagttgaataataaagtagatagtagaggaggttagaatttataatttttcatttaaatatttgaatttactttactgttctttgttattcttcatttaattttgctatttgttattttacttttcaaaaattataattttaattaccaaataaaaagtaaaatagatattggtacttgatattcactctttatgggaaccataataaactataaaagtcattatgtgtacttgctaccataggcacctatatcccacaaactagctaccatagccaatttcttgttttttgtttccaatatccaatttttattgatgccaaactattcaatgtacaatgtacatttattttcaataagggcttcacctaacaaaggtgcctaattttagttgatttattttttatcacatgtgactatttaatgactatcaaattataaaatatttaagtcaatttatttgccccaactccaatatataatattataaataggatagttctcaattcctaataattcaaaaaatcattaggactgaagaaaaatg

SEQ ID NO: 4 - DNA, 3000 bp, Cannabis sativa
atgccgaacaaagcaccacatacatttgctcggacgaagtcctcttagccgggcagtgagcaatccgcgcagcacgccaacacttagcaaattcagctttcgcatcgtgagtcaccaaagaaatccctataactttgggatcaggccatagagatatggcaagattcgagaaactctaataacaagaattaagattgtcccaaataaagtgataaacttttaaacaaagaagcaacaaggcgtacattgtcgacctcagaaggataaggaaaaaggctccagctcgctatcacggtcactggaaccaagctcctgtacggacatcctcagacacaataattaaagatgtattcaaatacacgatcattttgatccaataaatgcgggaggatcacatctatatgattctcaaataataaccgcatttacacaacgtgatatgtaatcaaatcccatgattttaggggatgatcgttgtaaaaagattacagtcaactttgtaattaactgcctactatgtgattataaatagtggcaagtaagatcaaaaaagtggacgaaaaaagcatacaaaaaaggctctgagaaaactgtcaaaaatctgaataagattgactcgtaaactatgcataattttaactgtaaaaccacgtaaaaaatcctgtgtgttcatattttcattgtacatcttttattttttctatgtgagattgtcactgttaggcttaaatttagttaacgaaaatttgcattaacacaatctaatttattttggattggttagttttaaatattaaattattgaatatttttttcacataactaataataaaataaaaagaacccaatattgttttatgtcgccaacaacaaattaaaataaataaaacaaaaaaataactaacccaagggaagaaaaaaattatatatataaagaaatattaacttttgttttattgtatgtacttgcataataaataaattgtaattatgtgcacttacacatccaataaatttttcaacaataaactattatattttgatgatattaatgccatgttatacgtcttataattattcaaagttatcatagagtattcaattttaatgagatcgagggtaatcgagatcatttaaatattttttactgcaaaacttttcatctatttattatgcatttttttatatgacaaaatttaatattcaattttttatttgacaaattttttcatttgattactattattattgcatgtgatattttattgttcactattacactttatgtaatacatccatcaatatatattaacaaataaaaacttaaaactttccacacataactttaaaattttataatataagaattgttccatagtaattaatcttaaaaaaaaatattatttagaaaaaagaaataaatttaaattattaattaatttgaaagcttaaattaaatataaataaaataataaaaattattatgttttttcaaatgattgaacttaagtcattgtacttgatgaaaaaaaaagaaattcaaattattaaggtggcgtttggtaacacttttttaatcagttttctgtttttaaaagtagaaaagtaaaaatatttttcaaaaacatgttctataaaactgtttttactttttaattttataattagaaatcaaaattttaaaaacaaaagaatcactttcaatatttttttaaacagtttttttttaatcaatgttttagattaggaccagacccggacccaaattccctccctacggccctggcgctaaacccgacttttgacttgaacccgaatccgacttgcacctaaattattttgaaagcttaaattaatacaaataaaataaaaaaaaaattatcatgttttttcgaatgatgaacttaagtcattgtacttgatgtaagataattttaccacttacaggatgattatattattgattgtattgattattataggattaaataacttgaatatactatacatttctagctacaattattaatgagtggaaaataaaattgaagtgtgttttttggcgataataatacctaagctataagtttgaaatatttataagtacaactgttaagtgttaagtaaattaccacgtgcccatttctaattggtctaagttattaaatttttattttttaaaaaaattaaattaaatataccaagaagaaaatgacacgtggataatgacttaatatttaatggtcggtacctacagagttcccaaaatattacttaggtattattagcgccaaaaataaaacgtaggtatttatttgcaactggaagttaaatttagatatttatgtcgcaaattactcttttttatccattttttttagatgttttgtggaccattttgtatatgagttggcgtgtagttttttagtgttacgtgttgaatctctagctcttttggtgtgaagtttgaagacgttatagctttattgtctaaatttcttggggtagttttgtcccatgttgctcgcttactttagaagttcaaagtttgacaaaacatgctattcggttatacaatgagctatcctagttcaaggagatttctgtgctatttgtggatgtctgcattgtaaattcatgagtgattcttataactttattaaaaaaagacaaatagatccatagataaatatttgcaagcgctttcttgtcatagcaaattttttttccataggaaatttaaaaaaatcatcttaactttttttcatcaataaatgatgctcaattattcaatgcaccatgtacatttatttttaataagggctgcacctaacaaaggtgcctaattttggtaaaaaagaaattacggcatgtgaatatttaatgagcatcaaattacaaaatatttagttaatttctttacccccactccaatctataaagctataaataggataattttccattcatagtaatttccaacattacgactaaagaaaa

SEQ ID NO: 5 - DNA, 3000 bp, Cannabis sativa
actactaaattacactaaagtgactctataggccatacgccgcgttccacgttaccagagataaaaatatgaaaaagcttaaaattcatgtataacacaaataacacacttaaattcaaatatatctccataaattcataaatcacaactaaaagtctaattctaaattagaccctaattatctatactaaaattaagcggtacaaatttaatcactaataatctactaattttcatgtctaattatacgatcattacaactaagacgtgtttagatgcattttctttgttccaatttgtctccgacttcaagtgcgagtaatgtaactcctgacatggaagtattggtttatgttatcttaataggtaaagctaatgatgtgaggaaactcatttccacccatactcataatgcctttatcaccttaaaaattcaatgactttcccatgtttgattattagcttgtgtaaggaagttggtgttttgatgaaaattatatctaaggacttatgatagctattttattaaccatgttgggactgtaggcccttcttcttcttcttctattgcaagtcttttctctaatgcaggttctttagggcccacttctaggcctcccctttattttttagtatatagtaggttagtccatatttagttttgtgctttattgattttttttttatttttagtttttcctttatgctaagccaagttgtttttaggatgttgtatttttatgtgaatcaagttgataatgataagctgagaaaagtgattgaatatattgtttgcatacttggatagaaaaagccatcgtattatttgtgtttcctgattgaattttttatttcgttcaaatgagtttgggtaaatatttgatagcctaatcatgattttcgaataaattccttatttttgaaagtttttattcttgttgaattttttgaagaaaaaaattattatgaaatgaaaaggtaattatcttagaactaggttgttttgtatttaaaatgaaatcctagatgacacgcttaggaatataatataggcattctttagaataattgagcttgttaagccaacttttgttatgttaattcttaacttgtctttttgagcttaaaattaacaattttatttatttgaagcccatttaagcctaattgaacctcttttaacattccattctcttgaataatgccatacgttttttattgattgattgggtatggttcgcgatggggtataatgaaaagttggtgtgaaaaaaaaagttgaaaaaatttaaaaagtaataagaaaaaaaaataagtacactccttgttaaaaaaaataaattaaaaaataagtttcaagaagtgtaattaagagagaaaaaaatatttgaattgaatagattttttaaatcttagaaatgaaagcagtttggggattgttattgggttttaaagtgatatcgtgttgattttgtagtgcttatcgtataattgcatctaaattacgtttttacctacccatacctaagctattgttacaagtctaaaaaagtaatttttatttttgaacaagagttgtctacattggtagagaatggtgagtaatgcaaagatatgaaatattaaatttacttctttggaataaaaattcagcaagtataagttgaaatatgaaaaaatagaagacttggccaaaaatgctgtaaaaattattgtttctaaaggaattctaagaatgcacacttatttggagtggtcgtgtaactatgctagaaatgcatatcaagagaactttttagtgaagactatttttgtctattcgattggcaaaaaagtcgagcttaggtcttaattgcgattttgagaggggagacttaattttttgacgactggaactggaaagaatcgttgagaagtgctttggatgaagactggatctagagtttagagttcttttcttttttttcttttacttataaatgttaatgtcatgtttatggatttcttagtgatgaacatgaactaaattttttttaatgatttttaattgaatctcttgaaactttattataaattaatgcaagttttctattttttcttcatctaagatctattcaatttgtgtatattgtgaatgttattaattacgcacctttttacatgctattaatgaattaaaattaaaacctgcgagaattgaatatgctaaaaaacaatttacatagattttaatgtagaacaaaagtatcaaatcggtctatataatttaggattgcgttcattgttttcaattgatttaatttcttatattgatataaagaatttgcaatacattgagtttcttaacccgaatattaaacggttttgaatatcttcatcattgattgaataataaagtggatagtagagggaattagaatccataattttttatttatatatttgaagttagtttattattctttgctatccttcatttaattttgctatttgttattttagtttcaaaaatttatttttcattacaaaataaaaaataagataggatattggtacttgataagtcttctttgtggaaacgataatcggtattattaggtatatttgcatatttcaatattattgcaaaaaataaatatattaataaataaagtggaaggtgccataggtacctatatccacaaactagcatattgaaagaaaatggatccatggataaatatttgcaagtctttttttttttgcatagccaatttctttttttattattattttaatagaatatttcaaaagggcatctaacatttatttttaataaggactgcacctaacaaaggtgcctaatttttgtgaacttttttttaccacatgtgactatttaatgagtactaaattatgaaatatttagttaatttctttgcccccgctccaatatataatgctataaatagcataattttctattcatagtaattcaaaaatcattaggactaaagaaag

SEQ ID NO: 6 - DNA, 3000 bp, Cannabis sativa
aatcgattccataggggtttcctataaattttttaaaatttttattaaaaatctaaatttatttattttaaattaaattaaaattaaattaaaattaaattaaattaaaaagttaaataaatacaaatttacattgctctaagtgtttgtatattaggtatttttttattaatattttatattttataatatgtgtttgtatttttccagtattttagtatcatttttataatttttaagttatgttttatataataattagtatattgaataataaataatgtgtaatattttaatttttatgtaatttaattttttcatacatttaaattttataatatgagtttgtattttatagtatattagtattatttttataattttttaagttatcttttgtataataattagtttattgaataataaataatgtgtaatattttaatttttatgtaatttaatatttttatacatttaacttttataatatgggtttgtatttttctagtatattattattatttttataattttttaagttatgttttgtataataattagtatattgaataaaaataatgtgtaatattttaattttgatgtaatttaatatctttatacatttactttttttaaaaaaaaacatttactctaagcaaatgtttacactaatattatcccaatagaccatttttgtaatttttttttggcttactatatctatcattaacactcaacaatatctatcctacgtattaatattattctaaaattactctaagtatttaaggattatctaaatatatataaaaaatatcttataaaagtaagttgaaaaaacataccttatacaaaaaattatgtgcttgacttccgataaagtcaatttcgataaaatcctaacaaccaagtttaagagaaactaaaataaaatattatcctaattctaccgaaatttcggcagcataacggctataatagaaagtttccaaaaaattcaaacctattaataacaacaaaacacaattcaaaataatattaaataatcataacaacatttagcaatataaaagtttaccacccatccgactacagtacatgtattcacagaacctacttcactacagatgaatatttaagatattcaaactcatctaacatgtatattgtttcacccatcgatccgacatagtacaatatattgtagaacctacttcactacaaatgaatatttaagattttcaaactctaagcattcataattaatgtataataaaagttagtaaatgtatagctcgagctacctagaaaccgatcaacacaaaataagttggatccttgcaaattgacctcacaaccttcaacataaagaaatactacaaaattaccaatttttttatttagctacttactagttatataatttatgagtaactagttactaacttgtttctaatgagtaagtaattagtaataaaaatatatgaacatgaatgaaaattagtagcttgttaagtagctgtaactaactatttactacctactaaagaaataaaacacatgaataaaattaaaaaaaaaaattgatacaaaatatacttaatttctaaacttttgtttctaaactattttttagaaattaatttctaaacttttattttaatactttcctatctcattctcattcacaatatttaacaaaactattagtttaaaatactcaatatatacatatacaatatataaaatactctcctatattacatttctcattcacaatatatatacacacaatacatatttgatacaacacaaaatacatatatatactcttctatatcacatttcccattcacaatagatatacacataatacatattcattacaacataatacatattcaataatatagaaaaccctaaaaaaatttataaaataacaaataaagcaatgtacattactaaaattagtagaaattagatttataccttaataggcgttgagatacaatgttttctccactaaaaacggtggccggaggtataatcaccactacctttactttttgtttaccccaaaaaatgaggaaaaatgtaatcttttcaggtatatagttttaggtatgaaaaatagaagatttagaaataaacatgagctaaaatggtgaaaaaatagtgaaatggaggtgatttttcgtggtggttggtggagagggtttggggtttctttggtttgggggtttctgggttggatggtcgaatgaggaagatgaagggctgggctagggttataaaaccttttggcgtcggttatcttctaactattgataatttcttttattagtttttaatataattgtaaggggataattcattatttttatcttatttttttgtagatattttgtggaccatttgttatatgagttggtgtgtagtttttcagtgttacaagttgaatctctagctcttttattgtgttgtttaaggatgcgtagctttattttctaaatttttagtaatttttttgtcccatgttgcttgcttactttagcaattcatagtttgacaaagcataacattcagttagacgatgatctatcctaattcgaggaggtttctgcaatatttttgtgaatatttgcatcgtaaattcatgagtgattcttatcacttcttaaaaaaaaagatataaacatccatagataaatatttgctagtcctttctttccatagtcaacttcaatatggcatcttaacttctttccatcaacaaatgatgctcaactacttaatgtacaatttatatttatttttagtaagggatgcacctaacaatgatgcctaattttggtgaatttttttttaccacatgtgacttaatgatatcaaattaggaaatatttagttaatttatttgcccctgctccattataaaaagctataaataggatagttcttaattcatagtaattcaaaaattattagaactaaagaaaa

SEQ ID NO: 7 - DNA, 20 bp, Cannabis_sativa
aattaataaaatatacataa

SEQ ID NO: 8 - DNA, 20 bp, Cannabis_sativa
atgccacttagactcttctt

SEQ ID NO: 9 - DNA, 20 bp, Cannabis_sativa
aagcctaagaagagtctaag

SEQ ID NO: 10 - DNA, 20 bp, Cannabis_sativa
aataggaaagaaaaaaaaat

SEQ ID NO: 11 - DNA, 20 bp, Cannabis_sativa
tgttagaataaaggaaaaat

SEQ ID NO: 12 - DNA, 20 bp, Cannabis_sativa
aaatagatgtgttagaataa

SEQ ID NO: 13 - DNA, 20 bp, Cannabis_sativa
acaaactaaaagtcccacat

SEQ ID NO: 14 - DNA, 20 bp, Cannabis_sativa
taaaagtttttaaccaatgt

SEQ ID NO: 15 - DNA, 20 bp, Cannabis_sativa
ttaaaagtttttaaccaatg

SEQ ID NO: 16 - DNA, 20 bp, Cannabis_sativa
actattatttataacttaca

SEQ ID NO: 17 - DNA, 20 bp, Cannabis_sativa
aatatttcttagacaaatgt

SEQ ID NO: 18 - DNA, 20 bp, Cannabis_sativa
ctaagaaatattatttttta

SEQ ID NO: 19 - DNA, 20 bp, Cannabis_sativa
taagaaatattattttttat

SEQ ID NO: 20 - DNA, 20 bp, Cannabis_sativa
gaaatattattttttatggg

SEQ ID NO: 21 - DNA, 20 bp, Cannabis_sativa
tttatgtataacaatttatt

SEQ ID NO: 22 - DNA, 20 bp, Cannabis_sativa
ctttatgtataacaatttat

SEQ ID NO: 23 - DNA, 20 bp, Cannabis_sativa
caataaattgttatacataa

SEQ ID NO: 24 - DNA, 20 bp, Cannabis_sativa
ataaaggaaagcctaagaag

SEQ ID NO: 25 - DNA, 20 bp, Cannabis_sativa
tgccacttagacctcttctt

SEQ ID NO: 26 - DNA, 20 bp, Cannabis_sativa
agcctaagaagaggtctaag

SEQ ID NO: 27 - DNA, 20 bp, Cannabis_sativa
aaataggaagaaaaaaaaat

SEQ ID NO: 28 - DNA, 20 bp, Cannabis_sativa
tgttagaataaaggaaaaat

SEQ ID NO: 29 - DNA, 20 bp, Cannabis_sativa
ataagagtgtgttagaataa

SEQ ID NO: 30 - DNA, 20 bp, Cannabis_sativa
atacaacaaaagtcccacat

SEQ ID NO: 31 - DNA, 20 bp, Cannabis_sativa
aaaaagtttttaaccaatgt

SEQ ID NO: 32 - DNA, 20 bp, Cannabis_sativa
taaaaagtttttaaccaatg

SEQ ID NO: 33 - DNA, 20 bp, Cannabis_sativa
actattatttataacttaca

SEQ ID NO: 34 - DNA, 20 bp, Cannabis_sativa
taatatttcttagacaatgt

SEQ ID NO: 35 - DNA, 20 bp, Cannabis_sativa
ctaagaaatattatttttta

SEQ ID NO: 36 - DNA, 20 bp, Cannabis_sativa
taagaaatattattttttat

SEQ ID NO: 37 - DNA, 20 bp, Cannabis_sativa
gaaatattattttttatggg

SEQ ID NO: 38 - DNA, 20 bp, Cannabis_sativa
atatatataacaaatttatt

SEQ ID NO: 39 - DNA, 20 bp, Cannabis_sativa
catatatataacaaatttat

SEQ ID NO: 40 - DNA, 20 bp, Cannabis_sativa
attaaaaatttatctataag

SEQ ID NO: 41 - DNA, 20 bp, Cannabis_sativa
ctcaaataatcttataagta

SEQ ID NO: 42 - DNA, 20 bp, Cannabis_sativa
tttagtttaaattatttaga

SEQ ID NO: 43 - DNA, 20 bp, Cannabis_sativa
aaaaaaacaaactatatttt

SEQ ID NO: 44 - DNA, 20 bp, Cannabis_sativa
attaatatcaatatatataa

SEQ ID NO: 45 - DNA, 20 bp, Cannabis_sativa
aaggaaagtctaaacaaaag

SEQ ID NO: 46 - DNA, 20 bp, Cannabis_sativa
gtattatttattttctatgt

SEQ ID NO: 47 - DNA, 20 bp, Cannabis_sativa
ttaaaaaaaatcaagtaatt

SEQ ID NO: 48 - DNA, 20 bp, Cannabis_sativa
cttaaaaaaaatcaagtaat

SEQ ID NO: 49 - DNA, 20 bp, Cannabis_sativa
atataatttaaattatttta

SEQ ID NO: 50 - DNA, 20 bp, Cannabis_sativa
tacataaaaagatatattta

SEQ ID NO: 51 - DNA, 20 bp, Cannabis_sativa
attgtgaatgctaaacttat

SEQ ID NO: 52 - DNA, 20 bp, Cannabis_sativa
aaaaaaaaattacaactaat

SEQ ID NO: 53 - DNA, 20 bp, Cannabis_sativa
ttttgtgataaatattaaaa

SEQ ID NO: 54 - DNA, 20 bp, Cannabis_sativa
atataaaatattacaaaagt

SEQ ID NO: 55 - DNA, 20 bp, Cannabis_sativa
gaactaagccgcgcttcgct

SEQ ID NO: 56 - DNA, 20 bp, Cannabis_sativa
ggacaacaccgagcgaagcg

SEQ ID NO: 57 - DNA, 20 bp, Cannabis_sativa
tctactttattattcaacta

SEQ ID NO: 58 - DNA, 20 bp, Cannabis_sativa
atctactttattattcaact

SEQ ID NO: 59 - DNA, 20 bp, Cannabis_sativa
aataataaagtagatagtag

SEQ ID NO: 60 - DNA, 20 bp, Cannabis_sativa
tatatattttattttttatt

SEQ ID NO: 61 - DNA, 20 bp, Cannabis_sativa
ataaaaaataaaatatatat

SEQ ID NO: 62 - DNA, 20 bp, Cannabis_sativa
tacttcatatttagttttta

SEQ ID NO: 63 - DNA, 20 bp, Cannabis_sativa
acttcatatttagtttttat

SEQ ID NO: 64 - DNA, 20 bp, Cannabis_sativa
tcattaatatatattttttt

SEQ ID NO: 65 - DNA, 20 bp, Cannabis_sativa
ttcattaatatatatttttt

SEQ ID NO: 66 - DNA, 20 bp, Cannabis_sativa
atatatattaatgaaaaaaa

SEQ ID NO: 67 - DNA, 20 bp, Cannabis_sativa
tatattaatgaaaaaaaagg

SEQ ID NO: 68 - DNA, 20 bp, Cannabis_sativa
ttaatgaaaaaaaaggtgga

SEQ ID NO: 69 - DNA, 20 bp, Cannabis_sativa
aaaaaggtggaaggtgccat

SEQ ID NO: 70 - DNA, 20 bp, Cannabis_sativa
ttgtgggatataggtgccta

SEQ ID NO: 71 - DNA, 20 bp, Cannabis_sativa
atagctagtttgtgggatat

SEQ ID NO: 72 - DNA, 20 bp, Cannabis_sativa
atatcttatagctagtttgt

SEQ ID NO: 73 - DNA, 20 bp, Cannabis_sativa
aatatcttatagctagtttg

SEQ ID NO: 74 - DNA, 20 bp, Cannabis_sativa
gacacgcaaatatttatcta

SEQ ID NO: 75 - DNA, 20 bp, Cannabis_sativa
gaaattagatatgaaaagaa

SEQ ID NO: 76 - DNA, 20 bp, Cannabis_sativa
tttttttcaatagccaattt

SEQ ID NO: 77 - DNA, 20 bp, Cannabis_sativa
agaagttaagctgccgaaat

SEQ ID NO: 78 - DNA, 20 bp, Cannabis_sativa
tagtttggcatcattatata

SEQ ID NO: 79 - DNA, 20 bp, Cannabis_sativa
aaattttacattgaatagtt

SEQ ID NO: 80 - DNA, 20 bp, Cannabis_sativa
atttagatttattttcatta

SEQ ID NO: 81 - DNA, 20 bp, Cannabis_sativa
tttagatttattttcattaa

SEQ ID NO: 82 - DNA, 20 bp, Cannabis_sativa
aaaaattaggcacatttgtt

SEQ ID NO: 83 - DNA, 20 bp, Cannabis_sativa
taaaaaaatccacaaaaatt

SEQ ID NO: 84 - DNA, 20 bp, Cannabis_sativa
acaaatgtgcctaatttttg

SEQ ID NO: 85 - DNA, 20 bp, Cannabis_sativa
agtcattaaatagtgacata

SEQ ID NO: 86 - DNA, 20 bp, Cannabis_sativa
acttaaatatttctataatt

SEQ ID NO: 87 - DNA, 20 bp, Cannabis_sativa
tataactttatattggagcg

SEQ ID NO: 88 - DNA, 20 bp, Cannabis_sativa
ctataactttatattggagc

SEQ ID NO: 89 - DNA, 20 bp, Cannabis_sativa
tctataactttatattggag

SEQ ID NO: 90 - DNA, 20 bp, Cannabis_sativa
tcctttctataactttatat

SEQ ID NO: 91 - DNA, 20 bp, Cannabis_sativa
tccaatataaagttatagaa

SEQ ID NO: 92 - DNA, 20 bp, Cannabis_sativa
ggtgaaatgggtgtggctag

SEQ ID NO: 93 - DNA, 20 bp, Cannabis_sativa
tgagtgaggtgaaatgggtg

SEQ ID NO: 94 - DNA, 20 bp, Cannabis_sativa
gagagtgagtgaggtgaaat

SEQ ID NO: 95 - DNA, 20 bp, Cannabis_sativa
tgagagtgagtgaggtgaaa

SEQ ID NO: 96 - DNA, 20 bp, Cannabis_sativa
ttgattggtgagagtgagtg

SEQ ID NO: 97 - DNA, 20 bp, Cannabis_sativa
aaagtggatgtgctcttgat

SEQ ID NO: 98 - DNA, 20 bp, Cannabis_sativa
acctataattccttataaag

SEQ ID NO: 99 - DNA, 20 bp, Cannabis_sativa
aagagcacatccactttata

SEQ ID NO: 100 - DNA, 20 bp, Cannabis_sativa
tccactttataaggaattat

SEQ ID NO: 101 - DNA, 20 bp, Cannabis_sativa
ataaggaattataggtcatt

SEQ ID NO: 102 - DNA, 20 bp, Cannabis_sativa
taaggaattataggtcattt

SEQ ID NO: 103 - DNA, 20 bp, Cannabis_sativa
tctctccaaattgggcatgt

SEQ ID NO: 104 - DNA, 20 bp, Cannabis_sativa
ttgggcctacatgcccaatt

SEQ ID NO: 105 - DNA, 20 bp, Cannabis_sativa
caactaattctctccaaatt

SEQ ID NO: 106 - DNA, 20 bp, Cannabis_sativa
acaactaattctctccaaat

SEQ ID NO: 107 - DNA, 20 bp, Cannabis_sativa
aattagttgtcttagcccaa

SEQ ID NO: 108 - DNA, 20 bp, Cannabis_sativa
attagttgtcttagcccaat

SEQ ID NO: 109 - DNA, 20 bp, Cannabis_sativa
acgtctttggtcagcccatt

SEQ ID NO: 110 - DNA, 20 bp, Cannabis_sativa
aacgtctttggtcagcccat

SEQ ID NO: 111 - DNA, 20 bp, Cannabis_sativa
gacacactctaaaacgtctt

SEQ ID NO: 112 - DNA, 20 bp, Cannabis_sativa
gacgttttagagtgtgtcca

SEQ ID NO: 113 - DNA, 20 bp, Cannabis_sativa
acgttttagagtgtgtccat

SEQ ID NO: 114 - DNA, 20 bp, Cannabis_sativa
actaacacgctcggggccca

SEQ ID NO: 115 - DNA, 20 bp, Cannabis_sativa
ataatgtactaacacgctcg

SEQ ID NO: 116 - DNA, 20 bp, Cannabis_sativa
cataatgtactaacacgctc

SEQ ID NO: 117 - DNA, 20 bp, Cannabis_sativa
acataatgtactaacacgct

SEQ ID NO: 118 - DNA, 20 bp, Cannabis_sativa
accatcgagccataatggga

SEQ ID NO: 119 - DNA, 20 bp, Cannabis_sativa
acattatgtccatcccatta

SEQ ID NO: 120 - DNA, 20 bp, Cannabis_sativa
tattaccatcgagccataat

SEQ ID NO: 121 - DNA, 20 bp, Cannabis_sativa
gtattaccatcgagccataa

SEQ ID NO: 122 - DNA, 20 bp, Cannabis_sativa
tccatcccattatggctcga

SEQ ID NO: 123 - DNA, 20 bp, Cannabis_sativa
tatatataatcggtaatgca

SEQ ID NO: 124 - DNA, 20 bp, Cannabis_sativa
taatgataactatatataat

SEQ ID NO: 125 - DNA, 20 bp, Cannabis_sativa
tcttgttagaatattttatg

SEQ ID NO: 126 - DNA, 20 bp, Cannabis_sativa
agtacaatagcagaaattct

SEQ ID NO: 127 - DNA, 20 bp, Cannabis_sativa
gtacaatagcagaaattctt

SEQ ID NO: 128 - DNA, 20 bp, Cannabis_sativa
aaagtgactttattgtgtag

SEQ ID NO: 129 - DNA, 20 bp, Cannabis_sativa
taacaatgacgtatacaaag

SEQ ID NO: 130 - DNA, 20 bp, Cannabis_sativa
attgttattattaagctttc

SEQ ID NO: 131 - DNA, 20 bp, Cannabis_sativa
aaagcaaacagttttataca

SEQ ID NO: 132 - DNA, 20 bp, Cannabis_sativa
tctttacttttattctttgt

SEQ ID NO: 133 - DNA, 20 bp, Cannabis_sativa
ttacacactactatacgaaa

SEQ ID NO: 134 - DNA, 20 bp, Cannabis_sativa
tacacactactatacgaaaa

SEQ ID NO: 135 - DNA, 20 bp, Cannabis_sativa
acacactactatacgaaaag

SEQ ID NO: 136 - DNA, 20 bp, Cannabis_sativa
ataaaatgcccataaaaatt

SEQ ID NO: 137 - DNA, 20 bp, Cannabis_sativa
tataaaatgcccataaaaat

SEQ ID NO: 138 - DNA, 20 bp, Cannabis_sativa
ggctttaatcccaattttta

SEQ ID NO: 139 - DNA, 20 bp, Cannabis_sativa
gctttaatcccaatttttat

SEQ ID NO: 140 - DNA, 20 bp, Cannabis_sativa
tttttgctaaaataaaaatt

SEQ ID NO: 141 - DNA, 20 bp, Cannabis_sativa
aaaaattaggatatatgtca

SEQ ID NO: 142 - DNA, 20 bp, Cannabis_sativa
ataaaagctcatataaaaag

SEQ ID NO: 143 - DNA, 20 bp, Cannabis_sativa
taaaagctcatataaaaagt

SEQ ID NO: 144 - DNA, 20 bp, Cannabis_sativa
tttttaatttcgattattat

SEQ ID NO: 145 - DNA, 20 bp, Cannabis_sativa
tttcgattattataggaaaa

SEQ ID NO: 146 - DNA, 20 bp, Cannabis_sativa
ttcgattattataggaaaac

SEQ ID NO: 147 - DNA, 20 bp, Cannabis_sativa
ctgagctttcaattaaaact

SEQ ID NO: 148 - DNA, 20 bp, Cannabis_sativa
tctgagctttcaattaaaac

SEQ ID NO: 149 - DNA, 20 bp, Cannabis_sativa
atggaaaaattggaattaaa

SEQ ID NO: 150 - DNA, 20 bp, Cannabis_sativa
tatggaaaaattggaattaa

SEQ ID NO: 151 - DNA, 20 bp, Cannabis_sativa
tccagtttttatggaaaaat

SEQ ID NO: 152 - DNA, 20 bp, Cannabis_sativa
agtattaattccagttttta

SEQ ID NO: 153 - DNA, 20 bp, Cannabis_sativa
tccaatttttccataaaaac

SEQ ID NO: 154 - DNA, 20 bp, Cannabis_sativa
taatgaagaacatagtatct

SEQ ID NO: 155 - DNA, 20 bp, Cannabis_sativa
gatatgatttcattaattaa

SEQ ID NO: 156 - DNA, 20 bp, Cannabis_sativa
tattgtaatgttaatattta

SEQ ID NO: 157 - DNA, 20 bp, Cannabis_sativa
tattaacattacaatatcaa

SEQ ID NO: 158 - DNA, 20 bp, Cannabis_sativa
gcttcatggttcatggttca

SEQ ID NO: 159 - DNA, 20 bp, Cannabis_sativa
tgacattgcttcatggttca

SEQ ID NO: 160 - DNA, 20 bp, Cannabis_sativa
tcgccattgacattgcttca

SEQ ID NO: 161 - DNA, 20 bp, Cannabis_sativa
gaaccatgaagcaatgtcaa

SEQ ID NO: 162 - DNA, 20 bp, Cannabis_sativa
agagagagagagagagcttc

SEQ ID NO: 163 - DNA, 20 bp, Cannabis_sativa
gagaaaactcttgctgaaaa

SEQ ID NO: 164 - DNA, 20 bp, Cannabis_sativa
ctatctttacccatcactgt

SEQ ID NO: 165 - DNA, 20 bp, Cannabis_sativa
agagttttctccgacagtga

SEQ ID NO: 166 - DNA, 20 bp, Cannabis_sativa
gagttttctccgacagtgat

SEQ ID NO: 167 - DNA, 20 bp, Cannabis_sativa
gtgatgggtaaagatagaag

SEQ ID NO: 168 - DNA, 20 bp, Cannabis_sativa
gggtaaagatagaagtggag

SEQ ID NO: 169 - DNA, 20 bp, Cannabis_sativa
agaagtggagaggaagaaga

SEQ ID NO: 170 - DNA, 20 bp, Cannabis_sativa
cgatctcagccacgcacgaa

SEQ ID NO: 171 - DNA, 20 bp, Cannabis_sativa
gcgatctcagccacgcacga

SEQ ID NO: 172 - DNA, 20 bp, Cannabis_sativa
agaagaaggcccttcgtgcg

SEQ ID NO: 173 - DNA, 20 bp, Cannabis_sativa
ttcgtgcgtggctgagatcg

SEQ ID NO: 174 - DNA, 20 bp, Cannabis_sativa
tgagatcgcggccaagaatc

SEQ ID NO: 175 - DNA, 20 bp, Cannabis_sativa
tttctgcattgccggattct

SEQ ID NO: 176 - DNA, 20 bp, Cannabis_sativa
cccaattctttctgcattgc

SEQ ID NO: 177 - DNA, 20 bp, Cannabis_sativa
ttcatcctcaaatagactta

SEQ ID NO: 178 - DNA, 20 bp, Cannabis_sativa
attgaccataagtctatttg

SEQ ID NO: 179 - DNA, 20 bp, Cannabis_sativa
ctatttgaggatgaattctt

SEQ ID NO: 180 - DNA, 20 bp, Cannabis_sativa
aggatgaattctttggcaag

SEQ ID NO: 181 - DNA, 20 bp, Cannabis_sativa
atgaattctttggcaagagg

SEQ ID NO: 182 - DNA, 20 bp, Cannabis_sativa
tgaattctttggcaagaggt

SEQ ID NO: 183 - DNA, 20 bp, Cannabis_sativa
gtgggaaaatgataatttgt

SEQ ID NO: 184 - DNA, 20 bp, Cannabis_sativa
tgggaaaatgataatttgtt

SEQ ID NO: 185 - DNA, 20 bp, Cannabis_sativa
ttttataagtaacaccccta

SEQ ID NO: 186 - DNA, 20 bp, Cannabis_sativa
cctctatacttcttccttag

SEQ ID NO: 187 - DNA, 20 bp, Cannabis_sativa
gcctctatacttcttcctta

SEQ ID NO: 188 - DNA, 20 bp, Cannabis_sativa
ggcctctatacttcttcctt

SEQ ID NO: 189 - DNA, 20 bp, Cannabis_sativa
cccctaaggaagaagtatag

SEQ ID NO: 190 - DNA, 20 bp, Cannabis_sativa
ctatcaataatatctatttc

SEQ ID NO: 191 - DNA, 20 bp, Cannabis_sativa
gatattattgatagtgatca

SEQ ID NO: 192 - DNA, 20 bp, Cannabis_sativa
atattattgatagtgatcaa

SEQ ID NO: 193 - DNA, 20 bp, Cannabis_sativa
aatagagaacgtagttatga

SEQ ID NO: 194 - DNA, 20 bp, Cannabis_sativa
gagaacgtagttatgatggc

SEQ ID NO: 195 - DNA, 20 bp, Cannabis_sativa
gatggctggtatagagtatt

SEQ ID NO: 196 - DNA, 20 bp, Cannabis_sativa
atggctggtatagagtatta

SEQ ID NO: 197 - DNA, 20 bp, Cannabis_sativa
atagagtattagggtatcca

SEQ ID NO: 198 - DNA, 20 bp, Cannabis_sativa
tagagtattagggtatccat

SEQ ID NO: 199 - DNA, 20 bp, Cannabis_sativa
caccaaacaataaataccca

SEQ ID NO: 200 - DNA, 20 bp, Cannabis_sativa
atccatgggtatttattgtt

SEQ ID NO: 201 - DNA, 20 bp, Cannabis_sativa
gaaaaagattctaatatctt

SEQ ID NO: 202 - DNA, 20 bp, Cannabis_sativa
ggaaaaagattctaatatct

SEQ ID NO: 203 - DNA, 20 bp, Cannabis_sativa
aatcatagaggcaacatgtt

SEQ ID NO: 204 - DNA, 20 bp, Cannabis_sativa
ttgaattctagaaatcatag

SEQ ID NO: 205 - DNA, 20 bp, Cannabis_sativa
tgatttctagaattcaaaat

SEQ ID NO: 206 - DNA, 20 bp, Cannabis_sativa
aaaattggtcaaatgatgtt

SEQ ID NO: 207 - DNA, 20 bp, Cannabis_sativa
aatctcaattccttcaaaca

SEQ ID NO: 208 - DNA, 20 bp, Cannabis_sativa
gatgcaaacgccatgtttga

SEQ ID NO: 209 - DNA, 20 bp, Cannabis_sativa
cttaacattaccatcttcag

SEQ ID NO: 210 - DNA, 20 bp, Cannabis_sativa
tttttatttgcctctgaaga

SEQ ID NO: 211 - DNA, 20 bp, Cannabis_sativa
atttataaatatcatgtaag

SEQ ID NO: 212 - DNA, 20 bp, Cannabis_sativa
ttagaactagagtataaaat

SEQ ID NO: 213 - DNA, 20 bp, Cannabis_sativa
aaaattggtctatgtaattt

SEQ ID NO: 214 - DNA, 20 bp, Cannabis_sativa
aaattggtctatgtaattta

SEQ ID NO: 215 - DNA, 20 bp, Cannabis_sativa
tatcaaaacatttaaacaac

SEQ ID NO: 216 - DNA, 20 bp, Cannabis_sativa
attcaaaagcgttaaatatt

SEQ ID NO: 217 - DNA, 20 bp, Cannabis_sativa
atatagatagatattgatga

SEQ ID NO: 218 - DNA, 20 bp, Cannabis_sativa
tatatctatatataatataa

SEQ ID NO: 219 - DNA, 20 bp, Cannabis_sativa
ggaaagcacaaaatgagttt

SEQ ID NO: 220 - DNA, 20 bp, Cannabis_sativa
aagcacaaaatgagtttagg

SEQ ID NO: 221 - DNA, 20 bp, Cannabis_sativa
acatcatttataaacaatgt

SEQ ID NO: 222 - DNA, 20 bp, Cannabis_sativa
tacatcatttataaacaatg

SEQ ID NO: 223 - DNA, 20 bp, Cannabis_sativa
acattgtttataaatgatgt

SEQ ID NO: 224 - DNA, 20 bp, Cannabis_sativa
gatgtaggcatcatccatgt

SEQ ID NO: 225 - DNA, 20 bp, Cannabis_sativa
aataatatttataacctaca

SEQ ID NO: 226 - DNA, 20 bp, Cannabis_sativa
cccatgaacaaaaatacttt

SEQ ID NO: 227 - DNA, 20 bp, Cannabis_sativa
tccaaaagtatttttgttca

SEQ ID NO: 228 - DNA, 20 bp, Cannabis_sativa
ccaaaagtatttttgttcat

SEQ ID NO: 229 - DNA, 20 bp, Cannabis_sativa
aaagtatttttgttcatggg

SEQ ID NO: 230 - DNA, 20 bp, Cannabis_sativa
taaaataataaaaacttata

SEQ ID NO: 231 - DNA, 20 bp, Cannabis_sativa
ataaaataataaaaacttat

SEQ ID NO: 232 - DNA, 20 bp, Cannabis_sativa
tttagtttgaatattgtttt

SEQ ID NO: 233 - DNA, 20 bp, Cannabis_sativa
atttagtttgaatattgttt

SEQ ID NO: 234 - DNA, 20 bp, Cannabis_sativa
atattaatttttggacaaat

SEQ ID NO: 235 - DNA, 20 bp, Cannabis_sativa
agagaaacaatattaatttt

SEQ ID NO: 236 - DNA, 20 bp, Cannabis_sativa
aatattgtttctctttatat

SEQ ID NO: 237 - DNA, 20 bp, Cannabis_sativa
atattgtttctctttatata

SEQ ID NO: 238 - DNA, 20 bp, Cannabis_sativa
tattgtttctctttatatag

SEQ ID NO: 239 - DNA, 20 bp, Cannabis_sativa
tatataaaattaattggata

SEQ ID NO: 240 - DNA, 20 bp, Cannabis_sativa
tataaatatataaaattaat

SEQ ID NO: 241 - DNA, 20 bp, Cannabis_sativa
ttacaaatattataagtaaa

SEQ ID NO: 242 - DNA, 20 bp, Cannabis_sativa
ttaattgagtttaaaaaatg

SEQ ID NO: 243 - DNA, 20 bp, Cannabis_sativa
atttgtatcaatttttaagt

SEQ ID NO: 244 - DNA, 20 bp, Cannabis_sativa
taacgtttttattgaatgag

SEQ ID NO: 245 - DNA, 20 bp, Cannabis_sativa
aaatgaatcaaatattataa

SEQ ID NO: 246 - DNA, 20 bp, Cannabis_sativa
gaatagatccgcgcttcacg

SEQ ID NO: 247 - DNA, 20 bp, Cannabis_sativa
tattaaaaccgcgtgaagcg

SEQ ID NO: 248 - DNA, 20 bp, Cannabis_sativa
tctactttattattcaacta

SEQ ID NO: 249 - DNA, 20 bp, Cannabis_sativa
atctactttattattcaact

SEQ ID NO: 250 - DNA, 20 bp, Cannabis_sativa
aataataaagtagatagtag

SEQ ID NO: 251 - DNA, 20 bp, Cannabis_sativa
aataaagtagatagtagagg

SEQ ID NO: 252 - DNA, 20 bp, Cannabis_sativa
tatctattttactttttatt

SEQ ID NO: 253 - DNA, 20 bp, Cannabis_sativa
ataaaaagtaaaatagatat

SEQ ID NO: 254 - DNA, 20 bp, Cannabis_sativa
tacttgatattcactcttta

SEQ ID NO: 255 - DNA, 20 bp, Cannabis_sativa
acttgatattcactctttat

SEQ ID NO: 256 - DNA, 20 bp, Cannabis_sativa
atgacttttatagtttatta

SEQ ID NO: 257 - DNA, 20 bp, Cannabis_sativa
ttatgtgtacttgctaccat

SEQ ID NO: 258 - DNA, 20 bp, Cannabis_sativa
ttgtgggatataggtgccta

SEQ ID NO: 259 - DNA, 20 bp, Cannabis_sativa
gtagctagtttgtgggatat

SEQ ID NO: 260 - DNA, 20 bp, Cannabis_sativa
ggctatggtagctagtttgt

SEQ ID NO: 261 - DNA, 20 bp, Cannabis_sativa
tggctatggtagctagtttg

SEQ ID NO: 262 - DNA, 20 bp, Cannabis_sativa
aaaaaacaagaaattggcta

SEQ ID NO: 263 - DNA, 20 bp, Cannabis_sativa
ggaaacaaaaaacaagaaat

SEQ ID NO: 264 - DNA, 20 bp, Cannabis_sativa
catcaataaaaattggatat

SEQ ID NO: 265 - DNA, 20 bp, Cannabis_sativa
agtttggcatcaataaaaat

SEQ ID NO: 266 - DNA, 20 bp, Cannabis_sativa
acattgtacattgaatagtt

SEQ ID NO: 267 - DNA, 20 bp, Cannabis_sativa
atgtacatttattttcaata

SEQ ID NO: 268 - DNA, 20 bp, Cannabis_sativa
tgtacatttattttcaataa

SEQ ID NO: 269 - DNA, 20 bp, Cannabis_sativa
ataagggcttcacctaacaa

SEQ ID NO: 270 - DNA, 20 bp, Cannabis_sativa
taaaattaggcacctttgtt

SEQ ID NO: 271 - DNA, 20 bp, Cannabis_sativa
aaaataaatcaactaaaatt

SEQ ID NO: 272 - DNA, 20 bp, Cannabis_sativa
aatattatatattggagttg

SEQ ID NO: 273 - DNA, 20 bp, Cannabis_sativa
taatattatatattggagtt

SEQ ID NO: 274 - DNA, 20 bp, Cannabis_sativa
ataatattatatattggagt

SEQ ID NO: 275 - DNA, 20 bp, Cannabis_sativa
ctatttataatattatatat

SEQ ID NO: 276 - DNA, 20 bp, Cannabis_sativa
caatatataatattataaat

SEQ ID NO: 277 - DNA, 20 bp, Cannabis_sativa
taatgattttttgaattatt

SEQ ID NO: 278 - DNA, 20 bp, Cannabis_sativa
taataattcaaaaaatcatt

SEQ ID NO: 279 - DNA, 20 bp, Cannabis_sativa
aatgtatgtggtgctttgtt

SEQ ID NO: 280 - DNA, 20 bp, Cannabis_sativa
ttcgtccgagcaaatgtatg

SEQ ID NO: 281 - DNA, 20 bp, Cannabis_sativa
aagcaccacatacatttgct

SEQ ID NO: 282 - DNA, 20 bp, Cannabis_sativa
tcggacgaagtcctcttagc

SEQ ID NO: 283 - DNA, 20 bp, Cannabis_sativa
ttgctcactgcccggctaag

SEQ ID NO: 284 - DNA, 20 bp, Cannabis_sativa
cggacgaagtcctcttagcc

SEQ ID NO: 285 - DNA, 20 bp, Cannabis_sativa
tgcgcggattgctcactgcc

SEQ ID NO: 286 - DNA, 20 bp, Cannabis_sativa
ctaagtgttggcgtgctgcg

SEQ ID NO: 287 - DNA, 20 bp, Cannabis_sativa
aagctgaatttgctaagtgt

SEQ ID NO: 288 - DNA, 20 bp, Cannabis_sativa
caaagttatagggatttctt

SEQ ID NO: 289 - DNA, 20 bp, Cannabis_sativa
ggcctgatcccaaagttata

SEQ ID NO: 290 - DNA, 20 bp, Cannabis_sativa
tggcctgatcccaaagttat

SEQ ID NO: 291 - DNA, 20 bp, Cannabis_sativa
caaagaaatccctataactt

SEQ ID NO: 292 - DNA, 20 bp, Cannabis_sativa
aaagaaatccctataacttt

SEQ ID NO: 293 - DNA, 20 bp, Cannabis_sativa
atccctataactttgggatc

SEQ ID NO: 294 - DNA, 20 bp, Cannabis_sativa
cgaatcttgccatatctcta

SEQ ID NO: 295 - DNA, 20 bp, Cannabis_sativa
gggatcaggccatagagata

SEQ ID NO: 296 - DNA, 20 bp, Cannabis_sativa
aaaagtttatcactttattt

SEQ ID NO: 297 - DNA, 20 bp, Cannabis_sativa
taaaagtttatcactttatt

SEQ ID NO: 298 - DNA, 20 bp, Cannabis_sativa
ttttaaacaaagaagcaaca

SEQ ID NO: 299 - DNA, 20 bp, Cannabis_sativa
cgtacattgtcgacctcaga

SEQ ID NO: 300 - DNA, 20 bp, Cannabis_sativa
cctttttccttatccttctg

SEQ ID NO: 301 - DNA, 20 bp, Cannabis_sativa
ttgtcgacctcagaaggata

SEQ ID NO: 302 - DNA, 20 bp, Cannabis_sativa
cctcagaaggataaggaaaa

SEQ ID NO: 303 - DNA, 20 bp, Cannabis_sativa
cagtgaccgtgatagcgagc

SEQ ID NO: 304 - DNA, 20 bp, Cannabis_sativa
aaggctccagctcgctatca

SEQ ID NO: 305 - DNA, 20 bp, Cannabis_sativa
cagctcgctatcacggtcac

SEQ ID NO: 306 - DNA, 20 bp, Cannabis_sativa
aggatgtccgtacaggagct

SEQ ID NO: 307 - DNA, 20 bp, Cannabis_sativa
actggaaccaagctcctgta

SEQ ID NO: 308 - DNA, 20 bp, Cannabis_sativa
gtgtctgaggatgtccgtac

SEQ ID NO: 309 - DNA, 20 bp, Cannabis_sativa
atctttaattattgtgtctg

SEQ ID NO: 310 - DNA, 20 bp, Cannabis_sativa
tgtgatcctcccgcatttat

SEQ ID NO: 311 - DNA, 20 bp, Cannabis_sativa
tcattttgatccaataaatg

SEQ ID NO: 312 - DNA, 20 bp, Cannabis_sativa
cattttgatccaataaatgc

SEQ ID NO: 313 - DNA, 20 bp, Cannabis_sativa
tttgatccaataaatgcggg

SEQ ID NO: 314 - DNA, 20 bp, Cannabis_sativa
catatcacgttgtgtaaatg

SEQ ID NO: 315 - DNA, 20 bp, Cannabis_sativa
cgatcatcccctaaaatcat

SEQ ID NO: 316 - DNA, 20 bp, Cannabis_sativa
acgatcatcccctaaaatca

SEQ ID NO: 317 - DNA, 20 bp, Cannabis_sativa
taatcaaatcccatgatttt

SEQ ID NO: 318 - DNA, 20 bp, Cannabis_sativa
aatcaaatcccatgatttta

SEQ ID NO: 319 - DNA, 20 bp, Cannabis_sativa
atcaaatcccatgattttag

SEQ ID NO: 320 - DNA, 20 bp, Cannabis_sativa
actatttataatcacatagt

SEQ ID NO: 321 - DNA, 20 bp, Cannabis_sativa
tactatgtgattataaatag

SEQ ID NO: 322 - DNA, 20 bp, Cannabis_sativa
ggcaagtaagatcaaaaaag

SEQ ID NO: 323 - DNA, 20 bp, Cannabis_sativa
acgaaaaaagcatacaaaaa

SEQ ID NO: 324 - DNA, 20 bp, Cannabis_sativa
aacacacaggattttttacg

SEQ ID NO: 325 - DNA, 20 bp, Cannabis_sativa
caatgaaaatatgaacacac

SEQ ID NO: 326 - DNA, 20 bp, Cannabis_sativa
tatgtgagattgtcactgtt

SEQ ID NO: 327 - DNA, 20 bp, Cannabis_sativa
taacacaatctaatttattt

SEQ ID NO: 328 - DNA, 20 bp, Cannabis_sativa
caatctaatttattttggat

SEQ ID NO: 329 - DNA, 20 bp, Cannabis_sativa
tggcgacataaaacaatatt

SEQ ID NO: 330 - DNA, 20 bp, Cannabis_sativa
ttggcgacataaaacaatat

SEQ ID NO: 331 - DNA, 20 bp, Cannabis_sativa
tatttattttaatttgttgt

SEQ ID NO: 332 - DNA, 20 bp, Cannabis_sativa
acaaaaaaataactaaccca

SEQ ID NO: 333 - DNA, 20 bp, Cannabis_sativa
caaaaaaataactaacccaa

SEQ ID NO: 334 - DNA, 20 bp, Cannabis_sativa
tataatttttttcttccctt

SEQ ID NO: 335 - DNA, 20 bp, Cannabis_sativa
atataatttttttcttccct

SEQ ID NO: 336 - DNA, 20 bp, Cannabis_sativa
tttattgttgaaaaatttat

SEQ ID NO: 337 - DNA, 20 bp, Cannabis_sativa
taattataagacgtataaca

SEQ ID NO: 338 - DNA, 20 bp, Cannabis_sativa
attcaattttaatgagatcg

SEQ ID NO: 339 - DNA, 20 bp, Cannabis_sativa
ttcaattttaatgagatcga

SEQ ID NO: 340 - DNA, 20 bp, Cannabis_sativa
tatttgttaatatatattga

SEQ ID NO: 341 - DNA, 20 bp, Cannabis_sativa
taaaattttaaagttatgtg

SEQ ID NO: 342 - DNA, 20 bp, Cannabis_sativa
ttttttaagattaattacta

SEQ ID NO: 343 - DNA, 20 bp, Cannabis_sativa
aaaagaaattcaaattatta

SEQ ID NO: 344 - DNA, 20 bp, Cannabis_sativa
agaaattcaaattattaagg

SEQ ID NO: 345 - DNA, 20 bp, Cannabis_sativa
caaattattaaggtggcgtt

SEQ ID NO: 346 - DNA, 20 bp, Cannabis_sativa
tttaatcaatgttttagatt

SEQ ID NO: 347 - DNA, 20 bp, Cannabis_sativa
ttttagattaggaccagacc

SEQ ID NO: 348 - DNA, 20 bp, Cannabis_sativa
agggaatttgggtccgggtc

SEQ ID NO: 349 - DNA, 20 bp, Cannabis_sativa
tagggagggaatttgggtcc

SEQ ID NO: 350 - DNA, 20 bp, Cannabis_sativa
gtagggagggaatttgggtc

SEQ ID NO: 351 - DNA, 20 bp, Cannabis_sativa
gggccgtagggagggaattt

SEQ ID NO: 352 - DNA, 20 bp, Cannabis_sativa
agggccgtagggagggaatt

SEQ ID NO: 353 - DNA, 20 bp, Cannabis_sativa
ggacccaaattccctcccta

SEQ ID NO: 354 - DNA, 20 bp, Cannabis_sativa
tagcgccagggccgtaggga

SEQ ID NO: 355 - DNA, 20 bp, Cannabis_sativa
ttagcgccagggccgtaggg

SEQ ID NO: 356 - DNA, 20 bp, Cannabis_sativa
ggtttagcgccagggccgta

SEQ ID NO: 357 - DNA, 20 bp, Cannabis_sativa
gggtttagcgccagggccgt

SEQ ID NO: 358 - DNA, 20 bp, Cannabis_sativa
aaattccctccctacggccc

SEQ ID NO: 359 - DNA, 20 bp, Cannabis_sativa
aaaagtcgggtttagcgcca

SEQ ID NO: 360 - DNA, 20 bp, Cannabis_sativa
caaaagtcgggtttagcgcc

SEQ ID NO: 361 - DNA, 20 bp, Cannabis_sativa
tcgggttcaagtcaaaagtc

SEQ ID NO: 362 - DNA, 20 bp, Cannabis_sativa
ttcgggttcaagtcaaaagt

SEQ ID NO: 363 - DNA, 20 bp, Cannabis_sativa
tttaggtgcaagtcggattc

SEQ ID NO: 364 - DNA, 20 bp, Cannabis_sativa
atttaggtgcaagtcggatt

SEQ ID NO: 365 - DNA, 20 bp, Cannabis_sativa
aaaataatttaggtgcaagt

SEQ ID NO: 366 - DNA, 20 bp, Cannabis_sativa
ttaagctttcaaaataattt

SEQ ID NO: 367 - DNA, 20 bp, Cannabis_sativa
aagataattttaccacttac

SEQ ID NO: 368 - DNA, 20 bp, Cannabis_sativa
taatataatcatcctgtaag

SEQ ID NO: 369 - DNA, 20 bp, Cannabis_sativa
ttgattgtattgattattat

SEQ ID NO: 370 - DNA, 20 bp, Cannabis_sativa
tagctacaattattaatgag

SEQ ID NO: 371 - DNA, 20 bp, Cannabis_sativa
taaaattgaagtgtgttttt

SEQ ID NO: 372 - DNA, 20 bp, Cannabis_sativa
atatttcaaacttatagctt

SEQ ID NO: 373 - DNA, 20 bp, Cannabis_sativa
accaattagaaatgggcacg

SEQ ID NO: 374 - DNA, 20 bp, Cannabis_sativa
aacttagaccaattagaaat

SEQ ID NO: 375 - DNA, 20 bp, Cannabis_sativa
taacttagaccaattagaaa

SEQ ID NO: 376 - DNA, 20 bp, Cannabis_sativa
accacgtgcccatttctaat

SEQ ID NO: 377 - DNA, 20 bp, Cannabis_sativa
tccacgtgtcattttcttct

SEQ ID NO: 378 - DNA, 20 bp, Cannabis_sativa
accaagaagaaaatgacacg

SEQ ID NO: 379 - DNA, 20 bp, Cannabis_sativa
gataatgacttaatatttaa

SEQ ID NO: 380 - DNA, 20 bp, Cannabis_sativa
atgacttaatatttaatggt

SEQ ID NO: 381 - DNA, 20 bp, Cannabis_sativa
taatattttgggaactctgt

SEQ ID NO: 382 - DNA, 20 bp, Cannabis_sativa
taatacctaagtaatatttt

SEQ ID NO: 383 - DNA, 20 bp, Cannabis_sativa
ataatacctaagtaatattt

SEQ ID NO: 384 - DNA, 20 bp, Cannabis_sativa
gagttcccaaaatattactt

SEQ ID NO: 385 - DNA, 20 bp, Cannabis_sativa
aaatacctacgttttatttt

SEQ ID NO: 386 - DNA, 20 bp, Cannabis_sativa
tagcgccaaaaataaaacgt

SEQ ID NO: 387 - DNA, 20 bp, Cannabis_sativa
cgtaggtatttatttgcaac

SEQ ID NO: 388 - DNA, 20 bp, Cannabis_sativa
cacaaaacatctaaaaaaaa

SEQ ID NO: 389 - DNA, 20 bp, Cannabis_sativa
cattttttttagatgttttg

SEQ ID NO: 390 - DNA, 20 bp, Cannabis_sativa
acgccaactcatatacaaaa

SEQ ID NO: 391 - DNA, 20 bp, Cannabis_sativa
ggaccattttgtatatgagt

SEQ ID NO: 392 - DNA, 20 bp, Cannabis_sativa
tgttgaatctctagctcttt

SEQ ID NO: 393 - DNA, 20 bp, Cannabis_sativa
gctttattgtctaaatttct

SEQ ID NO: 394 - DNA, 20 bp, Cannabis_sativa
ctttattgtctaaatttctt

SEQ ID NO: 395 - DNA, 20 bp, Cannabis_sativa
tttattgtctaaatttcttg

SEQ ID NO: 396 - DNA, 20 bp, Cannabis_sativa
ctaaagtaagcgagcaacat

SEQ ID NO: 397 - DNA, 20 bp, Cannabis_sativa
tctaaagtaagcgagcaaca

SEQ ID NO: 398 - DNA, 20 bp, Cannabis_sativa
gtttgacaaaacatgctatt

SEQ ID NO: 399 - DNA, 20 bp, Cannabis_sativa
caatgagctatcctagttca

SEQ ID NO: 400 - DNA, 20 bp, Cannabis_sativa
cacagaaatctccttgaact

SEQ ID NO: 401 - DNA, 20 bp, Cannabis_sativa
ggagatttctgtgctatttg

SEQ ID NO: 402 - DNA, 20 bp, Cannabis_sativa
cgcttgcaaatatttatcta

SEQ ID NO: 403 - DNA, 20 bp, Cannabis_sativa
atagcaaattttttttccat

SEQ ID NO: 404 - DNA, 20 bp, Cannabis_sativa
tgatttttttaaatttccta

SEQ ID NO: 405 - DNA, 20 bp, Cannabis_sativa
ttattaaaaataaatgtaca

SEQ ID NO: 406 - DNA, 20 bp, Cannabis_sativa
atgtacatttatttttaata

SEQ ID NO: 407 - DNA, 20 bp, Cannabis_sativa
tgtacatttatttttaataa

SEQ ID NO: 408 - DNA, 20 bp, Cannabis_sativa
ataagggctgcacctaacaa

SEQ ID NO: 409 - DNA, 20 bp, Cannabis_sativa
caaaattaggcacctttgtt

SEQ ID NO: 410 - DNA, 20 bp, Cannabis_sativa
ctaacaaaggtgcctaattt

SEQ ID NO: 411 - DNA, 20 bp, Cannabis_sativa
atttcttttttaccaaaatt

SEQ ID NO: 412 - DNA, 20 bp, Cannabis_sativa
ttttggtaaaaaagaaatta

SEQ ID NO: 413 - DNA, 20 bp, Cannabis_sativa
agctttatagattggagtgg

SEQ ID NO: 414 - DNA, 20 bp, Cannabis_sativa
tagctttatagattggagtg

SEQ ID NO: 415 - DNA, 20 bp, Cannabis_sativa
atagctttatagattggagt

SEQ ID NO: 416 - DNA, 20 bp, Cannabis_sativa
tatagctttatagattggag

SEQ ID NO: 417 - DNA, 20 bp, Cannabis_sativa
ctatttatagctttatagat

SEQ ID NO: 418 - DNA, 20 bp, Cannabis_sativa
caatctataaagctataaat

SEQ ID NO: 419 - DNA, 20 bp, Cannabis_sativa
atgttggaaattactatgaa

SEQ ID NO: 420 - DNA, 20 bp, Cannabis_sativa
ttacactaaagtgactctat

SEQ ID NO: 421 - DNA, 20 bp, Cannabis_sativa
gtaacgtggaacgcggcgta

SEQ ID NO: 422 - DNA, 20 bp, Cannabis_sativa
atctctggtaacgtggaacg

SEQ ID NO: 423 - DNA, 20 bp, Cannabis_sativa
tatttttatctctggtaacg

SEQ ID NO: 424 - DNA, 20 bp, Cannabis_sativa
ctttttcatatttttatctc

SEQ ID NO: 425 - DNA, 20 bp, Cannabis_sativa
agttgtgatttatgaattta

SEQ ID NO: 426 - DNA, 20 bp, Cannabis_sativa
aattttagtatagataatta

SEQ ID NO: 427 - DNA, 20 bp, Cannabis_sativa
taattttagtatagataatt

SEQ ID NO: 428 - DNA, 20 bp, Cannabis_sativa
ttatctatactaaaattaag

SEQ ID NO: 429 - DNA, 20 bp, Cannabis_sativa
acttgaagtcggagacaaat

SEQ ID NO: 430 - DNA, 20 bp, Cannabis_sativa
acattactcgcacttgaagt

SEQ ID NO: 431 - DNA, 20 bp, Cannabis_sativa
gagtaatgtaactcctgaca

SEQ ID NO: 432 - DNA, 20 bp, Cannabis_sativa
taaaccaatacttccatgtc

SEQ ID NO: 433 - DNA, 20 bp, Cannabis_sativa
aactcctgacatggaagtat

SEQ ID NO: 434 - DNA, 20 bp, Cannabis_sativa
ttggtttatgttatcttaat

SEQ ID NO: 435 - DNA, 20 bp, Cannabis_sativa
taggtaaagctaatgatgtg

SEQ ID NO: 436 - DNA, 20 bp, Cannabis_sativa
aaaggcattatgagtatggg

SEQ ID NO: 437 - DNA, 20 bp, Cannabis_sativa
gataaaggcattatgagtat

SEQ ID NO: 438 - DNA, 20 bp, Cannabis_sativa
tgataaaggcattatgagta

SEQ ID NO: 439 - DNA, 20 bp, Cannabis_sativa
ttgaatttttaaggtgataa

SEQ ID NO: 440 - DNA, 20 bp, Cannabis_sativa
ggaaagtcattgaattttta

SEQ ID NO: 441 - DNA, 20 bp, Cannabis_sativa
acaagctaataatcaaacat

SEQ ID NO: 442 - DNA, 20 bp, Cannabis_sativa
cacaagctaataatcaaaca

SEQ ID NO: 443 - DNA, 20 bp, Cannabis_sativa
tttgattattagcttgtgta

SEQ ID NO: 444 - DNA, 20 bp, Cannabis_sativa
attagcttgtgtaaggaagt

SEQ ID NO: 445 - DNA, 20 bp, Cannabis_sativa
tttgatgaaaattatatcta

SEQ ID NO: 446 - DNA, 20 bp, Cannabis_sativa
agctattttattaaccatgt

SEQ ID NO: 447 - DNA, 20 bp, Cannabis_sativa
gctattttattaaccatgtt

SEQ ID NO: 448 - DNA, 20 bp, Cannabis_sativa
aagggcctacagtcccaaca

SEQ ID NO: 449 - DNA, 20 bp, Cannabis_sativa
attaaccatgttgggactgt

SEQ ID NO: 450 - DNA, 20 bp, Cannabis_sativa
caatagaagaagaagaagaa

SEQ ID NO: 451 - DNA, 20 bp, Cannabis_sativa
gcaatagaagaagaagaaga

SEQ ID NO: 452 - DNA, 20 bp, Cannabis_sativa
gcaagtcttttctctaatgc

SEQ ID NO: 453 - DNA, 20 bp, Cannabis_sativa
ttctctaatgcaggttcttt

SEQ ID NO: 454 - DNA, 20 bp, Cannabis_sativa
tctctaatgcaggttcttta

SEQ ID NO: 455 - DNA, 20 bp, Cannabis_sativa
gttctttagggcccacttct

SEQ ID NO: 456 - DNA, 20 bp, Cannabis_sativa
taaaggggaggcctagaagt

SEQ ID NO: 457 - DNA, 20 bp, Cannabis_sativa
ataaaggggaggcctagaag

SEQ ID NO: 458 - DNA, 20 bp, Cannabis_sativa
tatactaaaaaataaagggg

SEQ ID NO: 459 - DNA, 20 bp, Cannabis_sativa
ctatatactaaaaaataaag

SEQ ID NO: 460 - DNA, 20 bp, Cannabis_sativa
actatatactaaaaaataaa

SEQ ID NO: 461 - DNA, 20 bp, Cannabis_sativa
tactatatactaaaaaataa

SEQ ID NO: 462 - DNA, 20 bp, Cannabis_sativa
tttattttttagtatatagt

SEQ ID NO: 463 - DNA, 20 bp, Cannabis_sativa
ataaagcacaaaactaaata

SEQ ID NO: 464 - DNA, 20 bp, Cannabis_sativa
aacaacttggcttagcataa

SEQ ID NO: 465 - DNA, 20 bp, Cannabis_sativa
acaacatcctaaaaacaact

SEQ ID NO: 466 - DNA, 20 bp, Cannabis_sativa
tgctaagccaagttgttttt

SEQ ID NO: 467 - DNA, 20 bp, Cannabis_sativa
gaatatattgtttgcatact

SEQ ID NO: 468 - DNA, 20 bp, Cannabis_sativa
aggaaacacaaataatacga

SEQ ID NO: 469 - DNA, 20 bp, Cannabis_sativa
acgaaataaaaaattcaatc

SEQ ID NO: 470 - DNA, 20 bp, Cannabis_sativa
ttatttcgttcaaatgagtt

SEQ ID NO: 471 - DNA, 20 bp, Cannabis_sativa
tatttcgttcaaatgagttt

SEQ ID NO: 472 - DNA, 20 bp, Cannabis_sativa
tttattcgaaaatcatgatt

SEQ ID NO: 473 - DNA, 20 bp, Cannabis_sativa
aataaaaactttcaaaaata

SEQ ID NO: 474 - DNA, 20 bp, Cannabis_sativa
aaaattattatgaaatgaaa

SEQ ID NO: 475 - DNA, 20 bp, Cannabis_sativa
aaggtaattatcttagaact

SEQ ID NO: 476 - DNA, 20 bp, Cannabis_sativa
tattcctaagcgtgtcatct

SEQ ID NO: 477 - DNA, 20 bp, Cannabis_sativa
aaatcctagatgacacgctt

SEQ ID NO: 478 - DNA, 20 bp, Cannabis_sativa
cacgcttaggaatataatat

SEQ ID NO: 479 - DNA, 20 bp, Cannabis_sativa
agaattaacataacaaaagt

SEQ ID NO: 480 - DNA, 20 bp, Cannabis_sativa
aggttcaattaggcttaaat

SEQ ID NO: 481 - DNA, 20 bp, Cannabis_sativa
gaggttcaattaggcttaaa

SEQ ID NO: 482 - DNA, 20 bp, Cannabis_sativa
atgttaaaagaggttcaatt

SEQ ID NO: 483 - DNA, 20 bp, Cannabis_sativa
agagaatggaatgttaaaag

SEQ ID NO: 484 - DNA, 20 bp, Cannabis_sativa
tatggcattattcaagagaa

SEQ ID NO: 485 - DNA, 20 bp, Cannabis_sativa
atcaatcaataaaaaacgta

SEQ ID NO: 486 - DNA, 20 bp, Cannabis_sativa
tacgttttttattgattgat

SEQ ID NO: 487 - DNA, 20 bp, Cannabis_sativa
acgttttttattgattgatt

SEQ ID NO: 488 - DNA, 20 bp, Cannabis_sativa
ttttattgattgattgggta

SEQ ID NO: 489 - DNA, 20 bp, Cannabis_sativa
tgattgggtatggttcgcga

SEQ ID NO: 490 - DNA, 20 bp, Cannabis_sativa
gattgggtatggttcgcgat

SEQ ID NO: 491 - DNA, 20 bp, Cannabis_sativa
attgggtatggttcgcgatg

SEQ ID NO: 492 - DNA, 20 bp, Cannabis_sativa
gatggggtataatgaaaagt

SEQ ID NO: 493 - DNA, 20 bp, Cannabis_sativa
ttaatttattttttttaaca

SEQ ID NO: 494 - DNA, 20 bp, Cannabis_sativa
tcttagaaatgaaagcagtt

SEQ ID NO: 495 - DNA, 20 bp, Cannabis_sativa
cttagaaatgaaagcagttt

SEQ ID NO: 496 - DNA, 20 bp, Cannabis_sativa
ttagaaatgaaagcagtttg

SEQ ID NO: 497 - DNA, 20 bp, Cannabis_sativa
agcagtttggggattgttat

SEQ ID NO: 498 - DNA, 20 bp, Cannabis_sativa
gcagtttggggattgttatt

SEQ ID NO: 499 - DNA, 20 bp, Cannabis_sativa
acaatagcttaggtatgggt

SEQ ID NO: 500 - DNA, 20 bp, Cannabis_sativa
tgtaacaatagcttaggtat

SEQ ID NO: 501 - DNA, 20 bp, Cannabis_sativa
ttgtaacaatagcttaggta

SEQ ID NO: 502 - DNA, 20 bp, Cannabis_sativa
tagacttgtaacaatagctt

SEQ ID NO: 503 - DNA, 20 bp, Cannabis_sativa
tgaacaagagttgtctacat

SEQ ID NO: 504 - DNA, 20 bp, Cannabis_sativa
ttgtctacattggtagagaa

SEQ ID NO: 505 - DNA, 20 bp, Cannabis_sativa
aaatattaaatttacttctt

SEQ ID NO: 506 - DNA, 20 bp, Cannabis_sativa
atatgaaaaaatagaagact

SEQ ID NO: 507 - DNA, 20 bp, Cannabis_sativa
aataatttttacagcatttt

SEQ ID NO: 508 - DNA, 20 bp, Cannabis_sativa
gtaaaaattattgtttctaa

SEQ ID NO: 509 - DNA, 20 bp, Cannabis_sativa
tctaagaatgcacacttatt

SEQ ID NO: 510 - DNA, 20 bp, Cannabis_sativa
gaatgcacacttatttggag

SEQ ID NO: 511 - DNA, 20 bp, Cannabis_sativa
actatttttgtctattcgat

SEQ ID NO: 512 - DNA, 20 bp, Cannabis_sativa
ttggcaaaaaagtcgagctt

SEQ ID NO: 513 - DNA, 20 bp, Cannabis_sativa
gtcttaattgcgattttgag

SEQ ID NO: 514 - DNA, 20 bp, Cannabis_sativa
tcttaattgcgattttgaga

SEQ ID NO: 515 - DNA, 20 bp, Cannabis_sativa
cttaattgcgattttgagag

SEQ ID NO: 516 - DNA, 20 bp, Cannabis_sativa
agacttaattttttgacgac

SEQ ID NO: 517 - DNA, 20 bp, Cannabis_sativa
aattttttgacgactggaac

SEQ ID NO: 518 - DNA, 20 bp, Cannabis_sativa
agaatcgttgagaagtgctt

SEQ ID NO: 519 - DNA, 20 bp, Cannabis_sativa
gaagtgctttggatgaagac

SEQ ID NO: 520 - DNA, 20 bp, Cannabis_sativa
aaatgttaatgtcatgttta

SEQ ID NO: 521 - DNA, 20 bp, Cannabis_sativa
ttcattaatagcatgtaaaa

SEQ ID NO: 522 - DNA, 20 bp, Cannabis_sativa
ttagcatattcaattctcgc

SEQ ID NO: 523 - DNA, 20 bp, Cannabis_sativa
gtagaacaaaagtatcaaat

SEQ ID NO: 524 - DNA, 20 bp, Cannabis_sativa
caaatcggtctatataattt

SEQ ID NO: 525 - DNA, 20 bp, Cannabis_sativa
ttcaaaaccgtttaatattc

SEQ ID NO: 526 - DNA, 20 bp, Cannabis_sativa
attcaaaaccgtttaatatt

SEQ ID NO: 527 - DNA, 20 bp, Cannabis_sativa
ttcttaacccgaatattaaa

SEQ ID NO: 528 - DNA, 20 bp, Cannabis_sativa
tcattgattgaataataaag

SEQ ID NO: 529 - DNA, 20 bp, Cannabis_sativa
aataataaagtggatagtag

SEQ ID NO: 530 - DNA, 20 bp, Cannabis_sativa
ataataaagtggatagtaga

SEQ ID NO: 531 - DNA, 20 bp, Cannabis_sativa
aatatataaataaaaaatta

SEQ ID NO: 532 - DNA, 20 bp, Cannabis_sativa
caaatagcaaaattaaatga

SEQ ID NO: 533 - DNA, 20 bp, Cannabis_sativa
tacaaaataaaaaataagat

SEQ ID NO: 534 - DNA, 20 bp, Cannabis_sativa
taaaaaataagataggatat

SEQ ID NO: 535 - DNA, 20 bp, Cannabis_sativa
tacttgataagtcttctttg

SEQ ID NO: 536 - DNA, 20 bp, Cannabis_sativa
ttctttgtggaaacgataat

SEQ ID NO: 537 - DNA, 20 bp, Cannabis_sativa
aaacgataatcggtattatt

SEQ ID NO: 538 - DNA, 20 bp, Cannabis_sativa
aaatatattaataaataaag

SEQ ID NO: 539 - DNA, 20 bp, Cannabis_sativa
atattaataaataaagtgga

SEQ ID NO: 540 - DNA, 20 bp, Cannabis_sativa
aataaagtggaaggtgccat

SEQ ID NO: 541 - DNA, 20 bp, Cannabis_sativa
tttgtggatataggtaccta

SEQ ID NO: 542 - DNA, 20 bp, Cannabis_sativa
atatgctagtttgtggatat

SEQ ID NO: 543 - DNA, 20 bp, Cannabis_sativa
tctttcaatatgctagtttg

SEQ ID NO: 544 - DNA, 20 bp, Cannabis_sativa
actagcatattgaaagaaaa

SEQ ID NO: 545 - DNA, 20 bp, Cannabis_sativa
attgaaagaaaatggatcca

SEQ ID NO: 546 - DNA, 20 bp, Cannabis_sativa
gacttgcaaatatttatcca

SEQ ID NO: 547 - DNA, 20 bp, Cannabis_sativa
ataataataaaaaaagaaat

SEQ ID NO: 548 - DNA, 20 bp, Cannabis_sativa
ttttaatagaatatttcaaa

SEQ ID NO: 549 - DNA, 20 bp, Cannabis_sativa
tttaatagaatatttcaaaa

SEQ ID NO: 550 - DNA, 20 bp, Cannabis_sativa
tctaacatttatttttaata

SEQ ID NO: 551 - DNA, 20 bp, Cannabis_sativa
ataaggactgcacctaacaa

SEQ ID NO: 552 - DNA, 20 bp, Cannabis_sativa
aaaaattaggcacctttgtt

SEQ ID NO: 553 - DNA, 20 bp, Cannabis_sativa
aaaaaaagttcacaaaaatt

SEQ ID NO: 554 - DNA, 20 bp, Cannabis_sativa
actcattaaatagtcacatg

SEQ ID NO: 555 - DNA, 20 bp, Cannabis_sativa
agcattatatattggagcgg

SEQ ID NO: 556 - DNA, 20 bp, Cannabis_sativa
tagcattatatattggagcg

SEQ ID NO: 557 - DNA, 20 bp, Cannabis_sativa
atagcattatatattggagc

SEQ ID NO: 558 - DNA, 20 bp, Cannabis_sativa
tatagcattatatattggag

SEQ ID NO: 559 - DNA, 20 bp, Cannabis_sativa
ctatttatagcattatatat

SEQ ID NO: 560 - DNA, 20 bp, Cannabis_sativa
atagtaattcaaaaatcatt

SEQ ID NO: 561 - DNA, 20 bp, Cannabis_sativa
aaatttataggaaaccccta

SEQ ID NO: 562 - DNA, 20 bp, Cannabis_sativa
aaaaattttaaaaaatttat

SEQ ID NO: 563 - DNA, 20 bp, Cannabis_sativa
gctctaagtgtttgtatatt

SEQ ID NO: 564 - DNA, 20 bp, Cannabis_sativa
taaaaatgatactaaaatac

SEQ ID NO: 565 - DNA, 20 bp, Cannabis_sativa
tacatttaacttttataata

SEQ ID NO: 566 - DNA, 20 bp, Cannabis_sativa
acatttaacttttataatat

SEQ ID NO: 567 - DNA, 20 bp, Cannabis_sativa
aattacaaaaatggtctatt

SEQ ID NO: 568 - DNA, 20 bp, Cannabis_sativa
aaattacaaaaatggtctat

SEQ ID NO: 569 - DNA, 20 bp, Cannabis_sativa
ccaaaaaaaaattacaaaaa

SEQ ID NO: 570 - DNA, 20 bp, Cannabis_sativa
ccatttttgtaatttttttt

SEQ ID NO: 571 - DNA, 20 bp, Cannabis_sativa
ttagaataatattaatacgt

SEQ ID NO: 572 - DNA, 20 bp, Cannabis_sativa
aaaattactctaagtattta

SEQ ID NO: 573 - DNA, 20 bp, Cannabis_sativa
agcacataattttttgtata

SEQ ID NO: 574 - DNA, 20 bp, Cannabis_sativa
tttatcgaaattgactttat

SEQ ID NO: 575 - DNA, 20 bp, Cannabis_sativa
ttctcttaaacttggttgtt

SEQ ID NO: 576 - DNA, 20 bp, Cannabis_sativa
attttagtttctcttaaact

SEQ ID NO: 577 - DNA, 20 bp, Cannabis_sativa
gccgaaatttcggtagaatt

SEQ ID NO: 578 - DNA, 20 bp, Cannabis_sativa
tcctaattctaccgaaattt

SEQ ID NO: 579 - DNA, 20 bp, Cannabis_sativa
ccgttatgctgccgaaattt

SEQ ID NO: 580 - DNA, 20 bp, Cannabis_sativa
ccgaaatttcggcagcataa

SEQ ID NO: 581 - DNA, 20 bp, Cannabis_sativa
attaataggtttgaattttt

SEQ ID NO: 582 - DNA, 20 bp, Cannabis_sativa
tgtgttttgttgttattaat

SEQ ID NO: 583 - DNA, 20 bp, Cannabis_sativa
atgtactgtagtcggatggg

SEQ ID NO: 584 - DNA, 20 bp, Cannabis_sativa
tacatgtactgtagtcggat

SEQ ID NO: 585 - DNA, 20 bp, Cannabis_sativa
atacatgtactgtagtcgga

SEQ ID NO: 586 - DNA, 20 bp, Cannabis_sativa
gtgaatacatgtactgtagt

SEQ ID NO: 587 - DNA, 20 bp, Cannabis_sativa
atattcatctgtagtgaagt

SEQ ID NO: 588 - DNA, 20 bp, Cannabis_sativa
ttgtactatgtcggatcgat

SEQ ID NO: 589 - DNA, 20 bp, Cannabis_sativa
attgtactatgtcggatcga

SEQ ID NO: 590 - DNA, 20 bp, Cannabis_sativa
tacaatatattgtactatgt

SEQ ID NO: 591 - DNA, 20 bp, Cannabis_sativa
atattcatttgtagtgaagt

SEQ ID NO: 592 - DNA, 20 bp, Cannabis_sativa
ttttgtgttgatcggtttct

SEQ ID NO: 593 - DNA, 20 bp, Cannabis_sativa
ccaacttattttgtgttgat

SEQ ID NO: 594 - DNA, 20 bp, Cannabis_sativa
ccgatcaacacaaaataagt

SEQ ID NO: 595 - DNA, 20 bp, Cannabis_sativa
ggttgtgaggtcaatttgca

SEQ ID NO: 596 - DNA, 20 bp, Cannabis_sativa
tctttatgttgaaggttgtg

SEQ ID NO: 597 - DNA, 20 bp, Cannabis_sativa
gtagtatttctttatgttga

SEQ ID NO: 598 - DNA, 20 bp, Cannabis_sativa
aagtagctaaataaaaaaat

SEQ ID NO: 599 - DNA, 20 bp, Cannabis_sativa
atgtgttttatttctttagt

SEQ ID NO: 600 - DNA, 20 bp, Cannabis_sativa
attgtgaatgagaatgagat

SEQ ID NO: 601 - DNA, 20 bp, Cannabis_sativa
gtgaatgagaaatgtaatat

SEQ ID NO: 602 - DNA, 20 bp, Cannabis_sativa
tgtgtatatctattgtgaat

SEQ ID NO: 603 - DNA, 20 bp, Cannabis_sativa
atgtgtatatctattgtgaa

SEQ ID NO: 604 - DNA, 20 bp, Cannabis_sativa
ttattttataaattttttta

SEQ ID NO: 605 - DNA, 20 bp, Cannabis_sativa
gttattttataaattttttt

SEQ ID NO: 606 - DNA, 20 bp, Cannabis_sativa
aattagatttataccttaat

SEQ ID NO: 607 - DNA, 20 bp, Cannabis_sativa
ttgtatctcaacgcctatta

SEQ ID NO: 608 - DNA, 20 bp, Cannabis_sativa
cctccggccaccgtttttag

SEQ ID NO: 609 - DNA, 20 bp, Cannabis_sativa
aatgttttctccactaaaaa

SEQ ID NO: 610 - DNA, 20 bp, Cannabis_sativa
gttttctccactaaaaacgg

SEQ ID NO: 611 - DNA, 20 bp, Cannabis_sativa
tctccactaaaaacggtggc

SEQ ID NO: 612 - DNA, 20 bp, Cannabis_sativa
ccactaaaaacggtggccgg

SEQ ID NO: 613 - DNA, 20 bp, Cannabis_sativa
ggtagtggtgattatacctc

SEQ ID NO: 614 - DNA, 20 bp, Cannabis_sativa
taaacaaaaagtaaaggtag

SEQ ID NO: 615 - DNA, 20 bp, Cannabis_sativa
ttggggtaaacaaaaagtaa

SEQ ID NO: 616 - DNA, 20 bp, Cannabis_sativa
acatttttcctcattttttg

SEQ ID NO: 617 - DNA, 20 bp, Cannabis_sativa
tacatttttcctcatttttt

SEQ ID NO: 618 - DNA, 20 bp, Cannabis_sativa
ttacatttttcctcattttt

SEQ ID NO: 619 - DNA, 20 bp, Cannabis_sativa
tttgtttaccccaaaaaatg

SEQ ID NO: 620 - DNA, 20 bp, Cannabis_sativa
aggaaaaatgtaatcttttc

SEQ ID NO: 621 - DNA, 20 bp, Cannabis_sativa
cttttcaggtatatagtttt

SEQ ID NO: 622 - DNA, 20 bp, Cannabis_sativa
gaaataaacatgagctaaaa

SEQ ID NO: 623 - DNA, 20 bp, Cannabis_sativa
atggtgaaaaaatagtgaaa

SEQ ID NO: 624 - DNA, 20 bp, Cannabis_sativa
gtgaaaaaatagtgaaatgg

SEQ ID NO: 625 - DNA, 20 bp, Cannabis_sativa
gaaatggaggtgatttttcg

SEQ ID NO: 626 - DNA, 20 bp, Cannabis_sativa
atggaggtgatttttcgtgg

SEQ ID NO: 627 - DNA, 20 bp, Cannabis_sativa
aggtgatttttcgtggtggt

SEQ ID NO: 628 - DNA, 20 bp, Cannabis_sativa
tgatttttcgtggtggttgg

SEQ ID NO: 629 - DNA, 20 bp, Cannabis_sativa
tttcgtggtggttggtggag

SEQ ID NO: 630 - DNA, 20 bp, Cannabis_sativa
ttcgtggtggttggtggaga

SEQ ID NO: 631 - DNA, 20 bp, Cannabis_sativa
ggtggttggtggagagggtt

SEQ ID NO: 632 - DNA, 20 bp, Cannabis_sativa
gtggttggtggagagggttt

SEQ ID NO: 633 - DNA, 20 bp, Cannabis_sativa
tggttggtggagagggtttg

SEQ ID NO: 634 - DNA, 20 bp, Cannabis_sativa
gagagggtttggggtttctt

SEQ ID NO: 635 - DNA, 20 bp, Cannabis_sativa
ggtttggggtttctttggtt

SEQ ID NO: 636 - DNA, 20 bp, Cannabis_sativa
gtttggggtttctttggttt

SEQ ID NO: 637 - DNA, 20 bp, Cannabis_sativa
tttggggtttctttggtttg

SEQ ID NO: 638 - DNA, 20 bp, Cannabis_sativa
ttggggtttctttggtttgg

SEQ ID NO: 639 - DNA, 20 bp, Cannabis_sativa
ttctttggtttgggggtttc

SEQ ID NO: 640 - DNA, 20 bp, Cannabis_sativa
tctttggtttgggggtttct

SEQ ID NO: 641 - DNA, 20 bp, Cannabis_sativa
tggtttgggggtttctgggt

SEQ ID NO: 642 - DNA, 20 bp, Cannabis_sativa
ttgggggtttctgggttgga

SEQ ID NO: 643 - DNA, 20 bp, Cannabis_sativa
ctgggttggatggtcgaatg

SEQ ID NO: 644 - DNA, 20 bp, Cannabis_sativa
tggtcgaatgaggaagatga

SEQ ID NO: 645 - DNA, 20 bp, Cannabis_sativa
ggtcgaatgaggaagatgaa

SEQ ID NO: 646 - DNA, 20 bp, Cannabis_sativa
gaatgaggaagatgaagggc

SEQ ID NO: 647 - DNA, 20 bp, Cannabis_sativa
aatgaggaagatgaagggct

SEQ ID NO: 648 - DNA, 20 bp, Cannabis_sativa
ggaagatgaagggctgggct

SEQ ID NO: 649 - DNA, 20 bp, Cannabis_sativa
gaagatgaagggctgggcta

SEQ ID NO: 650 - DNA, 20 bp, Cannabis_sativa
gctagggttataaaaccttt

SEQ ID NO: 651 - DNA, 20 bp, Cannabis_sativa
agaagataaccgacgccaaa

SEQ ID NO: 652 - DNA, 20 bp, Cannabis_sativa
gttataaaaccttttggcgt

SEQ ID NO: 653 - DNA, 20 bp, Cannabis_sativa
tagtttttaatataattgta

SEQ ID NO: 654 - DNA, 20 bp, Cannabis_sativa
agtttttaatataattgtaa

SEQ ID NO: 655 - DNA, 20 bp, Cannabis_sativa
gtttttaatataattgtaag

SEQ ID NO: 656 - DNA, 20 bp, Cannabis_sativa
atttttttgtagatattttg

SEQ ID NO: 657 - DNA, 20 bp, Cannabis_sativa
acaccaactcatataacaaa

SEQ ID NO: 658 - DNA, 20 bp, Cannabis_sativa
ggaccatttgttatatgagt

SEQ ID NO: 659 - DNA, 20 bp, Cannabis_sativa
ctcttttattgtgttgttta

SEQ ID NO: 660 - DNA, 20 bp, Cannabis_sativa
ctaaagtaagcaagcaacat

SEQ ID NO: 661 - DNA, 20 bp, Cannabis_sativa
gctaaagtaagcaagcaaca

SEQ ID NO: 662 - DNA, 20 bp, Cannabis_sativa
cgatgatctatcctaattcg

SEQ ID NO: 663 - DNA, 20 bp, Cannabis_sativa
tgcagaaacctcctcgaatt

SEQ ID NO: 664 - DNA, 20 bp, Cannabis_sativa
tgatctatcctaattcgagg

SEQ ID NO: 665 - DNA, 20 bp, Cannabis_sativa
gactagcaaatatttatcta

SEQ ID NO: 666 - DNA, 20 bp, Cannabis_sativa
gaagttgactatggaaagaa

SEQ ID NO: 667 - DNA, 20 bp, Cannabis_sativa
tgccatattgaagttgacta

SEQ ID NO: 668 - DNA, 20 bp, Cannabis_sativa
ttccatagtcaacttcaata

SEQ ID NO: 669 - DNA, 20 bp, Cannabis_sativa
agttgagcatcatttgttga

SEQ ID NO: 670 - DNA, 20 bp, Cannabis_sativa
atttatatttatttttagta

SEQ ID NO: 671 - DNA, 20 bp, Cannabis_sativa
tttatatttatttttagtaa

SEQ ID NO: 672 - DNA, 20 bp, Cannabis_sativa
caaaattaggcatcattgtt

SEQ ID NO: 673 - DNA, 20 bp, Cannabis_sativa
ctaacaatgatgcctaattt

SEQ ID NO: 674 - DNA, 20 bp, Cannabis_sativa
aaaaaaaattcaccaaaatt

SEQ ID NO: 675 - DNA, 20 bp, Cannabis_sativa
tgatatcattaagtcacatg

SEQ ID NO: 676 - DNA, 20 bp, Cannabis_sativa
tgacttaatgatatcaaatt

SEQ ID NO: 677 - DNA, 20 bp, Cannabis_sativa
agctttttataatggagcag

SEQ ID NO: 678 - DNA, 20 bp, Cannabis_sativa
tagctttttataatggagca

SEQ ID NO: 679 - DNA, 20 bp, Cannabis_sativa
atagctttttataatggagc

SEQ ID NO: 680 - DNA, 20 bp, Cannabis_sativa
ctatttatagctttttataa

SEQ ID NO: 681 - DNA, 20 bp, Cannabis_sativa
cattataaaaagctataaat

SEQ ID NO: 682 - DNA, 20 bp, Cannabis_sativa
tcgtaaagtttttgcctttt

SEQ ID NO: 683 - DNA, 20 bp, Cannabis_sativa
tgatattgtacataccaaaa

SEQ ID NO: 684 - DNA, 20 bp, Cannabis_sativa
taataactttatataaatat

SEQ ID NO: 685 - DNA, 20 bp, Cannabis_sativa
ataataactttatataaata

SEQ ID NO: 686 - DNA, 20 bp, Cannabis_sativa
tttaactagatcaaataatt

SEQ ID NO: 687 - DNA, 20 bp, Cannabis_sativa
gatctagttaaatgcttact

SEQ ID NO: 688 - DNA, 20 bp, Cannabis_sativa
tgtaatttgttttttaaaaa

SEQ ID NO: 689 - DNA, 20 bp, Cannabis_sativa
taataatataagctttacgt

SEQ ID NO: 690 - DNA, 20 bp, Cannabis_sativa
cactttattcttatgtaaaa

SEQ ID NO: 691 - DNA, 20 bp, Cannabis_sativa
actttattcttatgtaaaaa

SEQ ID NO: 692 - DNA, 20 bp, Cannabis_sativa
ggctttgtccgcttcaattt

SEQ ID NO: 693 - DNA, 20 bp, Cannabis_sativa
aaagggtaccaaaattgaag

SEQ ID NO: 694 - DNA, 20 bp, Cannabis_sativa
aaaagtcaatattttcttgt

SEQ ID NO: 695 - DNA, 20 bp, Cannabis_sativa
atagtagtcaaataaaaatt

SEQ ID NO: 696 - DNA, 20 bp, Cannabis_sativa
ttattcgttaaactcaatta

SEQ ID NO: 697 - DNA, 20 bp, Cannabis_sativa
ttcgttaaactcaattatgg

SEQ ID NO: 698 - DNA, 20 bp, Cannabis_sativa
taaactcaattatggtggat

SEQ ID NO: 699 - DNA, 20 bp, Cannabis_sativa
tattatatattaaaattaga

SEQ ID NO: 700 - DNA, 20 bp, Cannabis_sativa
aaatttgttaaaaaaatagc

SEQ ID NO: 701 - DNA, 20 bp, Cannabis_sativa
ataataatatatatatatat

SEQ ID NO: 702 - DNA, 20 bp, Cannabis_sativa
tgttataaatactagaaatt

SEQ ID NO: 703 - DNA, 20 bp, Cannabis_sativa
aaatactagaaatttggaac

SEQ ID NO: 704 - DNA, 20 bp, Cannabis_sativa
aatactagaaatttggaact

SEQ ID NO: 705 - DNA, 20 bp, Cannabis_sativa
attaaaaaataataaaaata

SEQ ID NO: 706 - DNA, 20 bp, Cannabis_sativa
ccgggataaccattaggaat

SEQ ID NO: 707 - DNA, 20 bp, Cannabis_sativa
ttaattcgtccaattcctaa

SEQ ID NO: 708 - DNA, 20 bp, Cannabis_sativa
atcaaaccgggataaccatt

SEQ ID NO: 709 - DNA, 20 bp, Cannabis_sativa
ccaattcctaatggttatcc

SEQ ID NO: 710 - DNA, 20 bp, Cannabis_sativa
aaattaactttgatcaaacc

SEQ ID NO: 711 - DNA, 20 bp, Cannabis_sativa
caaattaactttgatcaaac

SEQ ID NO: 712 - DNA, 20 bp, Cannabis_sativa
tattattcatttttaataga

SEQ ID NO: 713 - DNA, 20 bp, Cannabis_sativa
ttcaaataatacaatgtaag

SEQ ID NO: 714 - DNA, 20 bp, Cannabis_sativa
atactattaaattagttatg

SEQ ID NO: 715 - DNA, 20 bp, Cannabis_sativa
taaataaaaatactgagtca

SEQ ID NO: 716 - DNA, 20 bp, Cannabis_sativa
tttttagaattctcataata

SEQ ID NO: 717 - DNA, 20 bp, Cannabis_sativa
actaatgactcattgaatct

SEQ ID NO: 718 - DNA, 20 bp, Cannabis_sativa
ctaatgactcattgaatcta

SEQ ID NO: 719 - DNA, 20 bp, Cannabis_sativa
attttaaagataaacaaagt

SEQ ID NO: 720 - DNA, 20 bp, Cannabis_sativa
tagagttgggtgctaggcgt

SEQ ID NO: 721 - DNA, 20 bp, Cannabis_sativa
atagagttgggtgctaggcg

SEQ ID NO: 722 - DNA, 20 bp, Cannabis_sativa
cctcaatagagttgggtgct

SEQ ID NO: 723 - DNA, 20 bp, Cannabis_sativa
ttcacggcctcaatagagtt

SEQ ID NO: 724 - DNA, 20 bp, Cannabis_sativa
tttcacggcctcaatagagt

SEQ ID NO: 725 - DNA, 20 bp, Cannabis_sativa
cctagcacccaactctattg

SEQ ID NO: 726 - DNA, 20 bp, Cannabis_sativa
atttgatttttattttttca

SEQ ID NO: 727 - DNA, 20 bp, Cannabis_sativa
ctttaaaatatctttaatta

SEQ ID NO: 728 - DNA, 20 bp, Cannabis_sativa
ttaattcacataatatatat

SEQ ID NO: 729 - DNA, 20 bp, Cannabis_sativa
ttccatgaaagtacaatcac

SEQ ID NO: 730 - DNA, 20 bp, Cannabis_sativa
attccatgaaagtacaatca

SEQ ID NO: 731 - DNA, 20 bp, Cannabis_sativa
gtcccgtgattgtactttca

SEQ ID NO: 732 - DNA, 20 bp, Cannabis_sativa
ctaacggctgtcgtacatca

SEQ ID NO: 733 - DNA, 20 bp, Cannabis_sativa
caaataactccctcatctaa

SEQ ID NO: 734 - DNA, 20 bp, Cannabis_sativa
tgtacgacagccgttagatg

SEQ ID NO: 735 - DNA, 20 bp, Cannabis_sativa
gtacgacagccgttagatga

SEQ ID NO: 736 - DNA, 20 bp, Cannabis_sativa
gatgagggagttatttgatc

SEQ ID NO: 737 - DNA, 20 bp, Cannabis_sativa
atgagggagttatttgatct

SEQ ID NO: 738 - DNA, 20 bp, Cannabis_sativa
tgagggagttatttgatctg

SEQ ID NO: 739 - DNA, 20 bp, Cannabis_sativa
gagggagttatttgatctgg

SEQ ID NO: 740 - DNA, 20 bp, Cannabis_sativa
ctgggggctgagattgatct

SEQ ID NO: 741 - DNA, 20 bp, Cannabis_sativa
tgggggctgagattgatcta

SEQ ID NO: 742 - DNA, 20 bp, Cannabis_sativa
gggggctgagattgatctag

SEQ ID NO: 743 - DNA, 20 bp, Cannabis_sativa
agattgatctaggggtaatt

SEQ ID NO: 744 - DNA, 20 bp, Cannabis_sativa
gattgatctaggggtaatta

SEQ ID NO: 745 - DNA, 20 bp, Cannabis_sativa
tcaggggtacattagtgtca

SEQ ID NO: 746 - DNA, 20 bp, Cannabis_sativa
ctcaggggtacattagtgtc

SEQ ID NO: 747 - DNA, 20 bp, Cannabis_sativa
tggtattagatgggtctcag

SEQ ID NO: 748 - DNA, 20 bp, Cannabis_sativa
gtggtattagatgggtctca

SEQ ID NO: 749 - DNA, 20 bp, Cannabis_sativa
cgtggtattagatgggtctc

SEQ ID NO: 750 - DNA, 20 bp, Cannabis_sativa
tgtattccgtggtattagat

SEQ ID NO: 751 - DNA, 20 bp, Cannabis_sativa
atgtattccgtggtattaga

SEQ ID NO: 752 - DNA, 20 bp, Cannabis_sativa
ctgagacccatctaatacca

SEQ ID NO: 753 - DNA, 20 bp, Cannabis_sativa
ctttcacggaatgtattccg

SEQ ID NO: 754 - DNA, 20 bp, Cannabis_sativa
tttcccgtgatgtactttca

SEQ ID NO: 755 - DNA, 20 bp, Cannabis_sativa
cattccgtgaaagtacatca

SEQ ID NO: 756 - DNA, 20 bp, Cannabis_sativa
attccgtgaaagtacatcac

SEQ ID NO: 757 - DNA, 20 bp, Cannabis_sativa
aatagatatatatatatatt

SEQ ID NO: 758 - DNA, 20 bp, Cannabis_sativa
aaatagatatatatatatat

SEQ ID NO: 759 - DNA, 20 bp, Cannabis_sativa
agtttttgaaactcttctaa

SEQ ID NO: 760 - DNA, 20 bp, Cannabis_sativa
aaagtcaaatattataattt

SEQ ID NO: 761 - DNA, 20 bp, Cannabis_sativa
aattctcttaataaattata

SEQ ID NO: 762 - DNA, 20 bp, Cannabis_sativa
aaattctcttaataaattat

SEQ ID NO: 763 - DNA, 20 bp, Cannabis_sativa
acttattcaatcattaataa

SEQ ID NO: 764 - DNA, 20 bp, Cannabis_sativa
ataaaggttaacaatgatca

SEQ ID NO: 765 - DNA, 20 bp, Cannabis_sativa
taaaggttaacaatgatcat

SEQ ID NO: 766 - DNA, 20 bp, Cannabis_sativa
aaaggttaacaatgatcatg

SEQ ID NO: 767 - DNA, 20 bp, Cannabis_sativa
tgtgcctaatgttgtagtta

SEQ ID NO: 768 - DNA, 20 bp, Cannabis_sativa
ggggccttaactacaacatt

SEQ ID NO: 769 - DNA, 20 bp, Cannabis_sativa
aacattaggcacattttcaa

SEQ ID NO: 770 - DNA, 20 bp, Cannabis_sativa
cccataataaaagtggcttt

SEQ ID NO: 771 - DNA, 20 bp, Cannabis_sativa
tcatatccccataataaaag

SEQ ID NO: 772 - DNA, 20 bp, Cannabis_sativa
tccaaaagccacttttatta

SEQ ID NO: 773 - DNA, 20 bp, Cannabis_sativa
ccaaaagccacttttattat

SEQ ID NO: 774 - DNA, 20 bp, Cannabis_sativa
caaaagccacttttattatg

SEQ ID NO: 775 - DNA, 20 bp, Cannabis_sativa
tttttttagtcttaattaag

SEQ ID NO: 776 - DNA, 20 bp, Cannabis_sativa
tatgtgtattaaaattaaat

SEQ ID NO: 777 - DNA, 20 bp, Cannabis_sativa
gtaccgggttttaaataatt

SEQ ID NO: 778 - DNA, 20 bp, Cannabis_sativa
gaaccaaattatttaaaacc

SEQ ID NO: 779 - DNA, 20 bp, Cannabis_sativa
taggagggttaaagagtacc

SEQ ID NO: 780 - DNA, 20 bp, Cannabis_sativa
gtaggagggttaaagagtac

SEQ ID NO: 781 - DNA, 20 bp, Cannabis_sativa
tttttcagtgggtggtagga

SEQ ID NO: 782 - DNA, 20 bp, Cannabis_sativa
gtttttcagtgggtggtagg

SEQ ID NO: 783 - DNA, 20 bp, Cannabis_sativa
atagtttttcagtgggtggt

SEQ ID NO: 784 - DNA, 20 bp, Cannabis_sativa
taatatagtttttcagtggg

SEQ ID NO: 785 - DNA, 20 bp, Cannabis_sativa
gtgtaatatagtttttcagt

SEQ ID NO: 786 - DNA, 20 bp, Cannabis_sativa
agtgtaatatagtttttcag

SEQ ID NO: 787 - DNA, 20 bp, Cannabis_sativa
taatttgccttttattctca

SEQ ID NO: 788 - DNA, 20 bp, Cannabis_sativa
actttaaccatgagaataaa

SEQ ID NO: 789 - DNA, 20 bp, Cannabis_sativa
aataaaaggcaaattaagag

SEQ ID NO: 790 - DNA, 20 bp, Cannabis_sativa
ataaaaggcaaattaagagt

SEQ ID NO: 791 - DNA, 20 bp, Cannabis_sativa
aaaaaaaagtgaatttcaag

SEQ ID NO: 792 - DNA, 20 bp, Cannabis_sativa
atcaccaagattgaaaatgg

SEQ ID NO: 793 - DNA, 20 bp, Cannabis_sativa
cttatcaccaagattgaaaa

SEQ ID NO: 794 - DNA, 20 bp, Cannabis_sativa
aagtccaccattttcaatct

SEQ ID NO: 795 - DNA, 20 bp, Cannabis_sativa
tataggtatcgccaaattaa

SEQ ID NO: 796 - DNA, 20 bp, Cannabis_sativa
cttatatagtaccgttaatt

SEQ ID NO: 797 - DNA, 20 bp, Cannabis_sativa
tagttagagactcggagtat

SEQ ID NO: 798 - DNA, 20 bp, Cannabis_sativa
tcaaagaatagttagagact

SEQ ID NO: 799 - DNA, 20 bp, Cannabis_sativa
tgatataaatgatacattaa

SEQ ID NO: 800 - DNA, 20 bp, Cannabis_sativa
attacacatatttagaatga

SEQ ID NO: 801 - DNA, 20 bp, Cannabis_sativa
gaaaattttatttgcatccc

SEQ ID NO: 802 - DNA, 20 bp, Cannabis_sativa
aatatgagggtgtacttcct

SEQ ID NO: 803 - DNA, 20 bp, Cannabis_sativa
taatatgagggtgtacttcc

SEQ ID NO: 804 - DNA, 20 bp, Cannabis_sativa
attttttttttttaatatga

SEQ ID NO: 805 - DNA, 20 bp, Cannabis_sativa
tattttttttttttaatatg

SEQ ID NO: 806 - DNA, 20 bp, Cannabis_sativa
attttttttaaatattattt

SEQ ID NO: 807 - DNA, 20 bp, Cannabis_sativa
ttttttttaaatattatttt

SEQ ID NO: 808 - DNA, 20 bp, Cannabis_sativa
ataaaatttcacatgaattt

SEQ ID NO: 809 - DNA, 20 bp, Cannabis_sativa
ataatttatttttatttgat

SEQ ID NO: 810 - DNA, 20 bp, Cannabis_sativa
atttgataggtatatttttt

SEQ ID NO: 811 - DNA, 20 bp, Cannabis_sativa
taaaataatttgatgaaaat

SEQ ID NO: 812 - DNA, 20 bp, Cannabis_sativa
caagaaacatccaactcata

SEQ ID NO: 813 - DNA, 20 bp, Cannabis_sativa
acgaagatagccatatgagt

SEQ ID NO: 814 - DNA, 20 bp, Cannabis_sativa
aatataacttacgcgtcata

SEQ ID NO: 815 - DNA, 20 bp, Cannabis_sativa
aggttttgagataagtatga

SEQ ID NO: 816 - DNA, 20 bp, Cannabis_sativa
aacgtgaaactgaataaatt

SEQ ID NO: 817 - DNA, 20 bp, Cannabis_sativa
ataaatcaattacaattgaa

SEQ ID NO: 818 - DNA, 20 bp, Cannabis_sativa
aaagaataaaagaacataaa

SEQ ID NO: 819 - DNA, 20 bp, Cannabis_sativa
ttaatacaagtaaaaaataa

SEQ ID NO: 820 - DNA, 20 bp, Cannabis_sativa
ttgtattaattttcaagata

SEQ ID NO: 821 - DNA, 20 bp, Cannabis_sativa
acggttgtatatattattta

SEQ ID NO: 822 - DNA, 20 bp, Cannabis_sativa
aacttttttttttaaattta

SEQ ID NO: 823 - DNA, 20 bp, Cannabis_sativa
ttttttttaaatttacggtt

SEQ ID NO: 824 - DNA, 20 bp, Cannabis_sativa
tttttttaaatttacggttt

SEQ ID NO: 825 - DNA, 20 bp, Cannabis_sativa
tacggtttgggtttctaaag

SEQ ID NO: 826 - DNA, 20 bp, Cannabis_sativa
gttgcagcgctagttgcaat

SEQ ID NO: 827 - DNA, 20 bp, Cannabis_sativa
ttgcagcgctagttgcaata

SEQ ID NO: 828 - DNA, 20 bp, Cannabis_sativa
tgcagcgctagttgcaatag

SEQ ID NO: 829 - DNA, 20 bp, Cannabis_sativa
acgattttttgttgcaattt

SEQ ID NO: 830 - DNA, 20 bp, Cannabis_sativa
tttttttttttgcattttta

SEQ ID NO: 831 - DNA, 20 bp, Cannabis_sativa
atataataaataaataatag

SEQ ID NO: 832 - DNA, 20 bp, Cannabis_sativa
tatataataaataaataata

SEQ ID NO: 833 - DNA, 20 bp, Cannabis_sativa
atatataataaataaataat

SEQ ID NO: 834 - DNA, 20 bp, Cannabis_sativa
catatttctagagactttgt

SEQ ID NO: 835 - DNA, 20 bp, Cannabis_sativa
gattgactttgtgtcatata

SEQ ID NO: 836 - DNA, 20 bp, Cannabis_sativa
tgactttgtgtcatatatgg

SEQ ID NO: 837 - DNA, 20 bp, Cannabis_sativa
gtggcaaatatgtaccttga

SEQ ID NO: 838 - DNA, 20 bp, Cannabis_sativa
aatcaaccctagctccatca

SEQ ID NO: 839 - DNA, 20 bp, Cannabis_sativa
atatgtaccttgatggagct

SEQ ID NO: 840 - DNA, 20 bp, Cannabis_sativa
tatgtaccttgatggagcta

SEQ ID NO: 841 - DNA, 20 bp, Cannabis_sativa
agctagggttgattatgcta

SEQ ID NO: 842 - DNA, 20 bp, Cannabis_sativa
agggttgattatgctatggt

SEQ ID NO: 843 - DNA, 20 bp, Cannabis_sativa
ttgattatgctatggttggt

SEQ ID NO: 844 - DNA, 20 bp, Cannabis_sativa
attatgctatggttggtcgg

SEQ ID NO: 845 - DNA, 20 bp, Cannabis_sativa
gtggctagttgtaccatgtt

SEQ ID NO: 846 - DNA, 20 bp, Cannabis_sativa
aaaaaaatacccaccaaaca

SEQ ID NO: 847 - DNA, 20 bp, Cannabis_sativa
gctagttgtaccatgtttgg

SEQ ID NO: 848 - DNA, 20 bp, Cannabis_sativa
ctagttgtaccatgtttggt

SEQ ID NO: 849 - DNA, 20 bp, Cannabis_sativa
aattgtacaaaaaactaaca

SEQ ID NO: 850 - DNA, 20 bp, Cannabis_sativa
tatagttttgatgccttttt

SEQ ID NO: 851 - DNA, 20 bp, Cannabis_sativa
atagttttgatgccttttta

SEQ ID NO: 852 - DNA, 20 bp, Cannabis_sativa
atggtacaaatgccctaaaa

SEQ ID NO: 853 - DNA, 20 bp, Cannabis_sativa
taagagcaagccataaaata

SEQ ID NO: 854 - DNA, 20 bp, Cannabis_sativa
ggcatttgtaccatatttta

SEQ ID NO: 855 - DNA, 20 bp, Cannabis_sativa
aattgtttagcgtaaatttg

SEQ ID NO: 856 - DNA, 20 bp, Cannabis_sativa
tttagcgtaaatttgaggta

SEQ ID NO: 857 - DNA, 20 bp, Cannabis_sativa
aaaacccttctcttaatcat

SEQ ID NO: 858 - DNA, 20 bp, Cannabis_sativa
tatggccaatgattaagaga

SEQ ID NO: 859 - DNA, 20 bp, Cannabis_sativa
atggccaatgattaagagaa

SEQ ID NO: 860 - DNA, 20 bp, Cannabis_sativa
agggttttgttttgtagtct

SEQ ID NO: 861 - DNA, 20 bp, Cannabis_sativa
gggttttgttttgtagtctt

SEQ ID NO: 862 - DNA, 20 bp, Cannabis_sativa
acaatttaataaaaaaaaat

SEQ ID NO: 863 - DNA, 20 bp, Cannabis_sativa
tttgtaagtaatttttattt

SEQ ID NO: 864 - DNA, 20 bp, Cannabis_sativa
gctattttttttttttttgt

SEQ ID NO: 865 - DNA, 20 bp, Cannabis_sativa
ttttttttttttttgtaggt

SEQ ID NO: 866 - DNA, 20 bp, Cannabis_sativa
tttgtaggtcggttttgtta

SEQ ID NO: 867 - DNA, 20 bp, Cannabis_sativa
ttgtaggtcggttttgttaa

SEQ ID NO: 868 - DNA, 20 bp, Cannabis_sativa
tagtttttaatataattgtt

SEQ ID NO: 869 - DNA, 20 bp, Cannabis_sativa
agtttttaatataattgtta

SEQ ID NO: 870 - DNA, 20 bp, Cannabis_sativa
gtttttaatataattgttag

SEQ ID NO: 871 - DNA, 20 bp, Cannabis_sativa
atttttttgtagatattttg

SEQ ID NO: 872 - DNA, 20 bp, Cannabis_sativa
acaccaactcatataacaaa

SEQ ID NO: 873 - DNA, 20 bp, Cannabis_sativa
ggaccatttgttatatgagt

SEQ ID NO: 874 - DNA, 20 bp, Cannabis_sativa
tttattttctaaatttttag

SEQ ID NO: 875 - DNA, 20 bp, Cannabis_sativa
ctaaagtaagcaagcaacat

SEQ ID NO: 876 - DNA, 20 bp, Cannabis_sativa
gctaaagtaagcaagcaaca

SEQ ID NO: 877 - DNA, 20 bp, Cannabis_sativa
agtttgacaaagcatgctat

SEQ ID NO: 878 - DNA, 20 bp, Cannabis_sativa
cgatgatctatcctagttcg

SEQ ID NO: 879 - DNA, 20 bp, Cannabis_sativa
tgcagaaacttcctcgaact

SEQ ID NO: 880 - DNA, 20 bp, Cannabis_sativa
ggaagtttctgcaatatttg

SEQ ID NO: 881 - DNA, 20 bp, Cannabis_sativa
gaagttgactatggaaagaa

SEQ ID NO: 882 - DNA, 20 bp, Cannabis_sativa
tgccatattgaagttgacta

SEQ ID NO: 883 - DNA, 20 bp, Cannabis_sativa
ttccatagtcaacttcaata

SEQ ID NO: 884 - DNA, 20 bp, Cannabis_sativa
agttgagcatcatttgttga

SEQ ID NO: 885 - DNA, 20 bp, Cannabis_sativa
atttatatttatttttaata

SEQ ID NO: 886 - DNA, 20 bp, Cannabis_sativa
tttatatttatttttaataa

SEQ ID NO: 887 - DNA, 20 bp, Cannabis_sativa
ttaggcatcattgtattgtt

SEQ ID NO: 888 - DNA, 20 bp, Cannabis_sativa
aaaaaaaattcacaaaaatt

SEQ ID NO: 889 - DNA, 20 bp, Cannabis_sativa
tttgatatcattaagtcatg

SEQ ID NO: 890 - DNA, 20 bp, Cannabis_sativa
agctttatatattggagcag

SEQ ID NO: 891 - DNA, 20 bp, Cannabis_sativa
tagctttatatattggagca

SEQ ID NO: 892 - DNA, 20 bp, Cannabis_sativa
atagctttatatattggagc

SEQ ID NO: 893 - DNA, 20 bp, Cannabis_sativa
ctatttatagctttatatat

SEQ ID NO: 894 - DNA, 20 bp, Cannabis_sativa
caatatataaagctataaat

SEQ ID NO: 895 - DNA, 20 bp, Cannabis_sativa
taatgaattttgaattacta

SEQ ID NO: 896 - DNA, 20 bp, Cannabis_sativa
aaatttataggaaaccccta

SEQ ID NO: 897 - DNA, 20 bp, Cannabis_sativa
aaaaattttaaaaaatttat

SEQ ID NO: 898 - DNA, 20 bp, Cannabis_sativa
gctctaagtgtttgtatatt

SEQ ID NO: 899 - DNA, 20 bp, Cannabis_sativa
taaaaatgatactaaaatac

SEQ ID NO: 900 - DNA, 20 bp, Cannabis_sativa
tacatttaacttttataata

SEQ ID NO: 901 - DNA, 20 bp, Cannabis_sativa
acatttaacttttataatat

SEQ ID NO: 902 - DNA, 20 bp, Cannabis_sativa
aattacaaaaatggtctatt

SEQ ID NO: 903 - DNA, 20 bp, Cannabis_sativa
aaattacaaaaatggtctat

SEQ ID NO: 904 - DNA, 20 bp, Cannabis_sativa
ccaaaaaaaaattacaaaaa

SEQ ID NO: 905 - DNA, 20 bp, Cannabis_sativa
ccatttttgtaatttttttt

SEQ ID NO: 906 - DNA, 20 bp, Cannabis_sativa
ttagaataatattaatacgt

SEQ ID NO: 907 - DNA, 20 bp, Cannabis_sativa
aaaattactctaagtattta

SEQ ID NO: 908 - DNA, 20 bp, Cannabis_sativa
agcacataattttttgtata

SEQ ID NO: 909 - DNA, 20 bp, Cannabis_sativa
tttatcgaaattgactttat

SEQ ID NO: 910 - DNA, 20 bp, Cannabis_sativa
ttctcttaaacttggttgtt

SEQ ID NO: 911 - DNA, 20 bp, Cannabis_sativa
attttagtttctcttaaact

SEQ ID NO: 912 - DNA, 20 bp, Cannabis_sativa
gccgaaatttcggtagaatt

SEQ ID NO: 913 - DNA, 20 bp, Cannabis_sativa
tcctaattctaccgaaattt

SEQ ID NO: 914 - DNA, 20 bp, Cannabis_sativa
ccgttatgctgccgaaattt

SEQ ID NO: 915 - DNA, 20 bp, Cannabis_sativa
ccgaaatttcggcagcataa

SEQ ID NO: 916 - DNA, 20 bp, Cannabis_sativa
attaataggtttgaattttt

SEQ ID NO: 917 - DNA, 20 bp, Cannabis_sativa
tgtgttttgttgttattaat

SEQ ID NO: 918 - DNA, 20 bp, Cannabis_sativa
atgtactgtagtcggatggg

SEQ ID NO: 919 - DNA, 20 bp, Cannabis_sativa
tacatgtactgtagtcggat

SEQ ID NO: 920 - DNA, 20 bp, Cannabis_sativa
atacatgtactgtagtcgga

SEQ ID NO: 921 - DNA, 20 bp, Cannabis_sativa
gtgaatacatgtactgtagt

SEQ ID NO: 922 - DNA, 20 bp, Cannabis_sativa
atattcatctgtagtgaagt

SEQ ID NO: 923 - DNA, 20 bp, Cannabis_sativa
ttgtactatgtcggatcgat

SEQ ID NO: 924 - DNA, 20 bp, Cannabis_sativa
attgtactatgtcggatcga

SEQ ID NO: 925 - DNA, 20 bp, Cannabis_sativa
tacaatatattgtactatgt

SEQ ID NO: 926 - DNA, 20 bp, Cannabis_sativa
atattcatttgtagtgaagt

SEQ ID NO: 927 - DNA, 20 bp, Cannabis_sativa
ttttgtgttgatcggtttct

SEQ ID NO: 928 - DNA, 20 bp, Cannabis_sativa
ccaacttattttgtgttgat

SEQ ID NO: 929 - DNA, 20 bp, Cannabis_sativa
ccgatcaacacaaaataagt

SEQ ID NO: 930 - DNA, 20 bp, Cannabis_sativa
ggttgtgaggtcaatttgca

SEQ ID NO: 931 - DNA, 20 bp, Cannabis_sativa
tctttatgttgaaggttgtg

SEQ ID NO: 932 - DNA, 20 bp, Cannabis_sativa
gtagtatttctttatgttga

SEQ ID NO: 933 - DNA, 20 bp, Cannabis_sativa
aagtagctaaataaaaaaat

SEQ ID NO: 934 - DNA, 20 bp, Cannabis_sativa
atgtgttttatttctttagt

SEQ ID NO: 935 - DNA, 20 bp, Cannabis_sativa
attgtgaatgagaatgagat

SEQ ID NO: 936 - DNA, 20 bp, Cannabis_sativa
gtgaatgagaaatgtaatat

SEQ ID NO: 937 - DNA, 20 bp, Cannabis_sativa
tgtgtatatctattgtgaat

SEQ ID NO: 938 - DNA, 20 bp, Cannabis_sativa
atgtgtatatctattgtgaa

SEQ ID NO: 939 - DNA, 20 bp, Cannabis_sativa
ttattttataaattttttta

SEQ ID NO: 940 - DNA, 20 bp, Cannabis_sativa
gttattttataaattttttt

SEQ ID NO: 941 - DNA, 20 bp, Cannabis_sativa
aattagatttataccttaat

SEQ ID NO: 942 - DNA, 20 bp, Cannabis_sativa
ttgtatctcaacgcctatta

SEQ ID NO: 943 - DNA, 20 bp, Cannabis_sativa
cctccggccaccgtttttag

SEQ ID NO: 944 - DNA, 20 bp, Cannabis_sativa
aatgttttctccactaaaaa

SEQ ID NO: 945 - DNA, 20 bp, Cannabis_sativa
gttttctccactaaaaacgg

SEQ ID NO: 946 - DNA, 20 bp, Cannabis_sativa
tctccactaaaaacggtggc

SEQ ID NO: 947 - DNA, 20 bp, Cannabis_sativa
ccactaaaaacggtggccgg

SEQ ID NO: 948 - DNA, 20 bp, Cannabis_sativa
ggtagtggtgattatacctc

SEQ ID NO: 949 - DNA, 20 bp, Cannabis_sativa
taaacaaaaagtaaaggtag

SEQ ID NO: 950 - DNA, 20 bp, Cannabis_sativa
ttggggtaaacaaaaagtaa

SEQ ID NO: 951 - DNA, 20 bp, Cannabis_sativa
acatttttcctcattttttg

SEQ ID NO: 952 - DNA, 20 bp, Cannabis_sativa
tacatttttcctcatttttt

SEQ ID NO: 953 - DNA, 20 bp, Cannabis_sativa
ttacatttttcctcattttt

SEQ ID NO: 954 - DNA, 20 bp, Cannabis_sativa
tttgtttaccccaaaaaatg

SEQ ID NO: 955 - DNA, 20 bp, Cannabis_sativa
aggaaaaatgtaatcttttc

SEQ ID NO: 956 - DNA, 20 bp, Cannabis_sativa
cttttcaggtatatagtttt

SEQ ID NO: 957 - DNA, 20 bp, Cannabis_sativa
gaaataaacatgagctaaaa

SEQ ID NO: 958 - DNA, 20 bp, Cannabis_sativa
atggtgaaaaaatagtgaaa

SEQ ID NO: 959 - DNA, 20 bp, Cannabis_sativa
gtgaaaaaatagtgaaatgg

SEQ ID NO: 960 - DNA, 20 bp, Cannabis_sativa
gaaatggaggtgatttttcg

SEQ ID NO: 961 - DNA, 20 bp, Cannabis_sativa
atggaggtgatttttcgtgg

SEQ ID NO: 962 - DNA, 20 bp, Cannabis_sativa
aggtgatttttcgtggtggt

SEQ ID NO: 963 - DNA, 20 bp, Cannabis_sativa
tgatttttcgtggtggttgg

SEQ ID NO: 964 - DNA, 20 bp, Cannabis_sativa
tttcgtggtggttggtggag

SEQ ID NO: 965 - DNA, 20 bp, Cannabis_sativa
ttcgtggtggttggtggaga

SEQ ID NO: 966 - DNA, 20 bp, Cannabis_sativa
ggtggttggtggagagggtt

SEQ ID NO: 967 - DNA, 20 bp, Cannabis_sativa
gtggttggtggagagggttt

SEQ ID NO: 968 - DNA, 20 bp, Cannabis_sativa
tggttggtggagagggtttg

SEQ ID NO: 969 - DNA, 20 bp, Cannabis_sativa
gagagggtttggggtttctt

SEQ ID NO: 970 - DNA, 20 bp, Cannabis_sativa
ggtttggggtttctttggtt

SEQ ID NO: 971 - DNA, 20 bp, Cannabis_sativa
gtttggggtttctttggttt

SEQ ID NO: 972 - DNA, 20 bp, Cannabis_sativa
tttggggtttctttggtttg

SEQ ID NO: 973 - DNA, 20 bp, Cannabis_sativa
ttggggtttctttggtttgg

SEQ ID NO: 974 - DNA, 20 bp, Cannabis_sativa
ttctttggtttgggggtttc

SEQ ID NO: 975 - DNA, 20 bp, Cannabis_sativa
tctttggtttgggggtttct

SEQ ID NO: 976 - DNA, 20 bp, Cannabis_sativa
tggtttgggggtttctgggt

SEQ ID NO: 977 - DNA, 20 bp, Cannabis_sativa
ttgggggtttctgggttgga

SEQ ID NO: 978 - DNA, 20 bp, Cannabis_sativa
ctgggttggatggtcgaatg

SEQ ID NO: 979 - DNA, 20 bp, Cannabis_sativa
tggtcgaatgaggaagatga

SEQ ID NO: 980 - DNA, 20 bp, Cannabis_sativa
ggtcgaatgaggaagatgaa

SEQ ID NO: 981 - DNA, 20 bp, Cannabis_sativa
gaatgaggaagatgaagggc

SEQ ID NO: 982 - DNA, 20 bp, Cannabis_sativa
aatgaggaagatgaagggct

SEQ ID NO: 983 - DNA, 20 bp, Cannabis_sativa
ggaagatgaagggctgggct

SEQ ID NO: 984 - DNA, 20 bp, Cannabis_sativa
gaagatgaagggctgggcta

SEQ ID NO: 985 - DNA, 20 bp, Cannabis_sativa
gctagggttataaaaccttt

SEQ ID NO: 986 - DNA, 20 bp, Cannabis_sativa
agaagataaccgacgccaaa

SEQ ID NO: 987 - DNA, 20 bp, Cannabis_sativa
gttataaaaccttttggcgt

SEQ ID NO: 988 - DNA, 20 bp, Cannabis_sativa
tagtttttaatataattgta

SEQ ID NO: 989 - DNA, 20 bp, Cannabis_sativa
agtttttaatataattgtaa

SEQ ID NO: 990 - DNA, 20 bp, Cannabis_sativa
gtttttaatataattgtaag

SEQ ID NO: 991 - DNA, 20 bp, Cannabis_sativa
atttttttgtagatattttg

SEQ ID NO: 992 - DNA, 20 bp, Cannabis_sativa
acaccaactcatataacaaa

SEQ ID NO: 993 - DNA, 20 bp, Cannabis_sativa
ggaccatttgttatatgagt

SEQ ID NO: 994 - DNA, 20 bp, Cannabis_sativa
ctcttttattgtgttgttta

SEQ ID NO: 995 - DNA, 20 bp, Cannabis_sativa
ctaaagtaagcaagcaacat

SEQ ID NO: 996 - DNA, 20 bp, Cannabis_sativa
gctaaagtaagcaagcaaca

SEQ ID NO: 997 - DNA, 20 bp, Cannabis_sativa
cgatgatctatcctaattcg

SEQ ID NO: 998 - DNA, 20 bp, Cannabis_sativa
tgcagaaacctcctcgaatt

SEQ ID NO: 999 - DNA, 20 bp, Cannabis_sativa
tgatctatcctaattcgagg

SEQ ID NO: 1000 - DNA, 20 bp, Cannabis_sativa
gactagcaaatatttatcta

SEQ ID NO: 1001 - DNA, 20 bp, Cannabis_sativa
gaagttgactatggaaagaa

SEQ ID NO: 1002 - DNA, 20 bp, Cannabis_sativa
tgccatattgaagttgacta

SEQ ID NO: 1003 - DNA, 20 bp, Cannabis_sativa
ttccatagtcaacttcaata

SEQ ID NO: 1004 - DNA, 20 bp, Cannabis_sativa
agttgagcatcatttgttga

SEQ ID NO: 1005 - DNA, 20 bp, Cannabis_sativa
atttatatttatttttagta

SEQ ID NO: 1006 - DNA, 20 bp, Cannabis_sativa
tttatatttatttttagtaa

SEQ ID NO: 1007 - DNA, 20 bp, Cannabis_sativa
caaaattaggcatcattgtt

SEQ ID NO: 1008 - DNA, 20 bp, Cannabis_sativa
ctaacaatgatgcctaattt

SEQ ID NO: 1009 - DNA, 20 bp, Cannabis_sativa
aaaaaaaattcaccaaaatt

SEQ ID NO: 1010 - DNA, 20 bp, Cannabis_sativa
tgatatcattaagtcacatg

SEQ ID NO: 1011 - DNA, 20 bp, Cannabis_sativa
tgacttaatgatatcaaatt

SEQ ID NO: 1012 - DNA, 20 bp, Cannabis_sativa
agctttttataatggagcag

SEQ ID NO: 1013 - DNA, 20 bp, Cannabis_sativa
tagctttttataatggagca

SEQ ID NO: 1014 - DNA, 20 bp, Cannabis_sativa
atagctttttataatggagc

SEQ ID NO: 1015 - DNA, 20 bp, Cannabis_sativa
ctatttatagctttttataa

SEQ ID NO: 1016 - DNA, 20 bp, Cannabis_sativa
cattataaaaagctataaat

SEQ ID NO: 1017 - DNA, 20 bp, Cannabis_sativa
caaagttatagggatttctt

SEQ ID NO: 1018 - DNA, 20 bp, Cannabis_sativa
ggcctgatcccaaagttata

SEQ ID NO: 1019 - DNA, 20 bp, Cannabis_sativa
tggcctgatcccaaagttat

SEQ ID NO: 1020 - DNA, 20 bp, Cannabis_sativa
caaagaaatccctataactt

SEQ ID NO: 1021 - DNA, 20 bp, Cannabis_sativa
aaagaaatccctataacttt

SEQ ID NO: 1022 - DNA, 20 bp, Cannabis_sativa
atccctataactttgggatc

SEQ ID NO: 1023 - DNA, 20 bp, Cannabis_sativa
cgaatcttgccatatctcta

SEQ ID NO: 1024 - DNA, 20 bp, Cannabis_sativa
gggatcaggccatagagata

SEQ ID NO: 1025 - DNA, 20 bp, Cannabis_sativa
aaaagtttatcactttattt

SEQ ID NO: 1026 - DNA, 20 bp, Cannabis_sativa
taaaagtttatcactttatt

SEQ ID NO: 1027 - DNA, 20 bp, Cannabis_sativa
ttttaaacaaagaagcaaca

SEQ ID NO: 1028 - DNA, 20 bp, Cannabis_sativa
cgtacattgtcgacctcaga

SEQ ID NO: 1029 - DNA, 20 bp, Cannabis_sativa
cctttttccttatccttctg

SEQ ID NO: 1030 - DNA, 20 bp, Cannabis_sativa
ttgtcgacctcagaaggata

SEQ ID NO: 1031 - DNA, 20 bp, Cannabis_sativa
cctcagaaggataaggaaaa

SEQ ID NO: 1032 - DNA, 20 bp, Cannabis_sativa
cagtgaccgtgatagcgagc

SEQ ID NO: 1033 - DNA, 20 bp, Cannabis_sativa
aaggctccagctcgctatca

SEQ ID NO: 1034 - DNA, 20 bp, Cannabis_sativa
cagctcgctatcacggtcac

SEQ ID NO: 1035 - DNA, 20 bp, Cannabis_sativa
aggatgtccgtacaggagct

SEQ ID NO: 1036 - DNA, 20 bp, Cannabis_sativa
actggaaccaagctcctgta

SEQ ID NO: 1037 - DNA, 20 bp, Cannabis_sativa
gtgtctgaggatgtccgtac

SEQ ID NO: 1038 - DNA, 20 bp, Cannabis_sativa
atctttaattattgtgtctg

SEQ ID NO: 1039 - DNA, 20 bp, Cannabis_sativa
tgtgatcctcccgcatttat

SEQ ID NO: 1040 - DNA, 20 bp, Cannabis_sativa
tcattttgatccaataaatg

SEQ ID NO: 1041 - DNA, 20 bp, Cannabis_sativa
cattttgatccaataaatgc

SEQ ID NO: 1042 - DNA, 20 bp, Cannabis_sativa
tttgatccaataaatgcggg

SEQ ID NO: 1043 - DNA, 20 bp, Cannabis_sativa
catatcacgttgtgtaaatg

SEQ ID NO: 1044 - DNA, 20 bp, Cannabis_sativa
cgatcatcccctaaaatcat

SEQ ID NO: 1045 - DNA, 20 bp, Cannabis_sativa
acgatcatcccctaaaatca

SEQ ID NO: 1046 - DNA, 20 bp, Cannabis_sativa
taatcaaatcccatgatttt

SEQ ID NO: 1047 - DNA, 20 bp, Cannabis_sativa
aatcaaatcccatgatttta

SEQ ID NO: 1048 - DNA, 20 bp, Cannabis_sativa
atcaaatcccatgattttag

SEQ ID NO: 1049 - DNA, 20 bp, Cannabis_sativa
actatttataatcacatagt

SEQ ID NO: 1050 - DNA, 20 bp, Cannabis_sativa
tactatgtgattataaatag

SEQ ID NO: 1051 - DNA, 20 bp, Cannabis_sativa
ggcaagtaagatcaaaaaag

SEQ ID NO: 1052 - DNA, 20 bp, Cannabis_sativa
acgaaaaaagcatacaaaaa

SEQ ID NO: 1053 - DNA, 20 bp, Cannabis_sativa
aacacacaggattttttacg

SEQ ID NO: 1054 - DNA, 20 bp, Cannabis_sativa
caatgaaaatatgaacacac

SEQ ID NO: 1055 - DNA, 20 bp, Cannabis_sativa
tatgtgagattgtcactgtt

SEQ ID NO: 1056 - DNA, 20 bp, Cannabis_sativa
taacacaatctaatttattt

SEQ ID NO: 1057 - DNA, 20 bp, Cannabis_sativa
caatctaatttattttggat

SEQ ID NO: 1058 - DNA, 20 bp, Cannabis_sativa
tggcgacataaaacaatatt

SEQ ID NO: 1059 - DNA, 20 bp, Cannabis_sativa
ttggcgacataaaacaatat

SEQ ID NO: 1060 - DNA, 20 bp, Cannabis_sativa
tatttattttaatttgttgt

SEQ ID NO: 1061 - DNA, 20 bp, Cannabis_sativa
acaaaaaaataactaaccca

SEQ ID NO: 1062 - DNA, 20 bp, Cannabis_sativa
caaaaaaataactaacccaa

SEQ ID NO: 1063 - DNA, 20 bp, Cannabis_sativa
tataatttttttcttccctt

SEQ ID NO: 1064 - DNA, 20 bp, Cannabis_sativa
atataatttttttcttccct

SEQ ID NO: 1065 - DNA, 20 bp, Cannabis_sativa
tttattgttgaaaaatttat

SEQ ID NO: 1066 - DNA, 20 bp, Cannabis_sativa
taattataagacgtataaca

SEQ ID NO: 1067 - DNA, 20 bp, Cannabis_sativa
attcaattttaatgagatcg

SEQ ID NO: 1068 - DNA, 20 bp, Cannabis_sativa
ttcaattttaatgagatcga

SEQ ID NO: 1069 - DNA, 20 bp, Cannabis_sativa
tatttgttaatatatattga

SEQ ID NO: 1070 - DNA, 20 bp, Cannabis_sativa
taaaattttaaagttatgtg

SEQ ID NO: 1071 - DNA, 20 bp, Cannabis_sativa
ttttttaagattaattacta

SEQ ID NO: 1072 - DNA, 20 bp, Cannabis_sativa
aaaagaaattcaaattatta

SEQ ID NO: 1073 - DNA, 20 bp, Cannabis_sativa
agaaattcaaattattaagg

SEQ ID NO: 1074 - DNA, 20 bp, Cannabis_sativa
caaattattaaggtggcgtt

SEQ ID NO: 1075 - DNA, 20 bp, Cannabis_sativa
tttaatcaatgttttagatt

SEQ ID NO: 1076 - DNA, 20 bp, Cannabis_sativa
ttttagattaggaccagacc

SEQ ID NO: 1077 - DNA, 20 bp, Cannabis_sativa
agggaatttgggtccgggtc

SEQ ID NO: 1078 - DNA, 20 bp, Cannabis_sativa
tagggagggaatttgggtcc

SEQ ID NO: 1079 - DNA, 20 bp, Cannabis_sativa
gtagggagggaatttgggtc

SEQ ID NO: 1080 - DNA, 20 bp, Cannabis_sativa
gggccgtagggagggaattt

SEQ ID NO: 1081 - DNA, 20 bp, Cannabis_sativa
agggccgtagggagggaatt

SEQ ID NO: 1082 - DNA, 20 bp, Cannabis_sativa
ggacccaaattccctcccta

SEQ ID NO: 1083 - DNA, 20 bp, Cannabis_sativa
tagcgccagggccgtaggga

SEQ ID NO: 1084 - DNA, 20 bp, Cannabis_sativa
ttagcgccagggccgtaggg

SEQ ID NO: 1085 - DNA, 20 bp, Cannabis_sativa
ggtttagcgccagggccgta

SEQ ID NO: 1086 - DNA, 20 bp, Cannabis_sativa
gggtttagcgccagggccgt

SEQ ID NO: 1087 - DNA, 20 bp, Cannabis_sativa
aaattccctccctacggccc

SEQ ID NO: 1088 - DNA, 20 bp, Cannabis_sativa
aaaagtcgggtttagcgcca

SEQ ID NO: 1089 - DNA, 20 bp, Cannabis_sativa
caaaagtcgggtttagcgcc

SEQ ID NO: 1090 - DNA, 20 bp, Cannabis_sativa
tcgggttcaagtcaaaagtc

SEQ ID NO: 1091 - DNA, 20 bp, Cannabis_sativa
ttcgggttcaagtcaaaagt

SEQ ID NO: 1092 - DNA, 20 bp, Cannabis_sativa
tttaggtgcaagtcggattc

SEQ ID NO: 1093 - DNA, 20 bp, Cannabis_sativa
atttaggtgcaagtcggatt

SEQ ID NO: 1094 - DNA, 20 bp, Cannabis_sativa
aaaataatttaggtgcaagt

SEQ ID NO: 1095 - DNA, 20 bp, Cannabis_sativa
ttaagctttcaaaataattt

SEQ ID NO: 1096 - DNA, 20 bp, Cannabis_sativa
aagataattttaccacttac

SEQ ID NO: 1097 - DNA, 20 bp, Cannabis_sativa
taatataatcatcctgtaag

SEQ ID NO: 1098 - DNA, 20 bp, Cannabis_sativa
ttgattgtattgattattat

SEQ ID NO: 1099 - DNA, 20 bp, Cannabis_sativa
tagctacaattattaatgag

SEQ ID NO: 1100 - DNA, 20 bp, Cannabis_sativa
taaaattgaagtgtgttttt

SEQ ID NO: 1101 - DNA, 20 bp, Cannabis_sativa
atatttcaaacttatagctt

SEQ ID NO: 1102 - DNA, 20 bp, Cannabis_sativa
accaattagaaatgggcacg

SEQ ID NO: 1103 - DNA, 20 bp, Cannabis_sativa
aacttagaccaattagaaat

SEQ ID NO: 1104 - DNA, 20 bp, Cannabis_sativa
taacttagaccaattagaaa

SEQ ID NO: 1105 - DNA, 20 bp, Cannabis_sativa
accacgtgcccatttctaat

SEQ ID NO: 1106 - DNA, 20 bp, Cannabis_sativa
tccacgtgtcattttcttct

SEQ ID NO: 1107 - DNA, 20 bp, Cannabis_sativa
accaagaagaaaatgacacg

SEQ ID NO: 1108 - DNA, 20 bp, Cannabis_sativa
gataatgacttaatatttaa

SEQ ID NO: 1109 - DNA, 20 bp, Cannabis_sativa
atgacttaatatttaatggt

SEQ ID NO: 1110 - DNA, 20 bp, Cannabis_sativa
taatattttgggaactctgt

SEQ ID NO: 1111 - DNA, 20 bp, Cannabis_sativa
taatacctaagtaatatttt

SEQ ID NO: 1112 - DNA, 20 bp, Cannabis_sativa
ataatacctaagtaatattt

SEQ ID NO: 1113 - DNA, 20 bp, Cannabis_sativa
gagttcccaaaatattactt

SEQ ID NO: 1114 - DNA, 20 bp, Cannabis_sativa
aaatacctacgttttatttt

SEQ ID NO: 1115 - DNA, 20 bp, Cannabis_sativa
tagcgccaaaaataaaacgt

SEQ ID NO: 1116 - DNA, 20 bp, Cannabis_sativa
cgtaggtatttatttgcaac

SEQ ID NO: 1117 - DNA, 20 bp, Cannabis_sativa
cacaaaacatctaaaaaaaa

SEQ ID NO: 1118 - DNA, 20 bp, Cannabis_sativa
cattttttttagatgttttg

SEQ ID NO: 1119 - DNA, 20 bp, Cannabis_sativa
acgccaactcatatacaaaa

SEQ ID NO: 1120 - DNA, 20 bp, Cannabis_sativa
ggaccattttgtatatgagt

SEQ ID NO: 1121 - DNA, 20 bp, Cannabis_sativa
tgttgaatctctagctcttt

SEQ ID NO: 1122 - DNA, 20 bp, Cannabis_sativa
gctttattgtctaaatttct

SEQ ID NO: 1123 - DNA, 20 bp, Cannabis_sativa
ctttattgtctaaatttctt

SEQ ID NO: 1124 - DNA, 20 bp, Cannabis_sativa
tttattgtctaaatttcttg

SEQ ID NO: 1125 - DNA, 20 bp, Cannabis_sativa
ctaaagtaagcgagcaacat

SEQ ID NO: 1126 - DNA, 20 bp, Cannabis_sativa
tctaaagtaagcgagcaaca

SEQ ID NO: 1127 - DNA, 20 bp, Cannabis_sativa
gtttgacaaaacatgctatt

SEQ ID NO: 1128 - DNA, 20 bp, Cannabis_sativa
caatgagctatcctagttca

SEQ ID NO: 1129 - DNA, 20 bp, Cannabis_sativa
cacagaaatctccttgaact

SEQ ID NO: 1130 - DNA, 20 bp, Cannabis_sativa
ggagatttctgtgctatttg

SEQ ID NO: 1131 - DNA, 20 bp, Cannabis_sativa
cgcttgcaaatatttatcta

SEQ ID NO: 1132 - DNA, 20 bp, Cannabis_sativa
atagcaaattttttttccat

SEQ ID NO: 1133 - DNA, 20 bp, Cannabis_sativa
tgatttttttaaatttccta

SEQ ID NO: 1134 - DNA, 20 bp, Cannabis_sativa
ttattaaaaataaatgtaca

SEQ ID NO: 1135 - DNA, 20 bp, Cannabis_sativa
atgtacatttatttttaata

SEQ ID NO: 1136 - DNA, 20 bp, Cannabis_sativa
tgtacatttatttttaataa

SEQ ID NO: 1137 - DNA, 20 bp, Cannabis_sativa
ataagggctgcacctaacaa

SEQ ID NO: 1138 - DNA, 20 bp, Cannabis_sativa
caaaattaggcacctttgtt

SEQ ID NO: 1139 - DNA, 20 bp, Cannabis_sativa
ctaacaaaggtgcctaattt

SEQ ID NO: 1140 - DNA, 20 bp, Cannabis_sativa
atttcttttttaccaaaatt

SEQ ID NO: 1141 - DNA, 20 bp, Cannabis_sativa
ttttggtaaaaaagaaatta

SEQ ID NO: 1142 - DNA, 20 bp, Cannabis_sativa
agctttatagattggagtgg

SEQ ID NO: 1143 - DNA, 20 bp, Cannabis_sativa
tagctttatagattggagtg

SEQ ID NO: 1144 - DNA, 20 bp, Cannabis_sativa
atagctttatagattggagt

SEQ ID NO: 1145 - DNA, 20 bp, Cannabis_sativa
tatagctttatagattggag

SEQ ID NO: 1146 - DNA, 20 bp, Cannabis_sativa
ctatttatagctttatagat

SEQ ID NO: 1147 - DNA, 20 bp, Cannabis_sativa
caatctataaagctataaat

SEQ ID NO: 1148 - DNA, 20 bp, Cannabis_sativa
atgttggaaattactatgaa

SEQ ID NO: 1149 - DNA, 20 bp, Cannabis_sativa
tttttctttagtcgtaatgt

SEQ ID NO: 1150 - DNA, 2940 bp, Cannabis sativa
ttttcttcgtaaagtttttgcctttttggtatgtacaatatcatataagtctcccatatttatataaagttattatatgcttgcattgacagagtagtattatatgaatatatttaatatatatatctatgaaatttcaacgcgtataaagttcagattattaccaaattatttgatctagttaaatgcttactcggatgatgatactactagttcaacaattataaattaatactaacaagtgtgtaatttgttttttaaaaaaggatttatctgtatatcttttgaattaattctcgtattatcttattttaataatataagctttacgtaggcactttattcttatgtaaaaagggtaccaaaattgaagcggacaaagccgacaagaaaatattgactttttttttttttttttgaaatcaagaaaatattgactatatatacatataaatgaacatattattgtgtatatataatatatagtagtcaaataaaaatttggtaatataatttattcgttaaactcaattatggtggattggtagaagctttatatatatttattatatatatttcgagaaatatatattatatattaaaattagacggtttgaacacattaagatcacgcttctataatatattaaaatttgttaaaaaaatagctggatttattttttagatatctcttctgttcacaaagtgatattaattcaacaactaataactataaaacatttatttagaaagacacgtaatcgaaacatcaacgaagctgaaattcttttaaattgttcaattatgcaaatatatatgtttacatatattaatatacttgtcctatatatatatatattattatgtatataaatatatttaaatacatgtgatcaaacattgttataaatactagaaatttggaactgggtcagccgtatttttattattttttaattgaacaagctacgtacaagttacatgctttttataaattatattttttattctattttaaacgaaaattcaatatcaatgaattattctagttgaactctaaaaacgaaatgctgactttttctttttttttttctttttctggtagcgtgattcttattaattcgtccaattcctaatggttatcccggtttgatcaaagttaatttgtattttatttactcttttttttaaatctctgtaatgtattattcatttttaatagaaggcacaataataattacaccacttacattgtattatttgaaatatatgatactattaaattagttatgtggatttatctcaccatgactcagtatttttatttaattatttcatattcaacattctttttagaattctcataatatggtaactattttttgttcactaatgactcattgaatctagggtcgattttaaagataaacaaagtaggcccacgcctagcacccaactctattgaggccgtgaaaaaataaaaatcaaattatttatatatattagttaataatttatatatctatatatataaaagaaaaaaaaaaaaaaacggtgacataacactttaaaatatctttaattatggttagttgttcttaattctcttattttttttaaaactttttttttttttttttttttttttgaaaataaacttttattagtttcataaatgttaattcacataatatatatcggaagatgattttgtcccgtgattgtactttcatggaatgtattccgtgatgtacgacagccgttagatgagggagttatttgatctgggggctgagattgatctaggggtaattagggttaaaaaatagtaacgtaccctgacactaatgtacccctgagacccatctaataccacggaatacattccgtgaaagtacatcacgggaaaaaatcgtatcccaatatatatatatatctatttattttttttcttaattctttcatttttaatattttattaagtttattaacattttttttttaaaaaaatattttttgttagtgattttttcattagataccattagaagagtttcaaaaactctataaatatagtcaaagtcaaatattataatttaggaaccctataatttattaagagaatttcataatatgtttttttttagttgagagtttttaaacttattcaatcattaataaaggttaacaatgatcatggggccttaactacaacattaggcacattttcaatggaaacattccaaaagccacttttattatggggatatgaatactacataatttatatttttatattaaaattgttcatatgtcttgtcaagtattttttttttattttcttaatcacatttagattttcaaattgatctacatttttatatgtgtatgtgtgatttctattattgagtaactagagaactaagtcgcgcttcgcgcagtgttgttcaatatttgtaatctttttatattgttatttttttagtcttaattaagtggtataatataaaatatgacattgcgatgttagaattataaaaagaaaaaaaaagtaacatgatagaatgatagaattttagaaataaaattttgtattttacaaaaatatgtagaaagatgtacttatttttgacaaataaagaaaattattgaaataacatagagattgtatgtgtattaaaattaaataggagaaaagagagaaccaaattatttaaaacccggtactctttaaccctcctaccacccactgaaaaactatattacactaagactttaaccatgagaataaaaggcaaattaagagtgggagaaaaaataactacaaaaaaaaagtgaatttcaagaggtgctaaatattattttgtgttcaaaaaattataagatatatcataaaaaaaaagtccaccattttcaatcttggtgataagaaaaacattt

SEQ ID NO: 1151 - DNA, 3001 bp, Cannabis sativa
ttatgcatgtcttatatagtaccgttaatttggcgatacctatactccgagtctctaactattctttgagttctacaaatttagttttaactattttttgatataaatgatacattaatggttttaattatgctttgatattaagttttcttcattacacatatttagaatgaaggttaagatagtcatttgcttatatttatttactttaatttagtaataagatttatcaaagaaaattttatttgcatcccaggaagtacaccctcatattaaaaaaaaaaaataaacttaaaataatttttaaaatctttcatactattttttaattttttttaaatattattttgggttaattttaacacttattttaattttattttcataatttttttctaataaaatttcacatgaattttggctttttctcaaaataaaaaaaaaaaaattcacagaatttttttttaattttttttgttaaaatatttaaaatataatttatttttatttgataggtatattttttaggaatatgaattgaaagaaaaaaataaaaaaaaactaatacaattgaagacataattaattttgaaaataaatgaaattcctattttcatcaaattattttaaaattaattaatttaaaaaaaaaaactaaatttagtttaacgaaatttattcaagataaataatatttttatttttcttatttaactaaataaagaaattaacgaagatagccatatgagttggatgtttcttgttatccttatgacgcgtaagttatatttataagtttattttattagttactaatcagttatatatttaatttaaatctattattattttatttactttcgtaattatctcaaaacttaatttatattaccttcatacttatctcaaaacctaatttattcagtttcacgttttatttttcctttcaattgtaattgatttatgaaattaatttgcttttatttaatttaaattttattgatatcatatagtttcatacatatatattttccttttatgttcttttattctttatagctttcatgttttagatacacttgatcttaagtgttttgaatatttaaattttttatttttactattgatatattattattttaacatacatctttataaaattaatatatatattatatatatatattgagtcaacgattaattttatttcatttctattatttttactatatttatatatattagacaatatacttcatagtgtataattaatatttttattgtttttactcttttaattttagaataacctttattttttacttgtattaattttcaagatacggttgtatatattatttaaggaaattactacatatattatttttttaacttttttttttaaatttacggtttgggtttctaaagtggttgcagcgctagttgcaataggggattctgtacgattttttgttgcaatttaggttgcagcgctagttgtcataagagtttctatctagaattccgtaaaaatgcaaaaaaaaaaaaaaaaaaaaaaaaaactgttttaaagtgtaaaaataaaaaagcccctattatttatttattatatataaactttatgttaattttatattttatttatttatagtttataacaaattacacatatttctagagactttgttggtgattgactttgtgtcatatatggtggcaaatatgtaccttgatggagctagggttgattatgctatggttggtcggtggctagttgtaccatgtttggtgggtattttttttttatttttttgttttataatttcaaaaacaatataaattttttaaattcaaaatgactatctaaaaaagaatttaagtttgatgaccttgttagttttttgtacaattttcttatagttttgatgcctttttagggcatttgtaccatattttatggcttgctcttaattgtttagcgtaaatttgaggtatggccaatgattaagagaagggttttgttttgtagtcttgggttcgacaaacgacttttcaaatatgatatataagtatttcaagtcttgatattataatatagattattttacaatctttattcacattacctatttttttttattaaattgtgtagtaatagagttttatttttaattattttgtaagtaatttttatttaggtgatgtaatagagctattttttttttttttgtaggtcggttttgttaagggtctacgttatactgtatttgttgataagtttatatttttacatataatttatttgagagtttttatcttcttactattgataatttcttttattagtttttaatataattgttaggggataattcattatttttatcttatttttttgtagatattttgtggaccatttgttatatgagttggtgtgtagtttttcagtgttacaagttgaatttctagctcttttagtgtgttgtttgaagatgtgtagctttattttctaaatttttagtggtagttttgtcccatgttgcttgcttactttagcaattcatagtttgacaaagcatgctattggttagacgatgatctatcctagttcgaggaagtttctgcaatatttgtggatatttacatcgtaaattcatgagtgattcttatcacttcgtaaaaaaaagatagaaagatacataaataaatatttactagtcctttctttccatagtcaacttcaatatggcatcttagcttctttccatcaacaaatgatgctcaactacttaatgtacaatttatatttatttttaataagggatgcacctaacaatacaatgatgcctaatttttgtgaattttttttaccacatgacttaatgatatcaaattatgaaatatttagttaatttatttgcccctgctccaatatataaagctataaataggatagttcttaatccatagtaattcaaaattcattagaactaaagaaaaa

SEQ ID NO: 1152 - DNA, 3001 bp, Cannabis sativa
aatcgattccataggggtttcctataaattttttaaaatttttattaaaaatctaaatttatttattttaaattaaattaaaattaaattaaaattaaattaaattaaaaagttaaataaatacaaatttacattgctctaagtgtttgtatattaggtatttttttattaatattttatattttataatatgtgtttgtatttttccagtattttagtatcatttttataatttttaagttatgttttatataataattagtatattgaataataaataatgtgtaatattttaatttttatgtaatttaattttttcatacatttaaattttataatatgagtttgtattttatagtatattagtattatttttataattttttaagttatcttttgtataataattagtttattgaataataaataatgtgtaatattttaatttttatgtaatttaatatttttatacatttaacttttataatatgggtttgtatttttctagtatattattattatttttataattttttaagttatgttttgtataataattagtatattgaataaaaataatgtgtaatattttaattttgatgtaatttaatatctttatacatttactttttttaaaaaaaaacatttactctaagcaaatgtttacactaatattatcccaatagaccatttttgtaatttttttttggcttactatatctatcattaacactcaacaatatctatcctacgtattaatattattctaaaattactctaagtatttaaggattatctaaatatatataaaaaatatcttataaaagtaagttgaaaaaacataccttatacaaaaaattatgtgcttgacttccgataaagtcaatttcgataaaatcctaacaaccaagtttaagagaaactaaaataaaatattatcctaattctaccgaaatttcggcagcataacggctataatagaaagtttccaaaaaattcaaacctattaataacaacaaaacacaattcaaaataatattaaataatcataacaacatttagcaatataaaagtttaccacccatccgactacagtacatgtattcacagaacctacttcactacagatgaatatttaagatattcaaactcatctaacatgtatattgtttcacccatcgatccgacatagtacaatatattgtagaacctacttcactacaaatgaatatttaagattttcaaactctaagcattcataattaatgtataataaaagttagtaaatgtatagctcgagctacctagaaaccgatcaacacaaaataagttggatccttgcaaattgacctcacaaccttcaacataaagaaatactacaaaattaccaatttttttatttagctacttactagttatataatttatgagtaactagttactaacttgtttctaatgagtaagtaattagtaataaaaatatatgaacatgaatgaaaattagtagcttgttaagtagctgtaactaactatttactacctactaaagaaataaaacacatgaataaaattaaaaaaaaaaattgatacaaaatatacttaatttctaaacttttgtttctaaactattttttagaaattaatttctaaacttttattttaatactttcctatctcattctcattcacaatatttaacaaaactattagtttaaaatactcaatatatacatatacaatatataaaatactctcctatattacatttctcattcacaatatatatacacacaatacatatttgatacaacacaaaatacatatatatactcttctatatcacatttcccattcacaatagatatacacataatacatattcattacaacataatacatattcaataatatagaaaaccctaaaaaaatttataaaataacaaataaagcaatgtacattactaaaattagtagaaattagatttataccttaataggcgttgagatacaatgttttctccactaaaaacggtggccggaggtataatcaccactacctttactttttgtttaccccaaaaaatgaggaaaaatgtaatcttttcaggtatatagttttaggtatgaaaaatagaagatttagaaataaacatgagctaaaatggtgaaaaaatagtgaaatggaggtgatttttcgtggtggttggtggagagggtttggggtttctttggtttgggggtttctgggttggatggtcgaatgaggaagatgaagggctgggctagggttataaaaccttttggcgtcggttatcttctaactattgataatttcttttattagtttttaatataattgtaaggggataattcattatttttatcttatttttttgtagatattttgtggaccatttgttatatgagttggtgtgtagtttttcagtgttacaagttgaatctctagctcttttattgtgttgtttaaggatgcgtagctttattttctaaatttttagtaatttttttgtcccatgttgcttgcttactttagcaattcatagtttgacaaagcataacattcagttagacgatgatctatcctaattcgaggaggtttctgcaatatttttgtgaatatttgcatcgtaaattcatgagtgattcttatcacttcttaaaaaaaaagatataaacatccatagataaatatttgctagtcctttctttccatagtcaacttcaatatggcatcttaacttctttccatcaacaaatgatgctcaactacttaatgtacaatttatatttatttttagtaagggatgcacctaacaatgatgcctaattttggtgaatttttttttaccacatgtgacttaatgatatcaaattaggaaatatttagttaatttatttgcccctgctccattataaaaagctataaataggatagttcttaattcatagtaattcaaaaattattagaactaaagaaaaa

SEQ ID NO: 1153 - DNA, 2919 bp, Cannabis sativa
acttagcaaattcagctttcgcatcgtgagtcaccaaagaaatccctataactttgggatcaggccatagagatatggcaagattcgagaaactctaataacaagaattaagattgtcccaaataaagtgataaacttttaaacaaagaagcaacaaggcgtacattgtcgacctcagaaggataaggaaaaaggctccagctcgctatcacggtcactggaaccaagctcctgtacggacatcctcagacacaataattaaagatgtattcaaatacacgatcattttgatccaataaatgcgggaggatcacatctatatgattctcaaataataaccgcatttacacaacgtgatatgtaatcaaatcccatgattttaggggatgatcgttgtaaaaagattacagtcaactttgtaattaactgcctactatgtgattataaatagtggcaagtaagatcaaaaaagtggacgaaaaaagcatacaaaaaaggctctgagaaaactgtcaaaaatctgaataagattgactcgtaaactatgcataattttaactgtaaaaccacgtaaaaaatcctgtgtgttcatattttcattgtacatcttttattttttctatgtgagattgtcactgttaggcttaaatttagttaacgaaaatttgcattaacacaatctaatttattttggattggttagttttaaatattaaattattgaatatttttttcacataactaataataaaataaaaagaacccaatattgttttatgtcgccaacaacaaattaaaataaataaaacaaaaaaataactaacccaagggaagaaaaaaattatatatataaagaaatattaacttttgttttattgtatgtacttgcataataaataaattgtaattatgtgcacttacacatccaataaatttttcaacaataaactattatattttgatgatattaatgccatgttatacgtcttataattattcaaagttatcatagagtattcaattttaatgagatcgagggtaatcgagatcatttaaatattttttactgcaaaacttttcatctatttattatgcatttttttatatgacaaaatttaatattcaattttttatttgacaaattttttcatttgattactattattattgcatgtgatattttattgttcactattacactttatgtaatacatccatcaatatatattaacaaataaaaacttaaaactttccacacataactttaaaattttataatataagaattgttccatagtaattaatcttaaaaaaaaatattatttagaaaaaagaaataaatttaaattattaattaatttgaaagcttaaattaaatataaataaaataataaaaattattatgttttttcaaatgattgaacttaagtcattgtacttgatgaaaaaaaaagaaattcaaattattaaggtggcgtttggtaacacttttttaatcagttttctgtttttaaaagtagaaaagtaaaaatatttttcaaaaacatgttctataaaactgtttttactttttaattttataattagaaatcaaaattttaaaaacaaaagaatcactttcaatatttttttaaacagtttttttttaatcaatgttttagattaggaccagacccggacccaaattccctccctacggccctggcgctaaacccgacttttgacttgaacccgaatccgacttgcacctaaattattttgaaagcttaaattaatacaaataaaataaaaaaaaaattatcatgttttttcgaatgatgaacttaagtcattgtacttgatgtaagataattttaccacttacaggatgattatattattgattgtattgattattataggattaaataacttgaatatactatacatttctagctacaattattaatgagtggaaaataaaattgaagtgtgttttttggcgataataatacctaagctataagtttgaaatatttataagtacaactgttaagtgttaagtaaattaccacgtgcccatttctaattggtctaagttattaaatttttattttttaaaaaaattaaattaaatataccaagaagaaaatgacacgtggataatgacttaatatttaatggtcggtacctacagagttcccaaaatattacttaggtattattagcgccaaaaataaaacgtaggtatttatttgcaactggaagttaaatttagatatttatgtcgcaaattactcttttttatccattttttttagatgttttgtggaccattttgtatatgagttggcgtgtagttttttagtgttacgtgttgaatctctagctcttttggtgtgaagtttgaagacgttatagctttattgtctaaatttcttggggtagttttgtcccatgttgctcgcttactttagaagttcaaagtttgacaaaacatgctattcggttatacaatgagctatcctagttcaaggagatttctgtgctatttgtggatgtctgcattgtaaattcatgagtgattcttataactttattaaaaaaagacaaatagatccatagataaatatttgcaagcgctttcttgtcatagcaaattttttttccataggaaatttaaaaaaatcatcttaactttttttcatcaataaatgatgctcaattattcaatgcaccatgtacatttatttttaataagggctgcacctaacaaaggtgcctaattttggtaaaaaagaaattacggcatgtgaatatttaatgagcatcaaattacaaaatatttagttaatttctttacccccactccaatctataaagctataaataggataattttccattcatagtaatttccaacattacgactaaagaaaa